(19) United States
(12) Patent Application Publication
(10) Pub. No.: US 2021/0009575 A1
(43) Pub. Date: Jan. 14, 2021

(54) HYDROXYISOXAZOLINES AND DERIVATIVES THEREOF

(71) Applicants: Bayer Aktiengesellschaft, Leverkusen (DE); Bayer CropScience Aktiengesellschaft, IVIonheim am Rhein (DE)

(72) Inventors: Anne-Sophie REBSTOCK, Champagne au Mont d'or (FR); Sebastien NAUD, Collonges au Mont d'Or (FR); Stephane BRUNET, Saint Andre De Corcy (FR); Mathieu GOURGUES, Lyon (FR); Francois Michel VILLALBA, Albigny-sur-Saone (FR); Aurelia VERNAY, Saint Forgeux (FR); Birgit KUHN, Kelkheim (DE); Andreas GOERTZ, Dormagen (DE); Philippe DESBORDES, Lyon (FR); Jeremy DUFOUR, Lyon (FR); Emmanuelle HILT, Dizimieu (FR); Sophie DUCERF, Chasselay (FR)

(21) Appl. No.: 16/955,576

(22) PCT Filed: Dec. 21, 2019
(86) PCT No.: PCT/EP2018/086723

§ 371 Date: Jun. 18, 2020

(30) Foreign Application Priority Data
Dec. 22, 2017 (EP) 17210472.1

(51) Int. Cl. C07D 413/14; A01N 43/80; C07D 413/06; C07D 419/04; C07D 419/14

(52) U.S. Cl. CPC C07D 413/14; A01N 43/80; C07D 419/14; C07D 419/04; C07D 413/06

(57) ABSTRACT

The present disclosure relates to the use of hydroxyisoxazolines and derivatives thereof as fungicide. It also relates to new hydroxyisoxazolines derivatives, their use as fungicide and compositions comprising thereof.

### TECHNICAL FIELD

The present invention relates to the use of hydroxyisoxalines and derivatives thereof as fungicide. It also relates to new hydroxyisoxazolines derivatives, their use as fungicide and compositions comprising thereof.

### BACKGROUND

Isoxazole derivatives are known to be useful as crop protection agents to combat or prevent microorganisms' infestations. For instance, WO2015/129773 discloses isoxazole derivatives that may be used as fungicides. WO2006/031631 discloses substituted isoxazoles that may be used for the control of microbial pests, particularly fungal pests, on plants. However, WO2015/129773 and WO2006/031631 do not disclose hydroxyisoxazolines.

Numerous fungicidal agents have been developed until now. However, the need remains for the development of new fungicidal compounds as such, so as to provide compounds being effective against a broad spectrum of fungi, having lower toxicity, higher selectivity, being used at lower dosage rate to reduce or avoid unfavorable environmental or toxicological effects whilst still allowing effective pest control. It may also be desired to have new compounds to prevent the emergence of fungicides resistances.

The present invention provides new fungicidal compounds which have advantages over known compounds and compositions in at least some of these aspects.

### SUMMARY

The present invention relates compounds of the formula (I′):

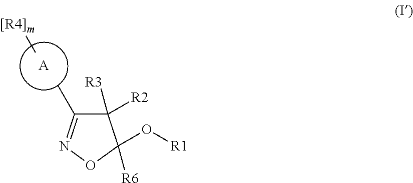

wherein R1, R2, R3, R4, A, m and R6 are as recited herein as well as their salts, N-oxides and solvates.

The present invention relates to a composition comprising at least one compound of formula (I′) as defined herein and at least one agriculturally suitable auxiliary.

The present invention also relates to the use of a compound of formula (I) as defined herein or a composition as defined herein for controlling phytopathogenic fungi.

The present invention relates to a method for controlling phytopathogenic fungi which comprises the step of applying at least one compound of formula (I) as defined herein or a composition as defined herein to the plants, plant parts, seeds, fruits or to the soil in which the plants grow.

### Definitions

The term “alkyl” as used herein in the context of alkyl or alkylsulfonyl, alkylsulfinyl, alkylthio, alkylamino, for example, is to be understood as preferably meaning branched and unbranched alkyl, meaning e.g. methyl, ethyl, n-propyl, iso-propyl, n-butyl, iso-butyl, tert-butyl, sec-butyl, pentyl, iso-pentyl, hexyl, heptyl, octyl, nonyl and decyl and the isomers thereof.

The term “haloalkyl” as used herein is to be understood as preferably meaning branched and unbranched alkyl, as defined supra, in which one or more of the hydrogen substituents is replaced in the same way or differently with halogen. Particularly preferably, said haloalkyl is, e.g. chloromethyl, fluoropropyl, fluoromethyl, difluoromethyl, trichloromethyl, 2,2,2-trifluoroethyl, pentafluoroethyl, bromobutyl, trifluoromethyl, iodoethyl, and isomers thereof.

The term “alkoxy” as used herein is to be understood as preferably meaning branched and unbranched alkoxy, meaning e.g. methoxy, ethoxy, propyloxy, iso-propyloxy, butyloxy, iso-butyloxy, tert-butyloxy, sec-butyloxy, pentyloxy, iso-pentyloxy, hexyloxy, heptyloxy, octyloxy, nonyloxy, decyloxy, undecyloxy and dodecyloxy and the isomers thereof.

The term “haloalkoxy” as used herein is to be understood as preferably meaning branched and unbranched alkoxy, as defined supra, in which one or more of the hydrogen substituents is replaced in the same way or differently with halogen, e.g. chloromethoxy, fluoromethoxy, pentafluoroethoxy, fluoropropyloxy, difluoromethyloxy, trichloromethoxy, 2,2,2-trifluoroethoxy, bromobutyloxy, trifluoromethoxy, iodoethoxy, and isomers thereof.

The term “carbocyclyl” as used herein refers to a non-aromatic mono- or polycyclic (fused, spiro or bridged) carbon containing ring, which may be saturated or unsaturated, having 3 to 10 ring carbon atoms. Examples of carbocyclyl include cycloalkyl and cycloalkenyl groups. Examples of saturated cycloalkyl include but are not limited to cyclopropyl, cyclobutyl, cyclopentyl, cyclohexyl, cycloheptyl, cyclooctyl, cyclononyl and cyclodecyl group. Examples of unsaturated carbocyclyl group include but are not limited to cyclopropenyl, cyclobutenyl, cyclopentenyl, cyclohexenyl, cycloheptenyl, cyclooctenyl, cyclononenyl, or cyclodecenyl group, wherein the linkage of said cyclolaklyl group to the rest of the molecule can be provided to the double or single bond.

The term “heterocyclyl” as used herein refers to three- to fifteen, or three to twelve, or three to ten-membered, preferably three- to nine-membered, saturated or partially unsaturated heterocycles (including mono-, bi- or tricyclic heterocycles) containing one to four heteroatoms independently selected from the group of oxygen, nitrogen and sulfur. If the ring contains more than one oxygen atom, they are not directly adjacent. A polycyclic heterocyclyl may contain fused, spiro or bridged ring junctions. Examples of heterocyclyl group include but are not limited to oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl, quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl. This definition also applies to heterocyclyl as part of a composite substituent, for example heterocyclylalkyl etc., unless defined elsewhere.

The term “halogen” or “Hal” as used herein is to be understood as meaning fluorine, chlorine, bromine or iodine.

The term “alkenyl” as used herein is to be understood as preferably meaning branched and unbranched alkenyl, e.g. a vinyl, propen-1-yl, propen-2-yl, but-1-en-1-yl, but-1-en-2-yl, but-2-en-1-yl, but-2-en-2-yl, but-1-en-3-yl, 2-methyl-prop-2-en-1-yl, or 2-methyl-prop-1-en-1-yl group.

The term “alkynyl” as used herein is to be understood as preferably meaning branched and unbranched alkynyl, e.g. an ethynyl, prop-1-yn-1-yl, but-1-yn-1-yl, but-2-yn-1-yl, or but-3-yn-1-yl group.

The term “aryl” as used herein refers to an aromatic, hydrocarbon, ring system, comprising from 6 to 15 carbon atoms, or from 6 to 12 carbon atoms, preferably from 6 to 10 carbon atoms. The ring system may be monocyclic or fused polycyclic (e.g. bicyclic or tricyclic) aromatic ring system. Examples of aryl include but are not limited to phenyl, azulenyl, naphthyl, biphenyl and fluorenyl. It is further understood that when said aryl group is substituted with one or more substituents, said substituent(s) may be at any positions on said aryl ring(s). Particularly, in the case of aryl being a phenyl group, said substituent(s) may occupy one or both ortho positions, one or both meta positions, or the para position, or any combination of these positions. This definition also applies to aryl as part of a composite substituent (e.g. aryloxy).

The term “heteroaryl” as used herein refers to an aromatic ring system containing from 5 to 15 member atoms, or from 5 to 12 member atoms, of which carbons and one or more heteroatoms which may be identical or different selected from O, N and S. If the ring contains more than one oxygen atom, they are not directly adjacent. Heteroaryl may be monocyclic or polycyclic (e.g. bicyclic or tricyclic). A monocyclic heteroaryl may have 1 to 4 heteroatoms in the ring, while a polycyclic heteroaryl ring may have 1 to 10 heteroatoms. Bicyclic heteroaryl rings may contain from 8 to 15, or from 8 to 12 member atoms (carbon and heteroatoms). Monocyclic heteroaryl may contain from 5 to 8 member atoms. Examples of heteroaryl include but are not limited to thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, e.g., benzofuranyl, benzothienyl, benzoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc. It is further understood that in the case in which said heteroaryl group is substituted with one or more substituents, said substituent(s) may occupy any one or more positions on said heteroaryl ring(s). Particularly, in the case of heteroaryl being a pyridyl group, for example, said substituent(s) may occupy any one or more of positions 2, 3, 4, 5, and/or 6 with respect to the nitrogen atom in the pyridine ring. This definition also applies to heteroaryl as part of a composite substituent (e.g. heteroaryloxy).

As used herein, the term “C₁-C₆”, e.g. in the context of the definition of “C₁-C₆-alkyl”, or “C₁-C₆-alkoxy”, is to be understood as meaning a group having a finite number of carbon atoms of 1 to 6, i.e. 1, 2, 3, 4, 5, or 6 carbon atoms.

The term “leaving group” as used herein is to be understood as meaning a group which is displaced from a compound in a substitution or an elimination reaction, for example a halogen atom, a trifluoromethanesulfonate (“triflate”) group, alkoxy, methanesulfonate, p-toluenesulfonate, etc.

The term “isomers” as used herein is to be understood as meaning chemical compounds with the same number and types of atoms as another chemical species. There are two main classes of isomers, constitutional isomers and stereoisomers.

The term “constitutional isomers” as used herein is to be understood as meaning chemical compounds with the same number and types of atoms, but they are connected in differing sequences. There are functional isomers, structural isomers, tautomers or valence isomers.

In stereoisomers, the atoms are connected sequentially in the same way, such that condensed formulae for two isomeric molecules are identical. The isomers differ, however, in the way the atoms are arranged in space. There are two major sub-classes of stereoisomers; conformational isomers, which interconvert through rotations around single bonds, and configurational isomers, which are not readily interconvertable.

Configurational isomers are, in turn, comprised of enantiomers and diastereomers. Enantiomers are stereoisomers which are related to each other as mirror images. Enantiomers can contain any number of stereogenic centers, as long as each center is the exact mirror image of the corresponding center in the other molecule. If one or more of these centers differs in configuration, the two molecules are no longer mirror images. Stereoisomers which are not enantiomers are called diastereomers. Diastereomers which still have a different constitution, are another sub-class of diastereomers, the best known of which are simple cis-trans isomers. Clearly, when it is possible for a compound of the present invention to exist in such isomeric forms, the present invention covers single isomers, and any mixture, e.g. racemic mixtures, of such isomers, whether they be isolated or not.

In order to limit different types of isomers from each other reference is made to IUPAC Rules Section E (Pure Appl Chem 45, 11-30, 1976).

### DETAILED DESCRIPTION

In some embodiments (referred herein as embodiment 1), the present invention provides for the use of a compound of the formula (I) for controlling phytopathogenic fungi:

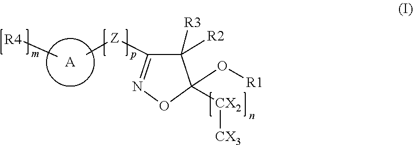

in which
R1 represents a substituent selected from the group consisting of hydrogen, sulfinyl, sulfonyl, C₁-C₆-alkyl, C₁-C₆-haloalkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3 to 10-membered-heterocyclyl, hydroxy-C₁-C₆-alkyl, aryl, heteroaryl, nitro, —C(═O)R^{a}, —C(═O)OR^{a}, —C(═O)N(R^{a})₂, —C(═S)N(R^{a})₂, —S(═O)₂R^{a}, —S(═O)₂N(R^{a})₂ and —P(═O)(OR^{a})₂,
   wherein said C₁-C₆-alkyl, C₁-C₆-haloalkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3 to 10-membered-heterocyclyl, hydroxy-C₁-C₆-alkyl, aryl, heteroaryl substituent is itself optionally substituted, one or more times, in the same way or differently, with R5;
R2 and R3 independently of each other, represent a substituent selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{a}, —C(═O)OR^{a}, —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —OC(═O)N(R^{a})₂, —NR^{a}S(═O)₂R^{a}, —S(═O)₂R^{a}, —S(═O)₂N(R^{a})₂, —P(═O)(OR^{a})₂, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═S)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂, —C₁-C₆-alkyl-P(═O)(OR^{a})₂ substituent is itself optionally substituted, one or more times, in the same way or differently, with R5;
   or,
   together, form a C₃-C₁₀-carbocyclyl or 3- to 10-membered-heterocyclyl;
A represents aryl, heteroaryl, C₃-C₁₀-carbocyclyl or 3- to 15-membered-heterocyclyl group, preferably aryl or heteroaryl group;
X represents, independently of each other, a substituent selected from the group consisting of hydrogen, fluorine, chlorine, bromine, and iodine atom, wherein at least one X substituent is a fluorine atom;
Z represents a C₂-C₆-alkenyl group, itself being optionally substituted, one or more times, in the same way or differently, with R5;
R4 represents independently a substituent selected from the group consisting of halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —SF₅, —C(═O)R^{a}, —C(═O)OR^{a}, —C(═NR^{a})R^{a}, —N(R^{a})₂, —NR^{a}C(═NR^{a})R^{a}, —N═CR^{a}—N(R^{a})₂, —OC(═O)R^{a}, —S(═O)₂R^{a}, —C(═NR^{a})N(R^{a})₂, —P(═O)(OR^{a})₂, —C₁-C₆-alkyl-N(R^{a})₂, —C₁-C₆-alkyl-C(═O)R^{a}, —C₁-C₆-alkyl-O—C(═O)R^{a}, —C₁-C₆-alkyl-C(═O)OR^{a}, —C₁-C₆-alkyl-S(═O)₂R^{a} and —C₁-C₆-alkyl-P(═O)(OR^{a})₂,
   wherein said C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, C₁-C₆-alkyl-N(R^{a})₂, —C₁-C₆-alkyl-C(═O)R^{a}, —C₁-C₆-alkyl-O—C(═O)R^{a}, —C₁-C₆-alkyl-C(═O)OR^{a}, —C₁-C₆-alkyl-S(═O)₂R^{a} and —C₁-C₆-alkyl-P(═O)(OR^{a})₂ substituent is itself optionally substituted, one or more times, in the same way or differently, with R5′,
   provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₁-alkyl (i.e. methyl),
   provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₃-C₁₀-carbocyclyl or a 3- to 10-membered-heterocyclyl and R5′ is attached to the carbon atom of R4 that bonds R4 and A;
   provided R4 is not attached to A via a nitrogen atom when R4 is a 3- to 10-membered-heterocyclyl,
   provided R5′ is not attached to R4 via a nitrogen atom when R5′ is a 3- to 10-membered-heterocyclyl and R4 is C₁-alkyl (i.e. methyl);
R5 represents a substituent selected from the group consisting of halogen, cyano, azide, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —C(═NR^{a})N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —Si(C₁-C₆-alkyl)₃, substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R5′ represents a substituent selected from the group consisting of halogen, cyano, azide, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —Si(C₁-C₆-alkyl)₃, substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, OC(═O)N(R^{b})₂, and —P(═O)(OR^{b})₂;
   wherein said C₁-C₆-alkyl, C₁-C₆-haloalkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy substituent is itself optionally substituted, one or more times, in the same way or differently with R^{b}; or
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂; wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
n represents an integer of 0, 1, 2, 3, 4, 5, or 6;
m represents an integer of 0, 1, 2, 3, 4, or 5; and p represents an integer of 0, 1, 2, 3, 4, or 5.

In some embodiments (referred herein as embodiment 2), the present invention provides for the use of a compound of the formula (I) for controlling phytopathogenic fungi:

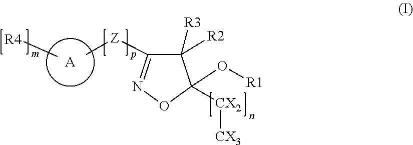

in which:
R1 represents a substituent selected from the group consisting of hydrogen, sulfinyl, sulfonyl, C₁-C₆-alkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, heteroaryl, nitro, —C(═O)R^{a}, —C(═O)OR^{a}, —C(═O)N(R^{a})₂, —C(═S)N(R^{a})₂, —S(═O)₂R^{a}, —S(═O)₂N(R^{a})₂ and —P(═O)(OR^{a})₂, wherein said C₁-C₆-alkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3- to 10-membered heterocyclyl, C₁-C₆-haloalkyl, hydroxy-C₁-C₆-alkyl, aryl, heteroaryl substituent is itself optionally substituted, one or more times, in the same way or differently, with R5;
R2 and R3 independently of each other, represent a substituent selected from the group consisting of hydrogen, halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —C(═O)R^{a}, —C(═O)OR^{a}, —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —OC(═O)N(R^{a})₂, —NR^{a}S(═O)₂R^{a}, —S(═O)₂R^{a}, —S(═O)₂N(R^{a})₂, —P(═O)(OR^{a})₂, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂, —C₁-C₆-alkyl-P(═O)(OR^{a})₂ substituent is itself optionally substituted, one or more times, in the same way or differently, with R5;
   or,
   together, form a C₃-C₁₀-carbocyclyl or 3- to 10-membered-heterocyclyl;
A represents aryl, heteroaryl, C₃-C₁₀-carbocyclyl or 3- to 15-membered-heterocyclyl, preferably aryl or heteroaryl group;
X represents, independently of each other, a substituent selected from the group consisting of hydrogen, fluorine, chlorine, bromine, and iodine atom, wherein at least one X substituent is a fluorine atom and provided that CX₃ is not CF₃ or CHF₂ when n=0;
Z represents a C₂-C₆-alkenyl group, itself being optionally substituted, one or more times, in the same way or differently, with R5;
R4 represents independently a substituent selected from the group consisting of halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —SF₅, —C(═O)R^{a}, —C(═O)OR^{a}, —C(═O)N(R^{a})₂, —C(═O)NR^{a}N(R^{a})₂, —C(═NR^{a})R^{a}, —C(═S)N(R^{a})₂, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —OC(═O)N(R^{a})₂, —OC(═O)R^{a}, —C(═O)N(OR^{a})R^{a}, —NR^{a}S(═O)₂R^{a}, —S(═O)₂R^{a}, —S(═O)₂N(R^{a})₂, —(C═NR^{a})N(R^{a})₂, —P(═O)(OR^{a})₂, —C₁-C₆-alkyl-3- to 10-membered-heterocyclyl, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-O—C(═O)R^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-N(OR^{a})C(═O)R^{a}, —C₁-C₆-alkyl-C(═O)N(OR^{a})R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —C₁-C₆-alkyl-3- to 10-membered-heterocyclyl, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-O—C(═O)R^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-N(OR^{a})C(═O)R^{a}, —C₁-C₆-alkyl-C(═O)N(OR^{a})R^{a}, —C₁-C₆-alkyl-S(═O)₂N(R^{a})₂ and —C₁-C₆-alkyl-P(═O)(OR^{a})₂ substituent is itself optionally substituted, one or more times, in the same way or differently, with R5;
R5 represents a substituent selected from the group consisting of halogen, cyano, azide, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂—C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
   wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy substituent is itself optionally substituted, one or more times, in the same way or differently with R^{b},
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
n represents an integer of 0, 1, 2, 3, 4, 5, or 6;
m represents an integer of 0, 1, 2, 3, 4, or 5; and
p represents an integer of 0, 1, 2, 3, 4, or 5.

In some embodiments (referred herein as embodiment 3), the present invention provides for the use of a compound of the formula (I) for controlling phytopathogenic fungi:

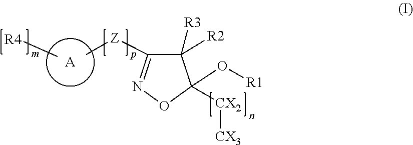

in which:
R1 represents a substituent selected from the group consisting of hydrogen, sulfinyl, sulfonyl, C₁-C₆-alkyl, C₁-C₆-haloalkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3- to 10-membered-heterocyclyl, hydroxy-C₁-C₆-alkyl, aryl, heteroaryl, nitro, —C(═O)R^{a}, —C(═O)OR^{a}, —C(═O)N(R^{a})₂, —C(═S)N(R^{a})₂, —S(═O)₂R^{a}, —S(═O)₂N(R^{a})₂ and —P(═O)(OR^{a})₂,
   wherein said C₁-C₆-alkyl, C₁-C₆-haloalkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3- to 10-membered-heterocyclyl, hydroxy-C₁-C₆-alkyl, aryl, heteroaryl substituent is itself optionally substituted, one or more times, in the same way or differently, with R5;
R2 and R3 independently of each other, represent a substituent selected from the group consisting of hydrogen, halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —C(═O)R^{a}, —C(═O)OR^{a}, —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —OC(═O)N(R^{a})₂, —NR^{a}S(═O)₂R^{a}, —S(═O)₂R^{a}, —S(═O)₂N(R^{a})₂, —P(═O)(OR^{a})₂, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂, —C₁-C₆-alkyl-P(═O)(OR^{a})₂ substituent is itself optionally substituted, one or more times, in the same way or differently, with R5;
   or,
   together, form a C₃-C₁₀-carbocyclyl or 3- to 10-membered-heterocyclyl;
A represents C₃-C₁₀-carbocyclyl or 3- to 15-membered-heterocyclyl;
X represents, independently of each other, a substituent selected from the group consisting of hydrogen, fluorine, chlorine, bromine, and iodine atom, wherein at least one X substituent is a fluorine atom;
Z represents a C₂-C₆-alkenyl group, itself being optionally substituted, one or more times, in the same way or differently, with R5;
R4 represents independently a substituent selected from the group consisting of halogen, cyano, hydroxy, mercapto, sulfinyl, sulfonyl, amino, C₁-C₆-alkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —SF₅, —C(═O)R^{a}, —C(═O)OR^{a}, —C(═O)N(R^{a})₂, —C(═O)NR^{a}N(R^{a})₂, —C(═NR^{a})R^{a}, —C(═S)N(R^{a})₂, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —OC(═O)N(R^{a})₂, —OC(═O)R^{a}, —C(═O)N(OR^{a})R^{a}, —NR^{a}S(═O)₂R^{a}, —S(═O)₂R^{a}, —S(═O)₂N(R^{a})₂, —(C═NR^{a})N(R^{a})₂, —P(═O)(OR^{a})₂, —C₁-C₆-alkyl-3- to 10-membered-heterocyclyl, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-N(OR^{a})C(═O)R^{a}, —C₁-C₆-alkyl-C(═O)N(OR^{a})R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —C₁-C₆-alkyl-3- to 10-membered-heterocyclyl, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-O—C(═O)R^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-N(OR^{a})C(═O)R^{a}, —C₁-C₆-alkyl-C(═O)N(OR^{a})R^{a}, —C₁-C₆-alkyl-S(═O)₂N(R^{a})₂ and —C₁-C₆-alkyl-P(═O)(OR^{a})₂ substituent is itself optionally substituted, one or more times, in the same way or differently, with R5;
R5 represents a substituent selected from the group consisting of halogen, cyano, azide, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
n represents an integer of 0, 1, 2, 3, 4, 5, or 6;
m represents an integer of 0, 1, 2, 3, 4, or 5; and
p represents an integer of 0, 1, 2, 3, 4, or 5.

In some embodiments (referred herein as embodiment 4), the present invention provides for the use of a compound of the formula (I) for controlling phytopathogenic fungi:

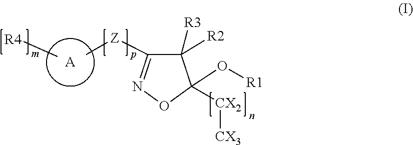

in which
R1 represents a substituent selected from the group consisting of hydrogen, sulfinyl, sulfonyl, C₁-C₆-alkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, heteroaryl, nitro, —C(═O)R^{a}, —C(═O)OR^{a}, —C(═O)N(R^{a})₂, —C(═S)N(R^{a})₂, —S(═O)₂R^{a}, —S(═O)₂N(R^{a})₂ and —P(═O)(OR^{a})₂,
   wherein said C₁-C₆-alkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, heteroaryl substituent is itself optionally substituted, one or more times, in the same way or differently, with R5;
R2 and R3 independently of each other, represent a substituent selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{a}, —C(═O)OR^{a}, —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —OC(═O)N(R^{a})₂, —NR^{a}S(═O)₂R^{a}, —S(═O)₂R^{a}, —S(═O)₂N(R^{a})₂, —P(═O)(OR^{a})₂, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂, —C₁-C₆-alkyl-P(═O)(OR^{a})₂ substituent is itself optionally substituted, one or more times, in the same way or differently, with R5;
   or,
   together, form a C₃-C₁₀-carbocyclyl or 3- to 10-membered-heterocyclyl;
A represents aryl, heteroaryl, C₃-C₁₀-carbocyclyl or 3- to 15-membered-heterocyclyl group, provided A is not phenyl nor a 5- or 6-membered aromatic heteroaryl; preferably represents aryl or heteroaryl group;
X represents, independently of each other, a substituent selected from the group consisting of hydrogen, fluorine, chlorine, bromine, and iodine atom, wherein at least one X substituent is a fluorine atom;
Z represents a C₂-C₆-alkenyl group, itself being optionally substituted, one or more times, in the same way or differently, with R5;
R4 represents independently a substituent selected from the group consisting of halogen, cyano, hydroxy, mercapto, sulfinyl, sulfonyl, amino, C₁-C₆-alkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —SF₅, —C(═O)R^{a}, —C(═O)OR^{a}, —C(═O)N(R^{a})₂, —C(═O)NR^{a}N(R^{a})₂, —C(═NR^{a})R^{a}, —C(═S)N(R^{a})₂, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —OC(═O)N(R^{a})₂, —OC(═O)R^{a}, —C(═O)N(OR^{a})R^{a}, —NR^{a}S(═O)₂R^{a}, —S(═O)₂R^{a}, —S(═O)₂N(R^{a})₂, —(C═NR^{a})N(R^{a})₂, —P(═O)(OR^{a})₂, —C₁-C₆-alkyl-3- to 10-membered-heterocyclyl, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-O—C(═O)R^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-N(OR^{a})C(═O)R^{a}, —C₁-C₆-alkyl-C(═O)N(OR^{a})R^{a}, —C₁-C₆-alkyl-S(═O)₂N(R^{a})₂ and —C₁-C₆-alkyl-P(═O)(OR^{a})₂,
   wherein said C₁-C₆-alkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —C₁-C₆-alkyl-3- to 10-membered-heterocyclyl, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-O—C(═O)R^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-N(OR^{a})C(═O)R^{a}, —C₁-C₆-alkyl-C(═O)N(OR^{a})R^{a}, —C₁-C₆-alkyl-S(═O)₂N(R^{a})₂ and —C₁-C₆-alkyl-P(═O)(OR^{a})₂ substituent is itself optionally substituted, one or more times, in the same way or differently, with R5;
R5 represents a substituent selected from the group consisting of halogen, cyano, azide, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
n represents an integer of 0, 1, 2, 3, 4, 5, or 6;
m represents an integer of 0, 1, 2, 3, 4, or 5; and
p represents an integer of 0, 1, 2, 3, 4, or 5.

The compounds of Formula (I) in accordance with embodiments 1, 2, 3 and 4 can be in their free form, salt form, N-oxides form or solvate form (e.g. hydrate).

In embodiments 1, 2, 3 or 4, R1 is preferably a substituent selected from the group consisting of hydrogen, C₁-C₆-alkyl and —C(═O)R^{a}, wherein said C₁-C₆-alkyl substituent is itself optionally substituted, one or more times, in the same way or differently, with R5; R^{a} and R5 being as disclosed above.

In embodiments 1, 2, 3 or 4, R1 is more preferably a substituent selected from the group consisting of hydrogen, C₁-C₆-alkyl and —C(═O)R^{a}, wherein:
R^{a} represent a substituent selected from the group consisting of C₁-C₆-alkyl (that may be substituted by a C₁-C₆-alkoxy), C₁-C₆-haloalkyl, C₃-C₁₀-carbocyclyl and aryl (preferably phenyl),
said C₁-C₆-alkyl substituent is itself optionally substituted, one or more times, in the same way or differently, with R5; R5 being halogen, cyano and C₁-C₆-alkoxy or —C(═O)R^{a}, wherein R^{a} represents a substituent selected from the group consisting of C₁-C₆-alkyl, C₁-C₆-alkyl substituted by a C₃-C₁₀-carbocyclyl, C₃-C₁₀-carbocyclyl (that may be substituted by a C₁-C₆-alkyl), aryl (preferably phenyl) and —C(═O)OR^{b} wherein R^{b} is a C₁-C₆-alkyl.

In embodiments 1, 2, 3 or 4, R2 and R3 are preferably independently selected from the group consisting of hydrogen, halogen and C₁-C₆-alkyl or R2 and R3 together form with the carbon atom to which they are attached a C₃-C₁₀-carbocyclyl, preferably a C₃-C₁₀-cycloalkyl, such as a cyclopropyl.

In embodiments 1, 2, 3 or 4, R2 and R3 are more preferably independently selected from the group consisting hydrogen, fluorine and methyl.

In embodiments 1, 2, 3 or 4, R2 and R3 are even more preferably a hydrogen atom.

In embodiments 1 or 2, A is preferably aryl or heteroaryl group, more preferably an aryl chosen from mono- or bicyclic aromatic group, preferably phenyl or naphthyl, more preferably phenyl or A represents a heteroaryl group chosen from mono- or bicyclic aromatic group containing at least one heteroatom chosen from S, N or O, preferably thienyl, thiazolyl, benzofuranyl, indazolyl, benzothiazolyl, benzothiophenyl, benzothiazolyl, pyridyl and pyrimidinyl, more preferably thienyl, benzofuranyl, pyridyl and pyrimidinyl.

In some embodiments in accordance with embodiments 1, 2 or 4, A is naphthyl or A represents a heteroaryl group chosen from benzofuranyl, indazolyl, benzothiazolyl, benzothiophenyl and benzothiazolyl.

In embodiments 1, 2, 3 or 4, p and n are preferably 0.

In embodiments 1, 2, 3 or 4, m is preferably 0, 1 or 2, more preferably 1.

In embodiments 1, 2, 3 or 4, X is preferably independently hydrogen, fluorine, chlorine or bromine.

In some embodiments in accordance with embodiment 1, R4 is preferably a substituent selected from the group consisting of halogen, cyano, hydroxy, amino, C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl (e.g. cyclopropyl, cyclohexyl), 3- to 10-membered-heterocyclyl (e.g. dihydrooxazolyl, dihydropyridinyl, dihydrobenzofuranyl), C₁-C₆-haloalkyl, hydroxy-C₁-C₆-alkyl, aryl (e.g. phenyl), aryloxy, heteroaryl (e.g. imidazolyl, pyridinyl, pyrimidinyl, isoxazolyl, oxadiazolyl, pyrazolyl), nitro, —SF₅, —C(═O)R^{a}, —C(═O)OR^{a}, —C(═NR^{a})R^{a}, —N(R^{a})₂, —C(═NR^{a})N(R^{a})₂, —C₁-C₆-alkyl-N(R^{a})₂ and —C₁-C₆-alkyl-C(═O)OR^{a} with R^{a} as described herein. R4 may be substituted as described herein.

In some embodiments in accordance with embodiments 2, 3 or 4, R4 is a substituent selected from the group consisting of halogen, hydroxy, cyano, —SF₅, C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, heteroaryl, —SO₂N(R^{a})₂, —OC(═O)R^{a}, —C(═O)N(OR^{a})R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —NR^{a}S(═O)₂R^{a}, —OC(═O)N(R^{a})₂, —C(═NR^{a})R^{a}, —C(═NR^{a})N(R^{a})₂, —C(═O)—O—R^{a}, —C(═O)N(R^{a})₂, —C(═O)NR^{a}N(R^{a})₂, —C(═S)N(R^{a})₂, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a},
wherein said C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, heteroaryl, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5; R5 and R^{a} being as disclosed above.

In some embodiments in accordance with embodiments 2, 3 or 4, R4 represents independently a substituent selected from the group consisting of halogen, cyano, hydroxy, amino, C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl (e.g. cyclopropyl, cyclohexyl), 3- to 10-membered-heterocyclyl (e.g. dihydrooxazolyl, dihydropyridinyl, dihydrobenzofuranyl), C₁-C₆-haloalkyl, hydroxy-C₁-C₆-alkyl, aryl (e.g. phenyl), aryloxy, heteroaryl (e.g. imidazolyl, pyridinyl, pyrimidinyl, isoxazolyl, oxadiazolyl, pyrazolyl), nitro, —SF₅, —C(═O)R^{a}, —C(═O)OR^{a}, —C(═NR^{a})R^{a}, —N(R^{a})₂, —C(═NR^{a})N(R^{a})₂, —C₁-C₆-alkyl-N(R^{a})₂, —C₁-C₆-alkyl- C(═O)OR^{a}, —C(═O)N(R^{a})₂, —C(═O)NR^{a}N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a} with R^{a} as described herein. R4 may be substituted as described herein, preferably R4 represents independently a substituent selected from the group consisting of amino, C₁-C₆-alkylthio heteroaryl (e.g. imidazolyl, pyridinyl, pyrimidinyl, isoxazolyl, oxadiazolyl, pyrazolyl), nitro, —C(═O)R^{a}, —C(═O)OR^{a}, —N(R^{a})₂, —C(═O)N(R^{a})₂, —C(═O)NR^{a}N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂ and —NR^{a}S(═O)₂R^{a},

In some embodiments in accordance with embodiment 1, the present invention relates to the use for controlling phytopathogenic fungi of compounds of formula I, supra, in which:
R1 is a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl, preferably R2 and R3 represent hydrogen;
A is a phenyl;
p and n are 0;
m is 1;
X is, independently of each other, hydrogen, fluorine, chlorine or bromine;
R4 is a substituent selected from the group consisting of halogen, hydroxy, cyano, —SF₅, C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, heteroaryl, —OC(═O)R^{a}, —N(R^{a})₂, —NR^{a}C(═NR^{a})R^{a}, —N═CR^{a}—N(R^{a})₂, —C(═NR^{a})R^{a}, C(═NR^{a})N(R^{a})₂, —C(═O)—O—R^{a}, —C₁-C₆-alkyl-N(R^{a})₂, wherein said C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, heteroaryl and —C₁-C₆-alkyl-N(R^{a})₂ substituent is itself optionally substituted, one or more times, in the same way or differently, with R5′;
R5′ and R^{a} are as disclosed above.

In some embodiments in accordance with embodiment 2, the present invention relates to the use for controlling phytopathogenic fungi of compounds of formula I, supra, in which:
R1 is a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl, preferably R2 and R3 represent hydrogen;
A is a phenyl;
p and n are 0;
m is 1;
X is, independently of each other, hydrogen, fluorine, chlorine or bromine;
R4 is a substituent selected from the group consisting of halogen, hydroxy, cyano, —SF₅, C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, heteroaryl, —SO₂N(R^{a})₂, —OC(═O)R^{a}, —C(═O)N(OR^{a})R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —NR^{a}S(═O)₂R^{a}, —OC(═O)N(R^{a})₂, —C(═NR^{a})R^{a}, C(═NR^{a})N(R^{a})₂, —C(═O)—O—R^{a}, —C(═O)N(R^{a})₂, —C(═O)NR^{a}N(R^{a})₂, —C(═S)N(R^{a})₂, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a}, wherein said C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, heteroaryl, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5;
R5 and R^{a} are as disclosed above.

In some embodiments (referred herein as embodiment 5), the present invention relates to compounds of formula (I′):

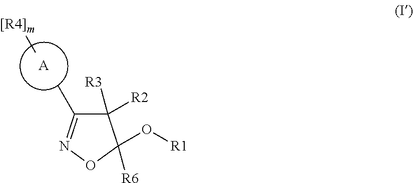

in which
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3 independently of each other, represent a substituent selected from the group consisting of hydrogen, halogen and C₁-C₆-alkyl, preferably R2 and R3 are hydrogen; or,
   together, form a C₃-C₁₀-carbocyclyl;
A represents aryl, heteroaryl, C₃-C₁₀-carbocyclyl or 3- to 15-membered-heterocyclyl group, preferably aryl or heteroaryl group;
R6 represents CX₃ wherein X represents, independently of each other, a substituent selected from the group consisting of hydrogen, fluorine, chlorine, bromine, and iodine atom, wherein at least one X substituent is a fluorine atom;
R4 represents independently a substituent selected from the group consisting of halogen, cyano, hydroxy, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —C(═O)R^{a}, —OC(═O)R^{a}, —N(R^{a})₂, —NR^{a}C(═NR^{a})R^{a}, —N═CR^{a}—N(R^{a})₂, —C(═NR^{a})R^{a}, —C(═NR^{a})N(R^{a})₂, —C(═O)OR^{a}, —C₁-C₆-alkyl-C(═O)OR^{a}, —C₁-C₆-alkyl-O—C(═O)R^{a} and —C₁-C₆-alkyl-N(R^{a})₂,
   wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-O—C(═O)R^{a} and —C₁-C₆-alkyl-N(R^{a})₂ substituent is itself optionally substituted, one or more times, in the same way or differently, with R5′,
   provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₁-alkyl (i.e. methyl),
   provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₃-C₁₀-carbocyclyl or a 3- to 10-membered-heterocyclyl and R5′ is attached to the carbon atom of R4 that bonds R4 and A;
   provided R4 is not attached to A via a nitrogen atom when R4 is a 3- to 10-membered-heterocyclyl,
   provided R5′ is not attached to R4 via a nitrogen atom when R5′ is a 3- to 10-membered-heterocyclyl and R4 is C₁-alkyl (i.e. methyl);
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered heterocyclyl, C₁-C₆-haloalkyl, hydroxy-C₁-C₆-alkyl, aryl, aryloxy, heteroaryl, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R5′ represents a substituent selected from the group consisting of halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃, substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —OC(═O)N(R^{b})₂, and —P(═O)(OR^{b})₂, wherein said C₁-C₆-alkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b}; or
   two Ra when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with CN, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
provided that:
   R6 does not represent CF₃ when A represents phenyl (as aryl), m is 1, R1 is hydrogen and R4 represents hydroxy, C₁-C₆-alkoxy, —N(R^{a})₂ with R^{a} is hydrogen, or —C₁-C₆-alkyl-N(R^{a})₂;
   R1 does not represent a hydrogen atom when A represents phenyl (as aryl), R6 represents CF₃ m is 1 and R4 represents hydroxy, C₁-C₆-alkoxy, —N(R^{a})₂ with R^{a} is hydrogen, or —C₁-C₆-alkyl-N(R^{a})₂;
   R5′ does not represent amino or C₁-C₆-alkylamino when A represents phenyl, R6 is CF₃, m is 1 and R4 represents C₁-C₆-alkyl;
   m is 1 or 2 when A represents phenyl (as aryl) and R1 represents hydrogen;
   R4 does not represent hydroxy, halogen, methyl or methoxy when A represents phenyl (as aryl) and m is 1;
   (R4; R4) do not represent (hydroxyl; halogen), (methoxy; methoxy), (methoxy; halogen), (hydroxyl; methyl), (fluor; fluorophenyl) or (methoxy; methoxypropoxy) when A represents phenyl (as aryl) and m is 2;
   R2 or R3 does not represent halogen when A represents phenyl (as aryl);
   compounds of formula (I′) is not
3-phenyl-5-(trifluoromethyl)-4,5-dihydro-1,2-oxazol-5-yl acetate (873694-74-7);
3-[1-(hydroxymethyl)cyclohexyl]-5-(trifluoromethyl)-4,5-dihydro-1,2-oxazol-5-ol (212615-88-8);
3-[4-(3-tert-butyl-4,4-dimethyl-4,5-dihydrofuran-2-yl)-2-methoxyphenyl]-5-(trifluoromethyl)-4,5-dihydro-1,2-oxazol-5-ol (7898-55-4);
3,3′-(1,4-phenylene)bis[5-(trifluoromethyl)-4,5-dihydro-1,2-oxazol-5-ol] (242461-20-7);
3-(3-thienyl)-5-(trifluoromethyl)-4,5-dihydro-1,2-oxazol-5-ol (1170114-77-8);
ethyl 4-[5-hydroxy-5-(trifluoromethyl)-4,5-dihydro-1,2-oxazol-3-yl]benzoate (1124198-92-0);
4-[5-hydroxy-5-(trifluoromethyl)-4,5-dihydro-1,2-oxazol-3-yl]-2-[(2,2,2-trifluoroethyl)sulfanyl]benzonitrile (1093847-08-5);
5-[bromo(difluoro)methyl]-3-(2-thienyl)-4,5-dihydro-1,2-oxazol-5-ol (1035637-61-6);
3-(5-chloro-3-methyl-1-benzothiophen-2-yl)-5-(trifluoromethyl)-4,5-dihydro-1,2-oxazol-5-ol (883055-08-1);
5-[5-hydroxy-5-(trifluoromethyl)-4,5-dihydro-1,2-oxazol-3-yl]thiophene-2-carbonitrile (656227-15-5);
5-[5-hydroxy-5-(trifluoromethyl)-4,5-dihydro-1,2-oxazol-3-yl]thiophene-2-carboxylic acid (656226-62-9);
3-(2-furyl)-5-(trifluoromethyl)-4,5-dihydro-1,2-oxazol-5-ol (501953-86-2);
3-(2-naphthyl)-5-(trifluoromethyl)-4,5-dihydro-1,2-oxazol-5-ol (328285-44-5);
3-(2-thienyl)-5-(trifluoromethyl)-4,5-dihydro-1,2-oxazol-5-ol (293759-12-3).

In the formula (I′), when m is 2, the two R4 substituents are designated as follows: (R4; R4). For instance, when A is substituted with hydroxyl and halogen, R4 substituents are designated as (hydroxyl; halogen).

In the formula (I′), when it is said that R2 or R3 does not represent halogen when A represents phenyl, it has to be understood that (R2;R3) are different from (hydrogen; halogen), (halogen; hydrogen), (C₁-C₃-alkyl; halogen), (halogen; C₁-C₃-alkyl), (halogen; halogen).

In some embodiments (referred herein as embodiment 6), the present invention relates to compounds of formula (I′):

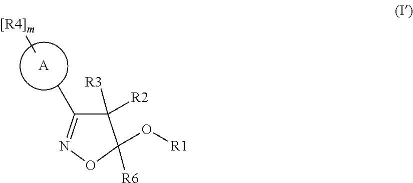

in which
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl, preferably R2 and R3 are hydrogen; or,
   together, form a C₃-C₁₀-carbocyclyl;
A represents aryl, heteroaryl, C₃-C₁₀-carbocyclyl or 3- to 15-membered-heterocyclyl group, preferably aryl or heteroaryl group;
R6 represents CX₃ wherein X represents, independently of each other, a substituent selected from the group consisting of hydrogen, fluorine, chlorine, bromine, and iodine atom, wherein at least one X substituent is a fluorine atom and provided R6 is not CF₃ or CHF₂;
R4 represents independently a substituent selected from the group consisting of halogen, hydroxy, cyano, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —SO₂N(R^{a})₂, —C(═O)R^{a}, —C(═NR^{a})N(R^{a})₂, —OC(═O)R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —NR^{a}═C—N(R^{a})₂, —NR^{a}S(═O)₂R^{a}, —OC(═O)N(R^{a})₂, —C(═NR^{a})R^{a}, —C(═O)—O—R^{a}, —C(═O)N(R^{a})₂, —C(═O)NR^{a}N(R^{a})₂, —C(═O)N(OR^{a})R^{a}, —C(═S)N(R^{a})₂, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a},
   wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5;
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
provided that compounds of formula (I′) is not:
5-[bromo(difluoro)methyl]-3-(2-thienyl)-4,5-dihydro-1,2-oxazol-5-ol (1035637-61-6);
5-[bromo(difluoro)methyl]-3-(4-methylphenyl)-4,5-dihydro-1,2-oxazol-5-ol (1224442-78-7);
5-[bromo(difluoro)methyl]-3-(4-methoxyphenyl)-4,5-dihydro-1,2-oxazol-5-ol (1035637-62-7);
5-[bromo(difluoro)methyl]-3-phenyl-4,5-dihydro-1,2-oxazol-5-ol (1035637-60-5).

In some embodiments (referred herein as embodiment 7), the present invention relates to compounds of formula (I′):

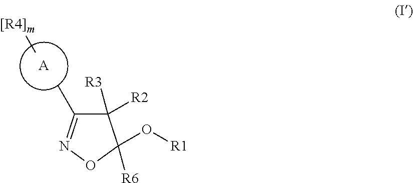

in which:
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl, preferably R2 and R3 are hydrogen;
   or,
   together, form a C₃-C₁₀-carbocyclyl;
A represents C₃-C₁₀-carbocyclyl or 3- to 15-membered-heterocyclyl;
R6 represents CX₃ wherein X represents, independently of each other, a substituent selected from the group consisting of hydrogen, fluorine, chlorine, bromine, and iodine atom, wherein at least one X substituent is a fluorine atom;
R4 represents independently a substituent selected from the group consisting of halogen, hydroxy, cyano, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —SO₂N(R^{a})₂, —C(═O)R^{a}, —C(═NR^{a})N(R^{a})₂, —OC(═O)R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —NR^{a}S(═O)₂R^{a}, —OC(═O)N(R^{a})₂, —C(═NR^{a})R^{a}, —C(═O)—O—R^{a}, —C(═O)N(R^{a})₂, —C(═O)NR^{a}N(R^{a})₂, —C(═O)N(OR^{a})R^{a}, —C(═S)N(R^{a})₂, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a},
   wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5;
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
   provided that compound of formula (I′) is not 3-[1-(hydroxymethyl)cyclohexyl]-5-(trifluoromethyl)-4,5-dihydro-1,2-oxazol-5-ol (212615-88-8).

In some embodiments (referred herein as embodiment 8), the present invention relates to compounds of formula (I′):

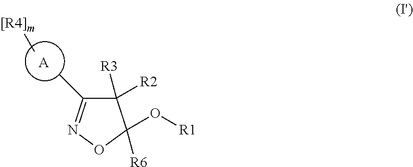

in which
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl, preferably R2 and R3 are hydrogen, or,
   together, form a C₃-C₁₀-carbocyclyl;
A represents aryl, heteroaryl, C₃-C₁₀-carbocyclyl or 3- to 15-membered-heterocyclyl group, provided A is not phenyl or not a 5- or 6-membered aromatic heteroaryl, preferably represents aryl or heteroaryl group;
R6 represents CX₃ wherein X represents, independently of each other, a substituent selected from the group consisting of hydrogen, fluorine, chlorine, bromine, and iodine atom, wherein at least one X substituent is a fluorine atom;
R4 represents independently a substituent selected from the group consisting of halogen, hydroxy, cyano, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —SO₂N(R^{a})₂, —C(═O)R^{a}, —C(═NR^{a})N(R^{a})₂, —OC(═O)R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —NR^{a}S(═O)₂R^{a}, —OC(═O)N(R^{a})₂, —C(═NR^{a})R^{a}, —C(═O)—O—R^{a}, —C(═O)N(R^{a})₂, —C(═O)NR^{a}N(R^{a})₂, —C(═O)N(OR^{a})R^{a}, —C(═S)N(R^{a})₂, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a},
   wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5;
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
   wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
   two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
   wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
   provided that compounds of formula (I′) is not
3-[1-(hydroxymethyl)cyclohexyl]-5-(trifluoromethyl)-4,5-dihydro-1,2-oxazol-5-ol (212615-88-8);
3-(5-chloro-3-methyl-1-benzothiophen-2-yl)-5-(trifluoromethyl)-4,5-dihydro-1,2-oxazol-5-ol (883055-08-1);
3-(2-naphthyl)-5-(trifluoromethyl)-4,5-dihydro-1,2-oxazol-5-ol (328285-44-5);
3-[1-(hydroxymethyl)cyclohexyl]-5-(trifluoromethyl)-4,5-dihydro-1,2-oxazol-5-ol (212615-88-8).

In embodiments 5, 6, 7 or 8, R1 is preferably a substituent selected from the group consisting of hydrogen, C₁-C₆-alkyl and —C(═O)R^{a}, wherein:
R^{a} represent a substituent selected from the group consisting of C₁-C₆-alkyl, C₁-C₆-haloalkyl, C₃-C₁₀-carbocyclyl, C₁-C₆-alkyl substituted by C₁-C₆-alkoxy and aryl (preferably phenyl),
said C₁-C₆-alkyl substituent is itself optionally substituted, one or more times, in the same way or differently, with R5; R5 being halogen, cyano and C₁-C₆-alkoxy or —C(═O)R^{a}, wherein R^{a} represents a substituent selected from the group consisting of C₁-C₆-alkyl, C₁-C₆-alkyl substituted by C₃-C₁₀-carbocyclyl, C₃-C₁₀-carbocyclyl (that may be substituted by a C₁-C₆-alkyl), aryl (preferably phenyl) and —C(═O)OR^{b} wherein R^{b} is a C₁-C₆-alkyl.

In embodiments 5, 6, 7 or 8, R2 and R3 are preferably independently selected from the group consisting of hydrogen, halogen and methyl or R2 and R3 together form a C₃-C₁₀-carbocyclyl, preferably a C₃-C₁₀-cycloalkyl, such as a cyclopropyl, more preferably R2 and R3 are hydrogen.

In embodiments 5, 6, 7 or 8, R2 and R3 are more preferably independently selected from the group consisting hydrogen, fluorine and methyl.

In embodiments 5, 6, 7 or 8, m is preferably 1 or 2.

In embodiments 5 or 6, A preferably represents an aryl or heteroaryl group, more preferably an aryl chosen from mono- or bicyclic aromatic group, preferably phenyl or naphtyl, or A represents a heteroaryl group chosen, from mono- or bicyclic aromatic group containing at least one heteroatom chosen from S, N or O, preferably thienyl, thiazolyl, benzofuranyl, indazolyl, benzothiazolyl, benzothiophenyl, benzothiazolyl, pyridyl and pyrimidinyl, more preferably thienyl, benzofuranyl, pyridyl and pyrimidinyl.

In embodiment 8, A preferably represents an aryl chosen from bicyclic aromatic group, preferably naphthyl, or A represents a heteroaryl group chosen from bicyclic aromatic group containing at least one heteroatom chosen from S, N or O, preferably benzofuranyl, indazolyl, benzothiazolyl, benzothiophenyl and benzothiazolyl,

In embodiments 5, 7 or 8, R6 preferably represents CF₃, CF₂CL, CF₂Br or CHF₂.

In some embodiments in accordance with embodiment 5, R4 represents a substituent selected from the group consisting of halogen, cyano, hydroxy, C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl (e.g. cyclopropyl, cyclohexyl), 3- to 10-membered-heterocyclyl (e.g. dihydrooxazolyl, dihydropyridinyl, dihydrobenzofuranyl), C₁-C₆-haloalkyl, hydroxy-C₁-C₆-alkyl, aryl (e.g. phenyl), aryloxy, heteroaryl (e.g. imidazolyl, pyridinyl, pyrimidinyl, isoxazolyl, oxadiazolyl, pyrazolyl), —SF₅, —C(═O)R^{a}, —C(═O)OR^{a}, —C(═NR^{a})R^{a}, —N(R^{a})₂, —C(═NR^{a})N(R^{a})₂, —C₁-C₆-alkyl-N(R^{a})₂ and —C₁-C₆-alkyl-C(═O)OR^{a} with R^{a} as described herein. R4 may be substituted as described herein.

In embodiment 5, R4 may represent a substituent selected from the group consisting of halogen, C₁-C₆-alkylthio, arylsulfenyl, C₁-C₆-alkylsulfinyl, arylsulfinyl, C₁-C₆-alkylsulfonyl, arylsulfonyl, aryl, heteroaryl, 3- to 10-membered-heterocyclyl wherein said C₁-C₆-alkylthio, arylsulfenyl, C₁-C₆-alkylsulfinyl, arylsulfinyl, C₁-C₆-alkylsulfonyl, arylsulfonyl, aryl, heteroaryl, 3- to 10-membered-heterocyclyl substituent is itself optionally substituted, one or more times, in the same way or differently, with R5′; R5′ being as disclosed above.

In some embodiments in accordance with embodiments 6, 7 or 8, R4 represents a substituent selected from the group consisting of halogen, SF₅, C₁-C₆-alkylthio, C₂-C₆-alkynyl, arylsulfenyl, C₁-C₆-alkylsulfinyl, arylsulfinyl, C₁-C₆-alkylsulfonyl, arylsulfonyl, aryl, heteroaryl, 3- to 10-membered-heterocyclyl, —C(═O)N(R^{a})₂, —N(R^{a})₂, —NR^{a}C(═O)OR^{a}, —NR^{a}C(═O)R^{a}, —OC(═O)N(R^{a})₂, —C(═O)N(OR^{a})R^{a}, —C₁-C₆-alkyl-3- to 10-membered-heterocyclyl and —C₁-C₆-alkyl-C(═O)N(R^{a})₂, wherein:
said C₁-C₆-alkylthio, C₂-C₆-alkynyl, arylsulfenyl, C₁-C₆-alkylsulfinyl, arylsulfinyl, C₁-C₆-alkylsulfonyl, arylsulfonyl, aryl, heteroaryl, 3- to 10-membered-heterocyclyl, —C₁-C₆-alkyl-3- to 10-membered-heterocyclyl, —C₁-C₆-alkyl-C(═O)N(R^{a})₂, substituent is itself optionally substituted, one or more times, in the same way or differently, with R5; R5 being as disclosed above;
Ra represents, independently from each other, a substituent, which is identical or different, selected from the group consisting of hydrogen, C₁-C₆-alkyl, C₂-C₆-alkynyl, C₁-C₆-alkoxy, C₃-C₁₀-carbocyclyl, 3- to 10-membered-heterocyclyl, C₁-C₆-haloalkyl, —C₁-C₆-alkyl-C₃-C₁₀-carbocyclyl, —C₁-C₆-alkyl-aryl, —C₁-C₆-alkyl-C₁-C₆-alkoxy, aryl, heteroaryl; wherein said C₁-C₆-alkyl, C₂-C₆-alkynyl, C₃-C₁₀-carbocyclyl, 3- to 10-membered-heterocyclyl, C₁-C₆-haloalkyl, —C₁-C₆-alkyl-C₃-C₁₀-carbocyclyl, —C₁-C₆-alkyl-aryl, —C₁-C₆-alkyl-C₁-C₆-alkoxy, aryl, heteroaryl substituent is itself optionally substituted, one or more times, in the same way or differently with R^{b}; R^{b} being as disclosed above, preferably R^{b} being independently from each other, a substituent, which is identical or different, selected from the group consisting of halogen, cyano, C₁-C₆-alkyl, C₁-C₆-alkoxy, C₃-C₁₀-carbocyclyl, C₁-C₆-haloalkyl and aryl (preferably phenyl).

In some embodiments in accordance with embodiments 6, 7 or 8, R4 represents independently a substituent selected from the group consisting of halogen, cyano, hydroxy, C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl (e.g. cyclopropyl, cyclohexyl), 3- to 10-membered-heterocyclyl (e.g. dihydrooxazolyl, dihydropyridinyl, dihydrobenzofuranyl), C₁-C₆-haloalkyl, hydroxy-C₁-C₆-alkyl, aryl (e.g. phenyl), aryloxy, heteroaryl (e.g. imidazolyl, pyridinyl, pyrimidinyl, isoxazolyl, oxadiazolyl, pyrazolyl), —SF₅, —C(═O)R^{a}, —C(═O)OR^{a}, —C(═NR^{a})R^{a}, —N(R^{a})₂, —C(═NR^{a})N(R^{a})₂, —C₁-C₆-alkyl-N(R^{a})₂, —C₁-C₆-alkyl-C(═O)OR^{a}, —C(═O)N(R^{a})₂, —C(═O)NR^{a}N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a} with R^{a} as described herein. R4 may be substituted as described herein, preferably R4 represents independently a substituent selected from the group consisting of amino, C₁-C₆-alkylthio heteroaryl (e.g. imidazolyl, pyridinyl, pyrimidinyl, isoxazolyl, oxadiazolyl, pyrazolyl), nitro, —C(═O)R^{a}, —C(═O)OR^{a}, —N(R^{a})₂, —C(═O)N(R^{a})₂, —C(═O)NR^{a}N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂ and —NR^{a}S(═O)₂R^{a},

In embodiments 5, 6, 7 or 8, when R4 represents a heteroaryl, it preferably represents a 5- or 6-membered heteroaryl.

In embodiments 5, 6, 7 or 8, when R4 represents a 3- to 10-membered-heterocycle, it preferably represents a 5- or 6-membered saturated or partially saturated heterocyclyl.

In embodiments 6, 7 or 8, when R4 represents —O—C(═O)N(R^{a})₂ or —C(═O)N(R^{a})₂, R^{a} preferably represents a substituent, which is identical or different, selected from the group consisting of hydrogen, C₁-C₆-haloalkyl, C₃-C₁₀-carbocyclyl (e.g. C₃-C₁₀-cycloalkyl), 3- to 10-membered-heterocyclyl, —C₁-C₆-alkyl-C₃-C₁₀-carbocyclyl, —C₁-C₆-alkyl-aryl, aryl (e.g. phenyl) and heteroaryl (e.g. pyridinyl); wherein said C₁-C₆-haloalkyl, C₃-C₁₀-carbocyclyl, 3- to 10-membered-heterocyclyl, —C₁-C₆-alkyl-C₃-C₁₀-carbocyclyl, —C₁-C₆-alkyl-aryl, aryl (e.g. phenyl), heteroaryl substituent is itself optionally substituted, one or more times, with R^{b}, R^{b} being independently selected from the group consisting of halogen, cyano, C₁-C₆-alkyl, C₁-C₆-alkoxy, C₁-C₆-haloalkyl and aryl (preferably phenyl).

In embodiments 5, 6, 7 or 8, when R4 represents —N(R^{a})₂, R^{a} preferably represents a substituent, which is identical or different, selected from the group consisting of hydrogen, C₃-C₁₀-carbocyclyl, 3- to 10-membered-heterocyclyl, aryl and heteroaryl, wherein said C₃-C₁₀-carbocyclyl, 3- to 10-membered-heterocyclyl, aryl, heteroaryl substituent is itself optionally substituted, one or more times, with R^{b}, R^{b} being independently selected from the group consisting of halogen, cyano, C₁-C₆-alkyl, C₁-C₆-alkoxy, C₁-C₆-haloalkyl and aryl.

In embodiments 6, 7 or 8, when R4 resents —C₁-C₆-alkyl-NR^{a}C(═O)R^{a} or —NR^{a}C(═O)OR^{a}, R^{a} preferably represents a substituent, which is identical or different, selected from the group consisting of hydrogen, C₁-C₆-alkyl, C₁-C₆-haloalkyl, —C₁-C₆-alkyl-C₃-C₁₀-carbocyclyl, —C₁-C₆-alkyl-C₁-C₆-alkoxy, C₂-C₆-alkynyl, C₃-C₁₀-carbocyclyl, —C₁-C₆-alkyl-aryl and aryl (e.g. phenyl, napthenyl, fluorenyl); wherein said C₁-C₆-alkyl, C₁-C₆-haloalkyl, —C₁-C₆-alkyl-C₃-C₁₀-carbocyclyl, —C₁-C₆-alkyl-C₁-C₆-alkoxy, C₂-C₆-alkynyl, C₃-C₁₀-carbocyclyl, —C₁-C₆-alkyl-aryl, aryl substituent is itself optionally substituted, one or more times, in the same way or differently with R^{b}, R^{b} being preferably independently selected from the group consisting of halogen, C₁-C₆-alkyl, C₁-C₆-alkoxy, C₁-C₆-haloalkyl and aryl.

In embodiments 6, 7 or 8, when R4 represents —NR^{a}C(═O)R^{a}, R^{a} preferably represents a substituent, which is identical or different, selected from the group consisting of hydrogen, C₁-C₆-alkyl, C₁-C₆-haloalkyl, —C₁-C₆-alkyl-C₁-C₆-alkoxy, —C₁-C₆-alkyl-C₃-C₁₀-carbocyclyl, C₃-C₁₀-carbocyclyl (e.g. cycloalkyl, cycloalkenyl), 3- to 10-membered-heterocyclyl, —C₁-C₆-alkyl-aryl, aryl (e.g. phenyl) and heteroaryl; wherein said C₁-C₆-alkyl, C₁-C₆-haloalkyl, —C₁-C₆-alkyl-C₁-C₆-alkoxy, —C₁-C₆-alkyl-C₃-C₁₀-carbocyclyl, C₃-C₁₀-carbocyclyl, 3- to 10-membered-heterocyclyl, —C₁-C₆-alkyl-aryl, aryl, heteroaryl substituent is itself optionally substituted, one or more times, in the same way or differently with R^{b}, R^{b} being preferably independently selected from the group consisting of halogen, C₁-C₆-alkyl, C₁-C₆-alkoxy and cyano.

In some embodiments in accordance with embodiment 5, A is phenyl and R4 is phenyl optionally substituted with R5′ as described herein, preferably R5′ is selected from the group consisting of halogen, cyano, C₁-C₆-alkoxy, —C(═O)OR^{a} and —C(═O)N(R^{a})₂.

In some embodiments in accordance with embodiment 6, A is phenyl and R4 is phenyl optionally substituted with R5 as described herein.

In some embodiments in accordance with embodiment 5, A is phenyl and R4 is C₂-C₆-alkynyl optionally substituted with R5′ as described herein.

In some embodiments in accordance with embodiment 6, A is phenyl and R4 is C₂-C₆-alkynyl optionally substituted with R5 as described herein.

The above specified definitions of R1, R2, R3, R4, R5, R6, m and A (broad definitions as well as preferred, more preferred definitions or embodiments) can be combined in various manners to provide sub-classes of compounds according to the invention.

The compounds of formula (I′) in accordance with embodiments 5, 6, 7 and 8 are useful for controlling phytopathogenic fungi (use as fungicide). Thus, the present invention relates to the use of a compound of formula (I′) in accordance with embodiments 5 6, 7 and 8 for controlling phytopathogenic fungi.

The present invention also relates to any compounds of formula (I) disclosed in Table 1.

The present invention also relates to any compound of formula (1b) disclosed in Table 4.

### Processes for Preparing Compounds of Formula I

The following table lists the abbreviations used in this paragraph and in the Examples section as far as they are not explained within the text body. NMR peak forms are stated as they appear in the spectra, possible higher order effects have not been considered.

The compounds and intermediates produced according to the disclosed processes may require purification. Purification of organic compounds is well known to the person skilled in the art and there may be several ways of purifying the same compound. In some cases, no purification may be necessary. In some cases, the compounds may be purified by crystallisation. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel or Isolute® Flash NH₂ silica gel in combination with a Flashmaster II autopurifier (Argonaut/Biotage) and eluants such as gradients of hexane/EtOAc or DCM/ethanol. In some cases, the compounds may be purified by preparative HPLC using for example a Waters autopurifier equipped with a diode array detector and/or on-line electrospray ionization mass spectrometer in combination with a suitable prepacked reverse phase column and eluants such as gradients of water and acetonitrile which may contain additives such as trifluoroacetic acid or aqueous ammonia.

| Abbreviation | Meaning |
|---|---|
| Ac | Acetyl |
| AIBN | α,α′-azobisisobutyronitrile |
| Boc | tert-butyloxycarbonyl |
| Br | Broad |
| c- | cyclo- |
| CI | chemical ionisation |
| D | Doublet |
| Dd | doublet of doublet |
| dt | double triplet |
| DCM | Dichloromethane |
| DIPEA | N,N-diisopropylethyl amine |
| DMAP | N,N-dimethylaminopyridine |
| DMF | N,N-dimethylformamide |
| DMSO | dimethyl sulfoxide |
| EDCI | 1-(3-dimethylaminopropyl)-3-ethylcarbodiimide |
| | hydrochloride |
| eq. | Equivalent |
| ESI | electrospray ionisation |
| GP | general procedure |
| h | Hours |
| HATU | O-(7-Azabenzotriazol-1-yl)-N,N,N′,N′- |
| | tetramethyluronium hexafluorophosphate |
| HPLC | high performance liquid chromatography |
| LC-MS | liquid chromatography mass spectrometry |
| M | Multiplet |
| Mc | centred multiplet |
| MS | mass spectrometry |
| NMR | nuclear magnetic resonance spectroscopy: chemical |
| | shifts (δ) are given in ppm. |
| OTf | trifluoromethylsulfonyl- |
| Pg | protecting group |
| Ppm | parts per million |
| Q | Quartet |
| Rf | at reflux |
| r.t. or rt | room temperature |
| S | Singlet |
| sept. | Septet |
| T | Triplet |
| T3P | 1-propanephosphonic acid cyclic anhydride |
| TEA | Triethylamine |
| TFA | trifluoroacetic acid |
| THF | Tetrahydrofuran |

It is understood that when in aqueous media, the compounds of formula (I′) wherein R1 is hydrogen and R2, R3, R4, R5, R^{a}, R^{b}, R, n, m, p, X and Z are as defined hereinabove, may be present in a reversible equilibrium with the corresponding opened form (i.e. the compounds of formula (I′-I)).

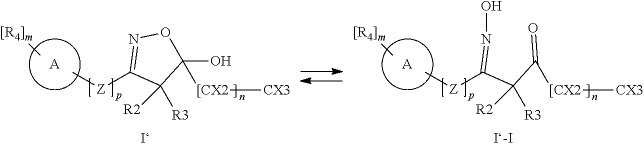

In the following, unless specified differently, R1, R2, R3, R4, R5, R^{a}, R^{b}, R^{c}, n, m, p, X and Z are as defined hereinabove.

### General Synthetic Routes:

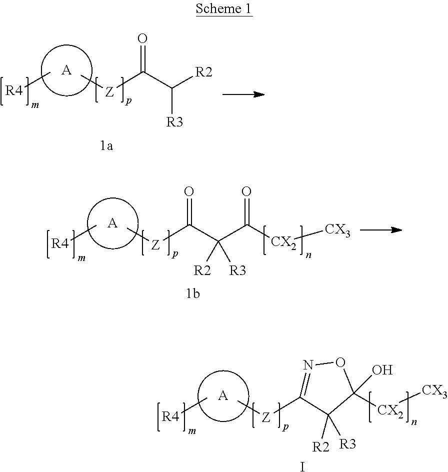

Ketones of formula 1a may be reacted with a suitable base, preferably in a solvent, for example sodium ethoxide in ethanol, followed by the addition of a fluoro-substituted acetyl electrophile, for example trifluoroacetic acid ethyl ester, to give intermediate substituted 1,3-diones of formula 1b. These, in turn, may be condensed with hydroxylamine to yield the substituted isoxazolines of formula I, for example by following literature procedures (J. Org. Chem., 1995, 60, 3907-3909).

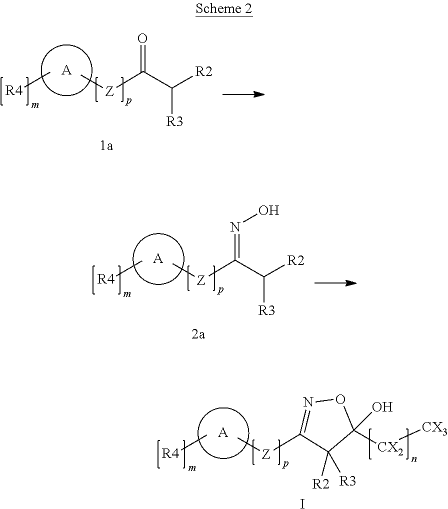

Ketones of formula 1a may be condensed with hydroxylamine to generate oximes of formula 2a. These, in turn, may be reacted with a suitable base, preferably in a solvent, for example n-butyllithium in THF, followed by the addition of a fluoro-substituted acetyl electrophile, for example trifluoroacetic acid ethyl ester, to give substituted isoxazolines of formula I, for example by following literature procedures (Bioorg. Med. Chem. Lett., 2005, 15, 5562-5566).

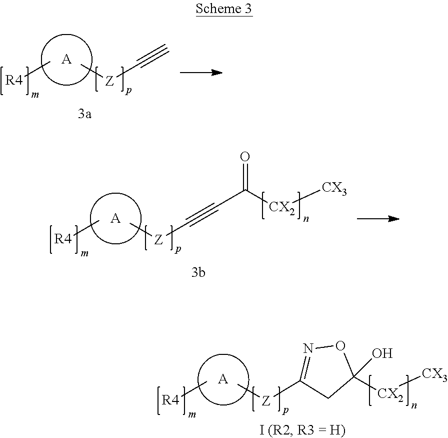

Monosubstituted acetylenes of formula 3a may be reacted with a suitable base, preferably in a solvent, for example n-butyllithium in THF, followed by the addition of a fluoro-substituted acetyl electrophile, for example trifluoroacetic acid ethyl ester, to give intermediate substituted alkynones of formula 3b. These, in turn, may be condensed with hydroxylamine to yield the substituted isoxazolines of formula I wherein R2, R3=H, for example by following literature procedures (Tetrahedron Lett., 1989, 30, 16, 2049-2052).

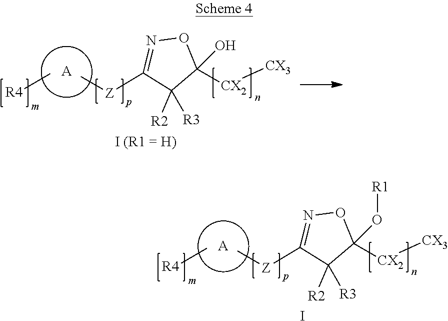

The above-generated substituted isoxazolines of formula I wherein R1=H may then be reacted with a suitable base, for example pyridine, followed by the addition of an electrophile, for example acetic anhydride, to give further substituted isoxazolines of formula I.

Compounds of general formula (I) supra may be prepared by a method A comprising the step of allowing a compound of general formula A:

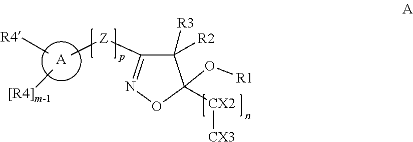

in which R4′ is selected from the group comprising hydroxy, mercapto, amino, C₁-C₆-alkylamino, hydroxy-C₁-C₆-alkyl, —C₁-C₆-alkyl-N(R^{a})₂, —C₁-C₆-alkyl-NR^{a}(OR^{a}), —N(R^{a})₂, preferably consisting of —OH, —NH₂ or —CH₂NH₂, and in which A, R1, R2, R3, R4, X, Z, m, n, and p, are given in each case supra,
to react with a compound of formula B

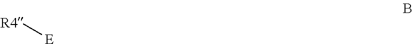

wherein R4″ represents a substituent selected from the group comprising, preferably consisting of C₁-C₆-alkyl, aryl, heteroaryl, R^{a}, —C(═O)R^{a}, —C(═O)OR^{a}, —C(═O)N(R^{a})₂, —C(═S)N(R^{a})₂, —S(═O)₂R^{a}, —S(═O)₂N(R^{a})₂, wherein said C₁-C₆-alkyl, aryl, heteroaryl, R^{a}, —C(═O)R^{a}, —C(═O)OR^{a}, —C(═O)N(R^{a})₂, —C(═S)N(R^{a})₂, —S(═O)₂R^{a}, —S(═O)₂N(R^{a})₂, substituent is itself optionally substituted, one or more times, in the same way or differently, with R5, wherein R^{a} and R5 are defined supra and E is a leaving group.
   to provide a compound of general formula I

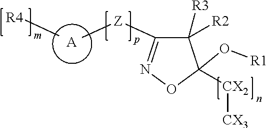

in which formula I, the definitions of A, R1, R2, R3, R4, X, Z, m, n, and p, are given for the main aspect, supra.

Alternatively compounds of general formula (I) supra may be prepared by a method B comprising the step of allowing a compound of general formula C:

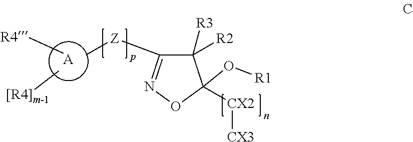

in which R4′″ is selected from the group comprising —C(═O)OR^{a}, —C₁-C₆-alkyl-C(═O)OR^{a}, —S(═O)₂R^{a}—C₁-C₆-alkyl-S(═O)₂R^{a}, preferably consisting of —COOH and in which A, R1, R2, R3, R4, X, Z, m, n, and p, are given in each case supra,
to react with a compound of formula D

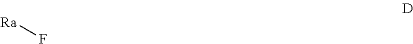

wherein R^{a} is defined supra and F is —NHR^{a}, —NH(OR^{a}) or —OH group.
to provide a compound of general formula I:

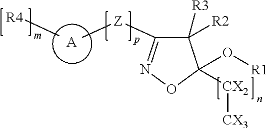

in which formula I, the definitions of A, R1, R2, R3, R4, X, Z, m, n, and p, are given for the main aspect, supra.

Alternatively compounds of general formula (I) supra may be prepared by a method C comprising the step of allowing a compound of general formula E:

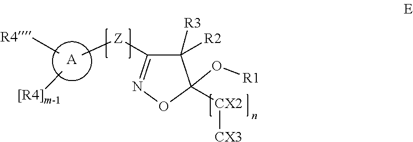

in which R4″″ is an halogen, preferably —Br and in which A, R1, R2, R3, X, R4, Z, m, n, and p, are given in each case supra,
to react with a compound of formula F:

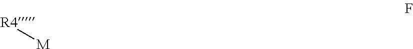

wherein R4′″″ is C₁-C₆-alkyl, aryl, heteroaryl, C₃-C₁₀-carbocyclyl, 3- to 10-membered-heterocyclyl wherein said C₁-C₆-alkyl, aryl, heteroaryl, C₃-C₁₀-carbocyclyl, 3- to 10-membered-heterocyclyl substituent is itself optionally substituted, one or more times, in the same way or differently, with R5, wherein R5 is defined supra and M is a metal or a metalloid (for example —B(OH)2 or —ZnCl)
   through a cross coupling reaction using a metal (for exemple palladium) in a suitable solvent to provide a compound of general formula I

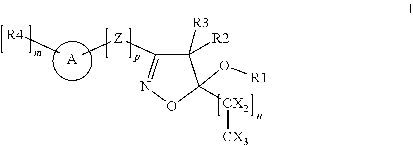

in which formula I, the definitions of A, R1, R2, R3, R4, X, Z, m, n, and p, are given for the main aspect, supra.

### Compositions and Formulations

The present invention further relates to a composition, in particular a composition for controlling unwanted microorganisms, comprising one or more compounds of formula (I′). The composition is preferably is a fungicidal composition.

The composition typically comprises one or more compounds of formula (I′) and one or more acceptable carriers, in particular one or more agriculturally acceptable carriers.

A carrier is a solid or liquid, natural or synthetic, organic or inorganic substance that is generally inert. The carrier generally improves the application of the compounds, for instance, to plants, plants parts or seeds. Examples of suitable solid carriers include, but are not limited to, ammonium salts, natural rock flours, such as kaolins, clays, talc, chalk, quartz, attapulgite, montmorillonite and diatomaceous earth, and synthetic rock flours, such as finely divided silica, alumina and silicates. Examples of typically useful solid carriers for preparing granules include, but are not limited to crushed and fractionated natural rocks such as calcite, marble, pumice, sepiolite and dolomite, synthetic granules of inorganic and organic flours and granules of organic material such as paper, sawdust, coconut shells, maize cobs and tobacco stalks. Examples of suitable liquid carriers include, but are not limited to, water, organic solvents and combinations thereof. Examples of suitable solvents include polar and nonpolar organic chemical liquids, for example from the classes of aromatic and nonaromatic hydrocarbons (such as cyclohexane, paraffins, alkylbenzenes, xylene, toluene alkylnaphthalenes, chlorinated aromatics or chlorinated aliphatic hydrocarbons such as chlorobenzenes, chloroethylenes or methylene chloride), alcohols and polyols (which may optionally also be substituted, etherified and/or esterified, such as butanol or glycol), ketones (such as acetone, methyl ethyl ketone, methyl isobutyl ketone or cyclohexanone), esters (including fats and oils) and (poly)ethers, unsubstituted and substituted amines, amides (such as dimethylformamide), lactams (such as N-alkylpyrrolidones) and lactones, sulfones and sulfoxides (such as dimethyl sulphoxide). The carrier may also be a liquefied gaseous extender, i.e. liquid which is gaseous at standard temperature and under standard pressure, for example aerosol propellants such as halohydrocarbons, butane, propane, nitrogen and carbon dioxide. The amount of carrier typically ranges from 1 to 99.99%, preferably from 5 to 99.9%, more preferably from 10 to 99.5%, and most preferably from 20 to 99% by weight of the composition.

The composition may further comprise one or more acceptable auxiliaries which are customary for formulating compositions (e.g. agrochemical compositions), such as one or more surfactants.

The surfactant can be an ionic (cationic or anionic) or non-ionic surfactant, such as ionic or non-ionic emulsifier(s), foam former(s), dispersant(s), wetting agent(s) and any mixtures thereof. Examples of suitable surfactants include, but are not limited to, salts of polyacrylic acid, salts of lignosulfonic acid, salts of phenolsulfonic acid or naphthalenesulfonic acid, polycondensates of ethylene and/or propylene oxide with fatty alcohols, fatty acids or fatty amines (polyoxyethylene fatty acid esters, polyoxyethylene fatty alcohol ethers, for example alkylaryl polyglycol ethers), substituted phenols (preferably alkylphenols or arylphenols), salts of sulfosuccinic esters, taurine derivatives (preferably alkyl taurates), phosphoric esters of polyethoxylated alcohols or phenols, fatty esters of polyols and derivatives of compounds containing sulfates, sulfonates, phosphates (for example, alkylsulfonates, alkyl sulfates, arylsulfonates) and protein hydrolysates, lignosulfite waste liquors and methylcellulose. A surfactant is typically used when the compound of the formula (I) and/or the carrier is insoluble in water and the application is made with water. Then, the amount of surfactants typically ranges from 5 to 40% by weight of the composition.

Further examples of auxiliaries which are customary for formulating agrochemical compositions include water repellents, siccatives, binders (adhesive, tackifier, fixing agent, such as carboxymethylcellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone, polyvinyl acetate, polyvinyl alcohol and tylose), thickeners, stabilizers (e.g. cold stabilizers, preservatives, antioxidants, light stabilizers, or other agents which improve chemical and/or physical stability), dyes or pigments (such as inorganic pigments, e.g. iron oxide, titanium oxide and Prussian Blue; organic dyes, e.g. alizarin, azo and metal phthalocyanine dyes), antifoams (e.g. silicone antifoams and magnesium stearate), preservatives (e.g. dichlorophene and benzyl alcohol hemiformal), secondary thickeners (cellulose derivatives, acrylic acid derivatives, xanthan, modified clays and finely divided silica), stickers, gibberellins and processing auxiliaries, mineral and vegetable oils, perfumes, waxes, nutrients (including trace nutrients, such as salts of iron, manganese, boron, copper, cobalt, molybdenum and zinc), protective colloids, thixotropic substances, penetrants, sequestering agents and complex formers.

The choice of the auxiliaries is related to the intended mode of application of the compound of the formula (I′) and/or on the physical properties. Furthermore, the auxiliaries may be chosen to impart particular properties (technical, physical and/or biological properties) to the compositions or use forms prepared therefrom. The choice of auxiliaries may allow customizing the compositions to specific needs.

The composition of the invention may be in any customary form, such as solutions (e.g aqueous solutions), emulsions, wettable powders, water- and oil-based suspensions, powders, dusts, pastes, soluble powders, soluble granules, granules for broadcasting, suspoemulsion concentrates, natural or synthetic products impregnated with the compound of the invention, fertilizers and also microencapsulations in polymeric substances. The compound of the invention may be present in a suspended, emulsified or dissolved form.

The composition of the invention may be provided to the end user as ready-for-use formulation, i.e. the compositions may be directly applied to the plants or seeds by a suitable device, such as a spraying or dusting device. Alternatively, the compositions may be provided to the end user in the form of concentrates which have to be diluted, preferably with water, prior to use.

The composition of the invention can be prepared in conventional manners, for example by mixing the compound of the invention with one or more suitable auxiliaries, such as disclosed herein above.

The composition according to the invention contains generally from 0.01 to 99% by weight, from 0.05 to 98% by weight, preferably from 0.1 to 95% by weight, more preferably from 0.5 to 90% by weight, most preferably from 1 to 80% by weight of the compound of the invention.

The compound and the composition of the invention can be mixed with other active ingredients like fungicides, bactericides, acaricides, nematicides, insecticides, herbicides, fertilizers, growth regulators, safeners or semiochemicals. This may allow to broaden the activity spectrum or to prevent development of resistance. Examples of known fungicides, insecticides, acaricides, nematicides and bactericides are disclosed in the Pesticide Manual, 17th Edition.

Examples of especially preferred fungicides which could be mixed with the compound and the composition of the invention are:

1) Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)-ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Ipfentrifluconazole.

2) Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034)N-(2-cyclopentyl-5-fluorobenzyl)-N-cyclopropyl-3-(difluoromethyl)-5-fluoro-1-methyl-1H-pyrazole-4-carboxamide, (2.035)N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-iH-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-1H-pyrazole-4-carboxamide, (2.053)N-cyclopropyl-3-(difluoromethyl)-N-(2-ethyl-5-methylbenzyl)-5-fluoro-1-methyl-1H-pyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055)N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide.

3) Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)-methyl]phenyl}-2-methoxy-N-methylacetamide, (3.027)N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate.

4) Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluoro-phenyl)-N-(2-chloro-6-fluorophenyl)-1,3-dimethyl-1H-pyrazol-5-amine, (4.016) 4-(2-bromo-4-fluoro-phenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluoro-phenyl)-1,3-dimethyl-1H-pyrazol-5-amine, (4.019) 4-(2-chloro-4-fluorophenyl)-N-(2-chloro-6-fluoro-phenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine.

5) Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.

6) Compounds capable to induce a host defence, for example (6.001) acibenzolar-S-methyl, (6.002) isotianil, (6.003) probenazole, (6.004) tiadinil.

7) Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

8) Inhibitors of the ATP production, for example (8.001) silthiofam.

9) Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

10) Inhibitors of the lipid and membrane synthesis, for example (10.001) propamocarb, (10.002) propamocarb hydrochloride, (10.003) tolclofos-methyl.

11) Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.

12) Inhibitors of the nucleic acid synthesis, for example (12.001) benalaxyl, (12.002) benalaxyl-M (kiralaxyl), (12.003) metalaxyl, (12.004) metalaxyl-M (mefenoxam).

13) Inhibitors of the signal transduction, for example (13.001) fludioxonil, (13.002) iprodione, (13.003) procymidone, (13.004) proquinazid, (13.005) quinoxyfen, (13.006) vinclozolin.

14) Compounds capable to act as an uncoupler, for example (14.001) fluazinam, (14.002) meptyldinocap.

15) Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoro-methyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one.

All named mixing partners of the classes (1) to (15) as described here above can be present in the form of the free compound and/or, if their functional groups enable this, an agriculturally acceptable salt thereof.

### Methods and Uses

The compounds of formula (I) or (I′) and the compositions of the invention have potent microbicidal activity. They can be used for controlling unwanted microorganisms, such as unwanted fungi and bacteria. They can be particularly useful in crop protection (they control microorganisms that cause plants diseases) or for protecting materials (e.g. industrial materials, timber, storage goods) as described in more details herein below. More specifically, the compounds of formula (I) or (I′) and the composition of the invention can be used to protect seeds, germinating seeds, emerged seedlings, plants, plant parts, fruits, harvest goods and/or the soil in which the plants grow from unwanted microorganisms.

Control or controlling as used herein encompasses protective, curative and eradicative treatment of unwanted microorganisms. Unwanted microorganisms may be pathogenic bacteria, pathogenic virus, pathogenic oomycetes or pathogenic fungi, more specifically phytopathogenic bacteria phytopathogenic virus, phytopathogenic oomycetes or phytopathogenic fungi. As detailed herein below, these phytopathogenic microorganims are the causal agents of a broad spectrum of plants diseases.

More specifically, the compound of formula (I) or (I′) and the composition of the invention can be used as fungicides. For the purpose of the specification, the term “fungicide” refers to a compound or composition that can be used in crop protection for the control of unwanted fungi, such as Plasmodiophoromycetes, Chytridiomycetes, Zygomycetes, Ascomycetes, Basidiomycetes and Deuteromycetes and/or for the control of Oomycetes, more preferably Basidiomycetes.

The present invention also relates to a method for controlling unwanted phytopathogenic microorganisms, such as unwanted fungi, oomycetes and bacteria, comprising the step of applying at least one compound of formula (I) or (I′) or at least one composition of the invention to the microorganisms and/or their habitat (to the plants, plant parts, seeds, fruits or to the soil in which the plants grow).

Typically, when the compound and the composition of the invention are used in curative or protective methods for controlling phytopathogenic fungi and/or phytopathogenic oomycetes, an effective and plant-compatible amount thereof is applied to the plants, plant parts, fruits, seeds or to the soil or substrates in which the plants grow. Suitable substrates that may be used for cultivating plants include inorganic based substrates, such as mineral wool, in particular stone wool, perlite, sand or gravel; organic substrates, such as peat, pine bark or sawdust; and petroleum based substrates such as polymeric foams or plastic beads. Effective and plant-compatible amount means an amount that is sufficient to control or destroy the fungi present or liable to appear on the cropland and that does not entail any appreciable symptom of phytotoxicity for said crops. Such an amount can vary within a wide range depending on the fungus to be controlled, the type of crop, the crop growth stage, the climatic conditions and the respective compound or composition of the invention used. This amount can be determined by systematic field trials that are within the capabilities of a person skilled in the art.

### Plants and Plant Parts

The compound of formula (I) or (I′) and the composition of the invention may be applied to any plants or plant parts.

Plants mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the genetically modified plants (GMO or transgenic plants) and the plant cultivars which are protectable and non-protectable by plant breeders' rights.

Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. The plant parts also include harvested material and vegetative and generative propagation material, for example cuttings, tubers, rhizomes, slips and seeds.

Plants which may be treated in accordance with the methods of the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

In some preferred embodiments, wild plant species and plant cultivars, or those obtained by conventional biological breeding methods, such as crossing or protoplast fusion, and also parts thereof, are treated in accordance with the methods of the invention.

In some other preferred embodiments, transgenic plants and plant cultivars obtained by genetic engineering methods, if appropriate in combination with conventional methods (Genetically Modified Organisms), and parts thereof are treated in accordance with the methods of the invention. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The methods according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plants and plant cultivars which can be treated by the above disclosed methods include all plants which have genetic material which impart particularly advantageous, useful traits to these plants (whether obtained by breeding and/or biotechnological means).

Plants and plant cultivars which can be treated by the above disclosed methods include plants and plant cultivars which are resistant against one or more biotic stresses, i.e. said plants show a better defense against animal and microbial pests, such as against nematodes, insects, mites, phytopathogenic fungi, bacteria, viruses and/or viroids.

Plants and plant cultivars which can be treated by the above disclosed methods include those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which can be treated by the above disclosed methods include those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants and plant cultivars which can be treated by the above disclosed methods include plants and plant cultivars which are hybrid plants that already express the characteristic of heterosis or hybrid vigor which results in generally higher yield, vigor, health and resistance towards biotic and abiotic stresses. Plants and plant cultivars (obtained by plant biotechnology methods such as genetic engineering) which can be treated by the above disclosed methods include plants and plant cultivars which are herbicide-tolerant plants, i.e. plants made tolerant to one or more given herbicides. Such plants can be obtained either by genetic transformation, or by selection of plants containing a mutation imparting such herbicide tolerance.

Plants and plant cultivars (obtained by plant biotechnology methods such as genetic engineering) which can be treated by the above disclosed methods include plants and plant cultivars which are insect-resistant transgenic plants, i.e. plants made resistant to attack by certain target insects. Such plants can be obtained by genetic transformation, or by selection of plants containing a mutation imparting such insect resistance.

Plants and plant cultivars (obtained by plant biotechnology methods such as genetic engineering) which can be treated by the above disclosed methods include plants and plant cultivars which are tolerant to abiotic stresses. Such plants can be obtained by genetic transformation, or by selection of plants containing a mutation imparting such stress resistance.

Plants and plant cultivars (obtained by plant biotechnology methods such as genetic engineering) which can be treated by the above disclosed methods include plants and plant cultivars which show altered quantity, quality and/or storage-stability of the harvested product and/or altered properties of specific ingredients of the harvested product.

Plants and plant cultivars (obtained by plant biotechnology methods such as genetic engineering) which can be treated by the above disclosed methods include plants and plant cultivars, such as cotton plants, with altered fiber characteristics. Such plants can be obtained by genetic transformation, or by selection of plants contain a mutation imparting such altered fiber characteristics.

Plants and plant cultivars (obtained by plant biotechnology methods such as genetic engineering) which can be treated by the above disclosed methods include plants and plant cultivars, such as oilseed rape or related Brassica plants, with altered oil profile characteristics. Such plants can be obtained by genetic transformation, or by selection of plants contain a mutation imparting such altered oil profile characteristics.

Plants and plant cultivars (obtained by plant biotechnology methods such as genetic engineering) which can be treated by the above disclosed methods include plants and plant cultivars, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants and plant cultivars (obtained by plant biotechnology methods such as genetic engineering) which can be treated by the above disclosed methods include plants and plant cultivars, such as Tobacco plants, with altered post-translational protein modification patterns.

### Pathogens and Diseases

The methods disclosed above can be used to control microorganisms, in particular phytopathogenic microorganisms such as phytopathogenic fungi, causing diseases, such as:

diseases caused by powdery mildew pathogens, such as Blumeria species (e.g. Blumeria graminis), Podosphaera species (e.g. Podosphaera leucotricha), Sphaerotheca species (e.g. Sphaerotheca fuliginea), Uncinula species (e.g. Uncinula necator);
diseases caused by rust disease pathogens, such as Gymnosporangium species (e.g. Gymnosporangium sabinae), Hemileia species (e.g. Hemileia vastatrix), Phakopsora species (e.g. Phakopsora pachyrhizi or Phakopsora meibomiae), Puccinia species (e.g. Puccinia recondita, Puccinia graminis or Puccinia striiformis), Uromyces species (e.g. Uromyces appendiculatus);
diseases caused by pathogens from the group of the Oomycetes, such as Albugo species (e.g. Albugo candida), Bremia species (e.g. Bremia lactucae), Peronospora species (e.g. Peronospora pisi or P. brassicae), Phytophthora species (e.g. Phytophthora infestans), Plasmopara species (e.g. Plasmopara viticola), Pseudoperonospora species (e.g. Pseudoperonospora humuli or Pseudoperonospora cubensis), Pythium species (e.g. Pythium ultimum);
leaf blotch diseases and leaf wilt diseases caused, for example, by Alternaria species (e.g. Alternaria solani), Cercospora species (e.g. Cercospora beticola), Cladiosporium species (e.g. Cladiosporium cucumerinum), Cochliobolus species (e.g. Cochliobolus sativus (conidial form: Drechslera, syn: Helminthosporium) or Cochliobolus miyabeanus), Colletotrichum species (e.g. Colletotrichum lindemuthanium), Cycloconium species (e.g. Cycloconium oleaginum), Diaporthe species (e.g. Diaporthe citri), Elsinoe species (e.g. Elsinoe fawcettii), Gloeosporium species (e.g. Gloeosporium laeticolor), Glomerella species (e.g. Glomerella cingulate), Guignardia species (e.g. Guignardia bidwelli), Leptosphaeria species (e.g. Leptosphaeria maculans), Magnaporthe species (e.g. Magnaporthe grisea), Microdochium species (e.g. Microdochium nivale), Mycosphaerella species (e.g. Mycosphaerella graminicola, Mycosphaerella arachidicola or Mycosphaerella fijiensis), Phaeosphaeria species (e.g. Phaeosphaeria nodorum), Pyrenophora species (e.g. Pyrenophora teres or Pyrenophora tritici repentis), Ramularia species (e.g. Ramularia collo-cygni or Ramularia areola), Rhynchosporium species (e.g. Rhynchosporium secalis), Septoria species (e.g. Septoria apii or Septoria lycopersici), Stagonospora species (e.g. Stagonospora nodorum), Typhula species (e.g. Typhula incarnate), Venturia species (e.g. Venturia inaequalis),
root and stem diseases caused, for example, by Corticium species (e.g. Corticium graminearum), Fusarium species (e.g. Fusarium oxysporum), Gaeumannomyces species, (e.g. Gaeumannomyces graminis), Plasmodiophora species, (e.g. Plasmodiophora brassicae), Rhizoctonia species, (e.g. Rhizoctonia solani), Sarocladium species, (e.g. Sarocladium oryzae), Sclerotium species, (e.g. Sclerotium oryzae), Tapesia species, (e.g. Tapesia acuformis), Thielaviopsis species, (e.g. Thielaviopsis basicola);
ear and panicle diseases (including corn cobs) caused, for example, by Alternaria species, (e.g. Alternaria spp.), Aspergillus species (e.g. Aspergillus flavus), Cladosporium species (e.g. Cladosporium cladosporioides, Claviceps species (e.g. Claviceps purpurea), Fusarium species, (e.g. Fusarium culmorum), Gibberella species (e.g. Gibberella zeae), Monographella species, (e.g. Monographella nivalis), Stagnospora species, (e.g. Stagnospora nodorum);
diseases caused by smut fungi, for example Sphacelotheca species (e.g. Sphacelotheca reiliana), Tilletia species (e.g. Tilletia caries or Tilletia controversa), Urocystis species (e.g. Urocystis occulta), Ustilago species (e.g. Ustilago nuda);
fruit rot caused, for example, by Aspergillus species (e.g. Aspergillus flavus), Botrytis species (e.g. Botrytis cinerea), Penicillium species (e.g. Penicillium expansum or Penicillium purpurogenum), Rhizopus species (e.g. Rhizopus stolonifer), Sclerotinia species (e.g. Sclerotinia sclerotiorum), Verticilium species (e.g. Verticilium alboatrum);
seed- and soil-borne rot and wilt diseases, and also diseases of seedlings, caused, for example, by Alternaria species (e.g. Alternaria brassicicola), Aphanomyces species (e.g. Aphanomyces euteiches), Ascochyta species (e.g. Ascochyta lentis), Aspergillus species (e.g. Aspergillus flavus), Cladosporium species (e.g. Cladosporium herbarum), Cochliobolus species (e.g. Cochliobolus sativus (conidial form: Drechslera, Bipolaris Syn: Helminthosporium)), Colletotrichum species (e.g. Colletotrichum coccodes), Fusarium species (e.g. Fusarium culmorum), Gibberella species (e.g. Gibberella zeae), Macrophomina species (e.g. Macrophomina phaseolina), Microdochium species (e.g. Microdochium nivale), Monographella species (e.g. Monographella nivalis), Penicillium species (e.g. Penicillium expansum), Phoma species (e.g. Phoma lingam), Phomopsis species (e.g. Phomopsis sojae), Phytophthora species (e.g. Phytophthora cactorum), Pyrenophora species (e.g. Pyrenophora graminea), Pyricularia species (e.g. Pyricularia oryzae), Pythium species (e.g. Pythium ultimum), Rhizoctonia species (e.g. Rhizoctonia solani), Rhizopus species (e.g. Rhizopus oryzae), Sclerotium species (e.g. Sclerotium rolfsii), Septoria species (e.g. Septoria nodorum), Typhula species (e.g. Typhula incarnate), Verticillium species (e.g. Verticillium dahlia);
cancers, galls and witches' broom caused, for example, by Nectria species (e.g. Nectria galligena); wilt diseases caused, for example, by Monilinia species (e.g. Monilinia laxa);
deformations of leaves, flowers and fruits caused, for example, by Exobasidium species (e.g. Exobasidium vexans), Taphrina species (e.g. Taphrina deformans);
degenerative diseases in woody plants, caused, for example, by Esca species (e.g. Phaeomoniella chlamydospora, Phaeoacremonium aleophilum or Fomitiporia mediterranea), Ganoderma species (e.g. Ganoderma boninense);
diseases of flowers and seeds caused, for example, by Botrytis species (e.g. Botrytis cinerea); diseases of plant tubers caused, for example, by Rhizoctonia species (e.g. Rhizoctonia solani), Helminthosporium species (e.g. Helminthosporium solani);
diseases caused by bacterial pathogens, for example Xanthomonas species (e.g. Xanthomonas campestris pv. Oryzae), Pseudomonas species (e.g. Pseudomonas syringae pv. Lachrymans), Erwinia species (e.g. Erwinia amylovora).

### Seed Treatment

The method for controlling unwanted microorganisms may be used to protect seeds from phytopathogenic microorganisms, such as fungi.

The term “seed(s)” as used herein include dormant seed, primed seed, pregerminated seed and seed with emerged roots and leaves.

Thus, the present invention also relates to a method for protecting seeds and/or crops from unwanted microorganisms, such as bacteria or fungi, which comprises the step of treating the seeds with one or more compounds of formula (I) or (I′) or a composition comprising thereof. The treatment of seeds with the compound(s) of formula (I) or (I′) or a composition comprising thereof not only protects the seeds from phytopathogenic microorganisms, but also the germinating plants, the emerged seedlings and the plants after emergence.

The seeds treatment may be performed prior to sowing, at the time of sowing or shortly thereafter.

When the seeds treatment is performed prior to sowing (e.g. so-called on-seed applications), the seeds treatment may be performed as follows: the seeds may be placed into a mixer with a desired amount of compound(s) of formula (I) or (I′) or a composition comprising thereof (either as such or after dilution), the seeds and the compound(s) of formula (I) or (I′) or the composition comprising thereof are mixed until a homogeneous distribution on seeds is achieved. If appropriate, the seeds may then be dried.

The invention also relates to seeds treated with one or more compounds of formula (I) or (I′) or a composition comprising thereof. As said before, the use of treated seeds allows not only protecting the seeds before and after sowing from unwanted microorganisms, such as phytopathogenic fungi, but also allows protecting the germinating plants and young seedlings emerging from said treated seeds. A large part of the damage to crop plants caused by harmful organisms is triggered by the infection of the seeds before sowing or after germination of the plant. This phase is particularly critical since the roots and shoots of the growing plant are particularly sensitive, and even small damage may result in the death of the plant.

Therefore, the present invention also relates to a method for protecting seeds, germinating plants and emerged seedlings, more generally to a method for protecting crop from phytopathogenic microorganisms, which comprises the step of using seeds treated by one or more compounds of formula (I) or (I′) or a composition comprising thereof.

Preferably, the seed is treated in a state in which it is sufficiently stable for no damage to occur in the course of treatment. In general, seeds can be treated at any time between harvest and shortly after sowing. It is customary to use seeds which have been separated from the plant and freed from cobs, shells, stalks, coats, hairs or the flesh of the fruits. For example, it is possible to use seeds which have been harvested, cleaned and dried down to a moisture content of less than 15% by weight. Alternatively, it is also possible to use seeds which, after drying, for example, have been treated with water and then dried again, or seeds just after priming, or seeds stored in primed conditions or pregerminated seeds, or seeds sown on nursery trays, tapes or paper.

The amount of compound(s) of formula (I) or (I′) or composition comprising thereof applied to the seed is typically such that the germination of the seed is not impaired, or that the resulting plant is not damaged. This must be ensured particularly in case the active ingredients would exhibit phytotoxic effects at certain application rates. The intrinsic phenotypes of transgenic plants should also be taken into consideration when determining the amount of compound(s) of formula (I) or (I′) or composition comprising thereof to be applied to the seed in order to achieve optimum seed and germinating plant protection with a minimum amount of compound(s) of formula (I) or (I′) or composition comprising thereof being employed.

As indicated above, the compounds of the formula (I) or (I′) can be applied, as such, directly to the seeds, i.e. without the use of any other components and without having been diluted, or a composition comprising the compounds of formula (I) or (I′) can be applied. Preferably, the compositions are applied to the seed in any suitable form. Examples of suitable formulations include solutions, emulsions, suspensions, powders, foams, slurries or combined with other coating compositions for seed, such as film forming materials, pelleting materials, fine iron or other metal powders, granules, coating material for inactivated seeds, and also ULV formulations. The formulations may be ready-to-use formulations or may be concentrates that need to be diluted prior to use.

These formulations are prepared in a known manner, for instance by mixing the active ingredient or mixture thereof with customary additives, for example customary extenders and solvents or diluents, dyes, wetting agents, dispersants, emulsifiers, antifoams, preservatives, secondary thickeners, adhesives, gibberellins, and also water.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1. Useful wetting agents which may be present in the seed dressing formulations are all substances which promote wetting and which are conventionally used for the formulation of active agrochemical ingredients. Usable with preference are alkylnaphthalenesulfonates, such as diisopropyl- or diisobutylnaphthalenesulfonates. Useful dispersants and/or emulsifiers which may be present in the seed dressing formulations are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulfated derivatives thereof. Suitable anionic dispersants are especially lignosulfonates, polyacrylic acid salts and arylsulfonate/formaldehyde condensates. Antifoams which may be present in the seed dressing formulations are all foam-inhibiting substances conventionally used for the formulation of active agrochemical ingredients. Silicone antifoams and magnesium stearate can be used with preference. Preservatives which may be present in the seed dressing formulations are all substances usable for such purposes in agrochemical compositions. Examples include dichlorophene and benzyl alcohol hemiformal. Secondary thickeners which may be present in the seed dressing formulations are all substances usable for such purposes in agrochemical compositions. Preferred examples include cellulose derivatives, acrylic acid derivatives, xanthan, modified clays and finely divided silica. Adhesives which may be present in the seed dressing formulations are all customary binders usable in seed dressing products. Preferred examples include polyvinylpyrrolidone, polyvinyl acetate, polyvinyl alcohol and tylose.

The compounds of the formula (I) or (I′) and the compositions comprising thereof are suitable for protecting seeds of any plant variety which is used in agriculture, in greenhouses, in forests or in horticulture. More particularly, the seed is that of cereals (such as wheat, barley, rye, millet, triticale, and oats), oilseed rape, maize, cotton, soybean, rice, potatoes, sunflower, beans, coffee, peas, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

The compounds of formula (I) or (I′) or the compositions comprising thereof can be used for treating transgenic seeds, in particular seeds of plants capable of expressing a protein which acts against pests, herbicidal damage or abiotic stress, thereby increasing the protective effect. Synergistic effects may also occur in interaction with the substances formed by expression.

### Application

The compound of the invention can be applied as such, or for example in the form of as ready-to-use solutions, emulsions, water- or oil-based suspensions, powders, wettable powders, pastes, soluble powders, dusts, soluble granules, granules for broadcasting, suspoemulsion concentrates, natural products impregnated with the compound of the invention, synthetic substances impregnated with the compound of the invention, fertilizers or microencapsulations in polymeric substances.

Application is accomplished in a customary manner, for example by watering, spraying, atomizing, broadcasting, dusting, foaming, spreading-on and the like. It is also possible to deploy the compound of the invention by the ultra-low volume method, via a drip irrigation system or drench application, to apply it in-furrow or to inject it into the soil stem or trunk. It is further possible to apply the compound of the invention by means of a wound seal, paint or other wound dressing.

The effective and plant-compatible amount of the compound of the invention which is applied to the plants, plant parts, fruits, seeds or soil will depend on various factors, such as the compound/composition employed, the subject of the treatment (plant, plant part, fruit, seed or soil), the type of treatment (dusting, spraying, seed dressing), the purpose of the treatment (curative and protective), the type of microorganisms, the development stage of the microorganisms, the sensitivity of the microorganisms, the crop growth stage and the environmental conditions.

When the compound of the invention is used as a fungicide, the application rates can vary within a relatively wide range, depending on the kind of application. For the treatment of plant parts, such as leaves, the application rate may range from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used). For the treatment of seeds, the application rate may range from 0.1 to 200 g per 100 kg of seeds, preferably from 1 to 150 g per 100 kg of seeds, more preferably from 2.5 to 25 g per 100 kg of seeds, even more preferably from 2.5 to 12.5 g per 100 kg of seeds. For the treatment of soil, the application rate may range from 0.1 to 10 000 g/ha, preferably from 1 to 5000 g/ha.

These application rates are merely examples and are not intended to limit the scope of the present invention.

### Material Protection

The compound and the composition of the invention may also be used in the protection of materials, especially for the protection of industrial materials against attack and destruction by unwanted microorganisms.

In addition, the compound and the composition of the invention may be used as antifouling compositions, alone or in combinations with other active ingredients.

Industrial materials in the present context are understood to mean inanimate materials which have been prepared for use in industry. For example, industrial materials which are to be protected from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compound and the composition of the invention may prevent adverse effects, such as rotting, decay, discoloration, decoloration or formation of mould.

In the case of treatment of wood the compound and the composition of the invention may also be used against fungal diseases liable to grow on or inside timber.

Timber means all types of species of wood, and all types of working of this wood intended for construction, for example solid wood, high-density wood, laminated wood, and plywood. In addition, the compound and the composition of the invention may be used to protect objects which come into contact with saltwater or brackish water, especially hulls, screens, nets, buildings, moorings and signalling systems, from fouling.

The compound and the composition of the invention may also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, may be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The compound and the composition of the invention may prevent adverse effects, such as rotting, decay, discoloration, decoloration or formation of mould.

Microorganisms capable of degrading or altering industrial materials include, for example, bacteria, fungi, yeasts, algae and slime organisms. The compound and the composition of the invention preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Aspects of the present teaching may be further understood in light of the following examples, which should not be construed as limiting the scope of the present teaching in anyway.

### Examples

In the subsequent paragraphs detailed procedures for the synthesis of intermediates for compounds of the present invention are described.

### Synthesis of Intermediates of Formula (1a)

### 1-(4-Bromomethyl-phenyl)-ethanone

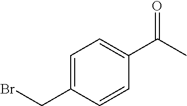

A solution of 1-p-tolyl-ethanone (16 ml, 120 mmol) in 1-butyl-3-methyl-3H-imidazol-1-ium hexafluorophosphate (20 ml) was degassed with a stream of Argon and sonication for 8 minutes. N-Bromosuccinimide (25.6 g, 144 mmol) and AIBN (0.97 g, 5.91 mmol) were added to the reaction mixture, which was similarly degassed for a further 8 minutes and then stirred at 60° C. for 2½ h. The reaction mixture was cooled, extracted with diethyl ether, and the organic phase washed with brine, dried (Na₂SO₄), filtered and concentrated in vacuo to give crude 1-(4-Bromomethyl-phenyl)-ethanone (27.3 g) which was used without further purification.

¹H-NMR (CDCl₃): δ=2.58 (m, 3H); 4.51 (s, 2H); 7.48 (d, 2H); 7.94 (d, 2H) ppm.

### 1-(4-Azidomethyl-phenyl)-ethanone

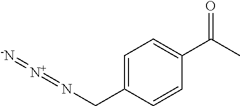

Sodium azide (8.43 g, 130 mmol) was added to a solution of crude 1-(4-Bromomethyl-phenyl)-ethanone in DMF (187 ml) at 0° C. After 3 h at 0° C., the reaction mixture was warmed to room temperature and stirred for a further 2 h, before being diluted with water and extracted with ethyl acetate. The organic phase was separated, washed with brine, dried (Na₂SO₄), filtered and concentrated in vacuo and the residue purified by flash column chromatography (hexane/ethyl acetate) to give 1-(4-Azidomethyl-phenyl)-ethanone (14.7 g, 70% over two steps). ¹H-NMR (CDCl₃): δ=2.62 (s, 3H); 4.42 (s, 2H); 7.41 (d, 2H); 7.97 (d, 2H) ppm.

### 1-[4-(3-Ethyl-1,2,4-oxadiazol-5-yl)phenyl]ethanone

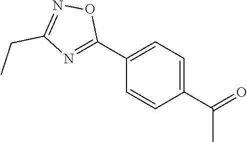 [SMILES: CCc1noc(-c2ccc(C(C)=O)cc2)n1]

Carbonyldiimidazole (1.09 g, 6.7 mmol) was added to a solution of 4-acetylbenzoic acid (1.0 g, 6.1 mmol) in acetonitrile (10 mL). The reaction mixture was stirred at 40° C. for 7 h. It was then cooled down to rt and left standing overnight. N-Hydroxypropanamidine hydrochloride (0.91 g, 7.3 mmol) was then added and the reaction mixture was heated at 140° C. under microwave irradiation for 45 min. The reaction mixture was then concentrated under reduced pressure, purified by column chromatography eluting with 50% EtOAc in heptane. Further purification by prep HPLC afforded 1-[4-(3-ethyl-1,2,4-oxadiazol-5-yl)phenyl]ethanone (198 mg, 15% yield).

MS (ESI): 217 ([M+H]⁺)

### 4-Acetyl-N-methyl-N-phenylbenzamide

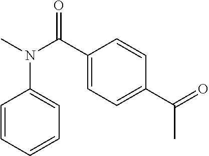 [SMILES: CC(=O)c1ccc(C(=O)N(C)c2ccccc2)cc1]

A solution of 4-acetylbenzoic acid (1.1 g, 6.8 mmol), N-Methylaniline (0.87 g, 8.2 mmol), (1-[Bis(dimethylamino)methylene]-1H-1,2,3-triazolo[4,5-b]pyridinium 3-oxid hexafluorophosphate) (2.6 g, 6.8 mmol) and DIPEA (2.4 mL, 13.6 mmol) in dichloromethane (6 mL) was stirred at 40° C. for 1 h. The reaction mixture was then concentrated under reduced pressure and purified by column chromatography eluting with 50% EtOAc in heptane to afford 4-acetyl-N-methyl-N-phenylbenzamide (1.5 g, 82% yield).

MS (ESI): 254 ([M+H]⁺)

### Synthesis of Intermediates of Formula (1b)

### 1-(4-Azidomethyl-phenyl)-4,4,4-trifluoro-butane-1,3-dione

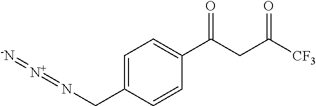

A solution of 1-(4-Azidomethyl-phenyl)-ethanone (10.5 g, 60.0 mmol) in ethanol (18 ml) was added slowly to a suspension of sodium (3.22 g, 140 mmol) in ethanol (110 ml), followed by dropwise addition of trifluoro-acetic acid ethyl ester (10.8 ml, 90.1 mmol). After stirring for 2 h, the reaction mixture was quenched with a 1N solution of aqueous hydrochloric acid and extracted with ethyl acetate. The organic phase was washed with water then brine, dried (Na₂SO₄), filtered and concentrated in vacuo to give crude 1-(4-Azidomethyl-phenyl)-4,4,4-trifluoro-butane-1,3-dione (18.4 g) which was used without further purification.

¹H-NMR (CDCl₃): δ=4.47 (s, 2H); 6.59 (s, 1H); 7.47 (d, 2H); 7.97 (d, 2H) ppm.

### 1-[4-(2,5-dimethyl-1H-pyrrol-1-yl)phenyl]-4,4,4-trifluoro-1,3-butanedione (intermediate 1b-035)

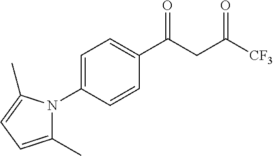 [SMILES: Cc1ccc(C)n1-c1ccc(C(=O)CC(=O)C(F)(F)F)cc1]

By proceeding in a similar manner to the previous procedure but using 1-[4-(2,5-dimethyl-1H-pyrrol-1-yl)phenyl]-ethanone, crude 1-[4-(2,5-dimethyl-1H-pyrrol-1-yl)phenyl]-4,4,4-trifluoro-1,3-butanedione was prepared.

¹H-NMR (CDCl₃): δ=2.08 (s, 6H); 5.96 (s, 2H); 6.62 (s, 1H); 7.37 (d, 2H); 8.07 (d, 2H) ppm.

### N-Methyl-N-phenyl-4-[(4,4,4-trifluoro-3-hydroxybut-2-enoyl]benzamide (Intermediate 1 b-01)

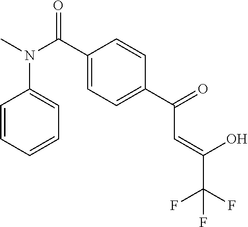 [SMILES: CN(C(=O)c1ccc(C(=O)/C=C(\O)C(F)(F)F)cc1)c1ccccc1]

Sodium methoxide (19 mL, 9.5 mmol, 0.5 M in MeOH) was added dropwise to a solution of 4-acetyl-N-methyl-N-phenylbenzamide (2.0 g, 7.9 mmol) and ethyl 2,2,2-trifluoroacetate (1.2 g, 8.7 mmol) in diethyl ether (30 mL). The reaction mixture was stirred for 3 d at rt. Aqueous hydrochloric acid solution (50 mL, 1 M) was then added. The aqueous layer was extracted with diethyl ether, the combined organic layers were dried (MgSO₄), filtered and concentrated under reduced pressure to give N-methyl-N-phenyl-4-[4,4,4-trifluoro-3-hydroxybut-2-enoyl]benzamide as a yellow oil (2.4 g, 78% yield) which was used in the next step without further purification.

MS (ESI): 350 ([M+H]⁺); 368 ([M+H₃O]⁺)

### 1-[4-(3-Ethyl-1,2,4-oxadiazol-5-yl)phenyl]-4,4,4-trifluoro-3-hydroxy-but-2-en-1-one (Intermediate 1b-02)

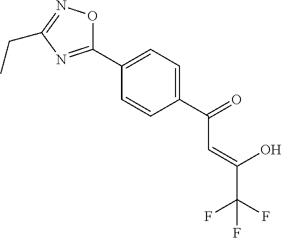 [SMILES: CCc1noc(-c2ccc(C(=O)/C=C(\O)C(F)(F)F)cc2)n1]

Sodium methoxide (2.2 mL, 1.1 mmol, 0.5 M in MeOH) was added dropwise to a solution of 1-[4-(3-ethyl-1,2,4-oxadiazol-5-yl)phenyl]ethanone (198 mg, 0.92 mmol) and ethyl 2,2,2-trifluoroacetate (143 mg, 1.0 mmol) in diethyl ether (2.8 mL). The reaction mixture was stirred for 4 h at rt. Aqueous hydrochloric acid solution (10 mL, 1 M) was then added. The aqueous layer was extracted with diethyl ether, the combined organic layers were dried (MgSO₄), filtered and concentrated under reduced pressure to give 1-[4-(3-ethyl-1,2,4-oxadiazol-5-yl)phenyl]-4,4,4-trifluoro-3-hydroxy-but-2-en-1-one as a yellow oil (251 mg, 83% yield) which was used in the next step without further purification.

MS (ESI): 313 ([M+H]⁺); 331 ([M+H₃O]⁺)

### 4-[4-Chloro-4,4-difluoro-3-hydroxybut-2-enoyl]phenyl dimethylcarbamate

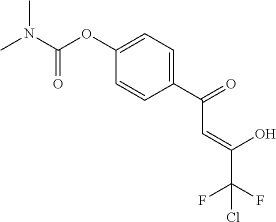 [SMILES: CN(C)C(=O)Oc1ccc(C(=O)/C=C(\O)C(F)(F)Cl)cc1]

Sodium hydride (116 mg, 2.9 mmol, 60% in mineral oil) was added to a suspension of 4-acetylphenyl dimethylcarbamate (300 mg, 1.4 mmol) in Tetrahydrofuran (3.0 mL) at 0° C. The reaction mixture was stirred at 0° C. for 30 min and ethyl 2-chloro-2,2-difluoroacetate (344 mg, 2.2 mmol) was then added. The reaction mixture was stirred for 3 h at rt. Water was then added and the organic solvent was concentrated under reduced pressure. Aqueous hydrochloric acid solution (1 M) was then added until pH 1. The aqueous layer was extracted with EtOAc, the combined organic layers were dried (MgSO₄), filtered and concentrated under reduced pressure to give 4-[4-chloro-4,4-difluoro-3-hydroxybut-2-enoyl]phenyl dimethylcarbamate as a yellow oil (449 mg, 20% yield, 20% purity) which was used in the next step without further purification.

MS (ESI): 320 ([M+H]⁺)

### methyl 4-(4-chloro-4,4-difluoro-3-oxobutanoyl)benzoate (intermediate 1 b-04)

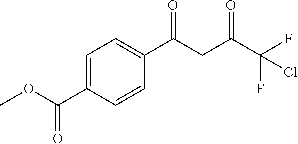 [SMILES: COC(=O)c1ccc(C(=O)CC(=O)C(F)(F)Cl)cc1]

To a suspension of methyl 4-acetylbenzoate (15 g, 84.1 mmol) in 250 ml diethylether was added ethyl difluorochloroacetate (15 g, 92.6 mmol) and a solution of sodium methoxide 0.5M in MeOH (202 ml, 101 mmol) was added dropwise. The mixture was stirred at rt for 24 h and HCl 1M (130 ml) was then added. The mixture was extracted 3 times with ethyl acetate, dried over MgSO4 and evaporated to afford methyl 4-(4-chloro-4,4-difluoro-3-oxobutanoyl)benzoate (21.8 g, 82% yield) as a mixture of diketone and ketoenol which was used in the next step without further purification.

MS (ESI): 291 ([M+H]⁺)

### Synthesis of Compounds of Formula (I)

### 3-(4-Azidomethyl-phenyl)-5-trifluoromethyl-4,5-dihydro-isoxazol-5-ol (Compound Ia-001)

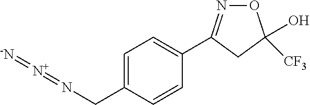

A solution of hydroxylamine hydrochloride (4.34 g, 62.5 mmol) in water (51 ml) and a 2N solution of aqueous sodium hydroxide (33 ml, 67 mmol) was slowly added to a solution of crude 1-(4-Azidomethyl-phenyl)-4,4,4-trifluoro-butane-1,3-dione in ethanol (150 ml). After stirring for 3 h at 60° C., the reaction mixture was quenched with an aqueous solution of ammonium chloride and extracted with ethyl acetate. The organic phase was separated, washed with brine, dried (Na₂SO₄), filtered and concentrated in vacuo to give crude 3-(4-Azidomethyl-phenyl)-5-trifluoromethyl-4,5-dihydro-isoxazol-5-ol (18.2 g) which was used without further purification.

¹H-NMR (CDCl₃): δ=3.52 (d, 1H); 3.74 (d, 1H); 4.41 (s, 2H); 7.42 (d, 2H); 7.70 (d, 2H) ppm.

### 3-(4-aminophenyl)-4,5-dihydro-5-(trifluoromethyl)-5-isoxazolol (Compound Ia-114)

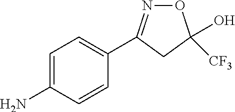 [SMILES: Nc1ccc(C2=NOC(O)(C(F)(F)F)C2)cc1]

A solution of hydroxylamine hydrochloride (0.85 g, 12.2 mmol) in water (10 ml) and a 2N solution of aqueous sodium hydroxide (6.5 ml, 13 mmol) was slowly added to a solution of crude 1-[4-(2,5-dimethyl-1H-pyrrol-1-yl)phenyl]-4,4,4-trifluoro-1,3-butanedione in ethanol (29 ml). After stirring for 7 h at 60° C., hydroxylamine hydrochloride (0.85 g, 12.2 mmol) was added and reaction mixture was heated to 125° C. After 24 h, the reaction mixture was cooled to room temperature, quenched with an aqueous solution of sodium hydrogencarbonate and extracted with ethyl acetate. The organic phase was separated, washed with brine, dried (Na₂SO₄), filtered, concentrated in vacuo and purified by flash column chromatography (hexane/ethyl acetate) to give 3-(4-aminophenyl)-4,5-dihydro-5-(trifluoromethyl)-5-isoxazolol (1.5 g, 52% over 2 steps).

¹H-NMR (DMSO-d6): δ=3.38 (d, 1H); 3.75 (d, 1H); 5.64 (s, 2H); 6.54 (d, 2H); 7.32 (d, 2H); 8.35 (s, 1H) ppm.

### 3-(4-Aminomethyl-phenyl)-5-trifluoromethyl-4,5-dihydro-isoxazol-5-ol (Compound Ia-112)

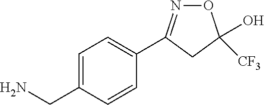 [SMILES: NCc1ccc(C2=NOC(O)(C(F)(F)F)C2)cc1]

Triphenylphosphine (19.5 g, 74.4 mmol) was added to a solution of crude 3-(4-Azidomethyl-phenyl)-5-trifluoromethyl-4,5-dihydro-isoxazol-5-ol in THF (93 ml) and water (65 ml) and vigorously stirred. After 4 hours, the reaction mixture was concentrated in vacuo and purified by flash column chromatography (MeOH/DCM 10-30%) to give 3-(4-Aminomethyl-phenyl)-5-trifluoromethyl-4,5-dihydro-isoxazol-5-ol (8.11 g, 49% over 3 steps).

¹H-NMR (DMSO-d6): δ=3.49 (d, 1H); 3.74 (s, 2H); 3.87 (d, 1H); 7.41 (d, 2H); 7.63 (d, 2H) ppm.

### 4-[5-Hydroxy-5-(trifluoromethyl)-4,5-dihydro-1,2-oxazol-3-yl]-N-methyl-N-phenylbenzamide

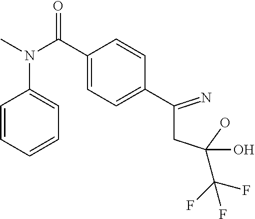 [SMILES: CN(C(=O)c1ccc(C(=N)CC(O)(O)C(F)(F)F)cc1)c1ccccc1]

Hydroxylamine hydrochloride (22 mg, 0.32 mmol) was added to a solution of N-methyl-N-phenyl-4-[4,4,4-trifluoro-3-hydroxybut-2-enoyl]benzamide (100 mg, 0.29 mmol) in acetic acid (1 mL). The reaction mixture was stirred for 2 h at 70° C. It was then cooled down to rt and purified by prep HPLC and then purified again by column chromatography eluting with 20% EtOAc in heptane to afford 4-[5-hydroxy-5-(trifluoromethyl)-4H-isoxazol-3-yl]-N-methyl-N-phenyl-benzamide (50 mg, 46% yield).

MS (ESI): 365 ([M+H]⁺)

### 3-[4-(3-Ethyl-1,2,4-oxadiazol-5-yl)phenyl]-5-(trifluoromethyl)-4H-isoxazol-5-ol (Compound Ia-093)

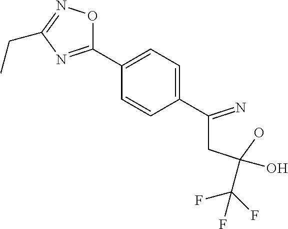 [SMILES: CCc1noc(-c2ccc(C(=N)CC(O)(O)C(F)(F)F)cc2)n1]

Hydroxylamine hydrochloride (24 mg, 0.35 mmol) was added to a solution of 1-[4-(3-ethyl-1,2,4-oxadiazol-5-yl)phenyl]-4,4,4-trifluoro-3-hydroxy-but-2-en-1-one (100 mg, 0.32 mmol) in acetic acid (1 mL). The reaction mixture was stirred for 2 h at 70° C. It was then poured onto water (10 mL). The solid was filtered off and dried to afford 3-[4-(3-ethyl-1,2,4-oxadiazol-5-yl)phenyl]-5-(trifluoromethyl)-4H-isoxazol-5-ol (72 mg, 65% yield).

MS (ESI): 328 ([M+H]⁺)

### [3-[4-(3-Ethyl-1,2,4-oxadiazol-5-yl)phenyl]-5-(trifluoromethyl)-4H-isoxazol-5-yl]acetate (Compound Ia-095)

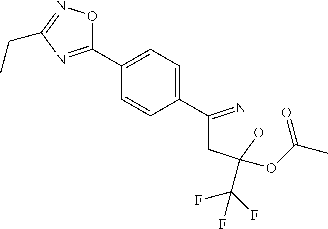 [SMILES: CCc1noc(-c2ccc(C(=N)CC(O)(OC(C)=O)C(F)(F)F)cc2)n1]

4-Dimethylaminopyridine (22 mg, 0.18 mmol) was added to a solution of 3-[4-(3-ethyl-1,2,4-oxadiazol-5-yl)phenyl]-5-(trifluoromethyl)-4H-isoxazol-5-ol (Compound I-19) (30 mg, 0.092 mmol) and acetic anhydride (0.13 mL, 1.37 mmol) in acetic acid (2 mL). The reaction mixture was stirred for 9 h at 60° C. and was then cooled down to rt and purified by prep HPLC to afford [3-[4-(3-ethyl-1,2,4-oxadiazol-5-yl)phenyl]-5-(trifluoromethyl)-4H-isoxazol-5-yl]acetate (23 mg, 69% yield).

MS (ESI): 370 ([M+H]⁺)

### Ethyl 2-[[3-[4-[methyl(phenyl)carbamoyl]phenyl]-5-(trifluoromethyl)-4H-isoxazol-5-yl]oxy]acetate

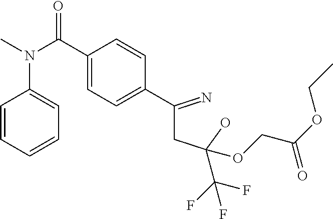 [SMILES: CCOC(=O)COC(O)(CC(=N)c1ccc(C(=O)N(C)c2ccccc2)cc1)C(F)(F)F]

N,N-Diisopropylethylamine (16 μL, 0.055 mmol) and ethyl bromoacetate (6 μL, 0.055 mmol) were added to a solution of 4-[5-hydroxy-5-(trifluoromethyl)-4H-isoxazol-3-yl]-N-methyl-N-phenyl-benzamide (Compound I-18) (10 mg, 0.027 mmol) in acetonitrile (1 mL). The reaction mixture was stirred for 2 h at 60° C. and was then refluxed for 6 h. It was then cooled down to rt and purified by prep HPLC to afford 2-[[3-[4-[methyl(phenyl)carbamoyl]phenyl]-5-(trifluoromethyl)-4H-isoxazol-5-yl]oxy]acetate (7 mg, 59% yield).

MS (ESI): 451 ([M+H]⁺)

### 4-{5-[Chloro(difluoro)methyl]-5-hydroxy-4,5-dihydro-1,2-oxazol-3-yl}phenyl dimethylcarbamate (Compound Ib-095)

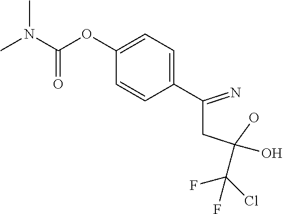 [SMILES: CN(C)C(=O)Oc1ccc(C(=N)CC(O)(O)C(F)(F)Cl)cc1]

Hydroxylamine hydrochloride (24 mg, 0.33 mmol) was added to a solution of 4-[4-chloro-4,4-difluoro-3-hydroxybut-2-enoyl]phenyl dimethylcarbamate (449 mg, 0.29 mmol, purity 20%) in acetic acid (3 mL). The reaction mixture was stirred for 4 h at 80° C. and then concentrated under reduced pressure. The residue was then solved in EtOAc and washed with water. The organic layer was then dried (MgSO₄), filtered, concentrated under reduced pressure and purified by column chromatography eluting with 10-50% EtOAc in heptane to afford 4-{5-[chloro(difluoro)methyl]-5-hydroxy-4,5-dihydro-1,2-oxazol-3-yl}phenyl dimethylcarbamateas a beige solid (47 mg, 45% yield).

MS (ESI): 335 ([M+H]⁺)

### 3-[4-(cvclopropvlethvnvl)phenyl]-5-(trifluoromethyl)-4,5-dihydro-1,2-oxazol-5-ol (Compound Ia-031)

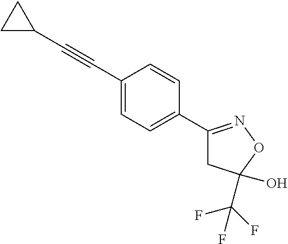 [SMILES: OC1(C(F)(F)F)CC(c2ccc(C#CC3CC3)cc2)=NO1]

To Copper(I) iodide (122 mg, 0.64 mmol) and Tetrakis(triphenylphosphine)palladium (0) (373 mg, 0.32 mmol) were added THF (5 ml), triethylamine (1.6 ml, 11.3 mmol), 3-(4-bromophenyl)-5-(trifluoromethyl)-4,5-dihydro-1,2-oxazol-5-ol (1.0 g, 3.2 mmol) and ethynylcyclopropane (597 mg, 9 mmol). The mixture was stirred at 60° C. for 5 h. The solvent was evaporated, water and dichloromethane were then added and the 2 layers were separated. The organic layer was dried over magnesium sulfate and evaporated. The residue was purified using flash chromatography (eluent: heptan:ethyl acetate 1:1) and re-purified using preparative HPLC to afford 3-[4-(cyclopropylethynyl)phenyl]-5-(trifluoromethyl)-4,5-dihydro-1,2-oxazol-5-ol (38 mg, 4% yield).

MS (ESI): 296 ([M+H]⁺)

### 1-{4-[5-hydroxy-5-(trifluoromethyl)-4,5-dihydro-1,2-oxazol-3-yl]phenyl}ethanone (Compound Ia-038)

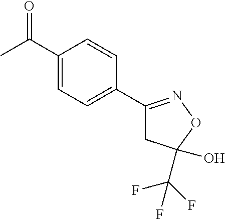 [SMILES: CC(=O)c1ccc(C2=NOC(O)(C(F)(F)F)C2)cc1]

To a solution of 3-(4-bromophenyl)-5-(trifluoromethyl)-4,5-dihydro-1,2-oxazol-5-ol (300 mg, 0.97 mmol) in dry degassed 1,4-dioxane (4 ml) was added, under argon, tributyl(1-ethoxyvinyl)stannane (524 mg, 1.5 mmol) and Bis(triphenylphosphine)palladium (II) dichloride (68 mg, 0.1 mmol). The mixture was stirred at 80° C. for 2 h and then cooled to rt. The solvent was evaporated and the residue was diluted in ethyl acetate, filtered through celite, washed with water and evaporated. The residue was diluted in acetic acid (3 ml) and hydrochloric acid 1M was added. The mixture was stirred at rt for 3 h30 and the solvent was evaporated. The residue was diluted in dichloromethane and filtered through celite. The filtrate was washed with a saturated solution of sodium hydrogen carbonate and with brine. The organic layers was dried over a separator phase filter and evaporated. The residue was purified by flash chromatography (eluent: heptan/ethyl acetate) to afford 1-{4-[5-hydroxy-5-(trifluoromethyl)-4,5-dihydro-1,2-oxazol-3-yl]phenyl}ethanone (150 mg, 54%) as a white solid.

MS (ESI): 274 ([M+H]⁺)

### 3-[4-(N-methoxvethanimidovl)phenyl]-5-(trifluoromethyl)-4,5-dihydro-1,2-oxazol-5-ol (compound Ia-037)

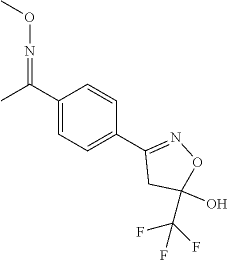 [SMILES: CON=C(C)c1ccc(C2=NOC(O)(C(F)(F)F)C2)cc1]

To a solution of 1-{4-[5-hydroxy-5-(trifluoromethyl)-4,5-dihydro-1,2-oxazol-3-yl]phenyl}ethanone (compound Ia-038) (200 mg, 0.73 mmol) in absolute ethanol (3 ml) were added methoxylamine hydrochloride (92 mg, 1.1 mmol) and sodium acetate (91 mg, 1.1 mmol). The mixture was stirred at 50° C. for 4 h. The solvent was evaporated and the residue was dissolved in ethyl acetate and water. The two layers were separated and the organic phase was washed with brine, dried through phase separator filter and evaporated. The residue was purified using preparative HPLC (eluent: acetonitrile/water (0.1% formic acid)) to afford 3-[4-(N-methoxyethanimidoyl)phenyl]-5-(trifluoromethyl)-4,5-dihydro-1,2-oxazol-5-ol (134 mg, 57%, mixture 95:5 of 2 isomers) as a white solid.

MS (ESI): 303 ([M+H]⁺)

### 3-[4-(1-hydroxyethyl)phenyl]-5-(trifluoromethyl)-4,5-dihydro-1,2-oxazol-5-ol (Compound Ia-044)

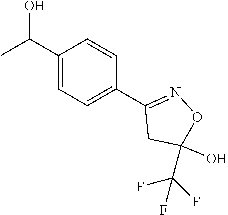 [SMILES: CC(O)c1ccc(C2=NOC(O)(C(F)(F)F)C2)cc1]

To a suspension of 1-{4-[5-hydroxy-5-(trifluoromethyl)-4,5-dihydro-1,2-oxazol-3-yl]phenyl}ethanone (compound Ia-038) (100 mg, 0.36 mmol) in methanol (2 ml) was added sodium borohydride (21 mg, 0.54 mmol). The mixture was stirred at rt for 16 h. Water and ethyl acetate were added. The two layers were separated and the organic phase was dried through phase separator filter and evaporated. The residue was purified using flash chromatography (eluent: heptan/ethyl acetate) to afford 3-[4-(1-hydroxyethyl)phenyl]-5-(trifluoromethyl)-4,5-dihydro-1,2-oxazol-5-ol (64 mg, 60%).

MS (ESI): 276 ([M+H]⁺)

### methyl 4-{5-[chloro(difluoro)methyl]-5-hydroxy-4,5-dihydro-1,2-oxazol-3-yl}benzoate (Compound Ib-049)

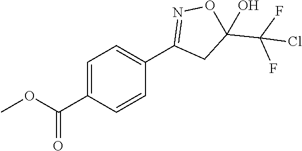 [SMILES: COC(=O)c1ccc(C2=NOC(O)(C(F)(F)Cl)C2)cc1]

To a mixture of methyl 4-(4-chloro-4,4-difluoro-3-oxobutanoyl)benzoate (21.7 g, 75 mmol) in acetic acid (130 ml) was added hydroxylamine hydrochloride (6.0 g, 86 mmol). The mixture was stirred at 80° C. for 2 h. The reaction mixture was cooled to RT, poured in water (600 ml) and stirred for 15 min. The precipitate was filtered, washed with heptan and dried to afford methyl 4-{5-[chloro(difluoro)methyl]-5-hydroxy-4,5-dihydro-1,2-oxazol-3-yl}benzoate (17.9 g, 75% yield) as a beige solid.

MS (ESI): 306 ([M+H]⁺)

### 4-{5-[chloro(difluoro)methyl]-5-hydroxy-4,5-dihydro-1,2-oxazol-3-yl}benzoic acid (compound Ib-048)

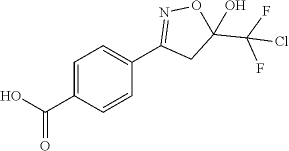 [SMILES: O=C(O)c1ccc(C2=NOC(O)(C(F)(F)Cl)C2)cc1]

To a mixture of methyl 4-{5-[chloro(difluoro)methyl]-5-hydroxy-4,5-dihydro-1,2-oxazol-3-yl}benzoate (17.8 g, 58.2 mmol) in methanol (138 ml), were added water (17 ml) and sodium hydroxide (4.7 g, 116 mmol). The mixture was stirred at 60° C. for 1.5 h. The mixture was poured into a cold mixture of HCl 1M (150 ml) and water (150 ml) and stirred for 5 min. The precipitate was filtered and dried to afford 4-{5-[chloro(difluoro)methyl]-5-hydroxy-4,5-dihydro-1,2-oxazol-3-yl}benzoic acid (16.2 g, 97% yield) as a beige solid.

MS (ESI): 292 ([M+H]⁺)

### General Procedure for the Synthesis of Compounds of Formula I According to Method A

To the amine of formula A (0.15 mmol) in dimethylformamide (0.4 mL) the compound of formula B (0.165 mmol) in dimethylformamide (0.3 mL) and 4-dimethylaminopyridine (0.16 mmol) in dimethylformamide (0.2 mL) were sucessively added, and the mixture was stirred at 100° C. bath temperature for 12 h. For work-up, the mixture was cooled to room temperature, diluted with methanol (1 mL) and concentrated. The residue was purified using preparative HPLC-MS and the products were characterized using analytical HPLC-MS using column LiChroCart Purospher Star 125-4 (125×4.5 mm, RP18e, 5 μm); gradient 5-95% acetonitrile (0.1% trifluoroacetic acid) in water (0.1% trifluoroacetic acid) (10 min.); flow rate 1.2 mL/min; MS ES+.

### N-{4-[5-hydroxy-5-(trifluoromethyl)-4,5-dihydro-1,2-oxazol-3-yl]phenyl}-1,5-dimethyl-1H-pyrazole-3-carboxamide

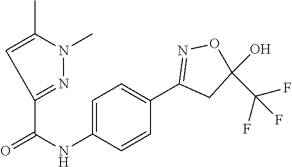 [SMILES: Cc1cc(C(=O)Nc2ccc(C3=NOC(O)(C(F)(F)F)C3)cc2)nn1C]

N-{4-[5-hydroxy-5-(trifluoromethyl)-4,5-dihydro-1,2-oxazol-3-yl]phenyl}-1,5-dimethyl-1H-pyrazole-3-carboxamide was prepared according to general procedure A starting from 3-(4-aminophenyl)-4,5-dihydro-5-(trifluoromethyl)-5-isoxazolol (A) and 1,5-dimethyl-1H-pyrazole-3-carbonyl chloride (B).

MS (ESI): 369 ([M+H]⁺)

### N-{4-[5-hydroxy-5-(trifluoromethyl)-4,5-dihydro-1,2-oxazol-3-yl]benzyl}isonicotinamide

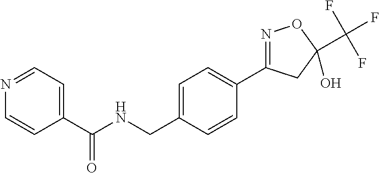 [SMILES: O=C(NCc1ccc(C2=NOC(O)(C(F)(F)F)C2)cc1)c1ccncc1]

N-{4-[5-hydroxy-5-(trifluoromethyl)-4,5-dihydro-1,2-oxazol-3-yl]benzyl}isonicotinamide was prepared according to general procedure A starting from 3-(4-Aminomethyl-phenyl)-5-trifluoromethyl-4,5-dihydro-isoxazol-5-ol (A) and isonicotinoyl chloride (B).

MS (ESI): 366 ([M+H]⁺)

### N-{4-[5-hydroxy-5-(trifluoromethyl)-4,5-dihydro-1,2-oxazol-3-yl]phenyl}-3-phenylpropanamide

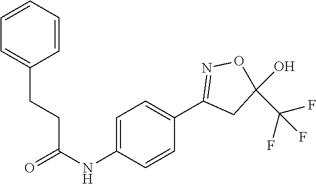 [SMILES: O=C(CCc1ccccc1)Nc1ccc(C2=NOC(O)(C(F)(F)F)C2)cc1]

N-{4-[5-hydroxy-5-(trifluoromethyl)-4,5-dihydro-1,2-oxazol-3-yl]phenyl}-3-phenylpropanamide was prepared according to general procedure A starting from 3-(4-aminophenyl)-4,5-dihydro-5-(trifluoromethyl)-5-isoxazolol (A) and 3-phenylpropanoyl chloride (B).

MS (ESI): 379 ([M+H]⁺)

### Synthesis of Compounds of Formula I According to Method B

### 4-{5-[chloro(difluoro)methyl]-5-hydroxy-4,5-dihydro-1,2-oxazol-3-vl}-N-(2,4-difluorophenyl)benzamide (Compound Ib-030)

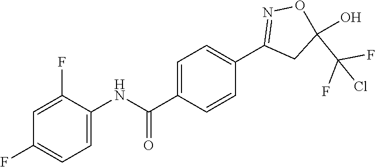 [SMILES: O=C(Nc1ccc(F)cc1F)c1ccc(C2=NOC(O)(C(F)(F)Cl)C2)cc1]

To a solution of 4-{5-[chloro(difluoro)methyl]-5-hydroxy-4,5-dihydro-1,2-oxazol-3-yl}benzoic acid (compound Ib-048) (100 mg, 0.34 mmol) in DMF (2 ml) were added, under argon, pyridine (33 μl, 0.41 mmol), 2,4-difluoroaniline (54 mg, 0.41 mmol) and at 0° C., HATU (261 mg, 0.69 mmol). The mixture was stirred at rt for 18 h and then poured into water. The aqueous phase was extracted three times with ethyl acetate. The combined organic layers were washed twice with a saturated solution of sodium hydrogen carbonate, once with brine, dried over magnesium sulfate and evaporated. The residue was purified by flash chromatography (eluent: heptan/ethyl acetate) to afford 4-{5-[chloro(difluoro)methyl]-5-hydroxy-4,5-dihydro-1,2-oxazol-3-yl}-N-(2,4-difluorophenyl)benzamide (126 mg, 87% yield) as a yellow solid.

MS (ESI): 403 ([M+H]⁺)

### Synthesis of Compounds of Formula I According to Method C

### 3-(4′-fluoro[biphenyl]-4-yl)-5-(trifluoromethyl)-4,5-dihydro-1,2-oxazol-5-ol (Compound Ia-075)

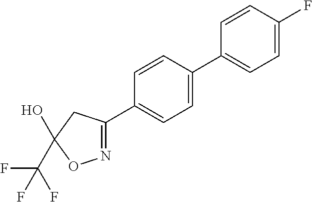 [SMILES: OC1(C(F)(F)F)CC(c2ccc(-c3ccc(F)cc3)cc2)=NO1]

To (4-fluorophenyl)boronic acid (54 mg, 0.39 mmol) and Tetrakis(triphenylphosphine)palladium (0) (20 mg, 0.018 mmol) were added a solution of cesium carbonate (125 mg, 0.39 mmol) in water (1 ml) and a solution of 3-(4-bromophenyl)-5-(trifluoromethyl)-4,5-dihydro-1,2-oxazol-5-ol (108 mg, 0.35 mmol) in 1,2-dimetoxyethane (3 ml). The mixture was stirred at 80° C. for 1 h and then cooled to rt. Water and dichloromethane were then added and the 2 layers were separated. The organic layer was filtered over a silica cartridge, eluted with dichloromethane and evaporated. The residue was purified using preparative HPLC-MS (SunFire Waters, 30*150, 5 μm, eluent: acetonitrile/water (0.1% formic acid)) to afford 3-(4′-fluoro[biphenyl]-4-yl)-5-(trifluoromethyl)-4,5-dihydro-1,2-oxazol-5-ol (57 mg, 50% yield).

MS (ESI): 326 ([M+H]⁺)

### 3-[4-(pyrimidin-5-yl)phenyl]-5-(trifluoromethyl)-4,5-dihydro-1,2-oxazol-5-ol (compound Ia-087)

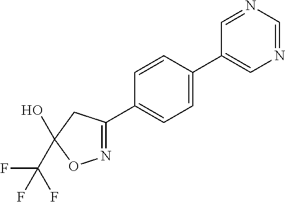 [SMILES: OC1(C(F)(F)F)CC(c2ccc(-c3cncnc3)cc2)=NO1]

To pyrimidin-5-ylboronic acid (47 mg, 0.39 mmol) and Tetrakis(triphenylphosphine)palladium (0) (20 mg, 0.018 mmol) were added a solution of cesium carbonate (125 mg, 0.39 mmol) in water (1 ml) and a solution of 3-(4-bromophenyl)-5-(trifluoromethyl)-4,5-dihydro-1,2-oxazol-5-ol (108 mg, 0.35 mmol) in 1,2-dimetoxyetane (3 ml). The mixture was stirred at 80° C. for 1 h and then cooled to rt. Water and dichloromethane were then added and the 2 layers were separated. The organic layer was filtered over a silica cartridge, eluted with dichloromethane and evaporated. The residue was purified using preparative HPLC-MS (SunFire Waters, 30*150, 5 μm, eluent: acetonitrile/water (0.1% formic acid)) to afford 3-[4-(pyrimidin-5-yl)phenyl]-5-(trifluoromethyl)-4,5-dihydro-1,2-oxazol-5-ol (45 mg, 42% yield).

MS (ESI): 310 ([M+H]⁺)

The compounds in table 1, 2 and 3 were prepared in analogy with the examples provided above.

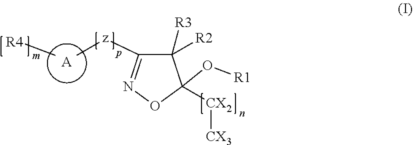

TABLE 1
Compounds according to formula (I) (in accordance with embodiment 1)

| Ex N^{o} | R1 | R2 | R3 | 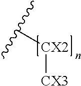 | 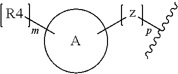 | LogP |
|---|---|---|---|---|---|---|
| Ia-001 | H | H | H | CF₃ | 4-(azidomethyl)phenyl | |
| Ia-002 | H | H | H | CF₃ | 4-methyl-3-nitrophenyl | 2.70^{[a]} |
| Ia-003 | H | H | H | CHF₂ | 4-nitrophenyl | 2.08^{[a]} |
| Ia-004 | H | H | H | CF₃ | 4-(2-trimethylsilylethyn-1-yl)phenyl | 4.34^{[a]} |
| Ia-005 | H | H | H | CF₃ | 4-bromophenyl | 2.90^{[a]} |
| Ia-006 | acetyl | H | H | CF₃ | 4-bromophenyl | 3.65^{[a]} |
| Ia-007 | tert-butoxycarbonyl | H | H | CF₃ | 4-bromophenyl | 4.53^{[a]} |
| Ia-008 | H | H | H | CF₃ | 5-bromo-2-thienyl | 2.80^{[a]} |
| Ia-009 | H | H | H | CF₃ | 4-[5-methyl-3-phenyl-4H-1,2-oxazol-5-yl]phenyl | 3.37^{[a]} |
| Ia-010 | H | H | H | CF₃ | 4-[3,5-dimethyl-4H-1,2-oxazol-5-yl]phenyl | 2.32^{[a]} |
| Ia-011 | H | H | H | CF₃ | 4-[2-(methoxycarbonyl)phenyl]phenyl | 3.19^{[a]} |
| Ia-012 | H | H | H | CF₃ | (E)-2-(3,4-dimethoxyphenyl)ethen-1-yl | 2.45^{[a]} |
| Ia-013 | H | H | H | CF₃ | (E)-2-phenylethen-1-yl | |
| Ia-014 | H | H | H | CF₃ | 4-ethoxy-2,3-difluorophenyl | 2.96^{[a]} |
| Ia-015 | H | H | H | CF₃ | 4-carboxy-2-fluorophenyl | 1.88^{[a]} |
| Ia-016 | H | H | H | CF₃ | 2-fluoro-4-(methoxycarbonyl)phenyl | 2.58^{[a]} |
| Ia-017 | acetyl | H | H | CF₃ | 2,4-dichlorophenyl | 3.94^{[a]} |
| Ia-018 | H | H | H | CF₃ | 5-chloro-2-hydroxyphenyl | |
| Ia-019 | acetyl | H | H | CF₃ | 2,6-difluorophenyl | 3.00^{[a]} |
| Ia-020 | H | H | H | CF₃ | 2-fluoro-6-hydroxyphenyl | 2.34^{[a]} |
| Ia-021 | H | H | H | CF₃ | 3-fluoro-4-(trifluoromethyl)phenyl | 3.19^{[a]} |
| Ia-022 | acetyl | H | H | CF₃ | 3,4-dichlorophenyl | 3.94^{[a]} |
| Ia-023 | H | H | H | CF₃ | 3-fluoro-4-(1H-imidazol-1-yl)phenyl | 1.03^{[a]} |
| Ia-024 | H | H | H | CF₃ | 3-fluoro-4-hydroxyphenyl | 1.81^{[a]} |
| Ia-025 | H | H | H | CF₃ | 3-(aminomethyl)phenyl | 0.65^{[a]} |
| Ia-026 | H | H | H | CF₃ | 3-aminophenyl | 1.31^{[a]} |
| Ia-027 | pyrrolidin-1-ylcarbonyl | H | H | CF₃ | 3-aminophenyl | 2.59^{[a]} |
| Ia-028 | H | H | H | CF₃ | 3-hydroxyphenyl | 1.78^{[a]} |
| Ia-029 | H | H | H | CF₃ | 4-(3-hydroxy-3-methylbut-1-yn-1-yl)phenyl | 2.37^{[a]} |
| Ia-030 | H | H | H | CF₃ | 4-[2-(2,4-difluorophenyl)ethyn-1-yl]phenyl | 3.99^{[a]} |
| Ia-031 | H | H | H | CF₃ | 4-(2-cyclopropylethyn-1-yl)phenyl | 3.41^{[a]} |
| Ia-032 | H | H | H | CF₃ | 4-(2-phenylethyn-1-yl)phenyl | 3.92^{[a]} |
| Ia-033 | H | H | H | CF₃ | 4-prop-1-en-2-ylphenyl | 3.15^{[a]} |
| Ia-034 | H | H | H | CF₃ | 4-(1-hydroxyiminoethyl)phenyl | 2.10^{[a]} |
| Ia-035 | H | H | H | CF₃ | 4-[anilino(hydroxyimino)methyl]phenyl | 2.31^{[a]} |
| Ia-036 | H | H | H | CF₃ | 4-[(benzylamino)-hydroxyiminomethyl]phenyl | 1.86^{[a]} |
| Ia-037 | H | H | H | CF₃ | 4-[1-(methoxyimino)ethyl]phenyl | 2.98^{[a]} |
| Ia-038 | H | H | H | CF₃ | 4-acetylphenyl | 2.11^{[a]} |
| Ia-039 | H | H | H | CF₃ | 4-(aminocarbonyl)phenyl | 1.33^{[a]} |
| Ia-040 | H | H | H | CF₃ | 4-carboxyphenyl | 1.75^{[a]} |
| Ia-041 | H | H | H | CF₃ | 4-(methoxycarbonyl)phenyl | 2.39^{[a]} |
| Ia-042 | H | H | H | CF₃ | 4-[carboxy(difluoro)methyl]phenyl | 1.34^{[a]} |
| Ia-043 | H | H | H | CF₃ | 4-[1-(methoxyamino)ethyl]phenyl | 1.96^{[a]} |
| Ia-044 | H | H | H | CF₃ | 4-[1-hydroxyethyl]phenyl | 1.78^{[a]} |
| Ia-045 | H | H | H | CF₃ | 4-methylphenyl | 2.64^{[a]} |
| Ia-046 | H | H | H | CF₃ | 4-vinylphenyl | 2.82^{[a]} |
| Ia-047 | H | H | H | CF₃ | 4-(2-oxo-1,2-dihydropyridin-3-yl)phenyl | 1.74^{[a]} |
| Ia-048 | H | H | H | CF₃ | 4-(3,4-dihydro-2H-pyran-6-yl)phenyl | 1.91^{[a]} |
| Ia-049 | H | H | H | CF₃ | 4-(2-cyanophenyl)phenyl | 2.96^{[a]} |
| Ia-050 | H | H | H | CF₃ | 4-(2-carboxyphenyl)phenyl | 2.51^{[a]} |
| Ia-051 | H | H | H | CF₃ | 4-(2-chlorophenyl)phenyl | |
| Ia-052 | H | H | H | CF₃ | 4-(2-chlorophenyl)phenyl | 3.67^{[a]} |
| Ia-053 | H | H | H | CF₃ | 4-(2-fluorophenyl)phenyl | 3.42^{[a]} |
| Ia-054 | H | H | H | CF₃ | 4-(2-methoxyphenyl)phenyl | 3.39^{[a]} |
| Ia-055 | H | H | H | CF₃ | 4-(3-fluoropyridin-4-yl)phenyl | 2.35^{[a]} |
| Ia-056 | H | H | H | CF₃ | 4-(3-methoxypyridin-4-yl)phenyl | 1.45^{[a]} |
| Ia-057 | H | H | H | CF₃ | 4-(2-fluoropyridin-3-yl)phenyl | 2.59^{[a]} |
| Ia-058 | H | H | H | CF₃ | 4-(2-methoxypyridin-3-yl)phenyl | 2.92^{[a]} |
| Ia-059 | H | H | H | CF₃ | 4-(3,5-dimethyl-1,2-oxazol-4-yl)phenyl | 2.62^{[a]} |
| Ia-060 | H | H | H | CF₃ | 4-(1H-pyrazol-4-yl)phenyl | 1.81^{[a]} |
| Ia-061 | H | H | H | CF₃ | 4-(4,5,6,7-tetrahydropyrazolo[1,5-a]pyridin-3-yl)phenyl | 2.54^{[a]} |
| Ia-062 | H | H | H | CF₃ | 4-(3-cyanophenyl)phenyl | 3.06^{[a]} |
| Ia-063 | H | H | H | CF₃ | 4-[3-(aminocarbonyl)phenyl]phenyl | 2.07^{[a]} |
| Ia-064 | H | H | H | CF₃ | 4-[3-(methoxycarbonyl)phenyl]phenyl | 3.46^{[a]} |
| Ia-065 | H | H | H | CF₃ | 4-(3-chlorophenyl)phenyl | 3.87^{[a]} |
| Ia-066 | H | H | H | CF₃ | 4-(3-fluorophenyl)phenyl | 3.48^{[a]} |
| Ia-067 | H | H | H | CF₃ | 4-(2-fluoropyridin-4-yl)phenyl | 2.66^{[a]} |
| Ia-068 | H | H | H | CF₃ | 4-[2-(acetylamino)pyridin-4-yl]phenyl | 1.81^{[a]} |
| Ia-069 | H | H | H | CF₃ | 4-cyclopropylphenyl | 3.02^{[a]} |
| Ia-070 | H | H | H | CF₃ | 4-(2,3-dihydro-1-benzofuran-5-yl)phenyl | 3.37^{[a]} |
| Ia-071 | H | H | H | CF₃ | 4-(4-cyanophenyl)phenyl | 3.04^{[a]} |
| Ia-072 | H | H | H | CF₃ | 4-[4-[(N-methylanilino)carbonyl]phenyl]phenyl | 3.19^{[a]} |
| Ia-073 | H | H | H | CF₃ | 4-[4-(aminocarbonyl)phenyl]phenyl | 1.98^{[a]} |
| Ia-074 | H | H | H | CF₃ | 4-(4-chlorophenyl)phenyl | 3.89^{[a]} |
| Ia-075 | H | H | H | CF₃ | 4-(4-fluorophenyl)phenyl | 3.46^{[a]} |
| Ia-076 | H | H | H | CF₃ | 4-(4-methoxyphenyl)phenyl | 3.33^{[a]} |
| Ia-077 | H | H | H | CF₃ | 4-cyclohexylphenyl | 4.30^{[a]} |
| Ia-078 | H | H | H | CF₃ | 4-phenylphenyl | 3.42^{[a]} |
| Ia-079 | H | H | H | CF₃ | 4-pyridin-4-ylphenyl | 1.20^{[a]} |
| Ia-080 | H | H | H | CF₃ | 4-(1-methyl-1H-pyrazol-4-yl)phenyl | 2.10^{[a]} |
| Ia-081 | H | H | H | CF₃ | 4-(1-ethyl-1H-pyrazol-4-yl)phenyl | 2.35^{[a]} |
| Ia-082 | H | H | H | CF₃ | 4-(6-fluoropyridin-3-yl)phenyl | 2.68^{[a]} |
| Ia-083 | H | H | H | CF₃ | 4-[6-(acetylamino)pyridin-3-yl]phenyl | 2.02^{[a]} |
| Ia-084 | H | H | H | CF₃ | 4-(6-methoxypyridin-3-yl)phenyl | 2.90^{[a]} |
| Ia-085 | H | H | H | CF₃ | 4-pyridin-3-ylphenyl | 1.42^{[a]} |
| Ia-086 | H | H | H | CF₃ | 4-(4-methoxypyridin-3-yl)phenyl | 1.14^{[a]} |
| Ia-087 | H | H | H | CF₃ | 4-pyrimidin-5-ylphenyl | 1.86^{[a]} |
| Ia-088 | H | H | H | CF₃ | 4-(1,2-oxazol-4-yl)phenyl | 2.04^{[a]} |
| Ia-089 | H | H | H | CF₃ | 4-(2-chloro-1-methyl-1H-imidazol-5-yl)phenyl | 2.20^{[a]} |
| Ia-090 | H | H | H | CF₃ | 4-(1-methyl-1H-pyrazol-5-yl)phenyl | 2.17^{[a]} |
| Ia-091 | H | H | H | CF₃ | 4-(1-ethyl-1H-pyrazol-5-yl)phenyl | 2.41^{[a]} |
| Ia-092 | H | H | H | CF₃ | 4-(1H-pyrazol-3-yl)phenyl | 1.93^{[a]} |
| Ia-093 | H | H | H | CF₃ | 4-(3-ethyl-1,2,4-oxadiazol-5-yl)phenyl | 2.88^{[a]} |
| Ia-094 | CH3 | H | H | CF₃ | 4-(3-ethyl-1,2,4-oxadiazol-5-yl)phenyl | 3.78^{[a]} |
| Ia-095 | acetyl | H | H | CF₃ | 4-(3-ethyl-1,2,4-oxadiazol-5-yl)phenyl | 3.68^{[a]} |
| Ia-096 | cyclopropylcarbonyl | H | H | CF₃ | 4-(3-ethyl-1,2,4-oxadiazol-5-yl)phenyl | 4.11^{[a]} |
| Ia-097 | benzoyl | H | H | CF₃ | 4-(3-ethyl-1,2,4-oxadiazol-5-yl)phenyl | 4.69^{[a]} |
| Ia-098 | propionyl | H | H | CF₃ | 4-(3-ethyl-1,2,4-oxadiazol-5-yl)phenyl | 4.06^{[a]} |
| Ia-099 | 2-fluoroacetyl | H | H | CF₃ | 4-(3-ethyl-1,2,4-oxadiazol-5-yl)phenyl | 3.58^{[a]} |
| Ia-100 | 2-methoxyacetyl | H | H | CF₃ | 4-(3-ethyl-1,2,4-oxadiazol-5-yl)phenyl | 3.57^{[a]} |
| Ia-101 | 2-cyclopropyl-2-oxoethyl | H | H | CF₃ | 4-(3-ethyl-1,2,4-oxadiazol-5-yl)phenyl | 3.83^{[a]} |
| Ia-102 | phenacyl | H | H | CF₃ | 4-(3-ethyl-1,2,4-oxadiazol-5-yl)phenyl | 4.25^{[a]} |
| Ia-103 | 2-oxobutyl | H | H | CF₃ | 4-(3-ethyl-1,2,4-oxadiazol-5-yl)phenyl | 3.83^{[a]} |
| Ia-104 | H | H | H | CF₃ | 4-(1H-indazol-4-yl)phenyl | 2.46^{[a]} |
| Ia-105 | H | H | H | CF₃ | 4-(1-methyl-1H-indazol-4-yl)phenyl | 2.96^{[a]} |
| Ia-106 | H | H | H | CF₃ | 4-(1H-indol-6-yl)phenyl | 3.17^{[a]} |
| Ia-107 | H | H | H | CF₃ | 4-(1-methyl-1H-indol-5-yl)phenyl | 3.59^{[a]} |
| Ia-108 | H | H | H | CF₃ | 4-(1-benzothiophene-5-yl)phenyl | 3.94^{[a]} |
| Ia-109 | H | H | H | CF₃ | 4-(1-methyl-1H-indazol-6-yl)phenyl | 2.94^{[a]} |
| Ia-110 | H | H | H | CF₃ | 4-chlorophenyl | |
| Ia-111 | acetyl | H | H | CF₃ | 4-chlorophenyl | 3.45^{[a]} |
| Ia-112 | H | H | H | CF₃ | 4-(aminomethyl)phenyl | 0.63^{[a]} |
| Ia-113 | H | H | H | CF₃ | 4-(hydroxymethyl)phenyl | 1.57^{[a]} |
| Ia-114 | H | H | H | CF₃ | 4-aminophenyl | 1.49^{[a]} |
| Ia-115 | (1-cyanocyclopropyl)carbonyl | H | H | CF₃ | 4-aminophenyl | 2.45^{[a]} |
| Ia-116 | pyrrolidin-1-ylcarbonyl | H | H | CF₃ | 4-aminophenyl | 2.73^{[a]} |
| Ia-117 | H | H | H | CF₃ | 4-(2,5-dimethyl-1H-pyrrol-1-yl)phenyl | 3.46^{[a]} |
| Ia-118 | H | H | H | CF₃ | 4-morpholin-4-ylphenyl | 2.21^{[a]} |
| Ia-119 | H | H | H | CF₃ | 4-[(4,6-dimethylpyrimidin-2-yl)amino]phenyl | 2.64^{[a]} |
| Ia-120 | H | H | H | CF₃ | 4-[(4-methylpyrimidin-2-yl)amino]phenyl | 2.44^{[a]} |
| Ia-121 | H | H | H | CF₃ | 4-(pyrimidin-2-ylamino)phenyl | 2.20^{[a]} |
| Ia-122 | H | H | H | CF₃ | 4-hydroxyphenyl | 1.66^{[a]} |
| Ia-123 | H | H | H | CF₃ | 4-(4-chlorophenoxy)phenyl | 3.87^{[a]} |
| Ia-124 | H | H | H | CF₃ | 4-methylsulfonylphenyl | 1.76^{[a]} |
| Ia-125 | H | H | H | CF₃ | 4-(benzenesulfonyl)phenyl | 2.69^{[a]} |
| Ia-126 | H | H | H | CF₃ | 4-methylsulfanylphenyl | 2.75^{[a]} |
| Ia-127 | H | H | H | CF₃ | 4-phenylsulfanylphenyl | 3.81^{[a]} |
| Ia-128 | H | H | H | CF₃ | phenyl | |
| Ia-129 | H | H | H | CF₃ | 2-[(1-phenylcyclopropyl)amino]pyrimidin-5-yl | 2.59^{[a]} |
| Ia-130 | H | H | H | CF₃ | 2-anilinopyrimidin-5-yl | 2.55^{[a]} |
| Ia-131 | CH3 | H | H | CF₃ | 2-anilinopyrimidin-5-yl | 3.31^{[a]} |
| Ia-132 | acetyl | H | H | CF₃ | 2-anilinopyrimidin-5-yl | 3.23^{[a]} |
| Ia-133 | H | H | H | CF₃ | 2-phenyl-1,3-thiazol-4-yl | 3.09^{[a]} |
| Ia-134 | H | H | H | CF₃ | 5-methyl-2-thienyl | 2.46^{[a]} |
| Ia-135 | H | H | H | CF₃ | 5-chloro-2-thienyl | 2.73^{[a]} |
| Ia-136 | H | H | H | CF₃ | 5-phenylsulfanyl-2-thienyl | |
| Ia-137 | H | H | H | CF₃ | 4-(trifluoromethoxy)phenyl | 3.14^{[a]} |
| Ia-138 | H | H | H | CF₃ | 4-(trifluoromethylthio)phenyl | 3.48^{[a]} |
| Ia-139 | H | H | H | CHF₂ | 4-carboxyphenyl | 1.40^{[a]} |
| Ia-140 | H | H | H | CHF₂ | 4-(methoxycarbonyl)phenyl | 2.03^{[a]} |
| Ia-141 | H | H | H | CHF₂ | 4-(3-ethyl-1,2,4-oxadiazol-5-yl)phenyl | 2.49^{[a]} |
| Ia-142 | H | H | H | CHF₂ | 4-aminophenyl | 0.91^{[a]} |
| Ia-143 | H | H | H | CHF₂ | 2-anilinopyrimidin-5-yl | 2.17^{[a]} |
| Ia-144 | H | F | F | CF₃ | 4-(3-ethyl-1,2,4-oxadiazol-5-yl)phenyl | 3.64^{[a]} |
| Ia-145 | H | H | F | CF₃ | 4-(3-ethyl-1,2,4-oxadiazol-5-yl)phenyl | 2.88^{[a]} |
| Ia-146 | H | H | H | CF₃ | 4-(2,2-difluoro-1,3-benzodioxol-4-yl)phenyl | 3.96^{[a]} |
| Ia-147 | H | H | H | CF₃ | 4-(pentafluoro-λ6-sulfanyl)phenyl | 3.31^{[a]} |
| Ia-148 | H | H | H | CF₃ | 4-(2-aminophenyl)phenyl | |
| Ia-149 | H | H | H | CF₃ | 4-[1-(cyclopropylamino)ethyl]phenyl | 1.10^{[a]} |
| Ia-150 | H | H | H | CF₃ | 4-[2-(ethoxycarbonyl)phenyl]phenyl | 3.48^{[a]} |
| Ia-151 | H | H | H | CF₃ | 4-(9H-carbazol-2-yl)phenyl | 3.92^{[a]} |
| Ia-152 | H | H | H | CF₃ | 4-[2-(tert-butoxycarbonyl)phenyl]phenyl | 3.99^{[a]} |
| Ia-153 | H | H | H | CF₃ | 5-(3-fluorophenyl)-2-thienyl | 3.46^{[a]} |
| Ia-154 | H | H | H | CF₃ | 5-[2-(acetylamino)pyridin-4-yl]-2-thienyl | 1.92^{[a]} |
| Ia-155 | H | H | H | CF₃ | 5-(1-ethyl-1H-pyrazol-4-yl)-2-thienyl | 2.47^{[a]} |
| Ia-156 | H | H | H | CF₃ | 4-[2-(methoxycarbonyl)phenyl]phenyl | 3.19[a] |
| Ia-157 | H | H | H | CF₃ | 4-[3-((2,4-difluoroanilino)carbonyl)phenyl]phenyl | 3.41[a] |
| Ia-158 | H | H | H | CF₃ | 4-[3-(methylaminocarbonyl)phenyl]phenyl | 2.26[a] |
| Ia-159 | H | H | H | CF₃ | 4-[3-(dimethylaminocarbonyl)phenyl]phenyl | 2.44[a] |
| Ia-160 | H | H | H | CF₃ | 4-[3-(2-fluorobenzylaminocarbonyl)phenyl]phenyl | 3.29[a] |
| Ia-161 | H | H | H | CF₃ | 4-(chloromethyl)phenyl | 2.84[a] |
| Ia-162 | H | H | H | CF₃ | 4-[(4-fluorophenoxy)methyl]phenyl | 3.48[a] |

TABLE 2
Compounds according to formula (I) (in accordance with embodiments 1 and 2)

| | |
|---|---|
| | (I) |
| 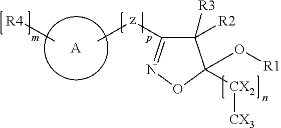 | |

| Ex N^{o} | R1 | R2 | R3 | 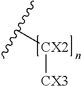 | 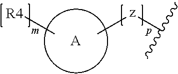 | LogP |
|---|---|---|---|---|---|---|
| Ib-001 | H | H | H | CF₂Br | 4-nitrophenyl | 2.68^{[a]} |
| Ib-002 | H | H | H | 1,1,2,2,2-pentafluoroethyl | 4-nitrophenyl | 2.96^{[a]} |
| Ib-003 | H | H | H | CF₂Br | 4-(dimethylaminocarbonyl)phenyl | 1.92^{[a]} |
| Ib-004 | H | H | H | CF₂Br | 4-[[(1-methylcyclopropyl)amino]carbonyl]phenyl | 2.22^{[a]} |
| Ib-005 | H | H | H | CF₂Br | 4-[(2,4-difluoroanilino)carbonyl]phenyl | 2.81^{[a]} |
| Ib-006 | H | H | H | CF₂Br | 4-[(cyclopropylmethylamino)carbonyl]phenyl | 2.32^{[a]} |
| Ib-007 | H | H | H | CF₂Br | 4-[[(4-fluorophenyl)methylamino]carbonyl]phenyl | 2.70^{[a]} |
| Ib-008 | H | H | H | CF₂Br | 4-[(2-methoxyethylamino)carbonyl]phenyl | 1.91^{[a]} |
| Ib-009 | H | H | H | CF₂Br | 4-carboxyphenyl | 1.94^{[a]} |
| Ib-010 | H | H | H | CF₂Br | 4-(methoxycarbonyl)phenyl | 2.59^{[a]} |
| Ib-011 | H | H | H | CF₂Br | 4-(diacetylamino)phenyl | 2.40^{[a]} |
| Ib-012 | H | H | H | CF₂Br | 4-aminophenyl | 1.79^{[a]} |
| Ib-013 | 3-fluorobenzoyl | H | H | CF₂Br | 4-aminophenyl | 3.60^{[a]} |
| Ib-014 | H | H | H | CF₂Br | 4-(acetylamino)phenyl | 2.45^{[a]} |
| Ib-015 | H | H | H | CF₂Br | 4-[[(1-methylcyclopropyl)carbonyl]amino]phenyl | 2.53^{[a]} |
| Ib-016 | H | H | H | CF₂Br | 4-[(3-fluorobenzoyl)amino]phenyl | 2.94^{[a]} |
| Ib-017 | 3-fluorobenzoyl | H | H | CF₂Br | 4-[(3-fluorobenzoyl)amino]phenyl | 4.54^{[a]} |
| Ib-018 | H | H | H | CF₂Br | 4-[(3-fluorophenyl)carbonothioylamino]phenyl | 3.37^{[a]} |
| Ib-019 | H | H | H | CF₂Cl | 4-(dimethylaminocarbonyl)phenyl | 1.79^{[a]} |
| Ib-020 | H | H | H | CF₂Cl | 4-[(N-methylanilino)carbonyl]phenyl | 2.60^{[a]} |
| Ib-021 | H | H | H | CF₂Cl | 4-[[2-methoxyethyl(methyl)amino]carbonyl]phenyl | 1.91^{[a]} |
| Ib-022 | H | H | H | CF₂Cl | 4-(piperidin-1-ylcarbonyl)phenyl | 2.43^{[a]} |
| Ib-023 | H | H | H | CF₂Cl | 4-(3,4-dihydro-1H-2,3-benzoxazin-3-ylcarbonyl)phenyl | 3.00^{[a]} |
| Ib-024 | H | H | H | CF₂Cl | 4-[[1-(4,5-dihydro-1,2-oxazol-3-yl)ethylamino]carbonyl]phenyl | 1.87^{[a]} |
| Ib-025 | H | H | H | CF₂Cl | 4-[[1-(2-oxo-1,2,3,4-tetrahydroquinolin-6-yl)ethylamino]carbonyl]phenyl | 2.07^{[a]} |
| Ib-026 | H | H | H | CF₂Cl | 4-[(butan-2-ylamino)carbonyl]phenyl | 2.37^{[a]} |
| Ib-027 | H | H | H | CF₂Cl | 4-(methylcarbamoyl)phenyl | 1.60^{[a]} |
| Ib-028 | H | H | H | CF₂Cl | 4-[[(1-methylcyclopropyl)amino]carbonyl]phenyl | 2.13^{[a]} |
| Ib-029 | H | H | H | CF₂Cl | 4-[[[1-(1-methylcyclopropyl)cyclopropyl]amino]carbonyl]phenyl | 2.80^{[a]} |
| Ib-030 | H | H | H | CF₂Cl | 4-[(2,4-difluoroanilino)carbonyl]phenyl | 2.76^{[a]} |
| Ib-031 | H | H | H | CF₂Cl | 4-[(3-fluoroanilino)carbonyl]phenyl | 2.96^{[a]} |
| Ib-032 | H | H | H | CF₂Cl | 4-[(cyclopentylamino)carbonyl]phenyl | 2.51^{[a]} |
| Ib-033 | H | H | H | CF₂Cl | 4-[[(3-chloropyridin-2-yl)amino]carbonyl]phenyl | 2.13^{[a]} |
| Ib-034 | H | H | H | CF₂Cl | 4-[(prop-2-yn-1-ylamino)carbonyl]phenyl | 1.91^{[a]} |
| Ib-035 | H | H | H | CF₂Cl | 4-[(2,2-dimethylpropylamino)carbonyl]phenyl | 2.71^{[a]} |
| Ib-036 | H | H | H | CF₂Cl | 4-[[(1-phenylcyclopropyl)methylamino]carbonyl]phenyl | 3.08^{[a]} |
| Ib-037 | H | H | H | CF₂Cl | 4-[[[2-methyloxolan-2-yl]methylamino]carbonyl]phenyl | 2.14^{[a]} |
| Ib-038 | H | H | H | CF₂Cl | 4-[[[2-bicyclo[2.2.1]heptanyl]methylamino]carbonyl]phenyl | 3.21^{[a]} |
| Ib-039 | H | H | H | CF₂Cl | 4-[[(3-cyanophenyl)methylamino]carbonyl]phenyl | 2.44^{[a]} |
| Ib-040 | H | H | H | CF₂Cl | 4-[[(3-fluorophenyl)methylamino]carbonyl]phenyl | 2.69^{[a]} |
| Ib-041 | H | H | H | CF₂Cl | 4-[(cyclopropylmethylamino)carbonyl]phenyl | 2.23^{[a]} |
| Ib-042 | H | H | H | CF₂Cl | 4-[[(4-fluorophenyl)methylamino]carbonyl]phenyl | 2.68^{[a]} |
| Ib-043 | H | H | H | CF₂Cl | 4-[(oxan-4-ylmethylamino)carbonyl]phenyl | 1.91^{[a]} |
| Ib-044 | H | H | H | CF₂Cl | 4-[(imidazo[1,2-a]pyridin-2-ylmethylamino)carbonyl]phenyl | 1.19^{[a]} |
| Ib-045 | H | H | H | CF₂Cl | 4-[(2,2-dimethylhydrazino)carbonyl]phenyl | 1.39^{[a]} |
| Ib-046 | H | H | H | CF₂Cl | 4-[(1-cyclopropylethoxyamino)carbonyl]phenyl | 2.27^{[a]} |
| Ib-047 | H | H | H | CF₂Cl | 4-[(methoxyamino)carbonyl]phenyl | 1.57^{[a]} |
| Ib-048 | H | H | H | CF₂Cl | 4-carboxyphenyl | 1.84^{[a]} |
| Ib-049 | H | H | H | CF₂Cl | 4-(methoxycarbonyl)phenyl | 2.55^{[a]} |
| Ib-050 | H | H | H | CF₂Cl | 4-(3-ethyl-1,2,4-oxadiazol-5-yl)phenyl | 3.04^{[a]} |
| Ib-051 | H | H | H | CF₂Cl | 4-aminophenyl | 1.63^{[a]} |
| Ib-052 | pyrrolidin-1-ylcarbonyl | H | H | CF₂Cl | 4-aminophenyl | 2.88^{[a]} |
| Ib-053 | H | H | H | CF₂Cl | 4-(2,2-dimethylpropanoylamino)phenyl | 2.66^{[a]} |
| Ib-054 | H | H | H | CF₂Cl | 4-(2-methylpropanoylamino)phenyl | 2.30^{[a]} |
| Ib-055 | H | H | H | CF₂Cl | 4-(acetylamino)phenyl | 1.74^{[a]} |
| Ib-056 | H | H | H | CF₂Cl | 4-[[(1-cyanocyclopropyl)carbonyl]amino]phenyl | 2.27^{[a]} |
| Ib-057 | H | H | H | CF₂Cl | 4-[[(1-methylcyclopropyl)carbonyl]amino]phenyl | 2.48^{[a]} |
| Ib-058 | H | H | H | CF₂Cl | 4-[(2-fluorobenzoyl)amino]phenyl | 2.75^{[a]} |
| Ib-059 | H | H | H | CF₂Cl | 4-[(3-chlorobenzoyl)amino]phenyl | 3.17^{[a]} |
| Ib-060 | H | H | H | CF₂Cl | 4-[(3-fluorobenzoyl)amino]phenyl | 2.86^{[a]} |
| Ib-061 | H | H | H | CF₂Cl | 4-[(4-cyanobenzoyl)amino]phenyl | 2.64^{[a]} |
| Ib-062 | H | H | H | CF₂Cl | 4-[(4-chlorobenzoyl)amino]phenyl | 3.15^{[a]} |
| Ib-063 | H | H | H | CF₂Cl | 4-[(4-fluorobenzoyl)amino]phenyl | 2.82^{[a]} |
| Ib-064 | H | H | H | CF₂Cl | 4-[(4-methoxybenzoyl)amino]phenyl | 2.73^{[a]} |
| Ib-065 | H | H | H | CF₂Cl | 4-[(cyclobutylcarbonyl)amino]phenyl | 2.48^{[a]} |
| Ib-066 | H | H | H | CF₂Cl | 4-[(cyclopentylcarbonyl)amino]phenyl | 2.75^{[a]} |
| Ib-067 | H | H | H | CF₂Cl | 4-(benzoylamino)phenyl | 2.68^{[a]} |
| Ib-068 | H | H | H | CF₂Cl | 4-[(cyclohexylcarbonyl)amino]phenyl | 3.04^{[a]} |
| Ib-069 | H | H | H | CF₂Cl | 4-[(oxan-4-ylcarbonyl)amino]phenyl | 1.98^{[a]} |
| Ib-070 | H | H | H | CF₂Cl | 4-[[(4-methyl-1,3-oxazol-5-yl)carbonyl]amino]phenyl | 2.10^{[a]} |
| Ib-071 | H | H | H | CF₂Cl | 4-[(1,2-oxazol-5-ylcarbonyl)amino]phenyl | 2.14^{[a]} |
| Ib-072 | H | H | H | CF₂Cl | 4-[(2-cyclopentylacetyl)amino]phenyl | 3.04^{[a]} |
| Ib-073 | H | H | H | CF₂Cl | 4-[(2-phenylacetyl)amino]phenyl | 2.71^{[a]} |
| Ib-074 | H | H | H | CF₂Cl | 4-[(2-methoxyacetyl)amino]phenyl | 1.93^{[a]} |
| Ib-075 | H | H | H | CF₂Cl | 4-[(pyrrolidin-1-ylcarbonyl)amino]phenyl | 3.11^{[a]} |
| Ib-076 | H | H | H | CF₂Cl | 4-[[(cyclopentylamino)carbonyl]amino]phenyl | 2.50^{[a]} |
| Ib-077 | H | H | H | CF₂Cl | 4-[(methoxycarbonyl)amino]phenyl | 2.13^{[a]} |
| Ib-078 | methoxycarbonyl | H | H | CF₂Cl | 4-[(methoxycarbonyl)amino]phenyl | 2.88^{[a]} |
| Ib-079 | H | H | H | CF₂Cl | 4-[(3-phenylprop-2-ynoxycarbonyl)amino]phenyl | 3.48^{[a]} |
| Ib-080 | H | H | H | CF₂Cl | 4-[(2-methylpropoxycarbonyl)amino]phenyl | 3.13^{[a]} |
| Ib-081 | H | H | H | CF₂Cl | 4-[(ethoxycarbonyl)amino]phenyl | 2.80^{[a]} |
| Ib-082 | H | H | H | CF₂Cl | 4-[[(2,6-dichlorophenyl)methoxycarbonyl]amino]phenyl | 3.73^{[a]} |
| Ib-083 | H | H | H | CF₂Cl | 4-[(cyclopentylmethoxycarbonyl)amino]phenyl | 3.60^{[a]} |
| Ib-084 | H | H | H | CF₂Cl | 4-(phenylmethoxycarbonylamino)phenyl | 3.19^{[a]} |
| Ib-085 | H | H | H | CF₂Cl | 4-(cyclopentylcarbamothioylamino)phenyl | 2.88^{[a]} |
| Ib-086 | H | H | H | CF₂Cl | 4-(phenylcarbamothioylamino)phenyl | 2.68^{[a]} |
| Ib-087 | H | H | H | CF₂Cl | 4-[(4,6-dimethylpyrimidin-2-yl)amino]phenyl | 2.77^{[a]} |
| Ib-088 | H | H | H | CF₂Cl | 4-[(4-methylpyrimidin-2-yl)amino]phenyl | 2.58^{[a]} |
| Ib-089 | H | H | H | CF₂Cl | 4-(pyrimidin-2-ylamino)phenyl | 2.33^{[a]} |
| Ib-090 | H | H | H | CF₂Cl | 4-(benzenesulfonamido)phenyl | 2.59^{[a]} |
| Ib-091 | H | H | H | CF₂Cl | 4-hydroxyphenyl | 1.79^{[a]} |
| Ib-092 | H | H | H | CF₂Cl | 4-[(diisopropylamino)carbonyl]oxyphenyl | 3.73^{[a]} |
| Ib-093 | H | H | H | CF₂Cl | 4-[(4-chloro-N-isopropylanilino)carbonyl]oxyphenyl | 4.20^{[a]} |
| Ib-094 | H | H | H | CF₂Cl | 4-[(N-isopropylanilino)carbonyl]oxyphenyl | 3.77^{[a]} |
| Ib-095 | H | H | H | CF₂Cl | 4-(dimethylaminocarbonyl)oxyphenyl | 2.35^{[a]} |
| Ib-096 | H | H | H | CF₂Cl | 4-[(N-methylanilino)carbonyl]oxyphenyl | 3.23^{[a]} |
| Ib-097 | H | H | H | CF₂Cl | 4-[[cyclohexyl(methyl)amino]carbonyl]oxyphenyl | 3.88^{[a]} |
| Ib-098 | H | H | H | CF₂Cl | 4-[[ethyl(methyl)amino]carbonyl]oxyphenyl | 2.71^{[a]} |
| Ib-099 | H | H | H | CF₂Cl | 4-[(N-ethyl-4-methylanilino)carbonyl]oxyphenyl | 3.86^{[a]} |
| Ib-100 | H | H | H | CF₂Cl | 4-[(N-ethylanilino)carbonyl]oxyphenyl | 3.52^{[a]} |
| Ib-101 | H | H | H | CF₂Cl | 4-(diethylaminocarbonyl)oxyphenyl | 3.06^{[a]} |
| Ib-102 | H | H | H | CF₂Cl | 4-[(N-benzyl-2-methylanilino)carbonyl]oxyphenyl | 4.27^{[a]} |
| Ib-103 | H | H | H | CF₂Cl | 4-[(dipropylamino)carbonyl]oxyphenyl | 3.81^{[a]} |
| Ib-104 | H | H | H | CF₂Cl | 4-(pyrrolidin-1-ylcarbonyl)oxyphenyl | 2.75^{[a]} |
| Ib-105 | H | H | H | CF₂Cl | 4-(piperidin-1-ylcarbonyl)oxyphenyl | 3.21^{[a]} |
| Ib-106 | H | H | H | CF₂Cl | 4-(morpholin-4-ylcarbonyl)oxyphenyl | 2.26^{[a]} |
| Ib-107 | H | H | H | CF₂Cl | 4-(11H-benzo[b][1]benzazepin-11-ylcarbonyl)oxyphenyl | 3.96^{[a]} |
| Ib-108 | H | H | H | CF₂Cl | 4-(6,11-dihydro-5H-benzo[b][1]benzazepin-11-ylcarbonyl)oxyphenyl | 4.03^{[a]} |
| Ib-109 | H | H | H | CF₂Cl | 4-(5,6-dihydrobenzo[b][1,4]benzoxazepin-5-ylcarbonyl)oxyphenyl | 4.11^{[a]} |
| Ib-110 | H | H | H | CF₂Cl | 4-(1,2,3,4-tetrahydroisoquinolin-2-ylcarbonyl)oxyphenyl | 3.62^{[a]} |
| Ib-111 | H | H | H | CF₂Cl | 4-methylsulfanylphenyl | 2.92^{[a]} |
| Ib-112 | H | H | H | CF₂Cl | 2-anilinopyrimidin-5-yl | 2.68^{[a]} |
| Ib-113 | H | H | H | 1,1,2,2,2-pentafluoroethyl | 4-aminophenyl | 2.04^{[a]} |
| Ib-114 | H | H | H | 1,1,2,2,2-pentafluoroethyl | 4-(acetylamino)phenyl | 2.08^{[a]} |
| Ib-115 | H | H | H | 1,1,2,2,2-pentafluoroethyl | 4-[[(1-methylcyclopropyl)carbonyl]amino]phenyl | 2.82^{[a]} |
| Ib-116 | H | H | H | 1,1,2,2,2-pentafluoroethyl | 4-[(3-fluorobenzoyl)amino]phenyl | 3.23^{[a]} |
| Ib-117 | H | H | H | 1,1,2,2,2-pentafluoroethyl | 4-[(3-fluorophenyl)carbonothioylamino]phenyl | 3.61^{[a]} |
| Ib-118 | H | H | H | CF₂Cl | 4-(2,2,2-trifluoroethylsulfonylamino)phenyl | 2.48^{[a]} |
| Ib-119 | 3-fluorobenzoyl | H | H | 1,1,2,2,2-pentafluoroethyl | 4-[(3-fluorobenzoyl)amino]phenyl | 4.74^{[a]} |

TABLE 3
Compounds according to formula (I) (in accordance with embodiments 1 and 4)

| | |
|---|---|
| | (I) |
| 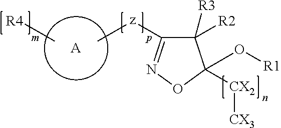 | |

| Ex N^{o} | R1 | R2 | R3 | 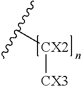 | 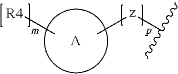 | LogP |
|---|---|---|---|---|---|---|
| Ic-001 | H | H | H | CF₃ | 1′-acetylspiro[3H-2-benzofuran-1,3′-azetidine]-5-yl | 1.84^{[a]} |
| Ic-002 | H | H | H | CF₃ | 1′-(cyclopropylcarbonyl)spiro[3H-2-benzofuran-1,3′-azetidine]-5-yl | 2.08^{[a]} |
| Ic-003 | H | H | H | CF₃ | 1′-benzoylspiro[3H-2-benzofuran-1,3′-azetidine]-5-yl | 2.53^{[a]} |
| Ic-004 | H | H | H | CF₃ | spiro[3H-2-benzofuran-1,3′-azetidine]-5-yl | 0.88^{[a]} |
| Ic-005 | H | H | H | CF₃ | naphthalen-2-yl | |
| Ic-006 | H | H | H | CF₃ | 10H-phenothiazin-2-yl | 3.50^{[a]} |
| Ic-007 | H | H | H | CF₃ | 1-[2-(2-fluorophenyl)acetyl]piperidin-4-yl | 2.20^{[a]} |
| Ic-008 | H | H | H | CF₃ | 5-[2-(acetylamino)pyridin-4-yl]-1-benzothiophen-3-yl | 2.27^{[a]} |
| Ic-009 | H | H | H | CF₃ | 1-benzothiophen-2-yl | 3.00^{[a]} |
| Ic-010 | H | H | H | CF₃ | 1H-indazol-5-yl | 1.65^{[a]} |
| Ic-011 | CH₃ | H | H | CF₃ | 1H-indazol-5-yl | 2.33^{[a]} |
| Ic-012 | H | H | H | CF₃ | 6-hydroxynaphthalen-2-yl | 2.22^{[a]} |
| Ic-013 | H | H | H | CF₃ | 6-[[isopropyl(methyl)amino]carbonyl]oxynaphthalen-2-yl | 3.48^{[a]} |
| Ic-014 | H | H | H | CF₃ | 6-(dimethylaminocarbonyl)oxynaphthalen-2-yl | 2.80^{[a]} |
| Ic-015 | H | H | H | CF₃ | 6-[[ethyl(methyl)amino]carbonyl]oxynaphthalen-2-yl | 3.15^{[a]} |
| Ic-016 | H | H | H | CF₃ | 1-methyl-1H-indazol-5-yl | 2.00^{[a]} |
| Ic-017 | CH₃ | H | H | CF₃ | 1-methyl-1H-indazol-5-yl | 2.73^{[a]} |
| Ic-018 | H | H | H | CF₃ | 1-(2-methoxyethyl)-1H-indazol-5-yl | 2.07^{[a]} |
| Ic-019 | H | H | H | CF₃ | 2-benzyl-2H-indazol-5-yl | 2.68^{[a]} |
| Ic-020 | benzyl | H | H | CF₃ | 2-benzyl-2H-indazol-5-yl | 4.37^{[a]} |
| Ic-021 | H | H | H | CF₃ | 2-(2-methoxyethyl)-2H-indazol-5-yl | 1.87^{[a]} |
| Ic-022 | H | H | H | CF₃ | 2-[(tert-butoxycarbonyl)amino]-1,3-benzothiazol-6-yl | 3.06^{[a]} |
| Ic-023 | H | H | H | CF₃ | 6-[(N-methylanilino)carbonyl]oxynaphthalen-2-yl | 3.61^{[a]} |
| Ic-024 | H | H | H | CF₃ | 6-[[cyclohexyl(methyl)amino]carbonyl]oxynaphthalen-2-yl | 4.32^{[a]} |
| Ic-025 | H | H | H | CF₃ | 1-benzyl-1H-indazol-5-yl | 2.96^{[a]} |
| Ic-026 | benzyl | H | H | CF₃ | 1-benzyl-1H-indazol-5-yl | 4.68^{[a]} |
| Ic-027 | H | H | H | CF₃ | 2-[(cyclopropylcarbonyl)amino]-1,3-benzothiazol-6-yl | 2.39^{[a]} |
| Ic-028 | H | H | H | CF₃ | 3-oxo-3,4-dihydro-1,4-benzoxazin-6-yl | 1.74^{[a]} |
| Ic-029 | H | H | H | CF₃ | 1-(tert-butoxycarbonyl)piperidin-4-yl | 2.60^{[a]} |
| Ic-030 | H | H | H | CF₃ | 5-(1-ethyl-1H-pyrazol-4-yl)-1-benzothiophen-3-yl | 2.94^{[a]} |
| Ic-031 | H | H | H | CF₃ | 5-(3-fluorophenyl)-1-benzothiophen-3-yl | 4.13^{[a]} |
| Ic-032 | H | H | H | CF₃ | 5-bromo-1-benzothiophen-3-yl | 3.52^{[a]} |
| Ic-033 | H | H | H | CF₃ | 2-(acetylamino)-1,3-benzothiazol-6-yl | 1.95^{[a]} |

Intermediates of formula (1b) as described in table 4 were prepared in analogy with the examples provided above.

TABLE 4
Compounds according to formula (1b)

| | |
|---|---|
| | (1b) |
| 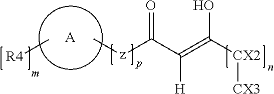 | |

| Ex N^{o} | 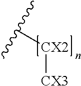 | 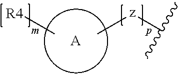 | LogP |
|---|---|---|---|
| 1b-01 | CF₃ | 4-[N-methylanilino)carbonyl]phenyl | 2.17^{[a]} |
| 1b-02 | CF₃ | 4-(3-ethyl-1,2,4-oxadiazol-5-yl)phenyl | 2.43^{[a]} |
| 1b-03 | CF₃ | 4-(3-ethyl-1,2,4-oxadiazol-5-yl)phenyl | 2.40^{[a]} |
| 1b-04 | CF₂Cl | 4-(methoxycarbonyl)phenyl | 2.11^{[a]} |
| 1b-05 | CF₃ | 4-methylsulfanylphenyl | 2.49^{[a]} |
| 1b-06 | CF₃ | 4-phenylsulfanylphenyl | 3.44^{[a]} |
| 1b-07 | CF₃ | 4-cyclohexylphenyl | 2.98^{[a]} |
| 1b-08 | CF₃ | 4-(trifluoromethylthio)phenyl | 3.94^{[a]} |
| 1b-09 | CF₃ | 4-morpholin-4-ylphenyl | 3.61^{[a]} |
| 1b-10 | CF₃ | 10H-phenothiazin-2-yl | 3.19^{[a]} |
| 1b-11 | CF₃ | 1-(tert-butoxycarbonyl)piperidin-4-yl | 2.37^{[a]} |
| 1b-12 | CF₃ | 2-[(tert-butoxycarbonyl)amino]-1,3-benzothiazol-6-yl | 2.75 + 4.56^{[a]} |
| 1b-13 | CF₃ | 3-fluoro-4-hydroxyphenyl | 1.42 + 2.92^{[a]} |
| 1b-14 | CF₃ | (E)-2-(3,4-dimethoxyphenyl)ethen-1-yl | 2.05 + 3.79^{[a]} |
| 1b-15 | CF₃ | 4-ethoxy-2,3-difluorophenyl | 2.58 + 4.41^{[a]} |
| 1b-16 | CF₃ | 5-methyl-2-thienyl | 1.96^{[a]} |
| 1b-17 | CF₃ | 5-chloro-2-thienyl | 2.15^{[a]} |
| 1b-18 | CF₃ | 1-benzothiophen-2-yl | 2.51^{[a]} |
| 1b-19 | CF₃ | 5-bromo-1-benzothiophen-3-yl | 3.02^{[a]} |
| 1b-20 | CF₃ | 5-phenylsulfanyl-2-thienyl | 3.29 + 5.24^{[a]} |
| 1b-21 | CF₃ | 4-(pentafluoro-λ6-sulfanyl)phenyl | 2.82 + 4.51^{[a]} |
| 1b-22 | CF₃ | 2-anilinopyrimidin-5-yl | 2.08^{[a]} |
| 1b-23 | CF₂Cl | 2-anilinopyrimidin-5-yl | 2.25 + 3.87^{[a]} |
| 1b-24 | CHF₂ | 2-anilinopyrimidin-5-yl | 1.76 + 3.11^{[a]} |
| 1b-25 | CF₃ | 1′-(tert-butoxycarbonyl)spiro[3H-2-benzofuran-1,3′- | 2.82 + 4.56^{[a]} |
| | | azetidine]-5-yl | |
| 1b-26 | CF₃ | 4-methyl-3-nitrophenyl | 2.26^{[a]} |
| 1b-27 | 1,1,2,2,2-pentafluoroethyl | 4-nitrophenyl | 2.51 + 4.01^{[a]} |
| 1b-28 | CF₂Br | 4-nitrophenyl | 2.17 + 3.74^{[a]} |
| 1b-29 | CHF₂ | 4-nitrophenyl | 2.46 + 3.00^{[a]} |
| 1b-30 | CF₃ | 2-fluoro-4-(methoxycarbonyl)phenyl | 2.08 + 3.81^{[a]} |
| 1b-31 | CF₃ | 1H-indazol-5-yl | 2.69^{[a]} |
| 1b-32 | CF₃ | 4-(1,1-difluoro-2-methoxy-2-oxoethyl)phenyl | 2.39^{[a]} |
| 1b-33 | CF₃ | 3-oxo-3,4-dihydro-1,4-benzoxazin-6-yl | 1.39^{[a]} |
| 1b-34 | CF₃ | 2-[(1-phenylcyclopropyl)amino]pyrimidin-5-yl | 2.23 + 3.79^{[a]} |
| 1b-35 | CF₃ | 4-(2,5-dimethyl-1H-pyrrol-1-yl)phenyl | 3.21 + 5.03^{[a]} |
| 1b-36 | CF₃ | 4-(methoxycarbonyl)phenyl | 1.98^{[a]} |
| 1b-37 | CF₃ | 4-[(N-methylanilino)carbonyl]phenyl | 3.54^{[a]} |
| 1b-38 | CF₃ | 4-[(tert-butoxycarbonyl)amino]phenyl | 4.46^{[a]} |

It is understood that, the compounds of formula (1b) according to the invention, wherein R2 is hydrogen and R1, R3, R4, R5, R^{a}, R^{b}, R^{c}, n, m, p, X and Z are as defined hereinabove, may be present as the diketone (ie, the compounds of formula (1b_a)) or as the ketoenol form (ie, the compounds of formula (1b_a)) or a mixture of both forms. The compounds shown in table 4 are present as a mixture of both forms.

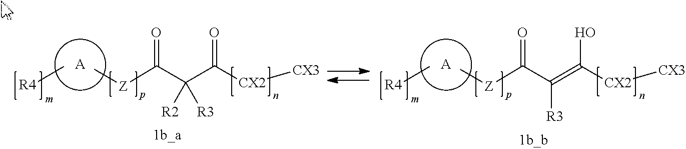

Measurement of LogP values was performed according to EEC directive 79/831 Annex V.A8 by HPLC (High Performance Liquid Chromatography) on reversed phase columns with the following methods:

[a] LogP value is determined by measurement of LC-UV, in anacidic range, with 0.1% formic acid in water and acetonitrile as eluent (linear gradient from 10% acetonitrile to 95% acetonitrile).
[b] LogP value is determined by measurement of LC-UV, in a neutral range, with 0.001 molar ammonium acetate solution in water and acetonitrile as eluent (linear gradient from 10% acetonitrile to 95% acetonitrile).
[c] LogP value is determined by measurement of LC-UV, in an acidic range, with 0.1% phosphoric acid and acetonitrile as eluent (linear gradient from 10% acetonitrile to 95% acetonitrile).

If more than one LogP value is available within the same method, all the values are given and separated by “+”.

Calibration was done with straight-chain alkan2-ones (with 3 to 16 carbon atoms) with known LogP values (measurement of LogP values using retention times with linear interpolation between successive alkanones). Lambda-max-values were determined using UV-spectra from 200 nm to 400 nm and the peak values of the chromatographic signals.

### NMR-Peak Lists

1H-NMR data of selected examples are written in form of 1H-NMR-peak lists. To each signal peak are listed the δ-value in ppm and the signal intensity in round brackets. Between the δ-value-signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within Patent Applications” of the Research Disclosure Database Number 564025.

### Compounds of Formula I

Ia-002: ¹H-NMR(300.2 MHz, CDCl3):
δ = 8.2209 (2.9); 8.2153 (3.1); 7.9217 (1.5); 7.9159 (1.6); 7.8949 (1.8); 7.8891 (1.8); 7.4968 (2.5); 7.4700 (2.2); 7.2990 (4.2);
4.1917 (0.5); 4.1679 (1.5); 4.1441 (1.5); 4.1204 (0.6); 3.9647 (0.7); 3.8050 (2.0); 3.7454 (3.5); 3.6042 (2.0); 3.6015 (2.2); 3.5418
(1.2); 2.6954 (16.0); 2.0847 (6.7); 1.7099 (2.2); 1.4939 (0.4); 1.3171 (1.7); 1.2932 (3.7); 1.2695 (1.7); 0.0356 (3.9)
Ia-003: ¹H-NMR(499.9 MHz, d₆-DMSO):
δ = 8.3295 (1.7); 8.3247 (14.4); 8.3206 (4.5); 8.3108 (4.7); 8.3067 (15.9); 8.3020 (1.9); 8.1285 (12.2); 8.0059 (2.1); 8.0012 (16.0);
7.9970 (4.8); 7.9873 (4.6); 7.9832 (14.3); 7.9784 (1.7); 6.3155 (2.8); 6.2060 (6.1); 6.0967 (3.3); 3.7903 (4.8); 3.7537 (5.8);
3.4210 (3.4); 3.4187 (3.5); 3.3842 (2.8); 3.3818 (2.9); 3.3274 (33.0); 2.5165 (2.1); 2.5128 (4.7); 2.5092 (6.5); 2.5055 (4.6);
2.5019 (2.1); 1.9952 (0.6); 1.2704 (0.5); 1.1819 (0.4)
Ia-004: ¹H-NMR(300.2 MHz, CDCl3):
δ = 7.6459 (0.7); 7.6402 (0.3); 7.6240 (0.4); 7.6175 (1.5); 7.5631 (1.6); 7.5567 (0.4); 7.5346 (0.7); 7.2990 (0.9); 3.7695 (0.5);
3.7101 (0.8); 3.5702 (0.5); 3.5668 (0.5); 0.3129 (0.6); 0.3013 (16.0); 0.2897 (0.6); 0.0378 (0.8)
Ia-005: ¹H-NMR(499.9 MHz, CDCl3):
δ = 7.6776 (0.4); 7.6415 (0.4); 7.5741 (9.4); 7.5704 (3.6); 7.5607 (5.1); 7.5569 (16.0); 7.5527 (2.6); 7.5136 (3.6); 7.5097 (16.0);
7.5058 (4.6); 7.4960 (3.9); 7.4925 (9.1); 7.4881 (1.3); 7.2586 (1.7); 4.0189 (0.4); 3.7088 (5.5); 3.6732 (7.8); 3.5117 (4.6); 3.4760
(3.2); 2.0450 (0.5); −0.0002 (1.6)
Ia-006: ¹H-NMR(300.2 MHz, CDCl3):
δ = 7.6338 (1.5); 7.6264 (0.7); 7.6120 (1.1); 7.6046 (5.0); 7.5989 (1.1); 7.5836 (1.2); 7.5783 (5.0); 7.5708 (1.1); 7.5562 (0.7);
7.5489 (1.5); 7.2986 (5.4); 3.9634 (0.7); 3.9586 (0.7); 3.9027 (1.2); 3.8979 (1.2); 3.7461 (2.2); 3.6856 (1.3); 2.2285 (0.4); 2.2180
(16.0); 1.5852 (5.8); 0.0380 (5.6)
Ia-007: ¹H-NMR(300.2 MHz, CDCl3):
δ = 7.6456 (0.5); 7.6434 (0.5); 7.6155 (2.3); 7.6004 (2.4); 7.5710 (0.5); 7.3000 (9.4); 4.0409 (0.4); 3.9848 (0.4); 3.9802 (0.5);
3.7531 (0.9); 3.6922 (0.6); 1.5846 (14.0); 1.5112 (16.0); 0.0499 (0.5); 0.0391 (12.0)
Ia-008: ¹H-NMR(300.2 MHz, d₆-DMSO):
δ = 8.8092 (6.6); 7.3895 (0.6); 7.3760 (16.0); 7.3626 (0.6); 3.9771 (2.2); 3.9155 (3.0); 3.6094 (1.7); 3.6061 (1.8); 3.5477 (1.3);
3.5442 (1.3); 3.3607 (3.0); 2.5342 (0.4); 2.5284 (0.9); 2.5224 (1.2); 2.5164 (0.9); 2.5106 (0.4); 0.0175 (1.4)
Ia-009: ¹H-NMR(499.9 MHz, d₆-DMSO):
δ = 8.6374 (3.0); 8.0532 (0.6); 8.0361 (0.7); 7.8771 (0.7); 7.8600 (0.6); 7.7488 (4.8); 7.7319 (6.2); 7.6920 (0.4); 7.6851 (2.6);
7.6804 (2.9); 7.6731 (3.5); 7.6657 (3.0); 7.6569 (0.4); 7.6025 (6.2); 7.5855 (5.0); 7.4644 (0.9); 7.4569 (7.0); 7.4516 (6.2); 7.4436
(4.4); 7.4374 (1.1); 4.0392 (0.4); 4.0249 (0.4); 3.9838 (0.4); 3.9455 (2.1); 3.9083 (2.5); 3.6873 (1.9); 3.6531 (4.5); 3.6123 (3.4);
3.5776 (1.5); 3.5670 (1.8); 3.5297 (1.5); 3.3206 (20.7); 2.6216 (2.9); 2.5111 (2.2); 2.5076 (4.8); 2.5040 (6.6); 2.5004 (4.8);
2.4969 (2.4); 1.9909 (1.7); 1.7314 (16.0); 1.2466 (0.8); 1.1917 (0.5); 1.1774 (0.9); 1.1632 (0.4); 0.8739 (0.5); 0.8604 (1.4);
0.8461 (0.6)
Ia-010: ¹H-NMR(499.9 MHz, d₆-DMSO):
δ = 8.6283 (1.7); 7.7152 (4.5); 7.6984 (5.2); 7.5153 (5.5); 7.4984 (4.6); 5.7552 (3.7); 3.9413 (2.0); 3.9043 (2.4); 3.5608 (1.5);
3.5236 (1.2); 3.3176 (9.2); 3.2061 (1.3); 3.2046 (1.3); 3.1715 (2.4); 3.1701 (2.4); 3.0963 (2.4); 3.0618 (1.3); 2.5092 (2.1); 2.5059
(3.8); 2.5024 (4.9); 2.4989 (3.5); 2.0776 (0.5); 1.9894 (1.0); 1.8947 (14.2); 1.6107 (1.5); 1.6010 (16.0); 1.1762 (0.6)
Ia-011: ¹H-NMR(400.1 MHz, d₆-DMSO):
δ = 8.6748 (1.2); 7.8149 (2.1); 7.7954 (2.7); 7.7855 (4.0); 7.7655 (4.3); 7.6868 (0.9); 7.6680 (2.1); 7.6492 (1.4); 7.5608 (1.5);
7.5419 (2.2); 7.5229 (1.0); 7.4825 (2.4); 7.4634 (2.0); 7.4182 (4.4); 7.3983 (3.9); 4.0046 (1.8); 3.9583 (2.2); 3.6228 (16.0);
3.5736 (1.4); 3.3110 (5.4); 2.5092 (7.4); 2.0798 (1.0)
Ia-012: ¹H-NMR(300.2 MHz, d₆-DMSO):
δ = 8.5698 (2.7); 7.3062 (1.4); 7.3001 (1.5); 7.1580 (0.7); 7.1517 (0.6); 7.1303 (0.9); 7.1240 (0.9); 7.0427 (4.3); 7.0059 (1.7);
6.9780 (1.2); 3.8262 (9.3); 3.8018 (9.0); 3.7351 (0.9); 3.6744 (1.2); 3.4310 (0.8); 3.3699 (0.6); 3.3474 (16.0); 2.5345 (1.3);
2.5286 (2.8); 2.5226 (3.9); 2.5166 (2.8); 2.5107 (1.3); 0.0207 (5.8)
Ia-014: ¹H-NMR(300.2 MHz, d₆-DMSO):
δ = 8.7014 (3.6); 7.5647 (0.5); 7.5568 (0.5); 7.5346 (0.8); 7.5305 (0.7); 7.5271 (0.7); 7.5082 (0.5); 7.5005 (0.5); 7.1873 (0.5);
7.1813 (0.6); 7.1563 (0.9); 7.1310 (0.4); 7.1253 (0.4); 4.2771 (0.8); 4.2538 (2.8); 4.2305 (2.8); 4.2073 (0.8); 3.9739 (1.0); 3.9120
(1.4); 3.5863 (0.9); 3.5242 (0.7); 3.3476 (16.0); 2.5346 (1.4); 2.5287 (2.9); 2.5226 (4.1); 2.5166 (3.0); 2.5107 (1.4); 1.4186 (2.9);
1.3954 (6.2); 1.3721 (2.8); 0.0200 (6.8)
Ia-015: ¹H-NMR(300.2 MHz, d₆-DMSO):
δ = 13.6912 (0.5); 13.5840 (1.9); 8.8273 (4.1); 8.1472 (0.5); 8.1229 (0.8); 8.0973 (0.7); 8.0371 (0.4); 8.0048 (1.3); 7.9734 (6.7);
7.9471 (12.8); 7.9230 (11.2); 7.8957 (1.7); 7.8791 (14.0); 7.8742 (16.0); 7.8521 (7.2); 7.8471 (10.6); 7.8354 (11.4); 7.7971
(10.1); 7.7928 (9.5); 4.0257 (8.4); 3.9635 (11.3); 3.6423 (8.3); 3.5797 (6.2); 3.3538 (13.1); 3.1903 (0.4); 2.5286 (22.2); 2.5226
(29.9); 2.5167 (22.4); 1.2531 (1.8); 0.0297 (1.6); 0.0189 (29.3); 0.0081 (1.4)
Ia-016: ¹H-NMR(300.2 MHz, CDCl3):
δ = 8.0631 (0.9); 8.0363 (1.6); 8.0124 (1.4); 7.9292 (1.7); 7.9240 (1.9); 7.9018 (1.1); 7.8966 (1.4); 7.8686 (1.5); 7.8642 (1.3);
7.8420 (0.3); 7.8303 (1.4); 7.8256 (1.3); 7.2993 (7.5); 3.9950 (16.0); 3.9834 (1.6); 3.9739 (2.0); 3.9504 (0.8); 3.8980 (0.7);
3.8902 (0.8); 3.8357 (1.3); 3.8280 (1.4); 3.6960 (1.2); 3.6911 (1.2); 3.6868 (1.1); 3.6338 (0.8); 3.6289 (0.8); 3.6246 (0.7); 2.3429
(0.8); 2.3339 (0.8); 1.6577 (0.4); 1.6292 (1.9); 1.3604 (0.5); 1.3327 (0.4); 1.3067 (0.4); 1.2916 (1.1); 0.9849 (0.5); 0.9606 (1.0);
0.9363 (0.5); 0.0487 (0.5); 0.0379 (9.5); 0.0272 (0.6)
Ia-017: ¹H-NMR(400.0 MHz, CDCl3):
δ = 7.7558 (1.8); 7.7347 (1.9); 7.4739 (1.8); 7.4688 (1.9); 7.3505 (1.5); 7.3453 (1.4); 7.3294 (1.4); 7.3242 (1.3); 7.2643 (0.5);
7.2593 (46.9); 4.0361 (0.9); 3.9895 (1.6); 3.8790 (0.9); 3.8753 (1.0); 3.8322 (0.5); 3.8285 (0.6); 2.1872 (16.0); 1.5330 (10.2);
0.0079 (0.5); −0.0002 (16.8); −0.0052 (0.6); −0.0060 (0.5); −0.0085 (0.8)
Ia-019: ¹H-NMR(400.0 MHz, CDCl3):
δ = 7.4432 (0.6); 7.4375 (0.5); 7.4220 (1.2); 7.4066 (0.5); 7.4008 (0.6); 7.2599 (10.1); 7.0402 (1.7); 7.0186 (3.0); 6.9971 (1.6);
3.9061 (1.0); 3.9031 (0.9); 3.8348 (1.3); 3.7877 (0.5); 2.1885 (16.0); 1.5378 (1.7); −0.0002 (3.6)
Ia-020: ¹H-NMR(400.0 MHz, CDCl3):
δ = 9.8540 (16.0); 7.3485 (2.0); 7.3321 (2.2); 7.3277 (4.2); 7.3113 (4.2); 7.3067 (2.4); 7.2904 (2.3); 7.2592 (64.9); 7.2550 (0.6);
6.8801 (3.8); 6.8775 (6.4); 6.8751 (3.7); 6.8589 (3.4); 6.8564 (5.9); 6.8539 (3.3); 6.7028 (3.5); 6.7001 (3.4); 6.6821 (3.3); 6.6794
(3.3); 6.6742 (3.5); 6.6715 (3.5); 6.6535 (3.3); 6.6508 (3.2); 3.9320 (2.1); 3.9236 (2.1); 3.8851 (3.9); 3.8769 (3.8); 3.7834 (1.0);
3.7804 (2.5); 3.7772 (2.6); 3.7738 (1.2); 3.7717 (1.2); 3.7684 (2.6); 3.7652 (2.6); 3.7620 (0.9); 3.7364 (0.5); 3.7333 (1.4); 3.7301
(1.5); 3.7268 (0.7); 3.7246 (0.6); 3.7214 (1.4); 3.7181 (1.4); 3.7149 (0.5); 3.5111 (4.1); 1.6003 (4.0); 0.0079 (0.9); −0.0002
(26.7); −0.0085 (0.7)
Ia-021: ¹H-NMR(300.2 MHz, CDCl3):
δ = 7.7648 (2.4); 7.7391 (4.6); 7.7141 (3.1); 7.6445 (0.4); 7.6159 (3.8); 7.5799 (4.1); 7.5661 (4.4); 7.5384 (3.2); 7.2988 (45.9);
3.7789 (4.8); 3.7193 (8.5); 3.5763 (5.1); 3.5726 (5.3); 3.5176 (9.7); 1.6061 (16.0); 1.5067 (0.4); 1.4863 (0.5); 0.0488 (2.0);
0.0381 (60.9); 0.0272 (2.4)
Ia-022: ¹H-NMR(400.0 MHz, CDCl3):
δ = 7.7394 (1.4); 7.7365 (2.7); 7.7336 (1.5); 7.5215 (5.6); 7.5186 (5.7); 7.2597 (12.6); 3.8960 (0.7); 3.8924 (0.7); 3.8505 (1.1);
3.8469 (1.1); 3.6851 (2.0); 3.6397 (1.3); 2.1845 (16.0); 1.5386 (2.1); 1.2563 (0.6); −0.0002 (4.6)
Ia-023: ¹H-NMR(300.2 MHz, d₆-DMSO):
δ = 8.8150 (4.6); 8.1581 (2.5); 7.8879 (1.6); 7.8822 (1.7); 7.8535 (1.4); 7.8480 (1.7); 7.8424 (1.8); 7.8264 (2.6); 7.7998 (2.0);
7.7510 (2.1); 7.7457 (2.0); 7.7229 (1.1); 7.7177 (1.1); 7.6839 (2.5); 7.6795 (2.5); 7.1858 (3.3); 5.7781 (0.4); 4.0524 (1.8); 3.9900
(2.5); 3.6708 (1.5); 3.6071 (1.2); 3.3529 (16.0); 2.5341 (1.5); 2.5284 (3.1); 2.5225 (4.1); 2.5166 (3.0); 1.9291 (0.7); 1.2543 (0.6);
0.0192 (6.0)
Ia-024: ¹H-NMR(300.2 MHz, d₆-DMSO):
δ = 10.5653 (0.7); 8.5903 (0.9); 7.5423 (3.2); 7.5356 (3.5); 7.5018 (3.2); 7.4951 (3.6); 7.4162 (2.6); 7.4119 (2.4); 7.3881 (3.0);
7.3839 (2.9); 7.0718 (3.4); 7.0426 (5.4); 7.0138 (3.0); 3.9143 (3.8); 3.8526 (5.2); 3.5375 (3.2); 3.4752 (2.4); 3.3520 (16.0);
2.5341 (4.0); 2.5284 (8.5); 2.5224 (11.6); 2.5165 (8.6); 2.5107 (4.2); 2.0093 (0.6); 0.0308 (0.6); 0.0199 (14.8); 0.0090 (0.6)
Ia-025: ¹H-NMR(400.1 MHz, d₆-DMSO):
δ = 7.7085 (5.2); 7.5635 (2.5); 7.5448 (3.4); 7.4780 (2.0); 7.4590 (3.9); 7.4304 (3.8); 7.4115 (4.5); 7.3926 (1.6); 3.9354 (3.5);
3.8890 (4.4); 3.7595 (16.0); 3.5767 (3.0); 3.5304 (2.4); 3.0841 (0.4); 2.5137 (3.7); 2.5097 (4.9); 2.5056 (3.8); 2.1565 (0.6);
1.1137 (1.0)
Ia-027: ¹H-NMR(300.2 MHz, CDCl3):
δ = 7.2987 (46.1); 7.2414 (4.2); 7.2154 (9.2); 7.1891 (5.9); 7.1244 (5.4); 7.1181 (8.3); 7.1117 (6.1); 7.0129 (5.9); 6.9875 (4.7);
6.8005 (4.2); 6.7948 (4.0); 6.7736 (3.6); 6.7680 (3.6); 4.1865 (3.9); 4.1817 (4.0); 4.1264 (4.9); 4.1216 (5.0); 3.7706 (8.6); 3.6914
(9.4); 3.6314 (7.4); 3.5571 (0.9); 3.5352 (1.9); 3.5207 (2.5); 3.4994 (4.9); 3.4860 (1.8); 3.4767 (3.1); 3.4650 (3.9); 3.4418 (5.8);
3.4286 (6.0); 3.4058 (4.0); 3.3928 (3.1); 3.3852 (1.8); 3.3703 (4.9); 3.3492 (2.7); 3.3340 (1.8); 3.3119 (0.8); 1.9956 (0.6); 1.9848
(1.1); 1.9753 (1.2); 1.9642 (3.2); 1.9422 (6.7); 1.9313 (7.9); 1.9219 (9.9); 1.9092 (8.3); 1.8991 (6.7); 1.8767 (3.0); 1.8559 (0.9);
1.8446 (0.6); 1.5857 (16.0); 1.2935 (1.2); 0.0489 (2.2); 0.0384 (59.2); 0.0274 (2.2)
Ia-029: ¹H-NMR(300.2 MHz, d₆-DMSO):
δ = 8.6892 (1.5); 7.7458 (2.0); 7.7178 (2.6); 7.5172 (2.7); 7.4892 (2.0); 5.5448 (2.4); 3.9929 (0.9); 3.9311 (1.2); 3.5941 (0.8);
3.5318 (0.6); 3.3687 (4.0); 2.5286 (0.4); 2.5227 (0.5); 2.5168 (0.4); 2.0928 (3.7); 1.4964 (16.0)
Ia-030: ¹H-NMR(300.2 MHz, CDCl3):
δ = 8.1192 (0.5); 7.7112 (6.2); 7.6830 (16.0); 7.6476 (15.9); 7.6194 (5.9); 7.5883 (1.5); 7.5785 (0.7); 7.5669 (2.2); 7.5585 (2.8);
7.5369 (2.9); 7.5109 (1.8); 7.2989 (8.3); 6.9728 (1.4); 6.9625 (4.2); 6.9538 (2.2); 6.9456 (2.4); 6.9370 (5.3); 6.9315 (5.3); 6.9244
(3.1); 6.9119 (2.5); 6.9008 (2.6); 6.8929 (1.4); 4.7119 (0.6); 3.7950 (10.8); 3.7342 (7.3); 3.5924 (4.8); 3.5331 (2.7); 3.5303 (2.7);
2.6656 (0.5); 2.0436 (0.5); 1.6977 (10.9); 1.2932 (0.9); 0.0384 (4.0)
Ia-031: ¹H-NMR(300.2 MHz, d₆-DMSO):
δ = 8.6757 (9.0); 7.7044 (6.5); 7.6765 (8.6); 7.4831 (8.5); 7.4552 (6.6); 3.9757 (3.0); 3.9138 (4.0); 3.5822 (2.4); 3.5199 (1.8);
3.3624 (16.0); 2.5345 (0.5); 2.5287 (1.1); 2.5227 (1.5); 2.5167 (1.1); 2.5109 (0.5); 2.0940 (1.5); 1.6359 (0.5); 1.6192 (1.1);
1.6084 (1.1); 1.6028 (0.8); 1.5918 (2.3); 1.5811 (0.7); 1.5751 (1.2); 1.5644 (1.3); 1.5477 (0.7); 0.9656 (1.2); 0.9506 (3.2); 0.9418
(4.8); 0.9298 (2.3); 0.9232 (3.3); 0.9144 (4.0); 0.9027 (1.8); 0.8560 (0.5); 0.8389 (0.3); 0.8080 (1.9); 0.7962 (4.7); 0.7877 (3.9);
0.7798 (4.5); 0.7714 (4.0); 0.7562 (1.1)
Ia-032: ¹H-NMR(300.2 MHz, CDCl3):
δ = 7.7109 (0.6); 7.6826 (1.6); 7.6417 (1.6); 7.6351 (0.4); 7.6136 (0.7); 7.6022 (0.5); 7.5909 (0.6); 7.5847 (0.5); 7.5788 (0.6);
7.5701 (0.6); 7.4183 (1.1); 7.4071 (1.1); 7.3968 (1.0); 7.2989 (9.5); 3.7986 (0.4); 3.7398 (0.7); 3.5975 (0.5); 3.5286 (0.9); 1.5912
(16.0); 0.0487 (0.6); 0.0385 (11.3); 0.0281 (0.5)
Ia-033: ¹H-NMR(300.2 MHz, CDCl3):
δ = 7.6768 (5.2); 7.6708 (2.1); 7.6550 (2.7); 7.6484 (9.7); 7.5773 (9.7); 7.5707 (2.8); 7.5548 (2.1); 7.5488 (5.2); 7.2988 (8.0);
5.4987 (5.4); 5.2268 (3.3); 5.2223 (4.7); 5.2178 (3.3); 3.7934 (2.8); 3.7341 (5.2); 3.5958 (3.2); 3.5920 (3.3); 3.5735 (4.7); 3.5364
(1.7); 3.5326 (1.8); 2.2060 (16.0); 2.0848 (0.4); 1.6566 (7.1); 1.2944 (0.7); 0.0502 (0.4); 0.0394 (9.6); 0.0285 (0.4)
Ia-034: ¹H-NMR(300.2 MHz, d₆-DMSO):
δ = 11.4361 (2.2); 8.6762 (1.1); 7.7986 (0.4); 7.7689 (3.2); 7.7610 (3.2); 7.7310 (0.4); 4.0006 (0.6); 3.9387 (0.8); 3.6108 (0.5);
3.5487 (0.4); 3.3416 (16.0); 2.5347 (1.6); 2.5288 (3.5); 2.5228 (4.9); 2.5168 (3.5); 2.5110 (1.6); 2.1957 (6.5); 0.0213 (5.5)
Ia-035: ¹H-NMR(400.2 MHz, d₆-DMSO):
δ = 10.7396 (14.6); 8.8043 (0.4); 8.7912 (0.3); 8.7358 (0.3); 8.7038 (0.4); 8.6377 (16.0); 8.3918 (9.6); 8.1344 (1.0); 7.6783 (11.5);
7.6575 (14.1); 7.4683 (14.2); 7.4475 (11.6); 7.0920 (5.6); 7.0727 (10.5); 7.0528 (7.1); 6.8131 (3.4); 6.7947 (5.9); 6.7763 (2.6);
6.6704 (10.4); 6.6509 (9.8); 3.9487 (5.2); 3.9024 (6.5); 3.7872 (0.4); 3.5489 (4.3); 3.5022 (3.5); 3.3299 (7.6); 3.2642 (1.0);
2.6861 (0.9); 2.5570 (0.8); 2.5059 (33.9); 2.5016 (44.8); 2.4973 (32.5); 2.0738 (0.9); 0.0001 (7.1)
Ia-036: ¹H-NMR(400.2 MHz, d₆-DMSO):
δ = 10.0375 (1.7); 8.1759 (1.4); 7.9957 (0.4); 7.8256 (0.4); 7.7421 (0.4); 7.7145 (13.0); 7.6937 (16.0); 7.6127 (0.5); 7.5916 (0.5);
7.4541 (15.7); 7.4333 (14.0); 7.3754 (0.4); 7.3573 (0.5); 7.3374 (0.4); 7.3192 (0.5); 7.3005 (0.4); 7.2811 (4.8); 7.2633 (13.2);
7.2444 (10.7); 7.2028 (5.0); 7.1847 (6.4); 7.1665 (2.1); 7.1250 (12.6); 7.1071 (10.4); 6.3989 (2.6); 6.3812 (5.7); 6.3636 (2.8);
4.2833 (0.4); 4.2684 (0.4); 4.1848 (10.6); 4.1672 (10.6); 4.0020 (0.4); 3.9956 (0.5); 3.9833 (0.4); 3.9577 (6.4); 3.9381 (0.6);
3.9114 (7.7); 3.7391 (0.5); 3.6100 (1.1); 3.5692 (6.3); 3.5227 (5.7); 3.3753 (4.6); 2.8901 (0.5); 2.7314 (0.4); 2.6713 (0.4); 2.5066
(30.1); 2.5024 (39.8); 2.4981 (29.9); 0.0001 (0.4)
Ia-037: ¹H-NMR(300.2 MHz, d₆-DMSO):
δ = 7.7804 (0.8); 7.7774 (0.8); 3.9664 (1.3); 3.3386 (16.0); 2.5348 (1.5); 2.5288 (3.2); 2.5228 (4.5); 2.5167 (3.2); 2.5108 (1.5);
2.2224 (1.3); 0.0218 (5.4)
Ia-038: ¹H-NMR(300.2 MHz, d₆-DMSO):
δ = 8.7711 (0.4); 8.0502 (0.4); 7.9008 (0.4); 3.3444 (16.0); 2.6409 (1.4); 2.5344 (1.6); 2.5286 (3.3); 2.5226 (4.5); 2.5166 (3.4);
2.5109 (1.7); 0.0211 (7.1); 0.0104 (0.4)
Ia-039: ¹H-NMR(300.2 MHz, d₆-DMSO):
δ = 8.8544 (0.6); 8.8499 (0.6); 8.8394 (0.6); 8.8350 (0.6); 8.7248 (1.5); 8.7203 (1.5); 8.6967 (1.2); 8.6926 (1.1); 8.1650 (0.6);
8.1480 (0.7); 8.1166 (3.6); 8.0719 (0.4); 7.9966 (11.0); 7.9685 (16.0); 7.8328 (15.7); 7.8047 (11.2); 7.6737 (0.6); 7.6588 (0.6);
7.6455 (0.6); 7.6307 (0.6); 7.5255 (3.9); 4.0287 (5.6); 3.9667 (7.6); 3.9263 (0.4); 3.6377 (8.4); 3.5758 (5.2); 3.4162 (0.4); 3.2715
(4.1); 3.0439 (4.0); 2.9103 (1.3); 2.8738 (0.4); 2.8171 (1.4); 2.7993 (0.7); 2.7513 (1.2); 2.5342 (5.5); 2.5284 (12.0); 2.5224
(16.7); 2.5164 (12.3); 2.5109 (5.9); 2.0941 (0.3); 0.0189 (4.2)
Ia-040: ¹H-NMR(499.9 MHz, d₆-DMSO):
δ = 13.2188 (0.5); 8.7586 (0.6); 8.1497 (0.9); 8.1179 (0.4); 8.1137 (0.6); 8.1016 (2.3); 8.0960 (2.6); 8.0785 (0.5); 8.0338 (12.6);
8.0170 (16.0); 7.8528 (15.2); 7.8360 (13.2); 3.9944 (6.3); 3.9574 (7.7); 3.6283 (5.0); 3.5911 (4.2); 3.3859 (0.4); 2.5077 (1.2);
2.5043 (1.6); 2.5009 (1.3); 1.9802 (0.8); 1.1666 (0.4)
Ia-041: ¹H-NMR(300.2 MHz, d₆-DMSO):
δ = 8.7743 (1.7); 8.0803 (3.5); 8.0743 (1.3); 8.0578 (1.6); 8.0517 (4.8); 7.9067 (4.9); 7.9004 (1.5); 7.8781 (3.4); 4.0512 (1.6);
3.9892 (2.1); 3.9040 (16.0); 3.6438 (1.3); 3.5778 (1.0); 3.3381 (14.5); 2.5341 (3.7); 2.5281 (7.7); 2.5220 (10.5); 2.5159 (7.5);
2.5100 (3.5); 0.0315 (0.7); 0.0207 (18.4); 0.0097 (0.7)
Ia-042: ¹H-NMR(300.2 MHz, CDCl3):
δ = 7.8318 (0.6); 7.8054 (1.7); 7.7770 (1.9); 7.7491 (0.6); 7.2987 (40.5); 3.7971 (0.6); 3.7375 (1.0); 3.5950 (0.6); 3.5388 (0.4);
2.6792 (0.7); 2.6447 (0.5); 2.6102 (0.5); 2.5447 (0.6); 2.4905 (0.5); 2.4830 (0.5); 2.3691 (0.5); 2.3599 (0.4); 2.1457 (16.0);
0.0490 (2.0); 0.0382 (52.7); 0.0272 (1.6)
Ia-043: ¹H-NMR(300.2 MHz, CDCl3):
δ = 7.6730 (2.8); 7.6453 (3.8); 7.4848 (3.6); 7.4572 (2.7); 7.2988 (1.4); 4.2542 (0.4); 4.2319 (1.3); 4.2095 (1.3); 4.1873 (0.7);
4.1635 (0.8); 4.1397 (0.8); 3.7639 (1.3); 3.7046 (2.3); 3.5516 (1.4); 3.4970 (16.0); 2.0824 (3.6); 1.4001 (6.2); 1.3778 (6.2);
1.3125 (1.0); 1.2887 (1.8); 1.2649 (0.9); 0.0362 (1.6)
Ia-044: ¹H-NMR(300.2 MHz, CDCl3):
δ = 7.6956 (4.2); 7.6680 (5.7); 7.4979 (5.3); 7.4701 (4.0); 7.2990 (11.8); 5.0124 (0.9); 5.0008 (0.9); 4.9909 (1.0); 4.9796 (0.9);
3.7872 (2.0); 3.7279 (3.6); 3.6800 (3.6); 3.5832 (2.3); 3.5233 (1.3); 2.0839 (0.4); 1.9454 (2.0); 1.9333 (1.9); 1.6251 (16.0);
1.5579 (10.5); 1.5363 (10.4); 1.2919 (1.0); 0.1075 (0.7); 0.0485 (0.6); 0.0379 (14.8)
Ia-045: ¹H-NMR(300.2 MHz, d₆-DMSO):
δ = 3.3418 (16.0); 2.5346 (1.0); 2.5287 (2.1); 2.5227 (2.9); 2.5167 (2.1); 2.5108 (1.0); 2.3743 (0.6); 0.0215 (1.6)
Ia-046: ¹H-NMR(300.2 MHz, d₆-DMSO):
δ = 3.3426 (16.0); 2.5346 (1.1); 2.5287 (2.3); 2.5227 (3.1); 2.5167 (2.2); 2.5109 (1.0); 1.1798 (0.7); 1.0898 (0.4); 0.0213 (1.6)
Ia-047: ¹H-NMR(400.2 MHz, d₆-DMSO):
δ = 11.8923 (2.1); 8.6530 (7.4); 7.9531 (2.1); 7.8910 (9.9); 7.8865 (3.6); 7.8742 (3.7); 7.8695 (13.5); 7.7696 (4.5); 7.7644 (5.0);
7.7522 (5.1); 7.7470 (5.1); 7.7359 (12.8); 7.7313 (4.0); 7.7190 (3.3); 7.7145 (10.1); 7.4495 (3.7); 7.4444 (3.8); 7.4335 (4.0);
7.4285 (3.8); 6.3427 (4.6); 6.3258 (7.0); 6.3093 (4.5); 3.9720 (4.6); 3.9258 (5.9); 3.5945 (3.4); 3.5479 (2.7); 3.3356 (65.2);
2.8911 (16.0); 2.7329 (13.1); 2.7318 (13.4); 2.6725 (0.4); 2.5259 (1.3); 2.5212 (1.8); 2.5125 (22.9); 2.5080 (46.7); 2.5035 (61.7);
2.4988 (44.6); 2.4943 (21.6); 2.3303 (0.4); 1.2394 (1.3); 0.8532 (0.3); −0.0002 (3.6)
Ia-048: ¹H-NMR(400.2 MHz, d₆-DMSO):
δ = 8.7469 (10.6); 8.6189 (0.4); 8.0557 (12.6); 8.0344 (16.0); 8.0186 (0.6); 7.9530 (1.6); 7.8690 (16.0); 7.8477 (13.3); 7.7231
(0.4); 7.7023 (0.5); 7.6897 (0.6); 7.6684 (1.1); 7.6454 (0.4); 7.6343 (0.6); 7.6283 (0.3); 7.6118 (0.4); 7.5909 (0.8); 7.5693 (0.6);
7.5212 (0.6); 7.5000 (0.5); 6.1402 (0.4); 5.9436 (0.4); 4.4152 (3.9); 4.4023 (8.2); 4.3894 (4.1); 4.3645 (0.4); 4.0213 (6.3); 4.0027
(0.6); 3.9895 (0.6); 3.9748 (7.8); 3.9499 (0.4); 3.9036 (0.5); 3.6250 (4.6); 3.5782 (3.8); 3.5624 (0.6); 3.5140 (0.4); 3.4487 (4.4);
3.4327 (10.2); 3.4196 (10.1); 3.4037 (4.6); 3.3934 (0.5); 3.3772 (0.7); 3.3641 (0.7); 3.3322 (55.2); 3.1989 (0.6); 3.0801 (7.2);
3.0621 (13.3); 3.0438 (7.5); 2.8915 (12.0); 2.7329 (10.0); 2.7318 (9.9); 2.6768 (0.4); 2.6721 (0.6); 2.6675 (0.4); 2.6296 (0.9);
2.5836 (0.5); 2.5742 (0.4); 2.5659 (0.9); 2.5577 (0.4); 2.5475 (0.7); 2.5256 (1.8); 2.5209 (2.6); 2.5122 (31.7); 2.5077 (64.0);
2.5031 (83.7); 2.4985 (60.2); 2.4940 (28.9); 2.3346 (0.4); 2.3300 (0.5); 2.3254 (0.4); 2.1550 (0.4); 2.1445 (0.4); 1.7649 (0.4);
1.7520 (0.5); 1.7392 (0.4); 1.6854 (1.6); 1.6675 (4.7); 1.6613 (2.1); 1.6494 (6.3); 1.6304 (4.9); 1.6114 (2.2); 1.5639 (0.7); 1.5333
(0.5); 1.5167 (2.5); 1.5011 (5.4); 1.4872 (4.5); 1.4802 (6.2); 1.4714 (2.1); 1.4637 (4.6); 1.4468 (1.4); 1.4045 (0.4); 1.3835 (0.3);
1.2594 (0.5); 1.2394 (2.0); 0.8531 (0.5); −0.0002 (6.9)
Ia-049: ¹H-NMR(400.2 MHz, d₆-DMSO):
δ = 8.7197 (6.2); 8.0019 (2.8); 7.9996 (3.1); 7.9826 (3.2); 7.9802 (3.3); 7.9538 (2.1); 7.8964 (6.9); 7.8920 (2.6); 7.8801 (2.6);
7.8753 (9.2); 7.8509 (1.5); 7.8476 (1.6); 7.8316 (3.2); 7.8283 (3.3); 7.8125 (2.4); 7.8091 (2.4); 7.7256 (1.4); 7.7210 (9.1); 7.7163
(3.0); 7.7044 (2.4); 7.6998 (7.5); 7.6922 (3.9); 7.6905 (4.2); 7.6784 (0.6); 7.6725 (2.9); 7.6709 (2.9); 7.6506 (2.5); 7.6477 (2.5);
7.6316 (3.9); 7.6287 (4.4); 7.6202 (0.4); 7.6163 (0.8); 7.6125 (2.1); 7.6096 (1.9); 7.5991 (0.7); 7.5747 (0.5); 7.5668 (0.6); 7.5600
(0.4); 7.5573 (0.4); 7.5516 (0.5); 7.5490 (0.5); 4.0374 (3.3); 3.9910 (4.2); 3.6503 (2.4); 3.6035 (1.9); 3.3340 (22.0); 3.1988 (0.5);
2.8917 (16.0); 2.7331 (13.0); 2.7323 (13.2); 2.6301 (0.8); 2.5262 (0.8); 2.5216 (1.2); 2.5129 (16.2); 2.5084 (33.3); 2.5038 (43.9);
2.4992 (31.6); 2.4946 (15.3); 1.2390 (1.4); 0.8529 (0.4); −0.0002 (3.0)
Ia-050: ¹H-NMR(300.2 MHz, d₆-DMSO):
δ = 8.6945 (0.5); 8.1602 (0.3); 7.8189 (0.5); 7.8149 (0.5); 7.7879 (1.8); 7.7597 (1.6); 7.6329 (0.6); 7.6280 (0.6); 7.6079 (0.5);
7.6031 (0.4); 7.5450 (0.4); 7.5406 (0.5); 7.5197 (0.6); 7.5156 (0.6); 7.4684 (1.6); 7.4408 (1.9); 7.4204 (0.5); 7.4173 (0.5); 4.0315
(0.5); 3.9696 (0.7); 3.6354 (0.5); 3.5740 (0.4); 3.3506 (16.0); 2.5345 (3.0); 2.5286 (6.4); 2.5225 (8.8); 2.5165 (6.5); 2.5107 (3.0);
0.0315 (0.4); 0.0207 (12.3); 0.0097 (0.4)
Ia-051: ¹H-NMR(400.1 MHz, d₆-DMSO):
δ = 8.6693 (5.4); 8.1200 (0.4); 7.8351 (7.7); 7.8151 (8.8); 7.6131 (2.8); 7.5990 (2.8); 7.5923 (3.0); 7.5713 (9.0); 7.5513 (7.6);
7.4902 (0.6); 7.4627 (16.0); 7.4500 (3.9); 7.4377 (1.4); 7.4273 (0.5); 4.0185 (3.7); 3.9720 (4.6); 3.6320 (3.5); 3.5854 (2.8);
3.3113 (14.4); 2.5087 (13.2); 2.2858 (1.0); 2.2424 (0.9)
Ia-052: ¹H-NMR(400.2 MHz, d₆-DMSO):
δ = 8.6848 (6.3); 7.9535 (1.4); 7.8342 (1.1); 7.8297 (7.6); 7.8251 (2.7); 7.8133 (2.7); 7.8085 (9.2); 7.8041 (1.5); 7.6160 (0.5);
7.6106 (1.8); 7.6068 (2.5); 7.5984 (1.6); 7.5966 (1.6); 7.5934 (1.8); 7.5915 (2.1); 7.5863 (2.6); 7.5700 (1.4); 7.5656 (9.3); 7.5608
(2.9); 7.5490 (2.5); 7.5444 (8.0); 7.5398 (1.2); 7.4660 (2.0); 7.4621 (3.6); 7.4567 (16.0); 7.4517 (10.7); 7.4421 (4.0); 7.4319
(1.1); 7.4285 (1.1); 7.4185 (0.6); 4.0165 (3.5); 3.9702 (4.3); 3.6261 (2.4); 3.5793 (2.0); 3.3356 (70.1); 3.1987 (0.6); 2.8915
(10.8); 2.7331 (8.9); 2.7321 (8.8); 2.6300 (1.0); 2.5259 (0.6); 2.5211 (0.9); 2.5125 (13.7); 2.5080 (28.4); 2.5034 (37.7); 2.4988
(27.0); 2.4943 (12.8); 1.2395 (0.9); −0.0002 (4.7)
Ia-053: ¹H-NMR(400.2 MHz, d₆-DMSO):
δ = 8.6918 (10.5); 7.8432 (11.8); 7.8387 (4.2); 7.8267 (4.6); 7.8220 (16.0); 7.8175 (2.7); 7.6904 (8.7); 7.6865 (9.8); 7.6693 (7.8);
7.6655 (6.6); 7.6157 (2.2); 7.6114 (2.5); 7.5962 (4.4); 7.5916 (4.6); 7.5837 (0.6); 7.5757 (2.7); 7.5718 (2.8); 7.4984 (1.1); 7.4940
(1.1); 7.4855 (1.2); 7.4806 (2.5); 7.4761 (2.1); 7.4735 (1.8); 7.4663 (2.4); 7.4633 (2.1); 7.4597 (3.1); 7.4551 (1.9); 7.4466 (1.9);
7.4421 (1.7); 7.3736 (2.9); 7.3713 (3.8); 7.3575 (3.9); 7.3547 (5.1); 7.3505 (2.9); 7.3459 (3.7); 7.3430 (4.3); 7.3386 (6.8); 7.3253
(2.5); 7.3206 (4.1); 7.3173 (2.5); 4.0076 (5.9); 3.9613 (7.4); 3.6238 (4.1); 3.5772 (3.3); 3.3367 (102.2); 3.1990 (0.8); 2.8917
(2.0); 2.7327 (1.7); 2.6730 (0.4); 2.6305 (1.2); 2.5264 (0.9); 2.5217 (1.3); 2.5130 (20.2); 2.5085 (41.8); 2.5040 (55.6); 2.4994
(39.8); 2.4948 (18.9); 2.3307 (0.3); 1.2386 (1.4); 0.8529 (0.4); −0.0002 (6.7)
Ia-054: ¹H-NMR(400.2 MHz, d₆-DMSO):
δ = 8.6589 (2.2); 7.7654 (0.5); 7.7608 (3.4); 7.7561 (1.2); 7.7443 (1.3); 7.7395 (4.5); 7.7350 (0.7); 7.6057 (0.7); 7.6013 (4.6);
7.5965 (1.3); 7.5846 (1.2); 7.5800 (3.5); 7.5753 (0.5); 7.4065 (0.7); 7.4021 (0.8); 7.3879 (0.9); 7.3856 (1.1); 7.3837 (1.2); 7.3815
(1.1); 7.3674 (0.9); 7.3630 (1.1); 7.3483 (1.5); 7.3440 (1.4); 7.3294 (1.9); 7.3251 (1.5); 7.1521 (1.7); 7.1505 (1.8); 7.1315 (1.5);
7.1298 (1.4); 7.0757 (1.1); 7.0732 (1.0); 7.0571 (1.9); 7.0545 (1.7); 7.0384 (0.9); 7.0359 (0.8); 3.9841 (1.6); 3.9379 (2.0); 3.7812
(16.0); 3.6057 (1.1); 3.5592 (0.9); 3.3331 (17.9); 2.8904 (1.0); 2.7325 (0.8); 2.7313 (0.8); 2.6294 (0.5); 2.5250 (0.4); 2.5203
(0.5); 2.5116 (7.0); 2.5071 (14.2); 2.5025 (18.6); 2.4978 (13.2); 2.4932 (6.3); 1.2391 (0.5); −0.0002 (1.3)
Ia-055: ¹H-NMR(400.2 MHz, d₆-DMSO):
δ = 8.7371 (5.9); 8.7019 (1.9); 8.6430 (0.5); 8.5507 (1.5); 8.5390 (1.5); 8.1326 (0.3); 7.9544 (2.1); 7.9027 (5.2); 7.8816 (8.4);
7.8112 (4.8); 7.7928 (3.2); 7.7385 (0.7); 7.7347 (0.8); 7.7194 (1.8); 7.7146 (1.4); 7.7032 (1.6); 7.6897 (1.2); 7.6490 (3.0); 7.6464
(4.3); 7.6421 (2.3); 7.6336 (4.1); 7.6285 (8.4); 7.6258 (5.8); 7.6201 (2.5); 7.6166 (5.7); 7.6128 (3.3); 7.6103 (2.3); 7.6080 (2.4);
7.6054 (1.6); 7.5994 (6.2); 7.5956 (3.3); 7.5746 (4.1); 7.5668 (5.2); 7.5601 (3.4); 7.5573 (3.5); 7.5516 (4.2); 7.5490 (4.3); 7.5466
(3.0); 7.5424 (1.2); 7.5383 (1.7); 7.5352 (1.4); 7.5307 (1.8); 7.5278 (1.1); 7.5182 (0.3); 7.5085 (0.5); 7.5037 (1.3); 7.4985 (0.7);
7.4844 (0.6); 4.0241 (3.1); 3.9777 (3.9); 3.9639 (0.5); 3.9173 (0.5); 3.6408 (2.3); 3.5941 (1.8); 3.5832 (0.4); 3.5798 (0.4); 3.3348
(50.4); 3.1992 (0.7); 2.8922 (16.0); 2.7338 (13.4); 2.7325 (13.1); 2.6780 (0.3); 2.6732 (0.4); 2.6306 (1.1); 2.5269 (1.4); 2.5221
(2.1); 2.5135 (25.7); 2.5090 (52.0); 2.5044 (68.1); 2.4997 (48.6); 2.4952 (23.1); 2.3311 (0.4); 1.2592 (0.4); 1.2391 (1.6); 0.8529
(0.4); −0.0002 (5.8)
Ia-056: ¹H-NMR(400.2 MHz, d₆-DMSO):
δ = 8.7114 (0.6); 8.5013 (4.1); 8.3157 (2.5); 8.3037 (2.5); 7.8179 (3.2); 7.8012 (1.4); 7.7967 (4.8); 7.7054 (4.8); 7.7009 (1.4);
7.6842 (3.3); 7.4058 (2.6); 7.3938 (2.5); 3.9987 (1.7); 3.9524 (2.2); 3.9144 (16.0); 3.6207 (1.2); 3.5739 (1.0); 3.3347 (13.0);
2.8914 (1.3); 2.7321 (1.1); 2.5258 (0.5); 2.5211 (0.7); 2.5124 (8.5); 2.5079 (17.0); 2.5034 (22.1); 2.4988 (15.9); 2.4943 (7.7);
1.2395 (0.6); −0.0002 (1.2)
Ia-057: ¹H-NMR(400.2 MHz, d₆-DMSO):
δ = 8.7066 (15.3); 8.2986 (2.6); 8.2943 (3.8); 8.2911 (3.2); 8.2866 (3.0); 8.2823 (3.8); 8.2791 (3.1); 8.2174 (2.5); 8.2126 (2.3);
8.1987 (2.8); 8.1937 (3.0); 8.1918 (3.1); 8.1868 (2.4); 8.1729 (2.8); 8.1681 (2.4); 7.9546 (0.3); 7.8758 (1.6); 7.8712 (10.6);
7.8665 (3.9); 7.8547 (4.4); 7.8498 (16.0); 7.8454 (2.7); 7.7608 (8.3); 7.7568 (9.3); 7.7395 (6.6); 7.7358 (5.6); 7.5295 (2.8);
7.5247 (2.8); 7.5174 (2.7); 7.5112 (3.6); 7.5060 (2.8); 7.4988 (2.6); 7.4940 (2.6); 4.0165 (5.5); 3.9701 (6.9); 3.6323 (3.8); 3.5856
(3.0); 3.3347 (52.0); 2.8926 (2.5); 2.7342 (2.0); 2.7330 (2.1); 2.6737 (0.4); 2.6309 (0.3); 2.5273 (1.1); 2.5226 (1.7); 2.5139
(21.2); 2.5093 (43.0); 2.5047 (56.3); 2.5001 (40.1); 2.4955 (18.9); 2.3316 (0.3); 1.2383 (1.3); 0.8527 (0.4); −0.0002 (4.6)
Ia-058: ¹H-NMR(400.2 MHz, d₆-DMSO):
δ = 8.6777 (2.7); 8.2248 (1.5); 8.2202 (1.6); 8.2125 (1.6); 8.2078 (1.6); 7.8285 (1.5); 7.8238 (1.6); 7.8101 (1.7); 7.8054 (1.6);
7.7939 (2.7); 7.7893 (1.0); 7.7774 (1.2); 7.7725 (4.2); 7.6917 (4.2); 7.6869 (1.2); 7.6749 (1.0); 7.6703 (2.8); 7.1427 (1.6); 7.1303
(1.6); 7.1243 (1.6); 7.1120 (1.6); 3.9925 (1.4); 3.9462 (1.8); 3.8993 (16.0); 3.6141 (1.0); 3.5673 (0.8); 3.3345 (22.7); 2.8914
(0.6); 2.7332 (0.5); 2.7320 (0.5); 2.5213 (0.4); 2.5126 (5.4); 2.5081 (11.2); 2.5035 (14.9); 2.4989 (10.7); 2.4944 (5.1); 1.2386
(0.4); −0.0002 (1.9)
Ia-059: ¹H-NMR(400.2 MHz, d₆-DMSO):
δ = 8.6804 (2.6); 7.8226 (3.6); 7.8183 (1.2); 7.8061 (1.4); 7.8015 (4.2); 7.5261 (4.2); 7.5216 (1.4); 7.5049 (3.7); 3.9980 (1.7);
3.9517 (2.1); 3.6181 (1.2); 3.5713 (1.0); 3.3407 (11.7); 2.8922 (1.0); 2.7334 (0.8); 2.7324 (0.8); 2.5215 (0.4); 2.5129 (5.8);
2.5084 (11.6); 2.5038 (15.2); 2.4993 (10.8); 2.4948 (5.1); 2.4337 (16.0); 2.2539 (15.7); 1.2397 (0.7); −0.0002 (1.6)
Ia-060: ¹H-NMR(400.2 MHz, d₆-DMSO):
δ = 13.0508 (1.0); 8.6114 (1.3); 8.3158 (0.8); 8.0317 (0.6); 7.7430 (5.1); 7.7382 (2.2); 7.7215 (16.0); 7.7013 (15.5); 7.6845 (2.2);
7.6797 (5.0); 3.9562 (4.4); 3.9101 (5.6); 3.5836 (3.2); 3.5368 (2.6); 3.3348 (18.6); 2.8911 (0.5); 2.7328 (0.5); 2.6725 (0.3);
2.5259 (1.1); 2.5211 (1.6); 2.5125 (20.2); 2.5081 (40.3); 2.5035 (52.2); 2.4989 (37.6); 2.4944 (18.2); 2.3303 (0.3); 1.2593 (0.3);
1.2394 (1.5); 0.8534 (0.4); −0.0002 (4.1)
Ia-061: ¹H-NMR(300.2 MHz, d₆-DMSO):
δ = 7.8476 (0.4); 7.7175 (0.3); 7.5926 (0.4); 5.7791 (0.5); 3.3469 (16.0); 2.5344 (1.0); 2.5285 (2.2); 2.5225 (3.1); 2.5165 (2.2);
2.5108 (1.0); 0.0207 (3.3)
Ia-062: ¹H-NMR(400.2 MHz, d₆-DMSO):
δ = 8.6923 (6.5); 8.2628 (3.5); 8.2590 (6.2); 8.2553 (3.7); 8.1204 (2.2); 8.1177 (2.8); 8.1161 (2.6); 8.1131 (2.3); 8.1005 (2.5);
8.0977 (2.9); 8.0959 (2.9); 8.0931 (2.4); 7.9543 (2.1); 7.9131 (5.8); 7.9085 (2.4); 7.8964 (3.3); 7.8915 (13.7); 7.8875 (5.6);
7.8840 (2.8); 7.8709 (2.8); 7.8676 (4.0); 7.8646 (2.9); 7.8530 (12.1); 7.8480 (3.1); 7.8362 (2.3); 7.8316 (5.9); 7.7239 (3.5);
7.7106 (0.5); 7.7043 (5.9); 7.6847 (2.6); 4.0172 (3.9); 3.9709 (4.9); 3.6328 (2.8); 3.5862 (2.2); 3.3347 (50.5); 3.1994 (0.5);
2.8924 (16.0); 2.7338 (13.4); 2.7329 (13.2); 2.6305 (0.8); 2.5269 (0.8); 2.5222 (1.3); 2.5135 (18.6); 2.5090 (37.6); 2.5044 (49.0);
2.4998 (34.8); 2.4953 (16.7); 1.2374 (1.3); −0.0002 (3.2)
Ia-063: ¹H-NMR(400.2 MHz, d₆-DMSO):
δ = 8.6721 (3.0); 8.3150 (4.5); 8.2256 (4.9); 8.1386 (2.2); 7.9523 (1.3); 7.9131 (5.9); 7.9087 (5.7); 7.8935 (8.0); 7.8895 (9.1);
7.8699 (11.6); 7.8479 (11.0); 7.8265 (3.7); 7.5974 (2.8); 7.5781 (4.7); 7.5590 (2.3); 7.4657 (2.1); 4.2015 (0.3); 4.1796 (0.3);
4.0490 (0.3); 4.0271 (0.9); 4.0147 (3.2); 3.9942 (0.6); 3.9681 (3.7); 3.8448 (0.3); 3.8105 (0.3); 3.7540 (0.4); 3.6978 (0.5); 3.6674
(0.4); 3.6312 (2.5); 3.5832 (2.2); 3.5086 (0.6); 3.4303 (1.0); 3.3754 (2.9); 3.3264 (3621.3); 3.2814 (0.8); 3.2753 (0.5); 3.2649
(0.4); 3.1988 (0.3); 2.8907 (10.1); 2.8271 (0.3); 2.7871 (0.4); 2.7307 (8.6); 2.7090 (1.5); 2.6930 (1.5); 2.6799 (5.4); 2.6753
(11.5); 2.6707 (16.0); 2.6661 (11.7); 2.6615 (5.6); 2.6416 (0.9); 2.6284 (1.3); 2.5893 (2.1); 2.5723 (3.0); 2.5242 (49.6); 2.5195
(72.3); 2.5108 (917.3); 2.5063 (1877.7); 2.5017 (2472.9); 2.4971 (1778.0); 2.4925 (857.1); 2.4163 (0.5); 2.3911 (0.3); 2.3504
(0.3); 2.3377 (4.7); 2.3331 (10.8); 2.3285 (15.2); 2.3239 (10.9); 2.3193 (5.0); 1.6460 (0.4); 1.5521 (0.4); 1.4758 (0.3); 1.3157
(0.4); 1.2976 (0.8); 1.2923 (0.7); 1.2581 (1.8); 1.2400 (9.5); 1.2005 (0.4); 1.1099 (0.4); 0.8697 (1.0); 0.8531 (2.6); 0.8356 (1.2);
0.8100 (0.5); 0.8050 (0.5); 0.7951 (0.5); 0.7881 (0.4); 0.1462 (0.6); 0.0081 (4.7); −0.0002 (165.9); −0.0085 (5.4); −0.1492 (0.5)
Ia-064: ¹H-NMR(300.2 MHz, d₆-DMSO):
δ = 8.7082 (1.2); 8.2747 (1.1); 8.2693 (2.0); 8.2639 (1.3); 8.0726 (0.9); 8.0667 (0.7); 8.0467 (1.0); 8.0400 (1.4); 8.0350 (1.2);
8.0131 (0.9); 8.0086 (1.2); 7.8692 (16.0); 7.7110 (1.1); 7.6850 (1.9); 7.6592 (0.9); 4.0603 (0.4); 4.0469 (1.2); 4.0368 (0.5);
3.9852 (1.6); 3.9225 (12.6); 3.8433 (1.2); 3.6547 (0.9); 3.5909 (0.7); 3.3489 (10.9); 2.5342 (1.4); 2.5283 (3.1); 2.5223 (4.2);
2.5163 (3.1); 2.5105 (1.5); 2.0088 (1.5); 1.2636 (1.9); 1.2179 (0.5); 1.1942 (0.8); 1.1705 (0.4); 0.8981 (0.6); 0.8763 (1.7); 0.8531
(0.6); 0.0188 (5.8)
Ia-065: ¹H-NMR(400.2 MHz, d₆-DMSO):
δ = 8.6813 (9.8); 7.8597 (2.8); 7.8538 (1.4); 7.8437 (2.3); 7.8377 (16.0); 7.8278 (15.1); 7.8190 (4.1); 7.8141 (6.8); 7.8093 (4.7);
7.8059 (3.1); 7.7363 (2.0); 7.7329 (2.9); 7.7291 (1.9); 7.7174 (2.5); 7.7134 (3.3); 7.7100 (2.2); 7.5467 (2.4); 7.5270 (5.7); 7.5078
(4.1); 7.4898 (3.0); 7.4866 (3.9); 7.4849 (3.5); 7.4818 (3.0); 7.4698 (1.5); 7.4666 (1.5); 7.4648 (1.7); 7.4618 (1.3); 4.0105 (3.9);
3.9641 (4.7); 3.6233 (2.6); 3.5767 (2.1); 3.3346 (46.2); 2.8917 (1.4); 2.7325 (1.2); 2.6303 (0.4); 2.5264 (0.9); 2.5217 (1.3);
2.5130 (15.8); 2.5086 (31.8); 2.5040 (41.3); 2.4994 (29.4); 2.4949 (13.9); 1.2369 (1.2); −0.0002 (1.6)
Ia-066: ¹H-NMR(400.2 MHz, d₆-DMSO):
δ = 8.6803 (11.2); 7.8645 (3.8); 7.8591 (1.8); 7.8482 (2.7); 7.8426 (16.0); 7.8281 (15.5); 7.8226 (2.9); 7.8118 (1.6); 7.8064 (4.0);
7.6333 (1.5); 7.6291 (2.5); 7.6231 (3.9); 7.6204 (3.3); 7.6165 (1.9); 7.6023 (6.5); 7.5982 (6.8); 7.5655 (2.0); 7.5509 (2.1); 7.5455
(3.1); 7.5302 (2.6); 7.5269 (1.9); 7.5098 (1.0); 7.2727 (1.2); 7.2702 (1.3); 7.2666 (1.2); 7.2640 (1.2); 7.2502 (2.4); 7.2440 (2.2);
7.2304 (1.2); 7.2259 (1.4); 7.2215 (1.0); 4.0083 (4.2); 3.9620 (5.2); 3.6239 (3.0); 3.5773 (2.4); 3.3342 (29.4); 2.8917 (0.6);
2.7327 (0.5); 2.5266 (0.8); 2.5219 (1.2); 2.5132 (15.2); 2.5087 (30.8); 2.5042 (40.6); 2.4996 (29.2); 2.4950 (14.1); 1.2370
(1.0); −0.0002 (3.0)
Ia-067: ¹H-NMR(400.2 MHz, d₆-DMSO):
δ = 8.7206 (10.1); 8.3504 (8.0); 8.3371 (8.4); 8.0161 (11.3); 8.0115 (4.1); 7.9994 (4.6); 7.9946 (16.0); 7.8892 (15.9); 7.8845 (4.7);
7.8724 (3.9); 7.8678 (11.4); 7.8188 (0.6); 7.7941 (2.9); 7.7896 (4.7); 7.7855 (3.1); 7.7809 (3.0); 7.7765 (4.5); 7.7722 (2.8);
7.6304 (9.3); 4.0297 (5.8); 3.9833 (7.2); 3.6444 (4.0); 3.5976 (3.2); 3.3343 (36.4); 2.8930 (0.6); 2.7344 (0.6); 2.7331 (0.6);
2.6740 (0.4); 2.5275 (1.2); 2.5227 (1.8); 2.5141 (23.3); 2.5096 (47.7); 2.5050 (62.6); 2.5004 (44.9); 2.4958 (21.5); 2.3319 (0.4);
1.2597 (0.3); 1.2377 (1.7); 0.8527 (0.5); −0.0002 (5.7)
Ia-068: ¹H-NMR(400.2 MHz, d₆-DMSO):
δ = 10.6161 (1.8); 8.7089 (2.4); 8.4308 (2.0); 8.4032 (2.1); 8.3901 (2.2); 7.8931 (2.4); 7.8721 (5.5); 7.8406 (4.6); 7.8195 (2.1);
7.4630 (1.5); 7.4499 (1.4); 4.0140 (1.0); 3.9680 (1.2); 3.6310 (1.4); 3.5845 (1.1); 3.3351 (24.1); 2.8937 (0.6); 2.7358 (0.5);
2.7345 (0.5); 2.5281 (0.6); 2.5234 (0.9); 2.5147 (9.8); 2.5102 (19.6); 2.5056 (25.6); 2.5010 (18.1); 2.4964 (8.4); 2.1315 (16.0);
1.2378 (0.7); −0.0002 (2.0)
Ia-069: ¹H-NMR(300.2 MHz, CDCl3):
δ = 8.0339 (1.9); 7.6056 (0.6); 7.5900 (6.5); 7.5843 (2.4); 7.5681 (2.4); 7.5621 (7.5); 7.2989 (17.3); 7.1596 (7.2); 7.1318 (6.3);
3.7613 (2.8); 3.7020 (5.1); 3.5653 (3.0); 3.5614 (3.2); 3.5060 (1.6); 3.5021 (1.8); 2.9900 (16.0); 2.9094 (13.7); 2.8259 (1.9);
2.6553 (0.3); 2.0088 (0.4); 1.9919 (0.9); 1.9805 (1.0); 1.9640 (2.0); 1.9473 (1.1); 1.9360 (1.1); 1.9191 (0.6); 1.6399 (1.9); 1.2926
(1.0); 1.1110 (1.3); 1.0954 (3.3); 1.0887 (3.6); 1.0737 (1.9); 1.0672 (3.7); 1.0605 (3.3); 1.0459 (1.6); 0.8114 (1.6); 0.7958 (4.3);
0.7899 (3.9); 0.7796 (3.5); 0.7735 (4.7); 0.7573 (1.3); 0.0376 (10.3); 0.0271 (0.4)
Ia-070: ¹H-NMR(300.2 MHz, d₆-DMSO):
δ = 8.6544 (0.6); 7.7649 (0.7); 7.7507 (0.7); 4.6060 (0.4); 3.3444 (16.0); 2.5343 (1.4); 2.5284 (3.2); 2.5223 (4.4); 2.5163 (3.2);
2.5104 (1.5); 0.0207 (7.3)
Ia-071: ¹H-NMR(400.2 MHz, d₆-DMSO):
δ = 8.7028 (1.3); 7.9828 (0.4); 7.9788 (0.4); 7.9644 (16.0); 7.9091 (1.4); 7.9042 (0.6); 7.8926 (0.9); 7.8874 (4.2); 7.8652 (4.0);
7.8600 (0.9); 7.8484 (0.6); 7.8435 (1.4); 4.0160 (1.2); 3.9697 (1.5); 3.6321 (0.9); 3.5853 (0.7); 3.3354 (11.0); 2.8924 (0.8);
2.7339 (0.7); 2.7330 (0.7); 2.5224 (0.4); 2.5137 (5.3); 2.5092 (10.6); 2.5047 (13.8); 2.5000 (9.9); 2.4955 (4.7); −0.0002 (0.9)
Ia-072: ¹H-NMR(499.9 MHz, CDCl3):
δ = 7.5412 (3.7); 7.5242 (4.9); 7.4022 (4.7); 7.3853 (3.7); 7.3014 (2.5); 7.2974 (2.5); 7.2597 (7.1); 7.2556 (1.6); 7.2520 (0.8);
7.2399 (4.2); 7.2381 (4.4); 7.2209 (6.3); 7.2066 (1.1); 7.1992 (5.1); 7.1823 (2.1); 7.1783 (1.2); 7.1760 (1.5); 7.1611 (1.9); 7.1570
(0.7); 7.1538 (0.5); 7.1514 (0.4); 7.1463 (0.7); 7.1389 (0.6); 7.0595 (3.0); 7.0451 (3.2); 7.0311 (0.8); 5.7689 (0.4); 5.2971 (7.3);
3.6808 (1.5); 3.6452 (3.0); 3.5758 (1.7); 3.5400 (0.9); 3.5091 (16.0); 3.4935 (5.0); 1.2570 (0.3); −0.0002 (8.1)
Ia-073: ¹H-NMR(400.2 MHz, d₆-DMSO):
δ = 8.7451 (0.4); 8.7334 (0.4); 8.3155 (0.5); 8.0559 (2.9); 8.0044 (8.5); 7.9833 (11.4); 7.9620 (2.1); 7.9534 (1.2); 7.8810 (5.0);
7.8594 (15.2); 7.8440 (12.1); 7.8375 (16.0); 7.8227 (9.8); 7.8160 (5.7); 7.4228 (2.8); 4.0113 (0.4); 3.9979 (3.7); 3.9518 (4.7);
3.6268 (3.1); 3.5804 (2.5); 3.3325 (13.3); 3.1990 (1.4); 2.8919 (8.8); 2.7332 (7.3); 2.6771 (0.4); 2.6725 (0.6); 2.6679 (0.4);
2.6301 (2.1); 2.5260 (1.6); 2.5213 (2.5); 2.5127 (34.1); 2.5082 (68.8); 2.5036 (89.6); 2.4990 (63.9); 2.4945 (30.7); 2.3350 (0.4);
2.3304 (0.6); 2.3258 (0.4); 1.2594 (0.5); 1.2393 (2.3); 0.8533 (0.6); −0.0002 (6.8)
Ia-074: ¹H-NMR(400.2 MHz, d₆-DMSO):
δ = 8.6780 (0.9); 7.9538 (0.6); 7.8129 (16.0); 7.7961 (0.4); 7.7892 (3.2); 7.7842 (1.0); 7.7727 (1.1); 7.7676 (3.8); 7.7610 (0.5);
7.5681 (0.4); 7.5614 (3.8); 7.5564 (1.1); 7.5447 (1.0); 7.5398 (3.2); 7.5331 (0.3); 3.9988 (1.3); 3.9525 (1.6); 3.6174 (1.0); 3.5711
(0.8); 3.3356 (19.2); 2.8923 (4.8); 2.7340 (4.0); 2.7328 (3.9); 2.5217 (0.4); 2.5131 (5.5); 2.5086 (11.4); 2.5041 (15.0); 2.4995
(10.7); 2.4949 (5.1); 1.2376 (0.4); −0.0002 (1.7)
Ia-075: ¹H-NMR(400.2 MHz, d₆-DMSO):
δ = 8.6754 (3.0); 7.9550 (1.7); 7.8228 (2.0); 7.8207 (1.7); 7.8122 (4.3); 7.8068 (3.1); 7.8006 (16.0); 7.7942 (15.8); 7.7819 (2.0);
7.7764 (4.7); 7.7720 (2.2); 7.3565 (0.4); 7.3485 (4.2); 7.3432 (1.3); 7.3316 (1.4); 7.3264 (7.9); 7.3210 (1.5); 7.3095 (1.2); 7.3042
(3.9); 7.2962 (0.4); 3.9989 (3.1); 3.9526 (3.9); 3.6177 (2.2); 3.5709 (1.8); 3.3460 (48.0); 3.1994 (0.9); 2.8927 (12.4); 2.7346
(10.5); 2.6314 (1.4); 2.5279 (0.4); 2.5232 (0.5); 2.5146 (7.7); 2.5101 (15.9); 2.5055 (21.0); 2.5009 (15.1); 2.4964 (7.2); 1.2369
(0.9); −0.0002 (2.1)
Ia-076: ¹H-NMR(400.2 MHz, d₆-DMSO):
δ = 8.6476 (2.8); 7.7865 (0.8); 7.7800 (0.4); 7.7644 (6.9); 7.7581 (6.7); 7.7425 (0.4); 7.7358 (0.8); 7.7146 (0.3); 7.7069 (3.4);
7.7018 (1.1); 7.6901 (1.1); 7.6848 (3.8); 7.6772 (0.4); 7.0704 (0.4); 7.0627 (3.7); 7.0575 (1.1); 7.0457 (1.0); 7.0406 (3.4); 7.0329
(0.3); 3.9837 (1.4); 3.9375 (1.8); 3.8113 (16.0); 3.6040 (1.0); 3.5575 (0.8); 3.3341 (15.3); 2.5256 (0.3); 2.5209 (0.5); 2.5124
(5.9); 2.5079 (11.8); 2.5033 (15.4); 2.4987 (11.0); 2.4942 (5.3); −0.0002 (1.0)
Ia-077: ¹H-NMR(300.2 MHz, d₆-DMSO):
δ = 8.6119 (1.6); 7.6621 (3.9); 7.6346 (4.8); 7.3644 (4.4); 7.3369 (3.7); 3.9526 (1.8); 3.8909 (2.4); 3.5697 (1.5); 3.5077 (1.1);
3.3488 (16.0); 2.6007 (0.4); 2.5736 (0.7); 2.5341 (3.0); 2.5282 (5.9); 2.5222 (8.0); 2.5162 (5.8); 2.5105 (2.8); 1.8208 (2.4);
1.7892 (2.6); 1.7466 (0.8); 1.7072 (0.7); 1.4580 (1.1); 1.4262 (2.1); 1.3982 (2.0); 1.3672 (1.1); 1.3222 (0.5); 1.2737 (0.5); 1.2335
(0.4); 0.0202 (7.4)
Ia-078: ¹H-NMR(400.2 MHz, d₆-DMSO):
δ = 8.6782 (0.5); 7.9534 (0.5); 7.8311 (0.4); 7.8226 (0.4); 7.8079 (16.0); 7.7928 (0.3); 7.7872 (0.4); 7.7844 (0.5); 7.7539 (2.2);
7.7505 (3.1); 7.7457 (0.9); 7.7326 (3.2); 7.7250 (0.4); 7.5193 (1.6); 7.5149 (0.6); 7.5012 (3.6); 7.4980 (1.8); 7.4853 (1.0); 7.4817
(2.2); 7.4355 (0.8); 7.4325 (1.4); 7.4295 (0.8); 7.4192 (0.6); 7.4141 (1.8); 7.4089 (0.5); 7.3988 (0.4); 7.3959 (0.6); 7.3929 (0.4);
3.9994 (1.7); 3.9532 (2.2); 3.6181 (1.2); 3.5716 (1.0); 3.3345 (11.2); 2.8910 (4.1); 2.7329 (3.3); 2.7318 (3.3); 2.5256 (0.4);
2.5209 (0.6); 2.5123 (8.3); 2.5078 (16.8); 2.5032 (22.0); 2.4986 (15.9); 2.4940 (7.7); 1.2383 (0.5); −0.0002 (1.4)
Ia-079: ¹H-NMR(400.2 MHz, d₆-DMSO):
δ = 8.7087 (15.7); 8.6856 (6.1); 8.6717 (6.3); 7.9597 (8.8); 7.9383 (15.4); 7.8802 (16.0); 7.8755 (4.6); 7.8587 (9.4); 7.7964 (9.7);
7.7927 (6.8); 7.7848 (6.6); 7.7811 (9.5); 7.6936 (0.5); 7.6787 (0.5); 4.0228 (5.6); 4.0119 (0.5); 3.9764 (7.0); 3.6384 (4.0); 3.5915
(3.2); 3.3382 (13.6); 2.8921 (2.2); 2.7336 (1.9); 2.7324 (1.9); 2.6731 (0.5); 2.6687 (0.3); 2.5525 (0.6); 2.5387 (0.4); 2.5267 (1.4);
2.5219 (2.2); 2.5133 (28.6); 2.5088 (57.4); 2.5042 (74.8); 2.4996 (53.9); 2.4951 (26.2); 2.3355 (0.3); 2.3310 (0.5); 2.3264 (0.3);
1.2595 (0.4); 1.2382 (2.3); 0.8528 (0.5); −0.0002 (5.2)
Ia-080: ¹H-NMR(400.2 MHz, d₆-DMSO):
δ = 8.6048 (5.6); 8.2489 (4.6); 7.9594 (4.8); 7.9579 (5.0); 7.7097 (0.9); 7.7030 (0.5); 7.6874 (9.8); 7.6826 (9.5); 7.6670 (0.5);
7.6603 (0.9); 3.9522 (1.9); 3.9061 (2.4); 3.8745 (16.0); 3.5778 (1.4); 3.5313 (1.1); 3.3337 (14.7); 2.5255 (0.4); 2.5207 (0.6);
2.5121 (6.8); 2.5076 (13.8); 2.5031 (18.1); 2.4985 (13.0); 2.4939 (6.2); −0.0002 (1.5)
Ia-081: ¹H-NMR(400.2 MHz, d₆-DMSO):
δ = 8.6068 (3.2); 8.3095 (3.1); 7.9679 (3.1); 7.6914 (16.0); 7.6280 (0.6); 7.6256 (0.4); 7.6161 (0.4); 7.5989 (0.4); 7.5663 (0.4);
4.1877 (0.8); 4.1695 (2.4); 4.1513 (2.4); 4.1331 (0.8); 3.9542 (1.4); 3.9081 (1.8); 3.5810 (1.1); 3.5350 (0.9); 3.3326 (8.9); 2.8914
(0.8); 2.7332 (0.6); 2.7321 (0.6); 2.5258 (0.3); 2.5211 (0.5); 2.5124 (6.5); 2.5079 (13.1); 2.5033 (17.1); 2.4987 (12.2); 2.4942
(5.9); 1.4315 (3.3); 1.4133 (7.1); 1.3951 (3.2); 1.2388 (0.5); −0.0002 (1.5)
Ia-082: ¹H-NMR(400.2 MHz, d₆-DMSO):
δ = 8.6890 (8.4); 8.6465 (4.3); 8.6401 (4.5); 8.3998 (1.7); 8.3932 (1.6); 8.3790 (2.7); 8.3724 (2.6); 8.3588 (1.8); 8.3521 (1.7);
7.8853 (3.5); 7.8799 (1.7); 7.8690 (2.6); 7.8635 (16.0); 7.8502 (15.7); 7.8445 (2.8); 7.8340 (1.6); 7.8285 (3.6); 7.3374 (2.9);
7.3302 (3.0); 7.3160 (2.9); 7.3088 (2.8); 4.0121 (4.1); 3.9657 (5.0); 3.6290 (2.9); 3.5823 (2.3); 3.3311 (67.1); 2.8917 (0.9);
2.7320 (0.8); 2.6724 (0.4); 2.5259 (1.2); 2.5213 (1.8); 2.5125 (24.6); 2.5080 (50.5); 2.5034 (66.8); 2.4988 (48.1); 2.4943 (23.2);
2.3302 (0.4); 1.2385 (1.1); −0.0002 (9.5)
Ia-083: ¹H-NMR(400.2 MHz, d₆-DMSO):
δ = 10.6489 (3.5); 8.7278 (2.3); 8.7240 (3.1); 8.7196 (2.5); 8.6742 (3.6); 8.2005 (0.3); 8.1777 (5.0); 8.1721 (3.0); 8.1556 (0.4);
8.1502 (0.4); 7.8652 (2.2); 7.8603 (1.0); 7.8436 (7.6); 7.8250 (7.3); 7.8084 (1.0); 7.8034 (2.3); 4.0041 (2.1); 3.9579 (2.6); 3.6224
(1.5) ; 3.5761 (1.2); 3.3337 (12.5); 2.5271 (0.5); 2.5223 (0.7); 2.5138 (9.3); 2.5093 (18.9); 2.5048 (24.6); 2.5002 (17.7); 2.4957
(8.5) ; 2.1264 (16.0); 1.2369 (0.5); −0.0002 (2.0)
Ia-084: ¹H-NMR(400.2 MHz, d₆-DMSO):
δ = 8.6698 (2.2); 8.5818 (1.7); 8.5764 (1.7); 8.1145 (1.2); 8.1080 (1.2); 8.0928 (1.2); 8.0863 (1.2); 7.8049 (16.0); 7.6931 (0.4);
7.6785 (0.4); 6.9527 (1.7); 6.9518 (1.7); 6.9312 (1.7); 6.9301 (1.7); 3.9951 (1.3); 3.9488 (1.6); 3.9108 (13.8); 3.6169 (0.9);
3.5715 (0.8); 3.3329 (11.1); 2.8920 (1.2); 2.7335 (1.1); 2.5265 (0.3); 2.5218 (0.5); 2.5131 (6.1); 2.5087 (12.3); 2.5041 (16.1);
2.4995 (11.7); 2.4950 (5.8); 1.2383 (0.5); −0.0002 (1.1)
Ia-085: ¹H-NMR(400.2 MHz, d₆-DMSO):
δ = 8.9753 (4.7); 8.9706 (4.6); 8.7152 (0.8); 8.6235 (3.6); 8.6198 (3.7); 8.6117 (3.7); 8.6079 (3.6); 8.1756 (2.3); 8.1715 (2.8);
8.1698 (2.8); 8.1656 (2.3); 8.1557 (2.5); 8.1516 (2.9); 8.1498 (3.0); 8.1457 (2.3); 7.9548 (0.4); 7.8942 (4.2); 7.8887 (2.2); 7.8724
(16.0); 7.8577 (15.3); 7.8521 (3.2); 7.8414 (2.2); 7.8360 (4.2); 7.8124 (0.7); 7.8030 (0.7); 7.5391 (2.8); 7.5375 (2.8); 7.5272
(2.7); 7.5256 (2.8); 7.5192 (2.7); 7.5175 (2.7); 7.5074 (2.6); 7.5056 (2.5); 4.0129 (4.0); 3.9666 (5.1); 3.6335 (3.0); 3.5870 (2.5);
3.3411 (13.5); 3.2873 (0.9); 2.8923 (1.4); 2.7342 (1.2); 2.7330 (1.3); 2.5756 (0.4); 2.5141 (13.0); 2.5096 (24.8); 2.5051 (32.1);
2.5005 (22.8); 2.4960 (10.8); 2.4454 (1.2); 1.2374 (0.7); −0.0002 (3.1)
Ia-086: ¹H-NMR(400.2 MHz, d₆-DMSO):
δ = 8.7221 (0.3); 8.4916 (1.8); 8.4773 (1.8); 8.4148 (3.4); 7.7971 (3.2); 7.7761 (4.4); 7.6478 (4.3); 7.6268 (3.2); 7.2034 (2.2);
7.1890 (2.1); 3.9875 (1.3); 3.9412 (1.7); 3.8757 (16.0); 3.6190 (1.2); 3.5723 (1.0); 3.3362 (5.5); 2.8925 (0.8); 2.7342 (0.7);
2.7332 (0.6); 2.5264 (0.4); 2.5216 (0.7); 2.5130 (8.6); 2.5085 (17.3); 2.5040 (22.5); 2.4993 (16.2); 2.4948 (7.8); 1.2395
(0.4); −0.0002 (1.6)
Ia-087: ¹H-NMR(400.2 MHz, d₆-DMSO):
δ = 9.2272 (16.0); 8.7178 (2.0); 7.9841 (0.5); 7.9797 (3.2); 7.9750 (1.2); 7.9631 (1.5); 7.9582 (5.4); 7.8965 (1.0); 7.8924 (5.2);
7.8874 (1.5); 7.8756 (1.2); 7.8709 (3.2); 4.0248 (1.8); 3.9784 (2.2); 3.6455 (1.2); 3.5986 (1.0); 3.3471 (22.7); 2.5160 (3.9);
2.5115 (8.0); 2.5069 (10.6); 2.5023 (7.5); 2.4978 (3.5); 1.2369 (0.4); −0.0002 (1.0)
Ia-088: ¹H-NMR(400.2 MHz, d₆-DMSO):
δ = 8.6311 (2.2); 8.2207 (7.6); 8.0120 (0.3); 7.9529 (2.1); 7.8562 (0.4); 7.8305 (6.0); 7.7817 (2.5); 7.7767 (1.3); 7.7598 (8.0);
7.7414 (2.9); 7.7369 (7.4); 7.7317 (1.7); 7.7199 (1.2); 7.7149 (2.8); 7.7111 (1.3); 7.7059 (5.4); 7.6891 (2.2); 7.6844 (7.1); 7.6482
(0.8); 7.6282 (0.8); 7.6066 (0.9); 7.5667 (0.4); 7.5367 (7.2); 7.5320 (2.4); 7.5152 (5.7); 7.4752 (1.3); 7.4541 (1.1); 7.4199 (0.6);
7.3983 (0.5); 4.1243 (2.8); 3.9620 (0.8); 3.9439 (3.3); 3.9153 (1.2); 3.8976 (4.1); 3.8674 (0.4); 3.5630 (3.6); 3.5416 (0.6); 3.5164
(3.0); 3.4952 (0.5); 3.4827 (0.4); 3.4463 (0.8); 3.3392 (1.5); 3.2389 (4.5); 3.1987 (1.5); 3.1764 (0.3); 3.1578 (0.3); 2.8915 (16.0);
2.7328 (13.4); 2.7318 (12.9); 2.6768 (0.4); 2.6722 (0.5); 2.6677 (0.4); 2.6296 (1.9); 2.5258 (1.5); 2.5210 (2.4); 2.5124 (30.1);
2.5079 (60.2); 2.5033 (78.0); 2.4987 (55.6); 2.4942 (26.4); 2.3345 (0.3); 2.3302 (0.5); 2.3256 (0.3); 1.2725 (0.8); 1.2592 (1.3);
1.2428 (2.2); 1.2252 (0.4); 0.8533 (0.5); −0.0002 (5.3)
Ia-089: ¹H-NMR(400.2 MHz, d₆-DMSO):
δ = 8.6902 (4.2); 7.8300 (3.2); 7.8258 (1.2); 7.8135 (1.2); 7.8089 (4.0); 7.6453 (0.7); 7.6411 (0.5); 7.6303 (4.6); 7.6140 (1.8);
7.6092 (3.6); 7.5982 (1.1); 7.5946 (0.6); 7.5739 (0.7); 7.5659 (0.8); 7.5592 (0.5); 7.5565 (0.6); 7.5506 (0.7); 7.5482 (0.7); 7.1649
(6.3); 3.9976 (1.6); 3.9512 (2.0); 3.6384 (16.0); 3.6178 (1.1); 3.5711 (0.9); 3.3334 (23.0); 2.8914 (1.0); 2.7320 (0.9); 2.5255
(0.4); 2.5208 (0.6); 2.5121 (7.3); 2.5077 (14.7); 2.5031 (19.2); 2.4985 (13.8); 2.4940 (6.6); 1.2393 (0.4); −0.0002 (1.0)
Ia-090: ¹H-NMR(300.2 MHz, d₆-DMSO):
δ = 8.7144 (1.4); 7.8670 (1.6); 7.8394 (2.3); 7.6998 (2.4); 7.6718 (1.7); 7.5246 (1.7); 7.5183 (1.8); 6.5306 (1.8); 6.5243 (1.9);
4.0400 (0.8); 3.9783 (1.0); 3.9147 (9.2); 3.6515 (0.6); 3.5893 (0.5); 3.3398 (16.0); 2.5344 (2.9); 2.5284 (6.3); 2.5224 (8.8);
2.5164 (6.4); 2.5105 (3.0); 0.0320 (0.4); 0.0212 (11.0); 0.0102 (0.4)
Ia-091: ¹H-NMR(400.2 MHz, d₆-DMSO):
δ = 8.7028 (6.6); 7.9529 (1.3); 7.8496 (6.8); 7.8451 (2.4); 7.8330 (2.6); 7.8284 (8.4); 7.6445 (1.3); 7.6406 (0.8); 7.6324 (1.3);
7.6274 (2.5); 7.6239 (2.0); 7.6146 (3.0); 7.6097 (9.1); 7.6050 (3.0); 7.5979 (2.5); 7.5932 (3.3); 7.5885 (7.1); 7.5741 (1.4); 7.5661
(1.7); 7.5595 (1.0); 7.5566 (1.2); 7.5509 (1.4); 7.5483 (1.5); 7.5461 (1.2); 7.5363 (6.2); 7.5317 (6.2); 6.4472 (6.4); 6.4426 (6.4);
4.1951 (1.7); 4.1771 (5.3); 4.1591 (5.4); 4.1411 (1.8); 4.0106 (3.3); 3.9642 (4.1); 3.6236 (2.3); 3.5772 (1.9); 3.3310 (55.4);
3.1986 (0.4); 2.8911 (10.6); 2.7324 (8.5); 2.7314 (8.7); 2.6716 (0.4); 2.6294 (0.6); 2.5252 (1.1); 2.5205 (1.6); 2.5117 (23.4);
2.5073 (48.5); 2.5027 (64.3); 2.4980 (46.2); 2.4935 (22.2); 2.3295 (0.4); 1.3222 (7.2); 1.3043 (16.0); 1.2862 (7.1); 1.2399
(1.2); −0.0002 (8.6)
Ia-092: ¹H-NMR(400.2 MHz, d₆-DMSO):
δ = 13.0226 (0.5); 8.6601 (0.6); 7.9529 (2.4); 7.9363 (1.1); 7.9173 (1.3); 7.8214 (0.7); 7.7625 (1.7); 7.7427 (1.4); 6.8237 (3.0);
6.8182 (3.0); 3.9777 (1.9); 3.9315 (2.4); 3.5996 (1.3); 3.5531 (1.0); 3.3353 (27.3); 2.8908 (16.0); 2.7319 (13.4); 2.5253 (0.7);
2.5205 (1.0); 2.5119 (13.2); 2.5075 (26.7); 2.5029 (34.9); 2.4983 (25.2); 2.4938 (12.2); 1.2394 (0.7); −0.0002 (3.0)
Ia-093: ¹H-NMR(499.9 MHz, MeOD):
δ = 8.1082 (6.5); 8.1048 (2.3); 8.0946 (2.4); 8.0912 (7.3); 7.8384 (7.5); 7.8348 (2.4); 7.8247 (2.3); 7.8214 (6.4); 5.3875 (1.1);
4.7465 (12.4); 3.8001 (3.4); 3.7636 (4.2); 3.4497 (2.2); 3.4477 (2.2); 3.4132 (1.8); 3.4110 (1.8); 3.2159 (5.7); 3.2127 (11.2);
3.2094 (15.9); 3.2062 (11.3); 3.2030 (5.9); 2.7575 (2.4); 2.7423 (7.5); 2.7271 (7.6); 2.7119 (2.6); 1.2893 (7.8); 1.2741 (16.0);
1.2590 (7.6)
Ia-094: ¹H-NMR(400.2 MHz, d₆-DMSO):
δ = 8.3202 (0.8); 8.2193 (6.0); 8.1979 (7.5); 7.9997 (7.2); 7.9783 (5.9); 7.9543 (2.2); 4.1443 (0.6); 4.0944 (3.3); 4.0760 (4.6);
4.0274 (0.9); 3.3850 (22.5); 3.3347 (38.7); 2.8948 (14.6); 2.8521 (2.2); 2.8333 (7.0); 2.8144 (7.1); 2.7955 (2.4); 2.7523 (0.6);
2.7355 (12.6); 2.5553 (0.4); 2.5145 (15.6); 2.5103 (29.2); 2.5058 (37.2); 2.5013 (27.4); 2.4970 (13.7); 1.3259 (7.7); 1.3143 (1.5);
1.3070 (16.0); 1.2956 (0.9); 1.2881 (7.4); 1.2377 (1.4)
Ia-095: ¹H-NMR(300.2 MHz, CDCl3):
δ = 8.2467 (4.2); 8.2190 (4.7); 7.8718 (4.9); 7.8440 (4.2); 7.2991 (4.0); 4.0255 (1.4); 3.9642 (2.3); 3.8033 (3.1); 3.7425 (1.8);
2.9301 (1.5); 2.9048 (4.1); 2.8796 (4.2); 2.8545 (1.6); 2.2343 (16.0); 1.6045 (5.4); 1.4646 (4.5); 1.4394 (8.2); 1.4142 (4.1);
0.0367 (3.9)
Ia-096: ¹H-NMR(400.2 MHz, d₆-DMSO):
δ = 8.7833 (0.4); 8.2171 (5.8); 8.2128 (2.1); 8.2002 (2.9); 8.1957 (7.0); 8.1835 (0.3); 8.1787 (0.6); 8.1590 (0.3); 7.9721 (0.6);
7.9507 (7.1); 7.9336 (2.1); 7.9292 (5.6); 4.3601 (1.0); 4.3117 (2.2); 4.2476 (3.5); 4.1996 (1.6); 3.3347 (28.4); 2.8943 (2.0);
2.8515 (2.0); 2.8431 (0.5); 2.8327 (6.5); 2.8244 (0.7); 2.8138 (6.7); 2.7949 (2.2); 2.7350 (1.7); 2.5280 (0.6); 2.5231 (1.0); 2.5145
(12.7); 2.5101 (25.2); 2.5055 (32.8); 2.5010 (24.1); 2.4965 (11.9); 1.8054 (0.4); 1.7940 (0.8); 1.7852 (1.0); 1.7742 (1.7); 1.7629
(1.1); 1.7545 (0.9); 1.7432 (0.4); 1.3319 (0.6); 1.3252 (7.6); 1.3132 (1.3); 1.3063 (16.0); 1.2943 (0.9); 1.2874 (7.4); 1.2373 (1.3);
1.0526 (0.3); 1.0417 (1.9); 1.0321 (2.8); 1.0218 (2.3); 1.0121 (2.7); 1.0023 (0.5); 0.9962 (0.4); 0.9753 (0.3); 0.9636 (0.3); 0.9522
(0.4); 0.9398 (1.5); 0.9302 (2.3); 0.9201 (2.4); 0.9089 (1.3); 0.8959 (0.4); 0.8529 (0.4)
Ia-097: ¹H-NMR(400.2 MHz, d₆-DMSO):
δ = 8.7841 (0.3); 8.2918 (0.4); 8.2709 (0.9); 8.2584 (6.2); 8.2371 (7.8); 8.2271 (1.4); 8.2048 (0.6); 8.1791 (0.4); 8.0162 (7.3);
7.9949 (6.3); 7.9681 (5.4); 7.9501 (6.1); 7.9471 (5.0); 7.7737 (1.2); 7.7551 (3.0); 7.7364 (1.9); 7.6166 (4.0); 7.5968 (6.0); 7.5778
(2.8); 4.6363 (1.6); 4.5878 (2.4); 4.3896 (3.5); 4.3413 (2.5); 3.3408 (64.8); 2.8943 (1.2); 2.8621 (2.3); 2.8432 (7.1); 2.8244 (7.4);
2.8054 (2.5); 2.7353 (1.1); 2.5107 (31.8); 2.5063 (40.9); 2.5018 (30.5); 1.3350 (7.8); 1.3161 (16.0); 1.3049 (1.9); 1.2972 (7.6);
1.2861 (0.8); 1.2369 (1.1)
Ia-098: ¹H-NMR(400.2 MHz, d₆-DMSO):
δ = 8.2334 (7.2); 8.2126 (8.0); 8.1795 (0.3); 7.9667 (8.1); 7.9461 (6.8); 4.3698 (1.4); 4.3215 (3.2); 4.2568 (4.6); 4.2088 (2.1);
3.3326 (31.2); 2.8947 (0.7); 2.8553 (2.3); 2.8364 (7.1); 2.8175 (7.3); 2.7987 (2.5); 2.7360 (0.7); 2.5101 (27.0); 2.5058 (33.9);
2.5018 (25.9); 2.4967 (17.6); 2.4766 (6.9); 2.4582 (2.3); 1.3286 (7.9); 1.3096 (16.0); 1.2908 (7.6); 1.2378 (1.3); 1.0327 (7.0);
1.0141 (14.5); 0.9956 (6.8); 0.8531 (0.4)
Ia-099: ¹H-NMR(400.2 MHz, d₆-DMSO):
δ = 8.7857 (0.4); 8.3202 (0.8); 8.2714 (0.6); 8.2271 (0.8); 8.2048 (1.0); 8.2006 (4.2); 8.1962 (1.6); 8.1836 (1.6); 8.1791 (5.1);
7.9724 (5.0); 7.9679 (1.8); 7.9511 (5.1); 4.9548 (3.0); 4.8375 (3.0); 4.0448 (1.8); 3.9984 (2.3); 3.6455 (1.4); 3.5985 (1.1); 3.3408
(3.1); 2.8948 (16.0); 2.8490 (1.8); 2.8431 (0.7); 2.8302 (5.2); 2.8245 (0.9); 2.8192 (0.5); 2.8114 (5.1); 2.7925 (1.6); 2.7361
(13.8); 2.7352 (13.4); 2.5286 (0.7); 2.5238 (1.1); 2.5152 (11.2); 2.5107 (22.0); 2.5062 (28.7); 2.5016 (20.9); 2.4971 (10.2);
1.3330 (1.0); 1.3235 (5.7); 1.3142 (2.1); 1.3104 (1.1); 1.3046 (12.1); 1.2952 (1.1); 1.2915 (0.6); 1.2857 (5.5); 1.2378 (1.0)
Ia-100: ¹H-NMR(499.9 MHz, d₆-DMSO):
δ = 8.2510 (6.2); 8.2474 (2.3); 8.2375 (2.6); 8.2339 (6.9); 7.9822 (6.9); 7.9785 (2.4); 7.9686 (2.4); 7.9650 (5.9); 5.7624 (0.4);
4.4782 (1.3); 4.4394 (2.2); 4.3394 (3.5); 4.3007 (2.2); 4.2493 (0.5); 4.2145 (9.7); 4.2126 (9.2); 4.1777 (0.4); 3.3229 (40.1);
3.3208 (39.4); 3.2936 (0.3); 2.8589 (2.2); 2.8439 (6.7); 2.8287 (6.8); 2.8136 (2.2); 2.5167 (3.8); 2.5131 (7.2); 2.5095 (9.3);
2.5058 (6.5); 2.5022 (3.0); 1.3311 (7.7); 1.3159 (16.0); 1.3008 (7.3)
Ia-101: ¹H-NMR(400.2 MHz, d₆-DMSO):
δ = 8.2271 (5.6); 8.2226 (1.9); 8.2101 (2.2); 8.2056 (6.8); 8.1686 (0.5); 8.1471 (0.7); 7.9872 (6.7); 7.9826 (2.1); 7.9656 (5.6);
5.4171 (0.8); 4.9297 (0.6); 4.6469 (3.0); 4.6051 (4.3); 4.4200 (4.6); 4.3782 (3.3); 4.2204 (1.1); 4.1719 (3.6); 4.1361 (2.3); 4.0867
(0.7); 3.3331 (29.0); 2.8947 (1.4); 2.8538 (2.0); 2.8481 (0.4); 2.8349 (6.4); 2.8292 (1.0); 2.8160 (6.6); 2.8104 (1.0); 2.7971 (2.2);
2.7914 (0.3); 2.7362 (1.2); 2.7351 (1.1); 2.5282 (0.5); 2.5234 (0.8); 2.5148 (10.7); 2.5103 (21.3); 2.5057 (27.7); 2.5011 (20.0);
2.4966 (9.6); 2.3364 (0.3); 2.1669 (0.4); 2.1552 (1.0); 2.1474 (1.2); 2.1442 (0.8); 2.1357 (1.8); 2.1241 (1.1); 2.1164 (1.1); 2.1047
(0.5); 1.3269 (7.5); 1.3081 (16.0); 1.2892 (7.4); 1.2376 (1.0); 0.9493 (0.7); 0.9401 (1.8); 0.9318 (4.1); 0.9243 (1.5); 0.9206 (2.1);
0.9121 (3.6); 0.9055 (1.4); 0.8969 (0.4); 0.8871 (1.8); 0.8827 (3.5); 0.8743 (3.6); 0.8716 (3.8); 0.8630 (2.2); 0.8528 (1.0); 0.8488
(0.5)
Ia-102: ¹H-NMR(400.2 MHz, d₆-DMSO):
δ = 8.2923 (0.4); 8.2708 (0.9); 8.2266 (2.1); 8.2224 (6.2); 8.2181 (2.2); 8.2053 (2.7); 8.2009 (7.4); 7.9890 (6.8); 7.9846 (2.3);
7.9719 (2.1); 7.9675 (5.8); 7.9548 (0.8); 7.9141 (4.6); 7.8963 (5.2); 7.8929 (4.3); 7.6648 (0.6); 7.6619 (1.1); 7.6589 (0.7); 7.6434
(2.8); 7.6279 (1.2); 7.6249 (2.0); 7.6219 (1.1); 7.5335 (3.8); 7.5138 (5.4); 7.4991 (1.0); 7.4950 (2.5); 5.2245 (2.7); 5.1830 (4.3);
5.0632 (4.6); 5.0216 (2.9); 4.2320 (0.7); 4.1834 (4.0); 4.1672 (2.8); 4.1177 (0.4); 3.3383 (36.8); 2.8944 (4.8); 2.8517 (2.1);
2.8427 (1.0); 2.8329 (6.6); 2.8238 (1.1); 2.8140 (6.8); 2.8051 (0.5); 2.7951 (2.3); 2.7359 (4.0); 2.5285 (0.7); 2.5237 (1.1); 2.5150
(13.0); 2.5106 (26.2); 2.5061 (34.5); 2.5015 (25.3); 2.4970 (12.4); 1.3328 (1.0); 1.3252 (7.6); 1.3139 (2.3); 1.3063 (16.0); 1.2950
(1.3); 1.2874 (7.4); 1.2771 (0.4); 1.2375 (0.9)
Ia-103: ¹H-NMR(400.2 MHz, d₆-DMSO):
δ = 8.2264 (2.1); 8.2050 (2.6); 7.9841 (2.5); 7.9627 (2.3); 7.9544 (2.6); 4.4793 (1.0); 4.4381 (1.5); 4.2776 (1.6); 4.2363 (1.1);
4.2079 (0.6); 4.1592 (1.3); 4.0918 (0.8); 4.0422 (0.4); 3.3372 (18.1); 2.8948 (16.0); 2.8536 (0.7); 2.8348 (2.4); 2.8159 (2.4);
2.7970 (0.8); 2.7353 (14.0); 2.5148 (4.3); 2.5105 (8.7); 2.5060 (11.5); 2.5014 (8.6); 2.4970 (4.3); 2.4673 (0.7); 2.4491 (2.2);
2.4309 (2.2); 2.4127 (0.7); 1.3264 (2.7); 1.3076 (5.7); 1.2887 (2.7); 1.2380 (0.5); 0.9443 (2.6); 0.9261 (5.2); 0.9079 (2.5)
Ia-104: ¹H-NMR(400.2 MHz, d₆-DMSO):
δ = 13.3051 (1.0); 8.7151 (0.5); 8.2077 (2.2); 7.9534 (2.2); 7.8975 (0.7); 7.8906 (0.4); 7.8753 (8.0); 7.8709 (8.1); 7.8555 (0.4);
7.8487 (0.8); 7.6458 (1.4); 7.6416 (0.8); 7.6331 (1.3); 7.6280 (2.8); 7.6158 (2.1); 7.6117 (2.2); 7.5986 (2.3); 7.5941 (1.6); 7.5898
(1.8); 7.5745 (1.5); 7.5665 (1.8); 7.5597 (1.2); 7.5571 (1.3); 7.5512 (1.5); 7.5488 (1.6); 7.5421 (0.5); 7.5380 (0.6); 7.5349 (0.5);
7.5305 (0.7); 7.5276 (0.4); 7.4867 (1.3); 7.4688 (1.7); 7.4481 (1.2); 7.3201 (2.2); 7.3037 (1.8); 4.0306 (1.5); 3.9844 (1.9); 3.6471
(1.1); 3.6006 (0.9); 3.3362 (46.2); 2.8910 (16.0); 2.7327 (13.6); 2.7317 (13.4); 2.6296 (0.4); 2.5258 (0.8); 2.5210 (1.2); 2.5124
(15.2); 2.5079 (30.8); 2.5034 (40.4); 2.4988 (28.9); 2.4943 (13.6); 1.2395 (1.1); −0.0002 (3.9)
Ia-105: ¹H-NMR(300.2 MHz, d₆-DMSO):
δ = 8.7173 (0.8); 8.1978 (0.6); 8.1951 (0.6); 7.8923 (1.5); 7.8867 (1.5); 7.7138 (0.4); 7.5422 (0.3); 7.3743 (0.4); 4.1278 (2.2);
4.0608 (0.4); 3.9979 (0.3); 3.3459 (16.0); 2.5343 (1.5); 2.5284 (3.2); 2.5223 (4.6); 2.5163 (3.4); 2.5104 (1.6); 2.0097 (0.7);
1.1951 (0.4); 0.0205 (5.5)
Ia-106: ¹H-NMR(400.2 MHz, d₆-DMSO):
δ = 11.2308 (1.2); 8.6500 (3.7); 7.9528 (1.5); 7.8229 (0.4); 7.8140 (0.4); 7.7997 (16.0); 7.7791 (0.3); 7.7764 (0.4); 7.7155 (2.5);
7.7137 (2.4); 7.6532 (2.0); 7.6324 (2.4); 7.4263 (1.7); 7.4192 (2.3); 7.4126 (1.8); 7.3900 (1.7); 7.3860 (1.7); 7.3693 (1.5); 7.3652
(1.5); 6.4740 (1.2); 6.4712 (1.3); 6.4689 (1.8); 6.4667 (1.3); 6.4637 (1.2); 6.4619 (1.0); 3.9967 (1.6); 3.9506 (2.0); 3.6162 (1.2);
3.5697 (0.9); 3.3325 (16.2); 2.8895 (11.6); 2.7321 (9.1); 2.7310 (9.6); 2.6292 (0.4); 2.5248 (0.5); 2.5201 (0.8); 2.5114 (10.2);
2.5069 (20.7); 2.5023 (27.3); 2.4977 (19.6); 2.4931 (9.4); 1.2383 (0.8); −0.0002 (2.3)
Ia-107: ¹H-NMR(300.2 MHz, d₆-DMSO):
δ = 8.6615 (0.4); 7.9422 (0.5); 7.8252 (1.0); 7.8115 (0.9); 7.5640 (0.9); 7.4075 (0.4); 7.3973 (0.4); 6.5303 (0.4); 6.5202 (0.4);
3.8478 (2.2); 3.3439 (16.0); 2.5341 (1.8); 2.5283 (3.7); 2.5223 (5.0); 2.5164 (3.7); 0.0208 (6.3)
Ia-108: ¹H-NMR(300.2 MHz, d₆-DMSO):
δ = 8.6948 (0.3); 8.2909 (0.5); 8.2859 (0.5); 8.1592 (0.4); 8.1311 (0.5); 7.9219 (0.4); 7.8932 (1.2); 7.8671 (1.7); 7.8482 (0.7);
7.8391 (0.4); 7.5712 (0.5); 7.5530 (0.4); 3.9840 (0.4); 3.3459 (16.0); 2.5342 (1.8); 2.5283 (3.8); 2.5223 (5.2); 2.5164 (3.8);
2.5108 (1.8); 0.0204 (6.2)
Ia-109: ¹H-NMR(300.2 MHz, d₆-DMSO):
δ = 8.6955 (1.2); 8.1008 (0.9); 8.0496 (0.6); 7.9731 (0.6); 7.9447 (1.1); 7.8904 (0.5); 7.8793 (1.1); 7.8631 (0.6); 7.8510 (0.6);
7.5612 (0.4); 7.5569 (0.4); 7.5331 (0.3); 7.5285 (0.4); 4.1436 (3.8); 4.0620 (0.4); 4.0554 (0.4); 4.0384 (0.3); 3.9934 (0.5); 3.3445
(16.0); 2.5347 (1.6); 2.5288 (3.4); 2.5228 (4.7); 2.5168 (3.5); 2.5110 (1.6); 2.0102 (1.4); 1.2196 (0.4); 1.1958 (0.7); 1.1721 (0.4);
0.0212 (4.5)
Ia-110: ¹H-NMR(400.0 MHz, d₆-DMSO):
δ = 8.6721 (13.6); 8.3040 (1.1); 7.7522 (12.7); 7.7307 (16.0); 7.5655 (14.8); 7.5442 (11.9); 3.9579 (5.3); 3.9113 (6.6); 3.8959
(2.5); 3.5710 (4.8); 3.5241 (3.8); 3.3321 (367.0); 2.6776 (0.7); 2.6733 (1.0); 2.6690 (0.8); 2.5087 (136.3); 2.5043 (175.2); 2.4999
(129.9); 2.3353 (0.7); 2.3310 (1.0); 2.3265 (0.7); −0.0002 (3.6)
Ia-112: ¹H-NMR(400.1 MHz, d₆-DMSO):
δ = 7.6709 (6.9); 7.6669 (7.6); 7.6507 (9.6); 7.6464 (8.8); 7.4492 (8.7); 7.4325 (7.6); 3.9394 (3.5); 3.9358 (3.6); 3.8931 (4.5);
3.8893 (4.6); 3.7652 (16.0); 3.7625 (16.0); 3.6847 (0.4); 3.6126 (0.6); 3.5970 (0.6); 3.5640 (4.4); 3.5177 (3.7); 3.3430 (1.8);
2.5133 (14.9); 2.5092 (15.9); 1.7719 (0.3); 1.7664 (0.3)
Ia-113: ¹H-NMR(300.2 MHz, CDCl3):
δ = 7.7160 (0.9); 7.6884 (1.1); 7.4946 (0.9); 7.4677 (0.8); 7.2993 (9.4); 4.8118 (0.8); 4.7936 (0.8); 3.7960 (0.4); 3.7367 (0.8);
3.5949 (0.5); 3.5911 (0.5); 1.6023 (16.0); 0.0487 (0.5); 0.0379 (10.5); 0.0272 (0.7)
Ia-114: ¹H-NMR(499.9 MHz, d₆-DMSO):
δ = 11.0060 (0.4); 8.3595 (16.0); 7.5609 (0.3); 7.3688 (12.7); 7.3516 (13.3); 7.3467 (1.6); 6.5936 (2.0); 6.5888 (13.9); 6.5852
(4.4) ; 6.5749 (4.5); 6.5715 (13.2); 6.5665 (1.5); 5.6850 (13.7); 3.7764 (5.8); 3.7400 (7.1); 3.6883 (0.5); 3.6699 (1.0); 3.4296
(4.2); 3.3931 (3.4); 3.3206 (48.3); 2.5223 (0.9); 2.5186 (1.2); 2.5080 (16.0); 2.5045 (31.3); 2.5009 (41.5); 2.4973 (29.3); 2.4938
(13.4) ; 2.1092 (1.0); 1.9879 (0.6); 1.4773 (0.8); −0.0002 (2.0)
Ia-115: ¹H-NMR(499.9 MHz, d₆-DMSO):
δ = 7.8045 (0.4); 7.7877 (0.6); 7.7681 (0.6); 7.7389 (0.7); 7.7213 (0.4); 7.6703 (0.6): 7.4325 (0.4); 7.4148 (1.3); 7.4130 (1.4);
7.3976 (1.4); 7.3954 (1.3); 7.3882 (1.4); 7.3844 (0.6); 7.3709 (1.6); 7.3651 (1.3); 7.3597 (1.6); 7.3470 (15.8); 7.3434 (4.8);
7.3332 (5.2); 7.3298 (14.7); 7.3247 (0.8); 6.6327 (1.7); 6.6233 (3.9); 6.6206 (3.9); 6.6156 (16.0); 6.6120 (5.4); 6.6017 (5.2);
6.5984 (13.4); 6.5934 (0.8); 4.4611 (0.3); 4.4301 (0.4); 4.3332 (0.5); 4.3171 (3.5): 4.2950 (0.6); 4.2785 (4.9); 4.1490 (0.7);
4.1381 (0.6); 4.1206 (7.9); 4.1000 (0.4); 4.0822 (5.6); 3.9506 (0.4); 3.7842 (0.5); 3.7478 (0.6): 3.4359 (0.4); 3.2645 (0.4); 3.2504
(0.6); 3.2419 (0.4); 2.5075 (3.8); 2.5039 (8.3); 2.5003 (11.6); 2.4967 (8.3); 2.4932 (3.9); 2.1446 (0.4): 2.1328 (0.4); 2.1225 (0.4);
2.0856 (0.9); 1.9035 (0.6); 1.8921 (4.6); 1.8836 (9.2); 1.8749 (11.4); 1.8669 (5.4); 1.8552 (0.5); 1.8348 (0.5); 1.7011 (0.4);
1.6783 (0.8); 1.6688 (5.7); 1.6607 (11.5); 1.6523 (11.8); 1.6432 (4.8); 1.5468 (0.6); 1.5393 (0.5); 1.4885 (0.5); −0.0002 (4.7)
Ia-116: ¹H-NMR(300.2 MHz, CDCl3):
δ = 7.5312 (1.1); 7.5227 (9.6); 7.5163 (3.4); 7.5005 (3.2); 7.4939 (10.4); 7.4857 (1.4); 7.2988 (25.6); 6.7117 (1.3); 6.7032 (11.1);
6.6965 (3.6); 6.6809 (3.2); 6.6742 (10.5); 6.6657 (1.3); 5.3376 (0.4); 4.1895 (2.6); 4.1846 (2.7); 4.1299 (3.2); 4.1250 (3.4);
3.9560 (6.3); 3.6827 (6.0); 3.6232 (4.8); 3.5536 (0.6); 3.5315 (1.2); 3.5170 (1.6); 3.4957 (3.2); 3.4729 (2.0); 3.4603 (2.2); 3.4417
(2.6); 3.4364 (3.1); 3.4282 (3.4); 3.4185 (2.3); 3.4050 (2.3); 3.3884 (1.9); 3.3804 (1.2); 3.3660 (3.1); 3.3448 (1.7); 3.3297 (1.2);
3.3078 (0.6); 1.9886 (0.4); 1.9784 (0.7); 1.9682 (0.8); 1.9582 (2.1); 1.9357 (4.2); 1.9252 (5.1); 1.9160 (6.3); 1.9028 (5.4); 1.8932
(4.3); 1.8706 (1.9); 1.8491 (0.6); 1.8392 (0.3); 1.5865 (16.0); 1.2930 (0.4); 0.0492 (1.2); 0.0384 (33.6); 0.0275 (1.2)
Ia-117: ¹H-NMR(300.2 MHz, CDCl3):
δ = 7.8331 (1.9); 7.8268 (0.7); 7.8112 (0.7); 7.8045 (2.4); 7.7974 (0.4); 7.3549 (2.2); 7.3485 (0.7); 7.3328 (0.6); 7.3263 (2.0);
7.2989 (2.8); 5.9674 (4.1); 3.8330 (0.8); 3.7736 (1.4); 3.6347 (1.0); 3.6310 (1.1); 3.5754 (0.5); 3.5715 (0.5); 2.2947 (0.6); 2.0897
(16.0); 1.6630 (0.5); 1.2977 (0.5); 0.0398 (3.3)
Ia-118: ¹H-NMR(300.2 MHz, d₆-DMSO):
δ = 8.4955 (1.4); 7.5929 (0.8); 7.5632 (0.9); 7.0344 (0.8); 7.0045 (0.8); 3.8972 (0.4); 3.8361 (0.5); 3.7721 (0.8); 3.7565 (1.0);
3.7398 (0.9); 3.3464 (16.0); 3.2489 (0.9); 3.2325 (1.0); 3.2164 (0.8); 2.5345 (1.2); 2.5285 (2.7); 2.5224 (3.8); 2.5164 (2.7);
2.5104 (1.3); 0.0209 (6.4)
Ia-119: ¹H-NMR(300.2 MHz, d₆-DMSO):
δ = 9.8520 (1.5); 8.5465 (2.4); 7.9686 (1.6); 7.9392 (2.0); 7.6444 (2.0); 7.6151 (1.7); 6.7226 (2.1); 3.9139 (0.7); 3.8527 (1.0);
3.5610 (0.6); 3.5002 (0.5); 3.3478 (16.0); 2.5347 (4.5); 2.5288 (9.7); 2.5228 (13.6); 2.5167 (10.0); 2.5108 (4.7); 2.3653 (12.9);
2.0102 (1.2); 1.2195 (0.3); 1.1958 (0.6); 0.0322 (0.4); 0.0213 (15.1); 0.0103 (0.6)
Ia-120: ¹H-NMR(400.1 MHz, d₆-DMSO):
δ = 9.8839 (4.1); 8.5215 (3.6); 8.4015 (3.1); 8.3891 (3.1); 7.9610 (0.4); 7.9276 (4.4); 7.9064 (5.1); 7.6393 (5.1); 7.6180 (4.4);
6.8228 (3.1); 6.8103 (3.0); 3.8926 (2.1); 3.8465 (2.7); 3.5446 (2.1); 3.4987 (1.6); 3.3111 (12.7); 2.8988 (1.2); 2.7401 (1.2);
2.5483 (0.7); 2.5092 (8.5); 2.3974 (16.0)
Ia-121: ¹H-NMR(300.2 MHz, d₆-DMSO):
δ = 9.9978 (0.5); 8.5715 (1.1); 8.5616 (0.6); 8.5556 (1.2); 7.9285 (0.5); 7.8990 (0.7); 7.6705 (0.7); 7.6412 (0.6); 6.9419 (0.6);
3.3448 (16.0); 2.5342 (1.7); 2.5283 (3.5); 2.5223 (4.8); 2.5163 (3.5); 2.5104 (1.6); 0.0206 (6.6)
Ia-123: ¹H-NMR(300.2 MHz, d₆-DMSO):
δ = 8.6413 (10.7); 7.8900 (1.0); 7.8832 (0.4); 7.8673 (0.3); 7.8604 (1.1); 7.7905 (1.5); 7.7813 (12.2); 7.7750 (4.4); 7.7585 (4.3);
7.7520 (13.6); 7.7430 (1.7); 7.5549 (1.1); 7.5477 (0.4); 7.5311 (1.4); 7.5194 (13.2); 7.5122 (4.5); 7.4971 (4.5); 7.4897 (15.7);
7.4782 (1.5); 7.2234 (1.2); 7.2162 (0.5); 7.2009 (0.5); 7.1937 (1.1); 7.1686 (1.6); 7.1641 (2.0); 7.1526 (16.0); 7.1451 (6.6);
7.1350 (14.6); 7.1305 (8.9); 7.1229 (14.5); 7.1115 (5.2); 7.1057 (12.7): 7.0967 (1.4); 3.9773 (5.0); 3.9156 (6.7); 3.5873 (4.1);
3.5249 (3.0); 3.3564 (13.4); 2.5342 (2.4); 2.5285 (5.0); 2.5225 (6.8); 2.5166 (5.0); 0.0184 (5.7)
Ia-124: ¹H-NMR(300.2 MHz, CDCl3):
δ = 8.0817 (1.2); 8.0594 (0.5); 8.0534 (1.8); 7.9276 (1.9); 7.9216 (0.6); 7.8992 (1.2); 7.2987 (19.8); 3.8121 (0.5); 3.7531 (1.0);
3.6132 (1.2); 3.5565 (0.3); 3.5525 (0.4); 3.1228 (7.2); 1.5882 (16.0); 1.2931 (0.9); 0.0489 (0.8); 0.0380 (24.5); 0.0270 (0.8)
Ia-125: ¹H-NMR(499.9 MHz, d₆-DMSO):
δ = 8.7835 (1.5); 8.0628 (11.4); 8.0458 (16.0); 7.9976 (10.2); 7.9869 (2.9); 7.9831 (12.4); 7.9803 (9.1); 7.9473 (14.0); 7.9303
(11.0); 7.7349 (1.2); 7.7328 (2.2); 7.7305 (1.4); 7.7220 (1.5); 7.7179 (6.4); 7.7141 (2.1); 7.7055 (2.8); 7.7031 (4.8); 7.7009 (2.7);
7.6583 (8.6); 7.6424 (12.4); 7.6310 (2.1); 7.6278 (5.0); 3.9872 (4.2); 3.9499 (5.1); 3.5913 (4.4); 3.5538 (3.7); 3.3162 (27.5);
2.5093 (8.2); 2.5058 (18.0); 2.5022 (25.1); 2.4986 (18.4); 2.4951 (8.9)
Ia-126: ¹H-NMR(300.2 MHz, CDCl3):
δ = 7.6201 (0.4); 7.6130 (3.1); 7.6068 (1.1); 7.5907 (1.2); 7.5844 (3.7); 7.5774 (0.6); 7.3220 (0.6); 7.3151 (3.8); 7.3089 (1.2);
7.2986 (3.6); 7.2929 (1.2); 7.2866 (3.0); 7.2796 (0.4); 3.7678 (1.3); 3.7086 (2.3); 3.5656 (1.4); 3.5618 (1.5); 3.5235 (0.6); 3.5065
(1.0); 3.5025 (1.0); 2.5538 (16.0); 0.0383 (4.5)
Ia-127: ¹H-NMR(300.2 MHz, d₆-DMSO):
δ = 7.7072 (4.0); 7.6849 (1.5); 7.6788 (4.7); 7.4845 (2.1); 7.4812 (3.1); 7.4745 (3.1); 7.4687 (16.0); 7.4612 (2.6); 7.4568 (2.5);
7.4490 (1.4); 7.4438 (1.0); 7.4285 (0.6); 7.3347 (4.8); 7.3122 (1.4); 7.3062 (4.0); 3.9475 (1.5); 3.8855 (2.1); 3.5645 (1.4); 3.5023
(1.0); 3.3421 (15.3); 2.5349 (4.1); 2.5289 (8.7); 2.5229 (12.0); 2.5169 (8.7); 2.5111 (4.1); 0.0321 (0.6); 0.0214 (15.6); 0.0105
(0.6)
Ia-129: ¹H-NMR(300.2 MHz, d₆-DMSO):
δ = 8.6863 (0.5); 8.5592 (0.5); 7.2554 (0.4); 7.2314 (0.4); 7.1729 (0.7); 7.1650 (0.4); 7.1489 (0.4); 7.1453 (0.4); 3.3488 (16.0);
2.5344 (1.5); 2.5286 (3.2); 2.5225 (4.5); 2.5165 (3.3); 1.3076 (0.5); 1.2691 (0.5); 0.0208 (5.3)
Ia-130: ¹H-NMR(300.2 MHz, d₆-DMSO):
δ = 10.1403 (3.5); 8.7980 (16.0); 8.6418 (6.9); 7.7942 (3.5); 7.7687 (3.8); 7.3685 (2.4); 7.3433 (3.5); 7.3156 (2.5); 7.0643 (1.3);
7.0397 (2.2); 7.0152 (1.0); 3.9827 (1.8); 3.9209 (2.5); 3.6271 (1.6); 3.5643 (1.2); 3.3388 (11.0); 2.5344 (5.1); 2.5284 (11.2);
2.5223 (15.7); 2.5163 (11.5); 2.5103 (5.5); 1.9298 (0.8); 0.0322 (0.6); 0.0213 (19.0); 0.0103 (0.7)
Ia-131: ¹H-NMR(499.9 MHz, d₆-DMSO):
δ = 10.1654 (3.2); 8.7981 (16.0); 7.7788 (2.8); 7.7767 (3.5); 7.7614 (3.6); 7.7597 (3.2); 7.3482 (2.5); 7.3445 (0.9); 7.3333 (3.5);
7.3314 (3.3); 7.3199 (0.9); 7.3164 (2.6); 7.3125 (0.4); 7.0492 (0.7); 7.0473 (1.2); 7.0453 (0.8); 7.0326 (2.2); 7.0199 (0.6); 7.0178
(1.0); 7.0158 (0.6); 4.0103 (3.0); 4.0056 (3.9); 3.3719 (16.7); 3.3251 (9.6); 2.5171 (0.9); 2.5135 (2.0); 2.5098 (2.7); 2.5062 (1.9);
2.5026 (0.9)
Ia-132: ¹H-NMR(499.9 MHz, d₆-DMSO):
δ = 10.1826 (3.2); 8.7680 (11.6); 7.7692 (3.3); 7.7536 (3.5); 7.3453 (2.1); 7.3299 (3.4); 7.3137 (2.2); 7.0468 (1.1); 7.0321 (2.0);
7.0174 (0.9); 4.2941 (0.9); 4.2558 (1.8); 4.1774 (2.6); 4.1393 (1.4); 3.3217 (5.7); 2.5077 (2.3); 2.5043 (3.1); 2.5009 (2.4); 2.1627
(16.0)
Ia-133: ¹H-NMR(300.2 MHz, d₆-DMSO):
δ = 8.7796 (0.6); 8.3195 (4.0); 8.0339 (1.1); 8.0297 (0.8); 8.0221 (1.3); 8.0166 (1.0); 8.0105 (1.3); 8.0019 (1.4); 7.5861 (0.6);
7.5781 (2.3); 7.5666 (2.6); 7.5565 (2.6); 4.0102 (1.0); 3.9481 (1.4); 3.6625 (0.8); 3.6002 (0.6); 3.3482 (16.0); 2.5343 (1.8);
2.5284 (4.0); 2.5224 (5.6); 2.5164 (4.1); 2.5105 (2.0); 0.0200 (8.1)
Ia-134: ¹H-NMR(300.2 MHz, d₆-DMSO):
δ = 8.6843 (13.5); 7.4214 (0.6); 7.4035 (0.8); 7.3286 (11.6); 7.3167 (12.4); 7.2647 (0.7); 7.2469 (0.6); 6.9130 (7.4); 6.9095 (7.8);
6.9010 (7.4); 6.8975 (7.3); 3.9748 (0.4); 3.9373 (8.0); 3.9133 (0.6); 3.8760 (10.9); 3.5747 (6.6); 3.5711 (6.8); 3.5133 (4.7);
3.5096 (4.9); 3.4146 (0.7); 3.3913 (0.7); 3.3547 (16.0); 2.6162 (3.7); 2.5650 (0.5); 2.5342 (2.8); 2.5283 (5.6); 2.5223 (7.8);
2.5162 (6.5); 2.5013 (46.1); 2.4997 (46.4); 1.1346 (0.6); 1.1113 (1.2); 1.0880 (0.6); 0.0198 (7.6)
Ia-135: ¹H-NMR(300.2 MHz, d₆-DMSO):
δ = 8.8156 (14.6); 7.4345 (12.9); 7.4212 (15.4); 7.2874 (16.0); 7.2741 (13.3); 3.9795 (7.2); 3.9178 (9.9); 3.6122 (5.8); 3.6088
(5.9); 3.5505 (4.2); 3.5469 (4.3); 3.4136 (0.5); 3.3902 (0.6); 3.3551 (15.1); 2.5339 (2.2); 2.5281 (4.7); 2.5222 (6.5); 2.5162 (4.7);
1.1336 (0.5); 1.1104 (0.9); 1.0870 (0.4); 0.0184 (6.7)
Ia-136: ¹H-NMR(300.2 MHz, d₆-DMSO):
δ = 8.8010 (15.0); 7.5347 (8.7); 7.5220 (12.1); 7.4606 (13.0); 7.4479 (9.4); 7.4344 (1.9); 7.4302 (3.1); 7.4245 (1.5); 7.4100 (5.0);
7.4057 (7.1); 7.4009 (5.5); 7.3946 (1.9); 7.3905 (2.7); 7.3824 (10.8); 7.3802 (9.3); 7.3690 (1.3); 7.3555 (16.0); 7.3465 (7.1);
7.3381 (4.6); 7.3325 (6.3); 7.3278 (5.0); 7.3196 (5.4); 7.3096 (1.1); 7.3027 (1.0); 7.2969 (1.6); 7.2917 (0.9); 4.0007 (5.0); 3.9391
(6.8); 3.6202 (4.0); 3.5583 (3.0); 3.3593 (14.7); 2.5342 (1.6); 2.5283 (3.6); 2.5222 (4.9); 2.5162 (3.6); 2.5104 (1.7); 0.0178 (5.8)
Ia-137: ¹H-NMR(300.2 MHz, d₆-DMSO):
δ = 8.7294 (1.3); 7.9053 (0.4); 7.8960 (4.3); 7.8892 (1.5); 7.8735 (1.5); 7.8664 (5.2); 7.8574 (0.6); 7.5285 (2.9); 7.5016 (2.4);
4.0297 (1.8); 3.9676 (2.4); 3.6250 (1.4); 3.5663 (1.0); 3.5627 (1.1); 3.3474 (16.0); 2.5346 (2.5); 2.5287 (5.5); 2.5226 (7.7);
2.5166 (5.6); 2.5107 (2.7); 0.0313 (0.4); 0.0203 (12.0); 0.0093 (0.4)
Ia-138: ¹H-NMR(300.2 MHz, d₆-DMSO):
δ = 8.7801 (0.5); 7.9088 (1.0); 7.9013 (0.5); 7.8876 (0.7); 7.8798 (4.6); 7.8628 (3.9); 7.8341 (0.8); 4.0487 (1.1); 3.9866 (1.4);
3.6379 (0.8); 3.5756 (0.6); 3.3465 (16.0); 2.5343 (2.3); 2.5283 (5.0); 2.5223 (7.1); 2.5162 (5.2); 2.5103 (2.5); 0.0312 (0.4);
0.0204 (11.5); 0.0094 (0.4)
Ia-139: ¹H-NMR(400.2 MHz, d₆-DMSO):
δ = 13.2034 (0.5); 8.0195 (13.2); 7.9985 (16.0); 7.8400 (14.8); 7.8191 (12.0); 6.3158 (2.8); 6.1791 (6.1); 6.0423 (3.3); 3.7403
(4.9); 3.6948 (6.3); 3.3725 (4.8); 3.3230 (11.2); 2.6711 (0.3); 2.5065 (41.0); 2.5022 (53.2); 2.4979 (39.1); 2.3289 (0.4); 2.0750
(0.8); 0.0004 (36.7); −0.0076 (1.9)
Ia-140: ¹H-NMR(400.2 MHz, d₆-DMSO):
δ = 8.0444 (3.7); 8.0232 (4.9); 7.8711 (4.7); 7.8500 (3.7); 6.3185 (0.9); 6.1817 (1.9); 6.0449 (1.0); 3.8805 (16.0); 3.7440 (1.4);
3.6982 (1.8); 3.3814 (1.3); 3.3323 (2.3); 2.5109 (4.5); 2.5067 (9.2); 2.5022 (12.3); 2.4978 (8.8); 2.4935 (4.2); 0.0082 (0.4);
0.0002 (9.0); −0.0080 (0.3)
Ia-141: ¹H-NMR(300.2 MHz, CDCl3):
δ = 8.1368 (6.0); 8.1307 (2.1); 8.1140 (2.6); 8.1080 (6.8); 7.7937 (7.0); 7.7875 (2.2); 7.7710 (2.1); 7.7648 (5.6); 7.2981 (2.3);
6.1631 (1.9); 5.9796 (4.0); 5.7962 (2.1); 3.7358 (1.9); 3.6762 (2.9); 3.4574 (2.0); 3.3972 (1.3); 3.3930 (1.0); 2.9283 (2.1); 2.9029
(6.8); 2.8932 (0.8); 2.8777 (7.1); 2.8680 (0.8); 2.8525 (2.4); 2.6970 (0.3); 2.3455 (2.1); 1.4581 (7.5); 1.4511 (1.1); 1.4328 (16.0);
1.4257 (1.9); 1.4075 (7.2); 1.4005 (0.9); 1.2825 (0.7); 0.0304 (1.5)
Ia-142: ¹H-NMR(300.2 MHz, d₆-DMSO):
δ = 7.6867 (2.6); 7.3853 (2.2); 7.3566 (2.4); 6.6077 (2.5); 6.5789 (2.4); 6.2918 (0.6); 6.1086 (1.2); 5.9255 (0.6); 5.6585 (2.5);
3.5806 (0.7); 3.5210 (1.0); 3.3475 (16.0); 3.2282 (0.7); 3.1676 (0.5); 2.5345 (1.5); 2.5285 (3.2); 2.5225 (4.5); 2.5165 (3.4);
2.5107 (1.6); 0.0211 (4.8)
Ia-144: ¹H-NMR(300.2 MHz, d₆-DMSO):
δ = 10.6929 (3.3); 8.3222 (5.6); 8.3161 (2.1); 8.3086 (0.6); 8.2995 (2.3); 8.2932 (6.9); 8.0705 (4.8); 8.0425 (4.0); 3.3854 (1.5);
2.8790 (2.0); 2.8539 (6.5); 2.8287 (6.7); 2.8036 (2.2); 2.5345 (0.4); 2.5286 (0.7); 2.5225 (1.0); 2.5164 (0.7); 2.5105 (0.3); 1.3433
(7.4); 1.3182 (16.0); 1.2930 (6.9); 0.0024 (0.7)
Ia-145: ¹H-NMR(300.2 MHz, d₆-DMSO):
δ = 9.7243 (2.4); 9.5057 (0.8); 8.2960 (3.6); 8.2887 (2.5); 8.2672 (5.0); 8.2601 (2.7); 8.1181 (1.6); 8.0978 (3.5); 8.0727 (2.4);
7.0328 (2.4); 6.8689 (2.8); 6.6973 (1.1); 3.3471 (16.0); 2.8853 (1.8); 2.8602 (5.9); 2.8350 (6.1); 2.8099 (2.0); 2.5344 (1.1);
2.5285 (2.4); 2.5224 (3.3); 2.5163 (2.4); 2.5104 (1.1); 1.3501 (6.7); 1.3250 (14.6); 1.2998 (6.4); 0.0170 (1.8)
Ia-146: ¹H-NMR(300.2 MHz, CDCl3):
δ = 7.8117 (16.0); 7.3591 (0.9); 7.3552 (1.0); 7.3322 (1.4); 7.3283 (1.4); 7.2986 (1.6); 7.2460 (1.0); 7.2195 (2.0); 7.1927 (1.1);
7.1333 (1.4); 7.1293 (1.4); 7.1069 (0.9); 7.1029 (0.8); 3.8288 (1.0); 3.7694 (2.2); 3.6343 (1.1); 3.6307 (1.1); 3.5748 (0.6); 3.5711
(0.6); 2.0891 (0.7); 1.3054 (0.4); 1.2938 (0.6); 0.9213 (0.4); 0.0396 (1.8)
Ia-147: ¹H-NMR(499.9 MHz, CDCl3):
δ = 7.9194 (1.0); 7.8236 (9.7); 7.8059 (16.0); 7.7524 (11.6); 7.7354 (7.2); 7.2647 (2.4); 3.6939 (5.5); 3.6581 (8.1); 3.5138 (5.5);
3.4780 (3.7); 3.2474 (0.7); 2.9593 (5.1); 2.8369 (4.6); 2.6811 (1.1); −0.0002 (2.1)
Ia-148: ¹H-NMR(400.1 MHz, d₆-DMSO):
δ = 8.6398 (16.0); 7.7906 (12.4); 7.7707 (14.4); 7.5460 (14.6); 7.5261 (12.3); 7.0906 (3.1); 7.0712 (6.2); 7.0524 (3.8); 7.0270
(6.2); 7.0083 (6.8); 6.7888 (7.8); 6.7687 (6.8); 6.6732 (4.2); 6.6547 (7.2); 6.6363 (3.4); 4.8515 (9.8); 3.9737 (6.0); 3.9273 (7.6);
3.5998 (5.7); 3.5534 (4.5); 3.3041 (20.9); 2.4996 (17.9); 1.0458 (1.1); 1.0308 (1.1); −0.0002 (6.0)
Ia-149: ¹H-NMR(300.2 MHz, d₆-DMSO):
δ = 8.5995 (0.9); 7.6917 (0.7); 7.6642 (1.0); 7.4825 (0.9); 7.4550 (0.7); 3.9693 (0.3); 3.9077 (0.5); 3.3374 (16.0); 2.5348 (1.5);
2.5288 (3.2); 2.5227 (4.5); 2.5166 (3.3); 2.5107 (1.6); 1.2579 (1.3); 1.2357 (1.3); 0.2760 (0.5); 0.2530 (0.5); 0.2220 (0.4); 0.0218
(5.2)
Ia-150: ¹H-NMR(300.2 MHz, d₆-DMSO):
δ = 8.7050 (1.8); 7.8248 (2.4); 7.8212 (2.7); 7.8012 (8.1); 7.7962 (5.5); 7.7743 (7.3); 7.7026 (1.1); 7.6980 (1.2); 7.6775 (2.8);
7.6729 (2.8); 7.6524 (2.1); 7.6477 (1.9); 7.5790 (1.9); 7.5750 (2.2); 7.5538 (2.8); 7.5499 (3.0); 7.5286 (1.2); 7.5247 (1.2); 7.4888
(3.2); 7.4861 (3.2); 7.4634 (2.7); 7.4344 (7.5); 7.4067 (6.5); 4.1230 (2.3); 4.0993 (7.3); 4.0757 (7.4); 4.0610 (0.5); 4.0520 (2.4);
4.0298 (2.6); 3.9679 (3.5); 3.6370 (2.3); 3.5747 (1.7); 3.3506 (8.0); 2.5283 (4.3); 2.5223 (5.9); 2.5164 (4.3); 2.0089 (1.0); 1.3005
(0.4); 1.2653 (2.8); 1.2182 (0.4); 1.1945 (0.6); 1.0487 (7.6); 1.0251 (16.0); 1.0014 (7.4); 0.8992 (0.8); 0.8774 (2.4); 0.8543 (0.9);
0.0195 (6.2)
Ia-151: ¹H-NMR(300.2 MHz, d₆-DMSO):
δ = 11.3831 (0.6); 8.6921 (1.2); 8.2528 (0.5); 8.2254 (0.5); 8.1847 (0.4); 8.1587 (0.4); 7.9331 (0.5); 7.9045 (1.2); 7.8714 (1.2);
7.8426 (0.5); 7.8122 (0.6); 7.8091 (0.6); 7.5787 (0.4); 7.5735 (0.4); 7.5516 (0.6); 7.5262 (0.5); 7.4279 (0.4); 7.2024 (0.4); 4.0530
(0.3); 3.9913 (0.4); 3.3482 (16.0); 2.5345 (3.0); 2.5285 (6.5); 2.5225 (9.0); 2.5165 (6.6); 2.5107 (3.1); 0.0208 (10.8); 0.0098 (0.4)
Ia-152: ¹H-NMR(300.2 MHz, d₆-DMSO):
δ = 8.7022 (1.0); 7.8162 (0.9); 7.7884 (1.1); 7.7698 (0.4); 7.7659 (0.4); 7.7444 (0.4); 7.7403 (0.5); 7.6398 (0.4); 7.6351 (0.4);
7.6147 (0.3); 7.5492 (0.4); 7.5282 (0.4); 7.5241 (0.5); 7.4386 (1.6); 7.4111 (1.3); 4.0343 (0.4); 3.9722 (0.5); 3.6373 (0.3); 3.3482
(16.0); 2.5344 (1.3); 2.5285 (2.8); 2.5225 (4.0); 2.5165 (2.9); 2.5108 (1.4); 1.2802 (12.1); 0.0205 (3.1)
Ia-153: ¹H-NMR(300.2 MHz, d₆-DMSO):
δ = 8.8045 (1.2); 7.7336 (2.7); 7.7206 (3.2); 7.6835 (0.7); 7.6767 (1.0); 7.6702 (0.8); 7.6489 (0.7); 7.6428 (1.0); 7.6356 (0.8);
7.6010 (0.9); 7.5747 (2.0); 7.5665 (3.5); 7.5533 (3.3); 7.5319 (0.9); 7.5251 (1.2); 7.5050 (1.2); 7.4994 (0.6); 7.4789 (0.5); 7.2743
(0.5); 7.2707 (0.6); 7.2662 (0.5); 7.2625 (0.5); 7.2444 (0.9); 7.2419 (0.9); 7.2362 (0.8); 7.2178 (0.4); 7.2147 (0.5); 7.2095 (0.4);
4.0180 (1.5); 3.9566 (2.0); 3.6510 (1.2); 3.5892 (0.9); 3.3499 (16.0); 2.5343 (1.8); 2.5283 (3.9); 2.5223 (5.4); 2.5163 (4.0);
2.5104 (1.9); 0.0192 (5.3)
Ia-154: ¹H-NMR(300.2 MHz, d₆-DMSO):
δ = 10.6549 (1.3); 8.8459 (0.8); 8.4132 (1.4); 8.3839 (1.3); 8.3654 (1.4); 7.8230 (1.6); 7.8100 (1.8); 7.6152 (1.8); 7.6022 (1.6);
7.5044 (1.0); 7.4989 (1.0); 7.4869 (1.0); 7.4813 (1.0); 4.0608 (0.6); 4.0442 (0.9); 4.0372 (0.7); 3.9828 (1.2); 3.6639 (0.8); 3.6027
(0.6); 3.3484 (16.0); 2.5342 (1.7); 2.5285 (3.5); 2.5225 (4.8); 2.5165 (3.5); 2.1434 (8.0); 2.0092 (2.4); 1.2183 (0.6); 1.1946 (1.3);
1.1708 (0.6); 0.0194 (4.5)
Ia-155: ¹H-NMR(300.2 MHz, d₆-DMSO):
δ = 8.8178 (2.8); 7.6007 (5.3); 7.5879 (6.3); 7.5534 (6.4); 7.5470 (6.6); 7.4513 (6.4); 7.4384 (5.5); 6.6064 (6.6); 6.6001 (6.6);
4.3530 (1.9); 4.3290 (5.9); 4.3050 (5.9); 4.2811 (1.9); 4.0387 (3.1); 3.9775 (4.0); 3.6671 (2.5); 3.6052 (1.8); 3.3509 (10.8);
2.5344 (2.1); 2.5285 (4.5); 2.5225 (6.2); 2.5165 (4.5); 2.0091 (1.2); 1.3884 (7.0); 1.3645 (16.0); 1.3405 (6.7); 1.2184 (0.3);
1.1947 (0.6); 1.1709 (0.3); 0.0193 (6.6)
Ia-156: ¹H-NMR(499.9 MHz, d₆-DMSO):
δ = 8.6658 (1.4); 7.8077 (1.5); 7.8056 (1.6); 7.7923 (1.8); 7.7901 (1.8); 7.7790 (3.5); 7.7656 (1.5); 7.7623 (3.8); 7.6777 (0.7);
7.6751 (0.7); 7.6626 (1.6); 7.6600 (1.5); 7.6475 (1.0); 7.6448 (0.9); 7.5521 (1.1); 7.5498 (1.1); 7.5368 (1.6); 7.5346 (1.6); 7.5217
(0.8); 7.5194 (0.8); 7.4758 (1.7); 7.4743 (1.7); 7.4604 (1.5); 7.4111 (3.9); 7.3944 (3.5); 4.0003 (1.6); 3.9633 (1.9); 3.6163 (16.0);
3.5746 (1.0); 3.3192 (3.2); 2.5098 (1.2); 2.5063 (2.2); 2.5027 (2.8); 2.4992 (2.0); 2.4957 (0.9)
Ia-157: ¹H-NMR(300.2 MHz, d₆-DMSO):
δ = 10.3163 (0.8); 8.3549 (0.8); 8.0281 (0.8); 8.0227 (0.8); 8.0013 (0.9); 7.9971 (0.9); 7.9598 (0.7); 7.9311 (1.8); 7.8942 (1.8);
7.8656 (0.7); 7.7105 (0.5); 7.6844 (0.9); 7.6592 (0.5); 7.6522 (0.5); 7.6315 (0.4); 4.0523 (0.5); 3.9904 (0.6); 3.6664 (0.4); 3.3487
(16.0); 2.5342 (2.0); 2.5282 (4.4); 2.5221 (6.0); 2.5160 (4.3); 2.5101 (2.0); 0.0199 (9.7); 0.0090 (0.3)
Ia-158: ¹H-NMR(300.2 MHz, d₆-DMSO):
δ = 8.1973 (0.5); 7.8997 (0.5); 7.8960 (0.5); 7.8848 (1.4); 7.8732 (1.5); 7.6032 (0.5); 3.9848 (0.3); 3.3480 (16.0); 2.8512 (1.3);
2.8361 (1.3); 2.5342 (1.7); 2.5281 (3.7); 2.5221 (5.1); 2.5160 (3.6); 2.5100 (1.7); 2.0094 (0.9); 1.1946 (0.5); 0.0201 (9.1); 0.0092
(0.3)
Ia-159: ¹H-NMR(300.2 MHz, d₆-DMSO):
δ = 7.8548 (2.4); 7.8481 (2.4); 7.8239 (0.5); 7.8205 (0.5); 7.7626 (0.4); 7.7579 (0.7); 7.7528 (0.4); 7.5788 (0.6); 7.5533 (0.4);
7.4604 (0.5); 7.4350 (0.3); 4.0375 (0.5); 3.9781 (0.5); 3.3482 (16.0); 3.0342 (1.0); 2.9719 (1.0); 2.5341 (1.8); 2.5281 (4.0);
2.5220 (5.5); 2.5159 (3.9); 2.5099 (1.8); 2.0093 (1.1); 1.1944 (0.6); 0.0308 (0.3); 0.0200 (9.0)
Ia-160: ¹H-NMR(300.2 MHz, d₆-DMSO):
δ = 9.2333 (0.3); 9.2141 (0.7); 9.1949 (0.3); 8.2773 (1.3); 7.9628 (1.1); 7.9356 (1.5); 7.9303 (1.1); 7.9257 (1.2); 7.8965 (3.2);
7.8743 (3.1); 7.8454 (0.9); 7.6534 (0.8); 7.6274 (1.3); 7.6018 (0.6); 7.4561 (0.3); 7.4365 (0.6); 7.4302 (0.7); 7.4050 (0.4); 7.3599
(0.4); 7.3404 (0.4); 7.3325 (0.5); 7.2486 (0.6); 7.2255 (1.0); 7.2172 (0.7); 7.2130 (0.6); 7.2042 (1.0); 7.2006 (0.8); 7.1900 (0.4);
7.1858 (0.4); 7.1799 (0.5); 7.1757 (0.4); 4.5949 (1.3); 4.5762 (1.4); 4.0401 (0.7); 3.9784 (1.0); 3.6570 (0.6); 3.5953 (0.5); 3.3483
(16.0); 2.5342 (3.0); 2.5282 (6.5); 2.5221 (9.0); 2.5160 (6.5); 2.5100 (3.0); 0.0309 (0.3); 0.0200 (11.3); 0.0091 (0.4)
Ia-161: ¹H-NMR(300.2 MHz, d₆-DMSO):
δ = 8.6867 (0.3); 7.7692 (0.8); 7.7413 (1.0); 7.5809 (1.0); 7.5530 (0.7); 4.8365 (2.0); 3.9951 (0.4); 3.9333 (0.5); 3.3448 (16.0);
2.5342 (1.7); 2.5282 (3.5); 2.5222 (4.8); 2.5161 (3.4); 2.5101 (1.6); 0.0206 (7.8)
Ia-162: ¹H-NMR(300.2 MHz, CDCl3):
δ = 7.7321 (6.8); 7.7247 (4.5); 7.7149 (5.0); 7.7082 (8.1); 7.7042 (8.4); 7.6970 (4.5); 7.5412 (8.2); 7.5336 (5.2); 7.5166 (6.2);
7.5146 (6.2); 7.3089 (1.8); 7.3023 (4.7); 7.2977 (6.1); 7.2899 (2.8); 7.0599 (1.0); 7.0523 (2.3); 7.0490 (2.8); 7.0405 (2.3); 7.0222
(6.9); 7.0181 (6.7); 7.0104 (4.1); 7.0032 (3.4); 6.9997 (3.2); 6.9952 (4.8); 6.9912 (5.6); 6.9833 (3.0); 6.9659 (2.4); 6.9585 (5.0);
6.9546 (6.1); 6.9446 (6.8); 6.9401 (6.6); 6.9318 (5.2); 6.9235 (4.2); 6.9134 (3.1); 6.9101 (2.9); 6.9016 (1.4); 5.1130 (16.0);
5.1042 (7.7); 3.7926 (3.2); 3.7838 (1.8); 3.7333 (6.1); 3.7248 (3.4); 3.5933 (4.8); 3.5893 (4.2); 3.5337 (2.8); 3.5297 (2.5); 2.9887
(0.8); 2.9855 (0.9); 2.9775 (0.5); 2.8907 (0.9); 2.8826 (0.4); 1.6818 (0.5); 1.4707 (0.4); 1.4634 (0.4); 1.4108 (0.3); 1.3646 (0.3);
1.3249 (0.7); 1.2961 (1.2); 1.2764 (0.7); 1.2718 (0.8); 1.2599 (0.6); 1.2530 (0.8); 1.2484 (0.9); 1.2406 (0.6); 1.2297 (0.5); 1.2251
(0.5); 1.2174 (0.3); 0.8712 (1.0); 0.0497 (1.9); 0.0433 (4.8); 0.0386 (6.4); 0.0309 (3.0)
Ib-001: ¹H-NMR(300.2 MHz, d₆-DMSO):
δ = 8.8513 (2.4); 8.3524 (1.9); 8.3459 (0.7); 8.3292 (0.7); 8.3225 (2.4); 8.3146 (0.3); 8.0441 (0.3); 8.0364 (2.3); 8.0297 (0.8);
8.0131 (0.6); 8.0064 (1.9); 4.0544 (0.7); 3.9916 (1.0); 3.6729 (0.7); 3.6100 (0.5); 3.3405 (16.0); 2.5347 (1.8); 2.5288 (3.9);
2.5227 (5.4); 2.5167 (3.9); 2.5108 (1.8); 0.0211 (6.1)
Ib-002: ¹H-NMR(300.2 MHz, d₆-DMSO):
δ = 9.0158 (1.6); 8.3623 (2.3); 8.3559 (0.8); 8.3391 (0.8); 8.3324 (2.8); 8.3247 (0.4); 8.0598 (0.4); 8.0520 (2.8); 8.0454 (0.9);
8.0288 (0.8); 8.0221 (2.3); 4.1392 (0.6); 4.0767 (0.8); 3.6962 (0.8); 3.6334 (0.6); 3.3449 (16.0); 2.5346 (1.6); 2.5287 (3.3);
2.5227 (4.6); 2.5166 (3.4); 2.5108 (1.6); 2.0097 (0.4); 0.0205 (5.6)
Ib-003: ¹H-NMR(400.2 MHz, d₆-DMSO):
δ = 8.6808 (15.3); 8.1355 (0.6); 7.7874 (13.4); 7.7666 (16.0); 7.5062 (16.0); 7.4853 (13.8); 3.9484 (5.6); 3.9018 (7.2); 3.5921
(4.8); 3.5452 (3.8); 3.3227 (42.8); 3.2989 (1.1); 2.9941 (14.2); 2.9005 (14.8); 2.6708 (0.3); 2.5104 (21.6); 2.5062 (43.0); 2.5018
(57.2); 2.4974 (41.5); 2.3288 (0.3); 0.0001 (5.0)
Ib-004: ¹H-NMR(400.2 MHz, d₆-DMSO):
δ = 8.7695 (3.4); 8.6867 (6.1); 7.9067 (4.0); 7.8858 (5.8); 7.7891 (5.9); 7.7682 (4.2); 3.9485 (2.0); 3.9018 (2.6); 3.5917 (1.8);
3.5448 (1.4); 3.3220 (6.8); 2.5065 (8.6); 2.5022 (11.4); 2.4978 (8.3); 2.0750 (0.5); 1.3716 (16.0); 0.7614 (1.1); 0.7445 (4.1);
0.7337 (1.7); 0.6310 (1.9); 0.6200 (4.2); 0.6154 (4.2); 0.6024 (1.3); 0.0080 (0.3); 0.0000 (8.1); −0.0081 (0.4)
Ib-005: ¹H-NMR(400.2 MHz, d₆-DMSO):
δ = 10.2681 (10.6); 10.1597 (0.4); 8.7354 (14.1); 8.1375 (1.8); 8.0745 (11.4); 8.0535 (14.3); 7.9721 (0.4); 7.9514 (0.6); 7.8977
(16.0); 7.8767 (12.9); 7.7464 (0.5); 7.7253 (0.5); 7.6308 (2.1); 7.6152 (2.6); 7.6086 (4.4); 7.5931 (4.4); 7.5866 (2.7); 7.5710
(2.2); 7.4066 (2.5); 7.3996 (2.6); 7.3835 (3.2); 7.3799 (3.8); 7.3769 (3.7); 7.3732 (3.3); 7.3573 (2.7); 7.3503 (2.6); 7.1647 (1.9);
7.1615 (2.1); 7.1575 (2.0); 7.1404 (3.8); 7.1358 (3.6); 7.1218 (1.9); 7.1186 (2.0); 7.1147 (1.8); 3.9891 (5.8); 3.9424 (7.5); 3.6328
(5.0); 3.5858 (4.0); 3.3235 (66.2); 3.3004 (1.6); 2.6754 (0.4); 2.6710 (0.5); 2.6665 (0.4); 2.5412 (1.4); 2.5105 (34.1); 2.5063
(67.8); 2.5019 (89.9); 2.4975 (64.7); 2.4934 (31.1); 2.3331 (0.4); 2.3288 (0.5); 2.3245 (0.4); 2.0745 (0.6); 1.1474 (0.3); 0.0081
(0.4); 0.0000 (8.5)
Ib-006: ¹H-NMR(400.2 MHz, d₆-DMSO):
δ = 8.4833 (0.7); 8.4705 (2.2); 8.4562 (0.6); 7.9159 (0.4); 7.7282 (2.4); 7.7071 (3.3); 7.5959 (3.3); 7.5748 (2.4); 3.7326 (1.1);
3.6860 (1.4); 3.3781 (1.0); 3.3311 (0.7); 3.0950 (6.4); 2.9419 (1.4); 2.9264 (2.2); 2.9109 (1.5); 2.2829 (5.9); 2.2786 (11.9);
2.2742 (16.0); 2.2698 (11.5); 2.2655 (5.4); 0.8310 (0.3); 0.8286 (0.4); 0.8234 (0.4); 0.8115 (0.6); 0.8033 (0.3); 0.7994 (0.4);
0.7946 (0.4); 0.7918 (0.4); 0.2349 (0.5); 0.2241 (1.6); 0.2198 (1.7); 0.2150 (0.8); 0.2097 (0.9); 0.2040 (1.6); 0.1997 (1.6); 0.1897
(0.7); 0.0260 (0.6); 0.0153 (1.8); 0.0123 (1.9); 0.0039 (1.7); 0.0002 (1.9); −0.0108 (0.5); −0.2277 (1.9)
Ib-007: ¹H-NMR(400.2 MHz, d₆-DMSO):
δ = 9.2049 (2.1); 9.1902 (4.5); 9.1752 (2.2); 8.7105 (16.0); 7.9882 (10.0); 7.9672 (13.8); 7.8389 (13.4); 7.8179 (10.5); 7.3884
(5.0); 7.3741 (6.2); 7.3669 (7.1); 7.3578 (2.9); 7.3528 (6.3); 7.1887 (0.9); 7.1814 (7.3); 7.1762 (2.5); 7.1646 (2.9); 7.1591 (12.5);
7.1536 (3.0); 7.1419 (2.2); 7.1369 (6.0); 7.1294 (0.7); 4.4781 (8.8); 4.4634 (8.8); 3.9626 (4.8); 3.9160 (6.2); 3.8832 (0.7); 3.6084
(4.2); 3.5615 (3.3); 3.3325 (10.0); 2.5113 (7.3); 2.5072 (14.8); 2.5028 (19.8); 2.4984 (14.3); 1.2346 (0.6); 0.0082 (0.5); 0.0001
(12.5); −0.0081 (0.5)
Ib-008: ¹H-NMR(400.2 MHz, d₆-DMSO):
δ = 8.7006 (12.9); 8.6747 (2.6); 8.6613 (4.7); 8.6498 (2.4); 8.1383 (1.1); 7.9505 (11.5); 7.9294 (15.6); 7.8407 (0.5); 7.8214 (16.0);
7.8004 (11.3); 7.6639 (0.3); 3.9579 (5.5); 3.9113 (7.1); 3.6026 (4.9); 3.5557 (3.9); 3.4884 (1.9); 3.4819 (3.3); 3.4688 (12.7);
3.4587 (14.3); 3.4417 (10.2); 3.4283 (6.6); 3.3283 (150.5); 3.3053 (2.8); 3.2713 (69.0); 3.0933 (0.4); 2.6712 (0.4); 2.5066 (53.4);
2.5023 (68.6); 2.4979 (49.7); 2.3289 (0.4); 0.0000 (4.4)
Ib-009: ¹H-NMR(400.2 MHz, d₆-DMSO):
δ = 13.2180 (4.9); 8.7304 (12.5); 8.0252 (12.2); 8.0040 (16.0); 7.8569 (15.2); 7.8358 (12.0); 3.9673 (5.4); 3.9207 (6.9); 3.6032
(4.6) ; 3.5562 (3.7); 3.3197 (93.2); 2.6747 (0.7); 2.6705 (0.9); 2.6659 (0.7); 2.5102 (54.8); 2.5059 (112.5); 2.5014 (151.6); 2.4969
(108.3); 2.4926 (50.7); 2.3328 (0.7); 2.3284 (0.9); 2.3237 (0.7); 2.0745 (1.3); 0.1464 (0.4); 0.0082 (3.9); 0.0001 (101.7); −0.0081
(3.7) ; −0.1492 (0.4)
Ib-010: ¹H-NMR(400.2 MHz, d₆-DMSO):
δ = 8.7461 (5.6); 8.0503 (3.7); 8.0461 (1.7); 8.0334 (1.6); 8.0291 (5.0); 7.8866 (4.8); 7.8654 (3.8); 4.3506 (0.4); 4.3328 (0.4);
3.9766 (1.7); 3.9299 (2.2); 3.8828 (16.0); 3.6088 (1.5); 3.5618 (1.2); 3.3250 (15.9); 2.5111 (4.6); 2.5068 (9.6); 2.5023 (12.8);
2.4978 (9.1); 2.4934 (4.2); 1.3568 (0.4); 1.3391 (0.9); 1.3213 (0.4); 1.2346 (0.4); 0.0082 (0.4); 0.0002 (8.7)
Ib-011: ¹H-NMR(300.2 MHz, CDCl3):
δ = 7.6567 (0.5); 7.6281 (7.0); 7.5935 (0.4); 7.4570 (0.8); 7.2989 (3.4); 5.3358 (0.4); 3.9705 (0.4); 3.9655 (0.7); 3.9598 (0.4);
3.9095 (0.9); 3.9044 (1.4); 3.8988 (0.9); 3.7987 (2.2); 3.7378 (1.0); 2.2331 (12.6); 2.2267 (16.0); 2.0830 (0.6); 1.6434 (2.9);
1.2959 (0.5); 0.0361 (3.6)
Ib-012: ¹H-NMR(300.2 MHz, d₆-DMSO):
δ = 8.3902 (2.0); 7.3927 (1.4); 7.3640 (1.6); 6.6098 (1.6); 6.5809 (1.5); 5.7086 (1.5); 3.7836 (0.5); 3.7226 (0.7); 3.4608 (0.5);
3.3994 (0.4); 3.3457 (16.0); 2.5345 (1.4); 2.5286 (3.0); 2.5225 (4.2); 2.5165 (3.0); 2.5105 (1.4); 2.0099 (0.4); 0.0211 (4.8)
Ib-013: ¹H-NMR(499.9 MHz, d₆-DMSO):
δ = 8.7463 (1.0); 8.0512 (1.0); 8.0345 (1.2); 7.9815 (0.6); 7.8751 (1.2); 7.8585 (1.1); 7.8199 (6.5); 7.8047 (7.6); 7.6963 (2.2);
7.6796 (5.7); 7.6688 (9.4); 7.6530 (8.7); 7.6288 (5.3); 7.6135 (2.1); 7.6092 (1.8); 7.4076 (15.1); 7.3906 (16.0); 6.6219 (15.9);
6.6048 (15.6); 5.7702 (15.1); 5.7560 (3.6); 4.3525 (4.7); 4.3141 (5.8); 4.0314 (8.0); 3.9931 (6.6); 3.9827 (1.1); 3.6252 (0.4);
3.5880 (0.4); 3.3177 (19.6); 2.6203 (4.0); 2.5028 (17.2); 1.2364 (1.0)
Ib-014: ¹H-NMR(300.2 MHz, CDCl3):
δ = 7.5178 (0.4); 7.5092 (3.2); 7.5029 (1.1); 7.4870 (1.1); 7.4805 (3.4); 7.4721 (0.4); 7.2991 (12.7); 6.7298 (0.5); 6.7214 (3.6);
6.7149 (1.2); 6.6990 (1.1); 6.6926 (3.4); 6.6843 (0.4); 3.9884 (1.8); 3.9515 (0.6); 3.9462 (0.8); 3.9404 (0.6); 3.8910 (1.0); 3.8855
(1.5); 3.8799 (1.0); 3.7753 (2.3); 3.7148 (1.1); 2.2157 (16.0); 1.5833 (14.4); 0.0497 (0.5); 0.0388 (15.8); 0.0282 (0.6)
Ib-015: ¹H-NMR(300.2 MHz, d₆-DMSO):
δ = 9.3885 (0.8); 8.5854 (1.7); 7.7953 (0.9); 7.7659 (1.6); 7.6783 (1.6); 7.6489 (1.0); 3.9014 (0.4); 3.8398 (0.6); 3.5575 (0.4);
3.3454 (16.0); 2.5345 (1.5); 2.5285 (3.3); 2.5225 (4.7); 2.5164 (3.4); 2.5105 (1.6); 2.0098 (0.6); 1.4344 (4.3); 1.2523 (0.7);
1.1955 (0.4); 1.1459 (0.4); 1.1336 (1.1); 1.1245 (1.1); 1.1128 (0.4); 0.6942 (0.5); 0.6819 (1.3); 0.6728 (1.3); 0.6597 (0.4); 0.0209
(4.4); −0.0435 (0.8)
Ib-016: ¹H-NMR(300.2 MHz, d₆-DMSO):
δ = 10.5523 (1.2); 8.6239 (2.2); 7.9358 (1.3); 7.9066 (1.9); 7.8605 (0.6); 7.8316 (1.0); 7.8236 (0.5); 7.7988 (0.4); 7.7907 (0.4);
7.7654 (1.8); 7.7363 (1.3); 7.6413 (0.5); 7.6219 (0.5); 7.6158 (0.4); 7.4974 (0.4); 7.4911 (0.4); 3.9319 (0.6); 3.8702 (0.8); 3.5907
(0.5); 3.5283 (0.4); 3.3458 (16.0); 2.5347 (2.9); 2.5288 (6.3); 2.5228 (8.8); 2.5168 (6.6); 2.5109 (3.2); 0.0322 (0.3); 0.0212
(10.4); 0.0102 (0.4)
Ib-017: ¹H-NMR(499.9 MHz, d₆-DMSO):
δ = 10.5716 (11.4); 7.9546 (12.5); 7.9371 (16.0); 7.8558 (5.5); 7.8388 (11.0); 7.8230 (10.0); 7.8030 (3.6); 7.7989 (4.4); 7.7954
(3.6); 7.7833 (15.4); 7.7659 (13.2); 7.7084 (1.8); 7.6916 (8.1); 7.6771 (6.8); 7.6695 (4.5); 7.6653 (4.4); 7.6624 (3.9); 7.6598
(3.8); 7.6560 (2.9); 7.6403 (5.8); 7.6284 (4.3); 7.6242 (5.8); 7.6125 (4.7); 7.6086 (3.4); 7.5967 (2.7); 7.4998 (2.4); 7.4955 (2.4);
7.4830 (4.2); 7.4782 (4.1); 7.4658 (2.1); 7.4614 (1.9); 4.5020 (3.9); 4.4632 (4.9); 4.2061 (7.1); 4.1676 (5.8); 3.3182 (24.2);
2.5074 (12.8); 2.5039 (17.2); 2.5004 (13.1); 1.9953 (0.6); 1.9907 (0.6)
Ib-018: ¹H-NMR(300.2 MHz, CDCl3):
δ = 9.1055 (0.5); 7.9078 (0.8); 7.7865 (2.2); 7.7631 (3.3); 7.7316 (2.2); 7.7090 (0.4); 7.7019 (0.8); 7.6915 (0.5); 7.6646 (1.0);
7.6548 (0.7); 7.6447 (0.7); 7.6330 (1.1); 7.6257 (1.2); 7.6018 (1.0); 7.5841 (0.6); 7.5744 (0.5); 7.5551 (0.6); 7.5464 (0.5); 7.5281
(0.5); 7.5014 (0.4); 7.4929 (0.4); 7.4669 (0.7); 7.4464 (0.7); 7.4213 (0.4); 7.3279 (0.4); 7.3227 (0.4); 7.2990 (28.6); 7.2604 (0.8);
7.2526 (0.8); 7.2320 (0.5); 4.1700 (0.4); 4.1462 (0.4); 3.8555 (1.7); 3.7959 (2.8); 3.7206 (0.3); 3.6111 (1.9); 3.5518 (1.1); 2.0832
(2.0); 1.6032 (16.0); 1.3206 (0.8); 1.2968 (2.0); 1.2730 (0.7); 0.9196 (0.8); 0.8956 (0.3); 0.0486 (1.0); 0.0376 (30.7); 0.0267 (1.2)
Ib-019: ¹H-NMR(400.2 MHz, d₆-DMSO):
δ = 8.7033 (14.6); 8.3155 (0.4); 8.0176 (0.8); 7.9963 (0.9); 7.9525 (0.5); 7.7921 (2.1); 7.7880 (13.1); 7.7834 (4.4); 7.7715 (4.6);
7.7668 (16.0); 7.7628 (2.5); 7.5394 (0.9); 7.5348 (0.3); 7.5228 (0.4); 7.5181 (1.2); 7.5121 (2.5); 7.5080 (15.9); 7.5033 (4.8);
7.4914 (4.3); 7.4868 (14.0); 7.4827 (2.0); 4.6574 (1.2); 3.9808 (5.6); 3.9341 (7.2); 3.6034 (4.4); 3.5565 (3.6); 3.4447 (0.3);
3.3531 (0.4); 3.3281 (135.2); 2.9944 (12.9); 2.9006 (13.2); 2.8912 (7.2); 2.8572 (1.3); 2.7321 (3.4); 2.7309 (3.6); 2.6803 (0.3);
2.6757 (0.8); 2.6710 (1.0); 2.6665 (0.7); 2.6619 (0.4); 2.5723 (0.3); 2.5494 (0.4); 2.5246 (3.0); 2.5200 (4.3); 2.5112 (59.0);
2.5067 (122.8); 2.5021 (162.2); 2.4975 (113.3); 2.4929 (52.2); 2.3335 (0.7); 2.3290 (1.0); 2.3243 (0.7); 1.2582 (0.4); 1.2403
(1.5); 0.8532 (0.4); 0.0081 (0.4); −0.0002 (12.7); −0.0084 (0.4)
Ib-020: ¹H-NMR(300.2 MHz, CDCl3):
δ = 8.1132 (0.5); 8.0843 (0.5); 7.7496 (0.6); 7.7208 (0.4); 7.3261 (2.5); 7.2980 (9.0); 7.2827 (0.7); 7.2654 (2.9); 7.2602 (1.6);
7.2354 (5.5); 7.2204 (2.2); 7.2152 (1.8); 7.2067 (2.8); 7.1975 (1.8); 7.1859 (0.3); 7.1766 (0.4); 7.1723 (0.4); 7.0487 (2.3); 7.0444
(3.0); 7.0209 (2.4); 5.6885 (1.4); 3.9862 (2.2); 3.6723 (1.4); 3.6122 (2.7); 3.5162 (16.0); 3.4987 (2.0); 3.4383 (0.9); 2.0418 (1.9);
1.6881 (1.3); 0.0357 (2.6)
Ib-021: ¹H-NMR(300.2 MHz, CDCl3):
δ = 7.6452 (0.3); 7.6087 (0.4); 7.4877 (5.7); 7.3750 (7.1); 7.3491 (4.7); 7.2989 (6.7); 3.7298 (8.5); 3.6721 (2.9); 3.6120 (5.7);
3.5363 (5.3); 3.4747 (3.6); 3.4331 (16.0); 3.3088 (8.6); 3.2455 (0.4); 3.1392 (7.8); 3.0326 (9.8); 0.0312 (3.6)
Ib-022: ¹H-NMR(400.2 MHz, d₆-DMSO):
δ = 8.7023 (9.4); 7.9530 (0.5); 7.7910 (13.7); 7.7865 (4.5); 7.7745 (4.7); 7.7699 (16.0); 7.7660 (2.6); 7.4750 (2.5); 7.4711 (16.0);
7.4665 (4.8); 7.4545 (4.4); 7.4500 (14.1); 7.4460 (2.2); 3.9770 (5.8); 3.9304 (7.4); 3.6024 (6.3); 3.5556 (4.5); 3.3314 (39.1);
3.3084 (0.5); 3.2473 (2.4); 2.8914 (4.0); 2.7328 (3.2); 2.7316 (3.2); 2.6717 (0.4); 2.5253 (1.1); 2.5206 (1.6); 2.5119 (23.2);
2.5074 (48.6); 2.5028 (64.0); 2.4982 (44.6); 2.4936 (20.5); 2.3296 (0.4); 1.6210 (3.8); 1.6107 (4.3); 1.5583 (2.3); 1.4577 (2.4);
1.2400 (0.7); −0.0002 (5.2)
Ib-023: ¹H-NMR(400.2 MHz, d₆-DMSO):
δ = 8.7435 (8.5); 7.8485 (6.7); 7.8438 (2.6); 7.8322 (3.8); 7.8272 (16.0); 7.7944 (14.2); 7.7895 (3.6); 7.7730 (6.2); 7.3530 (0.9);
7.3475 (1.5); 7.3300 (5.9); 7.3131 (4.3); 7.3092 (3.8); 7.3005 (3.0); 7.2956 (3.1); 7.2909 (2.3); 7.2824 (3.2); 7.2771 (2.6); 7.2650
(1.4) ; 7.2599 (1.1); 7.1720 (3.6); 7.1551 (2.9); 7.1512 (2.3); 5.0478 (13.0); 4.9506 (13.0); 4.0154 (4.5); 3.9688 (5.7); 3.6341
(3.5) ; 3.5873 (2.9); 3.3345 (76.1); 2.8905 (1.2); 2.7321 (1.0); 2.7311 (1.0); 2.6714 (0.4); 2.5249 (1.1); 2.5202 (1.7); 2.5115
(24.8); 2.5070 (51.1); 2.5024 (66.8); 2.4978 (46.4); 2.4932 (21.2); 2.3293 (0.4); 1.2392 (1.0); −0.0002 (3.8)
Ib-024: ¹H-NMR(400.2 MHz, d₆-DMSO):
δ = 8.8569 (3.0); 8.8366 (3.1); 8.7308 (9.6); 7.9768 (8.1); 7.9723 (2.8); 7.9599 (3.3); 7.9553 (11.3); 7.8361 (11.1); 7.8316 (3.1);
7.8192 (2.8); 7.8148 (8.3); 5.0093 (0.4); 4.9919 (1.7); 4.9738 (2.3); 4.9546 (1.7); 4.9369 (0.4); 4.2343 (3.9); 4.2314 (3.6); 4.2104
(4.5); 4.2074 (8.6); 4.1834 (4.1); 4.1815 (4.4); 3.9967 (2.9); 3.9497 (3.6); 3.6223 (2.9); 3.5753 (2.3); 3.3350 (42.7); 3.0597 (0.4);
3.0578 (0.4); 3.0340 (0.8); 3.0175 (2.0); 3.0157 (2.0); 3.0098 (0.5); 3.0077 (0.5); 2.9973 (2.2); 2.9918 (3.9); 2.9727 (3.6); 2.9569
(0.5); 2.9551 (0.5); 2.9481 (1.8); 2.9306 (0.8); 2.9044 (0.3); 2.8915 (1.5); 2.7330 (1.2); 2.7317 (1.2); 2.5258 (0.8); 2.5211 (1.2);
2.5124 (16.2); 2.5079 (33.4); 2.5033 (43.5); 2.4986 (30.2); 2.4940 (13.7); 1.4251 (16.0); 1.4077 (15.8); 1.2397 (0.6); −0.0002
(2.7)
Ib-025: ¹H-NMR(400.2 MHz, d₆-DMSO):
δ = 10.0253 (2.0); 8.8751 (1.0); 8.8551 (1.0); 8.7258 (2.2); 7.9801 (2.6); 7.9588 (3.8); 7.9534 (2.4); 7.8213 (3.5); 7.8000 (2.8);
7.1940 (1.8); 7.1725 (1.0); 7.1681 (0.7); 7.1522 (1.0); 7.1476 (0.9); 6.8071 (2.0); 6.7869 (1.9); 5.1197 (0.5); 5.1013 (0.8); 5.0827
(0.6); 3.9942 (0.7); 3.9890 (0.7); 3.9475 (0.9); 3.9424 (0.9); 3.6177 (0.9); 3.5710 (0.7); 3.3335 (21.2); 2.8908 (16.0); 2.8712
(1.1); 2.8533 (1.8); 2.8335 (1.3); 2.7324 (13.1); 2.7312 (13.2); 2.5251 (0.5); 2.5204 (0.7); 2.5118 (9.6); 2.5073 (20.0); 2.5027
(26.2); 2.4981 (18.3); 2.4935 (8.5); 2.4479 (1.8); 2.4325 (1.6); 2.4283 (2.1); 2.4102 (1.5); 1.4666 (3.7); 1.4490 (3.6); 1.2397
(0.4); −0.0002 (1.9)
Ib-026: ¹H-NMR(400.2 MHz, d₆-DMSO):
δ = 8.7182 (6.4); 8.2917 (2.2); 8.2712 (2.2); 7.9485 (6.8); 7.9441 (2.4); 7.9317 (2.7); 7.9271 (9.6); 7.8141 (9.4); 7.8095 (2.7);
7.7972 (2.3); 7.7928 (7.0); 3.9907 (2.4); 3.9643 (0.6); 3.9446 (4.0); 3.9289 (1.6); 3.9121 (1.2); 3.8932 (0.6); 3.6170 (2.3); 3.5698
(1.8) ; 3.3316 (29.7); 2.8915 (1.2); 2.7328 (1.0); 2.7316 (1.0); 2.5255 (0.9); 2.5208 (1.3); 2.5121 (17.5); 2.5076 (36.4); 2.5030
(47.8) ; 2.4984 (33.3); 2.4938 (15.3); 1.5867 (0.5); 1.5714 (0.5); 1.5679 (0.9); 1.5530 (1.9); 1.5494 (1.0); 1.5341 (2.9); 1.5276
(1.6) ; 1.5152 (2.2); 1.5120 (2.2); 1.5094 (1.9); 1.4937 (2.2); 1.4754 (1.0); 1.4599 (0.4); 1.2396 (0.6); 1.1514 (14.8); 1.1348
(14.6) ; 0.8900 (7.4); 0.8716 (16.0); 0.8530 (6.7); −0.0002 (3.3)
Ib-027: ¹H-NMR(400.2 MHz, d₆-DMSO):
δ = 8.7196 (3.0); 8.5792 (0.8); 8.5679 (0.8); 7.9537 (2.1); 7.9324 (3.0); 7.9278 (1.0); 7.9157 (1.2); 7.9109 (4.4); 7.9068 (0.8);
7.8209 (4.4); 7.8161 (1.2); 7.8040 (1.0); 7.7994 (3.0); 7.7952 (0.4); 3.9886 (1.5); 3.9419 (1.9); 3.6146 (1.2); 3.5676 (0.9); 3.3394
(18.0); 2.8922 (16.0); 2.8051 (6.4); 2.7938 (6.2); 2.7335 (13.9); 2.7321 (13.0); 2.5218 (0.4); 2.5132 (5.6); 2.5087 (11.6); 2.5041
(15.2); 2.4994 (10.6); 2.4948 (4.9); −0.0002 (1.2)
Ib-028: ¹H-NMR(400.2 MHz, d₆-DMSO):
δ = 8.7757 (3.0); 8.7128 (5.0); 7.9100 (4.3); 7.9058 (1.6); 7.8930 (1.8); 7.8886 (6.2); 7.7910 (6.3); 7.7865 (1.8); 7.7739 (1.6);
7.7696 (4.4); 3.9821 (2.2); 3.9354 (2.8); 3.6054 (1.8); 3.5585 (1.4); 3.3343 (20.0); 2.8919 (1.1); 2.7333 (0.9); 2.7321 (0.9);
2.5262 (0.4); 2.5215 (0.6); 2.5128 (9.0); 2.5083 (18.6); 2.5037 (24.4); 2.4991 (17.1); 2.4945 (8.0); 1.3728 (16.0); 1.2393 (0.4);
0.7627 (1.1); 0.7497 (3.4); 0.7454 (3.6); 0.7347 (1.5); 0.6320 (1.9); 0.6210 (4.0); 0.6161 (3.8); 0.6032 (1.3); −0.0002 (1.8)
Ib-029: ¹H-NMR(400.2 MHz, d₆-DMSO):
δ = 8.7680 (3.1); 8.7200 (3.6); 7.9531 (0.6); 7.9218 (4.2); 7.9004 (6.1); 7.7945 (6.0); 7.7732 (4.4); 3.9844 (2.1); 3.9378 (2.7);
3.6092 (1.7); 3.5623 (1.4); 3.3344 (38.8); 2.8916 (4.5); 2.7329 (3.8); 2.7317 (3.7); 2.5258 (0.6); 2.5211 (0.9); 2.5123 (12.7);
2.5078 (26.3); 2.5033 (34.6); 2.4986 (24.3); 2.4941 (11.3); 1.2396 (0.4); 1.1495 (16.0); 0.7264 (1.1); 0.7075 (3.6); 0.6971 (1.7);
0.6728 (0.4); 0.6594 (0.4); 0.6352 (1.8); 0.6243 (3.4); 0.6204 (3.0); 0.6055 (1.0); 0.5591 (1.3); 0.5484 (4.0); 0.5447 (4.2); 0.5346
(1.4); 0.1883 (1.7); 0.1782 (4.6); 0.1738 (4.8); 0.1632 (1.4); −0.0002 (1.7)
Ib-030: ¹H-NMR(300.2 MHz, d₆-DMSO):
δ = 10.2931 (0.5); 8.7826 (0.5); 8.1005 (0.5); 8.0723 (0.7); 7.9227 (0.8); 7.8945 (0.6); 3.9918 (0.4); 3.3478 (16.0); 2.5293 (2.5);
2.5233 (3.4); 2.5173 (2.6); 1.2675 (0.5); 0.8799 (0.4); 0.0216 (4.0)
Ib-031: ¹H-NMR(400.2 MHz, d₆-DMSO):
δ = 10.5451 (4.2); 8.7633 (3.9); 8.0687 (6.4); 8.0643 (2.1); 8.0518 (2.5); 8.0473 (8.2); 7.9534 (2.0); 7.9047 (8.1); 7.9002 (2.4);
7.8877 (2.2); 7.8834 (6.3); 7.7868 (1.3); 7.7813 (2.0); 7.7758 (1.4); 7.7574 (1.3); 7.7520 (2.0); 7.7463 (1.3); 7.5935 (1.7); 7.5916
(2.0); 7.5890 (1.8); 7.5871 (1.6); 7.5730 (2.2); 7.5710 (2.3); 7.5684 (2.3); 7.5666 (2.0); 7.4350 (1.4); 7.4176 (1.7); 7.4144 (2.6);
7.3972 (2.6); 7.3941 (1.5); 7.3766 (1.2); 6.9800 (1.0); 6.9780 (1.0); 6.9734 (1.0); 6.9583 (1.8); 6.9519 (1.7); 6.9376 (1.0); 6.9356
(1.0); 6.9310 (0.9); 6.9293 (0.8); 4.0257 (2.9); 3.9790 (3.7); 3.6511 (2.3); 3.6042 (1.9); 3.3449 (114.1); 2.8921 (16.0); 2.7335
(13.7); 2.7323 (12.8); 2.5267 (0.9); 2.5220 (1.3); 2.5133 (17.5); 2.5088 (36.1); 2.5043 (46.9); 2.4996 (32.5); 2.4951 (14.8);
1.2382 (0.6)
Ib-032: ¹H-NMR(300.2 MHz, d₆-DMSO):
δ = 8.7494 (2.0); 8.4484 (2.6); 8.4244 (2.6); 8.0180 (0.3); 7.9688 (5.9); 7.9405 (9.1); 7.8314 (8.9); 7.8031 (6.1); 4.2879 (0.8);
4.2644 (1.4); 4.2415 (1.4); 4.2179 (0.8); 4.0196 (2.9); 3.9573 (4.0); 3.6434 (2.8); 3.5806 (2.1); 3.3503 (16.0); 2.5292 (6.4);
2.5233 (8.7); 2.5173 (6.6); 1.9380 (1.4); 1.9271 (2.1); 1.9139 (2.5); 1.9041 (2.4); 1.7230 (3.1); 1.6834 (1.3); 1.6523 (0.6); 1.6137
(1.7); 1.5916 (2.9); 1.5571 (5.4); 0.0318 (0.5); 0.0210 (9.4); 0.0102 (0.5)
Ib-033: ¹H-NMR(400.2 MHz, d₆-DMSO):
δ = 10.9023 (7.5); 8.7725 (5.2); 8.4872 (6.1); 8.4833 (6.5); 8.4755 (6.4); 8.4716 (6.4); 8.0994 (12.4); 8.0876 (7.0); 8.0835 (10.3);
8.0783 (16.0); 8.0677 (6.7); 8.0638 (6.4); 7.9038 (15.4); 7.8827 (12.9); 7.4423 (6.0); 7.4306 (5.7); 7.4223 (5.5); 7.4106 (5.6);
4.0265 (5.9); 3.9798 (7.4); 3.6502 (4.8); 3.6032 (3.9); 3.3456 (129.1); 2.8908 (1.3); 2.7317 (1.2); 2.6771 (0.3); 2.6726 (0.4);
2.6682 (0.3); 2.5260 (1.3); 2.5212 (1.9); 2.5125 (25.8); 2.5082 (52.9); 2.5037 (69.0); 2.4991 (48.7); 2.4946 (22.8); 2.3304 (0.4);
1.2396 (0.7); −0.0002 (0.8)
Ib-034: ¹H-NMR(400.2 MHz, d₆-DMSO):
δ = 9.0923 (0.4); 9.0786 (0.9); 9.0647 (0.4); 8.7341 (1.9); 7.9605 (2.4); 7.9530 (2.2); 7.9440 (1.0); 7.9392 (3.3); 7.8378 (3.1);
7.8332 (0.9); 7.8208 (0.8); 7.8165 (2.3); 4.0831 (1.6); 4.0769 (1.7); 4.0693 (1.6); 4.0631 (1.6); 3.9963 (1.1); 3.9496 (1.4); 3.6181
(0.9); 3.5712 (0.7); 3.3443 (40.8); 3.1489 (0.9); 3.1428 (2.0); 3.1366 (0.9); 2.8918 (16.0); 2.7331 (12.9); 2.7321 (13.0); 2.5216
(0.4); 2.5130 (6.1); 2.5085 (12.6); 2.5039 (16.4); 2.4993 (11.4); 2.4947 (5.3)
Ib-035: ¹H-NMR(400.2 MHz, d₆-DMSO):
δ = 8.7231 (1.2); 8.4774 (0.5); 7.9504 (1.4); 7.9459 (0.5); 7.9335 (0.6); 7.9289 (2.0); 7.8197 (2.0); 7.8151 (0.6); 7.8027 (0.5);
7.7983 (1.4); 3.9913 (0.7); 3.9447 (0.9); 3.6186 (0.5); 3.5717 (0.4); 3.3312 (6.8); 3.1264 (1.5); 3.1104 (1.4); 2.5119 (4.0); 2.5074
(8.2); 2.5029 (10.8); 2.4982 (7.5); 2.4936 (3.5); 0.9062 (16.0); −0.0002 (0.8)
Ib-036: ¹H-NMR(400.2 MHz, d₆-DMSO):
δ = 8.7175 (6.8); 8.5526 (0.8); 8.5381 (1.7); 8.5234 (0.8); 7.9525 (2.0); 7.8773 (4.2); 7.8727 (1.6); 7.8605 (1.8); 7.8558 (7.2);
7.7952 (7.0); 7.7904 (1.9); 7.7782 (1.5); 7.7737 (4.2); 7.3366 (2.3); 7.3329 (3.3); 7.3279 (1.0); 7.3155 (5.5); 7.3125 (4.8); 7.3077
(0.9); 7.2856 (3.6); 7.2805 (1.0); 7.2673 (5.2); 7.2637 (2.1); 7.2517 (1.1); 7.2476 (2.4); 7.2432 (0.4); 7.1878 (1.1); 7.1843 (2.0);
7.1806 (1.1); 7.1715 (0.8); 7.1663 (2.4); 7.1611 (0.7); 7.1518 (0.6); 7.1484 (1.0); 7.1449 (0.5); 3.9763 (2.2); 3.9297 (2.9); 3.6045
(1.8); 3.5657 (4.2); 3.5573 (2.4); 3.5513 (4.4); 3.3315 (29.2); 2.8907 (16.0); 2.7323 (13.7); 2.7309 (12.9); 2.5248 (0.7); 2.5201
(1.0); 2.5114 (14.6); 2.5069 (30.8); 2.5023 (40.8); 2.4977 (28.8); 2.4931 (13.5); 1.2400 (0.6); 0.9894 (1.6); 0.9779 (4.2); 0.9735
(4.5); 0.9631 (1.9); 0.7892 (1.8); 0.7787 (4.2); 0.7743 (4.5); 0.7628 (1.4); −0.0002 (3.4)
Ib-037: ¹H-NMR(400.2 MHz, d₆-DMSO):
δ = 8.7238 (3.3); 8.5560 (0.7); 8.5405 (1.4); 8.5249 (0.6); 7.9611 (4.1); 7.9566 (1.4); 7.9442 (1.6); 7.9396 (5.7); 7.8166 (5.5);
7.8120 (1.6); 7.7997 (1.4); 7.7953 (4.2); 3.9897 (1.5); 3.9427 (1.9); 3.7721 (1.1); 3.7669 (1.4); 3.7550 (2.4); 3.7510 (2.6); 3.7395
(1.2); 3.7345 (1.3); 3.6158 (1.4); 3.5689 (1.1); 3.3932 (0.4); 3.3889 (0.3); 3.3772 (0.4); 3.3728 (0.4); 3.3597 (1.1); 3.3553 (1.2);
3.3436 (1.4); 3.3316 (24.4); 3.3198 (1.4); 3.3158 (1.2); 3.3018 (0.4); 3.2978 (0.4); 3.2824 (0.3); 2.5254 (0.6); 2.5208 (0.8);
2.5120 (11.3); 2.5075 (23.5); 2.5029 (30.9); 2.4983 (21.5); 2.4937 (9.9); 1.9146 (0.4); 1.9083 (0.8); 1.8938 (1.3); 1.8863 (1.6);
1.8756 (3.8); 1.8627 (2.8); 1.8469 (1.3); 1.8422 (0.6); 1.8304 (0.3); 1.5837 (0.5); 1.5727 (0.8); 1.5587 (0.9); 1.5523 (0.8); 1.5420
(0.9); 1.2394 (0.4); 1.1509 (16.0); −0.0002 (1.9)
Ib-038: ¹H-NMR(400.2 MHz, d₆-DMSO):
δ = 8.7252 (1.6); 8.6371 (0.4); 8.6233 (0.8); 8.6090 (0.4); 7.9530 (2.2); 7.9398 (2.3); 7.9228 (1.0); 7.9183 (3.4); 7.8176 (3.2);
7.8130 (1.1); 7.7963 (2.4); 3.9881 (1.2); 3.9415 (1.5); 3.6146 (1.0); 3.5678 (0.8); 3.3373 (34.3); 3.1004 (0.4); 3.0858 (0.3);
3.0819 (0.4); 3.0783 (0.3); 3.0362 (0.6); 3.0198 (0.4); 2.8916 (16.0); 2.7330 (13.5); 2.7317 (13.6); 2.5259 (0.4); 2.5212 (0.6);
2.5124 (7.9); 2.5079 (16.4); 2.5034 (21.6); 2.4987 (15.2); 2.4942 (7.1); 2.1947 (0.8); 2.0993 (0.9); 1.7263 (0.4); 1.4821 (0.6);
1.4734 (0.8); 1.4665 (0.6); 1.4554 (0.8); 1.4495 (0.7); 1.4409 (0.4); 1.3698 (0.3); 1.3646 (0.4); 1.3495 (0.7); 1.3400 (0.7); 1.3343
(0.9); 1.3193 (0.5); 1.3135 (0.4); 1.1231 (1.2); 1.1184 (1.2); 1.1048 (1.4); 1.0998 (1.4); 1.0874 (1.1); 1.0624 (0.9)
Ib-039: ¹H-NMR(400.2 MHz, d₆-DMSO):
δ = 9.2630 (2.1); 9.2483 (4.5); 9.2334 (2.2); 8.7379 (15.0); 8.0024 (11.9); 7.9979 (4.1); 7.9855 (4.6); 7.9809 (16.0); 7.9538 (0.4);
7.8507 (15.8); 7.8461 (4.6); 7.8337 (4.1); 7.8292 (12.2); 7.7793 (8.1); 7.7783 (8.1); 7.7756 (5.8); 7.7505 (3.0); 7.7474 (4.6);
7.7436 (2.7); 7.7315 (3.6); 7.7282 (5.5); 7.7246 (3.4); 7.6923 (3.7); 7.6757 (3.8); 7.6717 (5.0); 7.5825 (5.9); 7.5630 (8.8); 7.5438
(3.6) ; 4.5481 (9.4); 4.5334 (9.3); 3.9986 (5.6); 3.9520 (7.1); 3.6236 (4.4); 3.5766 (3.6); 3.3385 (138.6); 2.8919 (3.2); 2.7334
(2.6) ; 2.7321 (2.6); 2.6776 (0.4); 2.6729 (0.5); 2.6683 (0.4); 2.5265 (1.5); 2.5218 (2.1); 2.5131 (32.2); 2.5086 (67.4); 2.5040
(88.6) ; 2.4993 (61.8); 2.4947 (28.5); 2.3353 (0.4); 2.3307 (0.5); 2.3262 (0.4); 1.2390 (1.1); −0.0002 (4.4)
Ib-040: ¹H-NMR(400.2 MHz, d₆-DMSO):
δ = 9.2343 (2.0); 9.2195 (4.2); 9.2044 (2.0); 8.7355 (10.0); 8.0021 (11.8); 7.9977 (4.1); 7.9853 (4.7); 7.9807 (16.0); 7.8482 (15.4);
7.8437 (4.5); 7.8312 (4.0); 7.8269 (12.1); 7.4090 (2.2); 7.3937 (2.6); 7.3893 (4.2); 7.3740 (4.5); 7.3695 (3.3); 7.3541 (3.2);
7.1847 (5.1); 7.1654 (4.5); 7.1570 (2.7); 7.1509 (3.3); 7.1316 (2.3); 7.1256 (3.2); 7.1221 (2.5); 7.1025 (2.0); 7.0971 (1.6); 7.0815
(3.5); 7.0751 (2.8); 7.0594 (1.7); 7.0542 (1.4); 4.5154 (9.8); 4.5005 (9.7); 3.9986 (5.6); 3.9519 (7.0); 3.6239 (4.4); 3.5769 (3.6);
3.3377 (59.2); 2.8911 (2.2); 2.7320 (1.8); 2.6725 (0.4); 2.5260 (1.3); 2.5213 (1.8); 2.5126 (25.6); 2.5081 (52.7); 2.5035 (68.8);
2.4989 (47.9); 2.4943 (21.9); 2.3303 (0.4); 1.2391 (0.9); −0.0002 (2.0)
Ib-041: ¹H-NMR(300.2 MHz, d₆-DMSO):
δ = 8.7521 (1.8); 8.7221 (1.2); 8.7034 (0.6); 7.9825 (2.6); 7.9547 (4.1); 7.8488 (4.0); 7.8211 (2.7); 4.0246 (1.4); 3.9623 (1.9);
3.6482 (1.3); 3.5857 (0.9); 3.3486 (16.0); 3.1963 (1.6); 3.1752 (2.7); 3.1549 (1.7); 2.5292 (3.8); 2.5235 (5.4); 2.5178 (4.1);
1.0845 (0.4); 1.0768 (0.4); 1.0609 (0.7); 1.0446 (0.4); 1.0351 (0.4); 0.4921 (0.5); 0.4774 (1.7); 0.4718 (2.0); 0.4588 (1.0); 0.4508
(1.8); 0.4450 (1.8); 0.4322 (0.7); 0.2808 (0.7); 0.2629 (2.3); 0.2466 (2.2); 0.2314 (0.5); 0.0217 (5.4)
Ib-042: ¹H-NMR(499.9 MHz, d₆-DMSO):
δ = 9.1927 (2.2); 9.1809 (4.6); 9.1689 (2.2); 8.7243 (6.0); 7.9902 (12.4); 7.9868 (4.7); 7.9767 (5.0); 7.9731 (16.0); 7.8406 (15.5);
7.8370 (5.0); 7.8270 (4.6); 7.8235 (12.8); 7.3956 (0.7); 7.3896 (5.7); 7.3854 (2.6); 7.3784 (6.6); 7.3722 (7.4); 7.3653 (2.8);
7.3610 (6.6); 7.3550 (0.8); 7.1866 (1.0); 7.1805 (8.5); 7.1763 (2.6); 7.1673 (3.0); 7.1627 (14.6); 7.1580 (3.0); 7.1490 (2.4);
7.1448 (7.2); 7.1387 (0.7); 4.4816 (9.6); 4.4697 (9.6); 3.9924 (6.0); 3.9551 (7.3); 3.6193 (4.5); 3.5817 (3.8); 3.3222 (58.6);
2.5146 (7.0); 2.5109 (15.2); 2.5073 (21.1); 2.5036 (15.0); 2.5000 (6.8); 1.9945 (0.6); 1.1809 (0.3)
Ib-043: ¹H-NMR(400.2 MHz, d₆-DMSO):
δ = 8.7197 (12.3); 8.6344 (1.9); 8.6202 (3.8); 8.6057 (1.9); 7.9436 (11.0); 7.9393 (4.0); 7.9267 (4.6); 7.9222 (16.0); 7.8186 (15.3);
7.8142 (4.5); 7.7973 (11.3); 3.9888 (5.4); 3.9422 (6.9); 3.8643 (3.6); 3.8579 (3.6); 3.8359 (4.0); 3.8294 (4.0); 3.6152 (4.4);
3.5683 (3.5); 3.3338 (45.9); 3.2953 (3.3); 3.2906 (3.9); 3.2661 (6.6); 3.2616 (6.6); 3.2372 (3.7); 3.2324 (3.2); 3.1850 (5.4);
3.1690 (8.2); 3.1532 (5.5); 2.8915 (3.4); 2.7320 (2.8); 2.6723 (0.4); 2.5259 (1.1); 2.5212 (1.6); 2.5124 (23.2); 2.5079 (47.8);
2.5034 (62.6); 2.4987 (43.8); 2.4942 (20.2); 2.3301 (0.4); 1.8509 (0.3); 1.8423 (0.5); 1.8328 (0.9); 1.8228 (1.1); 1.8143 (1.3);
1.8048 (1.8); 1.7952 (1.4); 1.7866 (1.2); 1.7766 (1.1); 1.7675 (0.6); 1.7588 (0.4); 1.6142 (3.6); 1.6095 (3.5); 1.5820 (4.3); 1.5775
(4.0); 1.2515 (1.6); 1.2401 (2.2); 1.2217 (3.2); 1.2108 (3.4); 1.1891 (3.1); 1.1783 (2.8); 1.1594 (1.3); 1.1484 (1.1); −0.0002 (4.3)
Ib-044: ¹H-NMR(400.2 MHz, d₆-DMSO):
δ = 9.3499 (0.6); 9.3361 (1.3); 9.3220 (0.6); 8.7949 (1.4); 8.7781 (1.4); 8.7382 (2.3); 8.1871 (2.7); 8.0226 (3.4); 8.0057 (1.4);
8.0012 (4.5); 7.9529 (2.1); 7.8723 (4.3); 7.8510 (3.4); 7.8396 (0.8); 7.8169 (1.6); 7.7850 (0.8); 7.7674 (0.8); 7.7453 (0.4); 7.3656
(0.6); 7.3488 (1.1); 7.3318 (0.6); 4.7216 (2.5); 4.7078 (2.5); 4.0011 (1.6); 3.9545 (2.0); 3.6311 (1.3); 3.5842 (1.1); 3.3729 (0.3);
3.3690 (0.3); 3.3603 (0.3); 2.8916 (16.0); 2.7328 (13.2); 2.7317 (13.0); 2.5667 (1.6); 2.5529 (3.3); 2.5391 (1.6); 2.5261 (0.6);
2.5214 (0.9); 2.5127 (11.6); 2.5082 (24.0); 2.5036 (31.2); 2.4990 (21.8); 2.4944 (10.0); 1.2399 (0.4); −0.0002 (2.0)
Ib-045: ¹H-NMR(400.2 MHz, d₆-DMSO):
δ = 9.5582 (1.5); 8.7211 (2.8); 7.8742 (1.8); 7.8574 (1.0); 7.8528 (3.6); 7.8083 (3.4); 7.7870 (1.8); 3.9873 (1.2); 3.9407 (1.5);
3.6108 (1.0); 3.5640 (0.8); 3.3525 (3.5); 2.8916 (1.8); 2.7330 (1.4); 2.7317 (1.5); 2.6019 (16.0); 2.5675 (0.6); 2.5538 (1.2);
2.5400 (0.6); 2.5213 (0.4); 2.5125 (5.9); 2.5080 (12.4); 2.5034 (16.2); 2.4988 (11.4); 2.4942 (5.3); 2.4430 (0.4); −0.0002 (1.1)
Ib-046: ¹H-NMR(400.2 MHz, d₆-DMSO):
δ = 11.5906 (0.7); 8.7386 (0.7); 7.9531 (2.2); 7.8497 (0.4); 7.8277 (3.3); 7.8207 (3.1); 7.7987 (0.4); 3.9843 (0.8); 3.9376 (1.0);
3.6114 (0.6); 3.5645 (0.5); 3.3763 (0.3); 3.3356 (15.6); 2.8918 (16.0); 2.7330 (13.4); 2.7318 (13.4); 2.5211 (0.4); 2.5124 (5.3);
2.5079 (11.0); 2.5033 (14.4); 2.4987 (10.1); 2.4941 (4.7); 1.2699 (2.1); 1.2543 (2.0); 0.4507 (0.3); 0.4415 (0.4); 0.4225 (0.4)
Ib-047: ¹H-NMR(400.2 MHz, d₆-DMSO):
δ = 11.8923 (1.6); 8.7345 (3.5); 7.8547 (0.9); 7.8480 (0.5); 7.8323 (11.5); 7.8287 (11.2); 7.8128 (0.6); 7.8063 (0.9); 3.9884 (2.1);
3.9417 (2.6); 3.7227 (16.0); 3.6104 (1.7); 3.5635 (1.4); 3.3337 (39.1); 2.8912 (1.2); 2.7315 (1.0); 2.5254 (0.7); 2.5207 (1.0);
2.5120 (14.0); 2.5075 (28.7); 2.5030 (37.3); 2.4984 (26.0); 2.4938 (12.0); 1.2398 (0.4); −0.0002 (0.6)
Ib-048: ¹H-NMR(300.2 MHz, d₆-DMSO):
δ = 13.2442 (1.0); 8.7782 (3.8); 8.1243 (1.7); 8.0937 (0.4); 8.0787 (0.8); 8.0531 (7.1); 8.0255 (10.1); 7.8826 (9.6); 7.8549 (7.2);
4.0325 (3.3); 3.9701 (4.5); 3.6454 (3.2); 3.5829 (2.3); 3.3508 (16.0); 2.5292 (8.4); 2.5236 (11.7); 2.5179 (9.0); 0.0306 (0.4);
0.0209 (12.8)
Ib-049: ¹H-NMR(300.2 MHz, d₆-DMSO):
δ = 8.7899 (1.2); 8.0771 (0.8); 8.0492 (1.2); 7.9111 (1.1); 7.8830 (0.8); 4.0419 (0.4); 3.9794 (0.5); 3.9040 (3.5); 3.6482 (0.4);
3.3487 (16.0); 2.5291 (2.0); 2.5233 (2.8); 2.5174 (2.2); 0.0213 (3.4)
Ib-050: ¹H-NMR(300.2 MHz, CDCl3):
δ = 8.2463 (6.0); 8.2401 (2.2); 8.2238 (2.2); 8.2174 (7.1); 7.8736 (7.0); 7.8673 (2.4); 7.8509 (2.1); 7.8447 (6.0); 7.2986 (18.7);
3.8839 (2.4); 3.8241 (3.9); 3.7641 (1.8); 3.6379 (2.6); 3.6333 (1.8); 3.5780 (1.6); 3.5734 (1.2); 2.9348 (2.2); 2.9095 (7.2); 2.8842
(7.4); 2.8590 (2.5); 1.6108 (1.0); 1.4665 (7.7); 1.4413 (16.0); 1.4160 (7.3); 1.2901 (0.9); 0.0482 (0.9); 0.0373 (25.1); 0.0264 (0.9)
Ib-052: ¹H-NMR(300.2 MHz, CDCl3):
δ = 7.5275 (1.7); 7.5190 (14.2); 7.5127 (5.0); 7.4968 (4.8); 7.4903 (15.4); 7.4820 (2.1); 7.2988 (16.0); 6.7052 (1.9); 6.6967 (16.0);
6.6903 (5.3); 6.6743 (4.7); 6.6679 (15.0); 6.6595 (1.9); 4.1910 (2.6); 4.1857 (4.1); 4.1799 (2.7); 4.1311 (3.3); 4.1257 (5.3);
4.1199 (3.6); 3.9593 (10.2); 3.7344 (8.6); 3.6746 (6.7); 3.5808 (0.9); 3.5589 (1.9); 3.5441 (2.4); 3.5229 (4.6); 3.5002 (2.8);
3.4901 (1.6); 3.4806 (2.1); 3.4667 (3.3); 3.4571 (5.0); 3.4458 (3.3); 3.4330 (5.5); 3.4246 (3.3); 3.4099 (2.2); 3.4006 (1.8); 3.3926
(2.8); 3.3702 (4.6); 3.3491 (2.5); 3.3337 (1.9); 3.3121 (0.8); 1.9930 (0.6); 1.9822 (1.0); 1.9722 (1.2); 1.9618 (3.1); 1.9394 (6.4);
1.9285 (7.5); 1.9188 (9.6); 1.9064 (8.0); 1.8961 (6.5); 1.8736 (2.8); 1.8525 (0.9); 1.8426 (0.5); 1.6037 (9.7); 1.2926 (0.4); 0.0485
(0.7); 0.0379 (20.4); 0.0270 (0.8)
Ib-053: ¹H-NMR(400.2 MHz, d₆-DMSO):
δ = 9.4153 (0.8); 8.5876 (0.7); 7.7881 (1.0); 7.7835 (0.3); 7.7709 (0.4); 7.7660 (1.5); 7.6607 (1.4); 7.6559 (0.4); 7.6433 (0.3);
7.6387 (1.0); 3.9019 (0.5); 3.8556 (0.6); 3.5426 (0.4); 3.3314 (12.0); 2.8912 (1.2); 2.7324 (1.0); 2.7311 (1.0); 2.5117 (4.2);
2.5072 (8.6); 2.5026 (11.3); 2.4980 (8.0); 2.4934 (3.7); 1.2346 (16.0); −0.0002 (0.8)
Ib-054: ¹H-NMR(400.2 MHz, d₆-DMSO):
δ = 10.0706 (2.1); 8.5842 (3.6); 7.7304 (0.3); 7.7251 (2.6); 7.7201 (0.9); 7.7080 (1.2); 7.7028 (5.4); 7.6978 (0.8); 7.6641 (0.8);
7.6592 (5.2); 7.6540 (1.2); 7.6420 (0.9); 7.6370 (2.7); 3.8934 (1.6); 3.8471 (2.0); 3.5381 (1.2); 3.4915 (1.0); 3.3298 (19.9);
2.8909 (0.9); 2.7325 (0.8); 2.7311 (0.8); 2.6435 (0.4); 2.6265 (1.0); 2.6094 (1.4); 2.5924 (1.1); 2.5752 (0.4); 2.5250 (0.6); 2.5204
(0.9); 2.5117 (11.9); 2.5071 (24.8); 2.5026 (32.6); 2.4979 (22.8); 2.4933 (10.5); 1.2397 (0.5); 1.1172 (16.0); 1.1001
(15.6); −0.0002 (3.4)
Ib-055: ¹H-NMR(300.2 MHz, d₆-DMSO):
δ = 10.1947 (0.6); 7.6873 (2.0); 7.6781 (2.1); 3.9202 (0.4); 3.8584 (0.6); 3.5636 (0.4); 3.3411 (16.0); 2.5344 (1.9); 2.5288 (3.8);
2.5228 (5.1); 2.5168 (3.7); 2.0901 (3.8); 0.0216 (6.4)
Ib-056: ¹H-NMR(300.2 MHz, d₆-DMSO):
δ = 7.7192 (1.1); 7.7154 (1.1); 3.3418 (16.0); 2.5349 (1.0); 2.5290 (2.2); 2.5229 (3.1); 2.5169 (2.2); 2.5110 (1.0); 1.7173 (1.9);
0.0217 (3.8)
Ib-057: ¹H-NMR(400.2 MHz, d₆-DMSO):
δ = 9.3692 (2.9); 8.5848 (4.2); 7.7781 (0.4); 7.7723 (4.0); 7.7676 (1.4); 7.7550 (1.5); 7.7502 (6.0); 7.7447 (0.8); 7.6609 (0.8);
7.6553 (5.8); 7.6505 (1.6); 7.6380 (1.3); 7.6333 (4.1); 7.6277 (0.5); 3.9016 (2.0); 3.8553 (2.5); 3.5421 (1.6); 3.4955 (1.3); 3.3297
(26.8) ; 2.8907 (0.8); 2.7321 (0.7); 2.7311 (0.7); 2.5247 (0.7); 2.5201 (1.0); 2.5113 (14.6); 2.5069 (30.3); 2.5023 (39.8); 2.4977
(27.8) ; 2.4931 (12.9); 1.4144 (16.0); 1.2394 (0.5); 1.1222 (1.3); 1.1128 (3.9); 1.1060 (4.0); 1.0970 (1.4); 0.6693 (1.7); 0.6600
(5.1); 0.6532 (5.0); 0.6433 (1.5); −0.0002 (2.8)
Ib-058: ¹H-NMR(400.2 MHz, d₆-DMSO):
δ = 10.6676 (5.6); 8.6250 (7.3); 7.9522 (2.6); 7.8459 (4.8); 7.8243 (6.9); 7.7313 (9.0); 7.7095 (7.9); 7.6909 (3.0); 7.6875 (3.2);
7.6727 (1.5); 7.6685 (1.7); 7.6310 (0.8); 7.6267 (0.8); 7.6173 (1.0); 7.6126 (1.8); 7.5922 (2.0); 7.5875 (1.3); 7.5783 (1.1); 7.5741
(1.0); 7.3928 (2.2); 7.3669 (5.5); 7.3472 (6.4); 7.3297 (1.9); 7.3277 (2.0); 3.9263 (3.4); 3.8800 (4.4); 3.5704 (3.1); 3.5239 (2.4);
3.3303 (48.4); 3.1981 (0.5); 2.8905 (16.0); 2.7310 (14.6); 2.6756 (0.4); 2.6711 (0.5); 2.6290 (0.8); 2.5065 (62.8); 2.5021 (80.0);
2.4977 (61.0); 2.3291 (0.5); 2.3245 (0.4); 1.2393 (1.3); −0.0002 (3.6)
Ib-059: ¹H-NMR(400.2 MHz, d₆-DMSO):
δ = 10.5665 (3.0); 8.6240 (4.4); 8.0286 (1.9); 8.0240 (3.3); 8.0193 (2.0); 7.9529 (2.0); 7.9414 (1.2); 7.9386 (1.6); 7.9347 (1.2);
7.9221 (1.4); 7.9183 (1.9); 7.9154 (1.7); 7.9096 (4.0); 7.9051 (1.4); 7.8924 (1.4); 7.8876 (5.4); 7.8820 (0.7); 7.7477 (0.7); 7.7422
(5.1) ; 7.7375 (1.5); 7.7248 (1.3); 7.7202 (4.2); 7.7145 (0.5); 7.7038 (1.0); 7.7013 (1.2); 7.6986 (1.1); 7.6960 (1.0); 7.6838 (1.5);
7.6812 (1.6); 7.6786 (1.6); 7.6760 (1.5); 7.6082 (2.3); 7.5887 (3.3); 7.5689 (1.4); 3.9322 (1.8); 3.8859 (2.4); 3.5757 (1.5); 3.5292
(1.2) ; 3.3302 (36.4); 3.1986 (0.7); 2.8910 (16.0); 2.7326 (12.8); 2.7313 (13.4); 2.6293 (1.1); 2.5253 (0.8); 2.5206 (1.2); 2.5118
(18.2) ; 2.5073 (37.8); 2.5028 (49.8); 2.4981 (34.7); 2.4935 (16.0); 1.2387 (0.9); −0.0002 (3.3)
Ib-060: ¹H-NMR(400.2 MHz, d₆-DMSO):
δ = 10.5330 (8.8); 9.5418 (1.7); 8.7872 (1.8); 8.7207 (0.8); 8.7162 (1.0); 8.6243 (9.1); 8.5874 (0.8); 8.5649 (0.9); 8.2347 (0.6);
8.2296 (0.6); 8.2123 (0.5); 8.2071 (0.6); 7.9530 (1.4); 7.9141 (12.0); 7.9095 (4.0); 7.8968 (4.4); 7.8920 (16.0); 7.8864 (2.0);
7.8409 (3.0); 7.8376 (4.5); 7.8350 (3.8); 7.8216 (3.5); 7.8181 (5.8); 7.8157 (4.6); 7.8055 (3.0); 7.7993 (3.5); 7.7956 (2.5); 7.7810
(2.9); 7.7748 (3.3); 7.7708 (2.6); 7.7481 (2.2); 7.7426 (15.4); 7.7379 (4.4); 7.7253 (4.0); 7.7207 (12.5); 7.7149 (1.4); 7.6375
(2.3); 7.6229 (2.5); 7.6172 (4.2); 7.6027 (4.2); 7.5977 (2.8); 7.5829 (2.6); 7.4982 (1.9); 7.4962 (2.2); 7.4915 (1.8); 7.4897 (1.9);
7.4747 (3.5); 7.4691 (3.1); 7.4554 (1.5); 7.4535 (1.6); 7.4489 (1.4); 7.4470 (1.4); 3.9577 (7.2); 3.9325 (5.5); 3.8862 (7.1); 3.5765
(4.5) ; 3.5299 (3.6); 3.3329 (80.3); 3.1991 (0.8); 2.8915 (10.9); 2.8782 (0.5); 2.8598 (1.5); 2.8414 (1.6); 2.8229 (0.5); 2.7330
(8.5) ; 2.7319 (9.2); 2.6770 (0.6); 2.6724 (0.8); 2.6678 (0.6); 2.6298 (1.3); 2.5736 (0.4); 2.5260 (2.4); 2.5212 (3.5); 2.5126 (48.8);
2.5081 (100.1); 2.5035 (130.4); 2.4989 (90.5); 2.4943 (41.6); 2.3349 (0.6); 2.3303 (0.8); 2.3257 (0.6); 1.2616 (0.6); 1.2593 (0.5);
1.2385 (2.6); 1.0112 (1.7); 0.9929 (3.5); 0.9744 (1.6); 0.8526 (0.6); −0.0001 (9.6)
Ib-061: ¹H-NMR(400.2 MHz, d₆-DMSO):
δ = 10.7021 (7.5); 8.6283 (9.7); 8.1321 (8.5); 8.1274 (3.4); 8.1156 (4.3); 8.1106 (16.0); 8.0568 (15.1); 8.0520 (4.1); 8.0400 (3.2);
8.0355 (8.6); 7.9529 (0.4); 7.9130 (9.4); 7.9086 (3.3); 7.8956 (3.6); 7.8909 (12.7); 7.7510 (12.4); 7.7464 (3.6); 7.7337 (3.2);
7.7291 (10.0); 7.7234 (1.2); 3.9337 (4.4); 3.8875 (5.8); 3.5770 (3.6); 3.5305 (2.9); 3.3323 (78.3); 2.8912 (2.7); 2.7327 (2.1);
2.7315 (2.2); 2.6766 (0.4); 2.6721 (0.6); 2.6675 (0.4); 2.5256 (1.7); 2.5209 (2.5); 2.5122 (37.2); 2.5077 (77.1); 2.5031 (101.0);
2.4985 (70.6); 2.4939 (32.8); 2.3345 (0.4); 2.3299 (0.6); 2.3253 (0.4); 1.2590 (0.4); 1.2384 (1.8); 0.8526 (0.5); −0.0002 (7.0)
Ib-062: ¹H-NMR(400.2 MHz, d₆-DMSO):
δ = 10.5355 (4.8); 8.6216 (5.4); 8.0182 (0.9); 8.0119 (7.7); 8.0069 (2.4); 7.9952 (2.6); 7.9902 (8.8); 7.9839 (1.0); 7.9531 (2.0);
7.9146 (0.8); 7.9090 (6.1); 7.9044 (2.0); 7.8917 (2.3); 7.8869 (8.1); 7.7415 (1.1); 7.7360 (7.8); 7.7313 (2.2); 7.7187 (2.0); 7.7140
(6.3); 7.7083 (0.7); 7.6472 (1.0); 7.6409 (9.0); 7.6359 (2.5); 7.6242 (2.4); 7.6192 (8.0); 7.6129 (0.8); 3.9295 (2.8); 3.8833 (3.6);
3.5740 (2.2); 3.5274 (1.8); 3.3313 (45.6); 3.1986 (0.4); 2.8914 (16.0); 2.7328 (13.7); 2.7314 (13.3); 2.6720 (0.4); 2.6294 (0.6);
2.5255 (1.2); 2.5207 (1.8); 2.5121 (25.1); 2.5076 (51.8); 2.5030 (67.4); 2.4983 (46.8); 2.4937 (21.5); 2.3298 (0.4); 1.2389
(1.2); −0.0002 (3.9)
Ib-063: ¹H-NMR(400.2 MHz, d₆-DMSO):
δ = 10.4870 (1.7); 8.6193 (2.4); 8.0721 (1.4); 8.0666 (0.6); 8.0584 (1.6); 8.0552 (0.8); 8.0528 (0.8); 8.0497 (1.6); 8.0415 (0.6);
8.0361 (1.5); 7.9534 (2.0); 7.9076 (2.3); 7.9030 (0.8); 7.8902 (0.9); 7.8855 (3.0); 7.8799 (0.4); 7.7333 (2.9); 7.7286 (0.8); 7.7159
(0.8); 7.7113 (2.4); 7.4109 (1.6); 7.4056 (0.5); 7.3938 (0.6); 7.3887 (3.0); 7.3835 (0.5); 7.3717 (0.5); 7.3665 (1.5); 3.9295 (1.0);
3.8833 (1.4); 3.5739 (0.9); 3.5272 (0.7); 3.3347 (32.2); 2.8919 (16.0); 2.7331 (13.7); 2.7317 (12.7); 2.6296 (0.4); 2.5258 (0.4);
2.5211 (0.7); 2.5124 (9.2); 2.5079 (18.7); 2.5034 (24.3); 2.4987 (16.9); 2.4942 (7.8); 1.2391 (0.4); −0.0002 (0.7)
Ib-064: ¹H-NMR(400.2 MHz, d₆-DMSO):
δ = 10.3109 (2.0); 8.6070 (2.4); 7.9869 (3.2); 7.9817 (1.0); 7.9698 (1.0); 7.9646 (3.5); 7.9528 (1.0); 7.9114 (2.6); 7.9068 (0.9);
7.8941 (0.9); 7.8893 (3.4); 7.7152 (3.2); 7.7106 (1.0); 7.6979 (0.9); 7.6932 (2.7); 7.0968 (0.3); 7.0895 (3.4); 7.0844 (1.0); 7.0723
(0.9); 7.0671 (3.2); 3.9244 (1.2); 3.8782 (1.5); 3.8480 (16.0); 3.5690 (1.0); 3.5225 (0.8); 3.3286 (24.8); 2.8910 (7.3); 2.7323
(6.2); 2.7310 (6.1); 2.5249 (0.7); 2.5202 (1.1); 2.5115 (15.0); 2.5070 (31.4); 2.5024 (41.3); 2.4977 (28.9); 2.4931 (13.4); 1.2392
(0.5); −0.0002 (3.6)
Ib-065: ¹H-NMR(400.2 MHz, d₆-DMSO):
δ = 9.9657 (6.9); 8.5812 (11.8); 7.7227 (7.1); 7.7181 (2.8); 7.7006 (14.5); 7.6556 (16.0); 7.6505 (4.0); 7.6384 (2.9); 7.6335 (8.4);
3.8919 (4.9); 3.8457 (6.3); 3.5363 (3.9); 3.4898 (3.1); 3.3298 (66.9); 3.2842 (0.6); 3.2633 (2.3); 3.2438 (3.6); 3.2422 (3.7);
3.2227 (2.6); 3.2017 (0.7); 2.8909 (0.6); 2.7324 (0.5); 2.7311 (0.6); 2.6760 (0.5); 2.6714 (0.7); 2.6669 (0.5); 2.5250 (1.8); 2.5203
(2.5); 2.5116 (39.1); 2.5071 (82.5); 2.5025 (108.9); 2.4979 (76.7); 2.4933 (35.8); 2.3339 (0.5); 2.3293 (0.7); 2.3247 (0.5); 2.2771
(0.8); 2.2720 (0.6); 2.2540 (2.6); 2.2487 (2.2); 2.2325 (3.3); 2.2303 (3.4); 2.2282 (3.4); 2.2251 (4.4); 2.2087 (2.7); 2.2038 (4.0);
2.1870 (1.0); 2.1814 (1.3); 2.1533 (1.1); 2.1465 (1.3); 2.1440 (1.4); 2.1316 (2.6); 2.1233 (3.5); 2.1166 (2.2); 2.1105 (2.3); 2.1015
(3.3); 2.0942 (1.9); 2.0868 (1.0); 2.0810 (1.0); 2.0777 (1.0); 2.0722 (0.9); 2.0026 (0.7); 1.9811 (1.3); 1.9762 (1.1); 1.9583 (2.1);
1.9544 (2.1); 1.9375 (1.2); 1.9318 (3.0); 1.9106 (1.6); 1.8879 (0.4); 1.8542 (0.5); 1.8451 (0.9); 1.8321 (1.0); 1.8217 (1.8); 1.8111
(1.2); 1.8082 (1.2); 1.7976 (1.4); 1.7951 (1.3); 1.7885 (0.8); 1.7858 (0.7); 1.7739 (0.5); 1.2589 (0.4); 1.2396 (1.4); 0.8532
(0.4); −0.0002 (11.1)
Ib-066: ¹H-NMR(400.1 MHz, d₆-DMSO):
δ = 10.0911 (9.4); 8.5682 (12.9); 7.9617 (2.9); 7.7277 (8.6); 7.7063 (16.0); 7.6617 (15.8); 7.6404 (8.4); 3.8973 (5.5); 3.8511 (7.0);
3.5436 (5.5); 3.4973 (4.3); 3.3124 (80.9); 2.8994 (15.3); 2.8387 (0.8); 2.8196 (2.5); 2.8007 (3.9); 2.7810 (3.1); 2.7615 (1.1);
2.7406 (14.5); 2.5089 (26.1); 1.9021 (1.4); 1.8745 (4.4); 1.8558 (5.4); 1.7702 (1.5); 1.7535 (3.8); 1.7355 (5.0); 1.7164 (5.6);
1.6979 (8.3); 1.6681 (3.5); 1.6108 (1.5); 1.5675 (5.2); 1.5549 (4.0)
Ib-067: ¹H-NMR(400.1 MHz, d₆-DMSO):
δ = 10.4685 (3.7); 8.6022 (4.5); 7.9869 (4.2); 7.9643 (5.5); 7.9317 (4.3); 7.9103 (5.2); 7.7384 (5.1); 7.7171 (4.2); 7.6415 (0.7);
7.6239 (2.1); 7.6055 (1.9); 7.5760 (3.4); 7.5568 (4.3); 7.5384 (1.6); 3.9347 (2.1); 3.8884 (2.7); 3.5818 (2.0); 3.5357 (1.6); 3.3124
(29.4); 2.8993 (16.0); 2.7406 (15.0); 2.5091 (9.6)
Ib-068: ¹H-NMR(400.2 MHz, d₆-DMSO):
δ = 10.0529 (6.8); 8.5826 (11.6); 7.7173 (7.9); 7.7124 (2.8); 7.7001 (3.7); 7.6950 (16.0); 7.6901 (2.4); 7.6535 (2.5); 7.6486 (15.2);
7.6435 (3.7); 7.6313 (2.8); 7.6264 (8.2); 7.6213 (1.0); 3.8879 (4.6); 3.8416 (6.0); 3.5335 (3.7); 3.4869 (3.0); 3.3330 (167.7);
2.8909 (0.5); 2.7325 (0.4); 2.7312 (0.4); 2.6762 (0.5); 2.6716 (0.7); 2.6670 (0.5); 2.5251 (2.1); 2.5204 (3.0); 2.5118 (42.6);
2.5072 (88.9); 2.5026 (116.8); 2.4980 (81.4); 2.4934 (37.5); 2.3781 (0.6); 2.3699 (1.1); 2.3623 (0.7); 2.3494 (1.3); 2.3411 (2.4);
2.3336 (1.8); 2.3298 (1.4); 2.3246 (0.9); 2.3208 (1.0); 2.3124 (1.4); 2.3040 (0.7); 1.8207 (2.7); 1.7846 (4.2); 1.7651 (2.1); 1.7479
(2.7); 1.7413 (3.2); 1.6679 (1.6); 1.6408 (1.4); 1.4486 (1.0); 1.4237 (2.5); 1.4179 (2.7); 1.3883 (2.9); 1.3634 (1.4); 1.3158 (1.1);
1.2852 (2.6); 1.2539 (3.2); 1.2397 (2.3); 1.2237 (2.9); 1.1978 (1.6); 1.1906 (1.0); 1.1670 (1.0); 0.8531 (0.5); −0.0002 (9.4)
Ib-069: ¹H-NMR(400.2 MHz, d₆-DMSO):
δ = 10.1368 (6.7); 8.5868 (11.1); 7.9522 (0.4); 7.7226 (7.1); 7.7177 (2.6); 7.7055 (3.7); 7.7003 (16.0); 7.6618 (15.2); 7.6567 (3.5);
7.6445 (2.6); 7.6396 (7.4); 3.9284 (3.0); 3.9232 (3.2); 3.9173 (2.8); 3.9046 (3.3); 3.8941 (7.9); 3.8474 (5.9); 3.5375 (3.7); 3.4909
(2.9); 3.3847 (2.6); 3.3775 (2.7); 3.3564 (5.8); 3.3488 (5.2); 3.3299 (56.2); 3.3211 (3.5); 2.8907 (3.3); 2.7322 (2.6); 2.7310 (2.8);
2.6759 (0.5); 2.6714 (0.6); 2.6667 (0.5); 2.6488 (0.6); 2.6372 (1.0); 2.6290 (0.8); 2.6216 (1.6); 2.6102 (2.4); 2.5983 (1.3); 2.5946
(1.3); 2.5831 (1.5); 2.5718 (0.8); 2.5249 (1.8); 2.5202 (2.6); 2.5115 (36.9); 2.5070 (76.5); 2.5024 (100.3); 2.4978 (69.7); 2.4931
(32.1); 2.3338 (0.4); 2.3292 (0.6); 2.3246 (0.4); 1.7276 (1.5); 1.7050 (5.7); 1.6960 (6.5); 1.6796 (3.6); 1.6688 (3.8); 1.6514 (1.7);
1.6464 (1.7); 1.6402 (2.1); 1.6182 (0.7); 1.6074 (0.6); 1.2588 (0.4); 1.2396 (1.6); 0.8531 (0.4); −0.0002 (9.7)
Ib-070: ¹H-NMR(400.2 MHz, d₆-DMSO):
δ = 10.5052 (2.7); 8.6188 (4.0); 8.5661 (5.2); 7.9532 (1.3); 7.9089 (0.4); 7.9032 (3.6); 7.8985 (1.2); 7.8859 (1.3); 7.8811 (4.7);
7.8754 (0.6); 7.7195 (0.6); 7.7139 (4.5); 7.7091 (1.3); 7.6966 (1.2); 7.6919 (3.8); 7.6862 (0.4); 3.9241 (1.6); 3.8777 (2.1); 3.5676
(1.3); 3.5210 (1.0); 3.3305 (23.3); 3.1990 (0.6); 2.8915 (10.3); 2.7329 (8.6); 2.7316 (8.6); 2.6295 (0.9); 2.5256 (0.8); 2.5209
(1.1); 2.5122 (14.7); 2.5077 (30.4); 2.5031 (39.9); 2.4985 (27.8); 2.4939 (12.7); 2.4461 (16.0); 1.2389 (0.6); −0.0002 (3.9)
Ib-071: ¹H-NMR(400.2 MHz, d₆-DMSO):
δ = 10.9657 (6.9); 8.8437 (13.4); 8.8390 (13.6); 8.6412 (5.5); 7.9529 (0.8); 7.9076 (11.5); 7.9031 (3.9); 7.8903 (4.4); 7.8855
(16.0); 7.7635 (2.2); 7.7580 (15.7); 7.7533 (4.5); 7.7407 (3.8); 7.7360 (12.3); 7.7303 (1.4); 7.3010 (13.0); 7.2963 (13.0); 6.5360
(0.4); 3.9333 (5.6); 3.8870 (7.2); 3.5770 (4.5); 3.5304 (3.6); 3.3320 (69.6); 3.1990 (0.6); 2.8914 (6.7); 2.7328 (5.2); 2.7317 (5.5);
2.6768 (0.5); 2.6722 (0.8); 2.6676 (0.6); 2.6296 (1.0); 2.5258 (2.3); 2.5211 (3.2); 2.5124 (47.0); 2.5079 (98.4); 2.5033 (129.7);
2.4987 (91.1); 2.4941 (42.5); 2.3347 (0.6); 2.3301 (0.8); 2.3255 (0.6); 1.2387 (1.5); 0.8528 (0.4); 0.0080 (0.3); −0.0002
(12.6); −0.0086 (0.4)
Ib-072: ¹H-NMR(400.2 MHz, d₆-DMSO):
δ = 10.0945 (4.8); 8.5844 (7.8); 7.9528 (1.9); 7.7077 (4.6); 7.7027 (1.8); 7.6907 (2.6); 7.6854 (11.6); 7.6541 (11.5); 7.6487 (2.6);
7.6369 (1.8); 7.6318 (5.0); 3.8913 (3.3); 3.8450 (4.3); 3.5364 (2.6); 3.4899 (2.1); 3.3296 (55.7); 3.3151 (0.3); 3.1986 (0.7);
2.8910 (16.0); 2.7324 (13.0); 2.7311 (13.2); 2.6761 (0.3); 2.6714 (0.5); 2.6668 (0.3); 2.6293 (1.1); 2.5250 (1.4); 2.5203 (2.0);
2.5116 (29.5); 2.5071 (61.5); 2.5025 (81.1); 2.4979 (56.6); 2.4933 (26.1); 2.4785 (0.5); 2.4736 (0.3); 2.3408 (5.3); 2.3342 (1.1);
2.3228 (9.6); 2.2690 (0.7); 2.2505 (1.4); 2.2321 (1.7); 2.2131 (1.3); 2.1942 (0.6); 1.7919 (0.7); 1.7887 (0.6); 1.7746 (1.4); 1.7630
(2.0); 1.7484 (1.8); 1.7453 (2.2); 1.7319 (1.6); 1.7249 (0.8); 1.7171 (0.9); 1.6442 (0.4); 1.6322 (0.7); 1.6254 (1.2); 1.6178 (1.5);
1.6047 (2.3); 1.5985 (1.5); 1.5937 (1.6); 1.5880 (2.0); 1.5797 (1.2); 1.5720 (0.8); 1.5664 (0.9); 1.5592 (0.7); 1.5542 (1.6); 1.5451
(0.6); 1.5403 (1.1); 1.5306 (1.9); 1.5212 (1.9); 1.5138 (2.1); 1.5010 (1.6); 1.4946 (1.3); 1.4840 (0.7); 1.2399 (1.5); 1.2253 (0.8);
1.2136 (0.7); 1.2073 (1.8); 1.1933 (1.6); 1.1880 (2.0); 1.1765 (1.8); 1.1730 (1.6); 1.1681 (1.3); 1.1578 (1.5); 1.1388 (0.6); 0.8533
(0.4); −0.0002 (5.9)
Ib-073: ¹H-NMR(400.2 MHz, d₆-DMSO):
δ = 10.4150 (2.0); 8.5882 (3.6); 7.9526 (2.0); 7.7155 (1.8); 7.7103 (0.7); 7.6985 (1.1); 7.6931 (5.2); 7.6682 (5.1); 7.6629 (1.1);
7.6511 (0.7); 7.6459 (1.9); 7.3514 (0.3); 7.3394 (9.2); 7.3305 (5.0); 7.3275 (5.2); 7.3195 (0.4); 7.3085 (0.4); 7.3057 (0.4); 7.2720
(0.7); 7.2642 (0.7); 7.2601 (0.7); 7.2570 (0.6); 7.2502 (1.0); 7.2445 (0.4); 7.2385 (0.4); 7.2362 (0.3); 3.8935 (1.4); 3.8472 (1.8);
3.6710 (7.3); 3.5351 (1.1); 3.4884 (0.9); 3.3304 (20.9); 2.8902 (16.0); 2.7319 (13.1); 2.7308 (12.7); 2.5246 (0.6); 2.5199 (0.9);
2.5112 (13.0); 2.5067 (26.7); 2.5021 (34.9); 2.4975 (24.2); 2.4929 (11.1); 1.2398 (0.5); −0.0002 (3.3)
Ib-074: ¹H-NMR(400.2 MHz, d₆-DMSO):
δ = 10.0125 (3.4); 8.6010 (3.7); 7.8030 (0.6); 7.7973 (4.8); 7.7926 (1.6); 7.7801 (1.9); 7.7752 (7.1); 7.7697 (0.9); 7.6829 (1.1);
7.6773 (7.0); 7.6724 (1.8); 7.6600 (1.6); 7.6553 (5.0); 7.6495 (0.5); 7.3485 (0.3); 6.5875 (0.3); 6.5658 (0.3); 4.0280 (16.0);
3.9023 (2.4); 3.8560 (3.0); 3.5559 (0.4); 3.5475 (1.9); 3.5010 (1.5); 3.3913 (0.4); 3.3813 (37.1); 3.3328 (39.2); 2.5251 (0.8);
2.5204 (1.2); 2.5117 (17.4); 2.5072 (35.9); 2.5026 (46.9); 2.4980 (32.6); 2.4934 (15.0); 1.2399 (0.6); −0.0002 (1.4)
Ib-075: ¹H-NMR(300.2 MHz, CDCl3):
δ = 7.7738 (1.0); 7.7449 (1.4); 7.6479 (0.5); 7.6283 (6.8); 7.6211 (3.3); 7.5991 (13.0); 7.5375 (12.8); 7.5080 (7.0); 7.4828 (0.5);
7.4764 (1.4); 7.2989 (9.4); 6.9372 (1.2); 6.7763 (0.3); 6.7479 (0.3); 6.6860 (1.4); 6.6574 (1.3); 6.4371 (0.6); 6.4051 (5.1); 5.3326
(2.1); 4.1966 (2.7); 4.1775 (0.5); 4.1364 (3.5); 4.1178 (0.6); 3.9738 (0.9); 3.7575 (5.7); 3.7312 (0.9); 3.6974 (4.4); 3.6712 (0.7);
3.5747 (0.8); 3.5525 (1.8); 3.5381 (3.1); 3.5167 (9.3); 3.5000 (16.0); 3.4782 (7.5); 3.4567 (4.1); 3.4357 (3.2); 3.4178 (4.0);
3.3948 (2.1); 3.3883 (2.2); 3.3656 (3.7); 3.3445 (2.0); 3.3290 (1.4); 3.3072 (0.6); 2.0370 (2.8); 2.0281 (6.1); 2.0177 (7.4); 2.0061
(14.7); 1.9965 (6.8); 1.9837 (5.6); 1.9604 (2.8); 1.9378 (5.2); 1.9254 (5.8); 1.9155 (7.7); 1.9044 (6.1); 1.8928 (5.0); 1.8700 (2.3);
1.8493 (0.7); 1.8394 (0.5); 1.7626 (1.4); 0.0446 (0.4); 0.0337 (10.5); 0.0231 (0.4)
Ib-076: ¹H-NMR(499.9 MHz, d₆-DMSO):
δ = 8.5589 (9.4); 8.5261 (16.0); 7.5830 (10.7); 7.5654 (15.5); 7.4806 (15.0); 7.4630 (11.0); 6.2746 (4.8); 6.2602 (4.9); 3.9783
(0.5); 3.9649 (1.9); 3.9516 (3.6); 3.9382 (3.7); 3.9249 (1.9); 3.9115 (0.5); 3.8685 (5.6); 3.8317 (6.9); 3.5171 (4.5); 3.4801 (3.8);
3.3218 (22.8); 2.5150 (6.0); 2.5115 (12.1); 2.5079 (16.5); 2.5043 (12.1); 2.5008 (5.9); 1.8795 (1.0); 1.8767 (1.1); 1.8656 (2.5);
1.8635 (2.6); 1.8527 (3.7); 1.8390 (3.8); 1.8274 (3.0); 1.8152 (1.4); 1.8134 (1.4); 1.6852 (0.5); 1.6738 (0.7); 1.6650 (1.5); 1.6594
(2.3); 1.6508 (3.6); 1.6427 (4.1); 1.6290 (3.1); 1.6068 (1.3); 1.5924 (0.4); 1.5830 (1.3); 1.5760 (1.0); 1.5732 (0.9); 1.5604 (3.0);
1.5518 (3.2); 1.5464 (4.2); 1.5414 (2.8); 1.5380 (3.9); 1.5307 (2.2); 1.5238 (1.5); 1.5159 (0.7); 1.5121 (0.7); 1.5088 (0.5); 1.5030
(0.5); 1.4084 (1.5); 1.3962 (2.9); 1.3938 (3.0); 1.3814 (3.7); 1.3704 (3.6); 1.3616 (1.8); 1.3576 (2.4); 1.3537 (2.2); 1.3451 (0.9);
1.3413 (1.0); 1.2602 (0.5)
Ib-077: ¹H-NMR(400.2 MHz, d₆-DMSO):
δ = 9.9536 (1.8); 8.5794 (1.5); 7.9519 (0.4); 7.6531 (2.6); 7.6484 (0.9); 7.6361 (1.2); 7.6311 (4.4); 7.6256 (0.6); 7.5624 (3.6);
7.5404 (2.2); 6.5322 (0.3); 3.8836 (1.4); 3.8373 (1.8); 3.6871 (16.0); 3.5321 (1.1); 3.4856 (0.9); 3.3276 (142.4); 3.1987 (0.6);
2.8910 (2.8); 2.7319 (2.2); 2.7309 (2.2); 2.6756 (0.6); 2.6710 (0.9); 2.6665 (0.6); 2.6288 (0.9); 2.5245 (2.6); 2.5198 (3.7); 2.5111
(52.3); 2.5066 (108.8); 2.5021 (143.0); 2.4974 (100.1); 2.4929 (46.4); 2.3334 (0.6); 2.3288 (0.9); 2.3243 (0.6); 1.2587 (0.4);
1.2402 (1.6); 0.8535 (0.4); −0.0002 (4.3)
Ib-078: ¹H-NMR(300.2 MHz, CDCl3):
δ = 7.6845 (1.2); 7.6782 (0.4); 7.6618 (0.5); 7.6553 (1.7); 7.5255 (1.3); 7.4962 (0.9); 7.2990 (11.9); 6.7777 (0.5); 5.3386 (0.4);
4.0617 (0.3); 4.0001 (0.6); 3.9944 (0.4); 3.8876 (1.0); 3.8597 (7.4); 3.8407 (6.8); 3.8263 (0.6); 1.5941 (16.0); 0.1065 (0.9);
0.0480 (0.4); 0.0372 (11.8); 0.0263 (0.4)
Ib-079: ¹H-NMR(400.2 MHz, d₆-DMSO):
δ = 10.2115 (3.2); 8.5912 (4.8); 7.9528 (2.0); 7.6829 (0.6); 7.6774 (4.4); 7.6726 (1.5); 7.6603 (1.9); 7.6553 (7.6); 7.6499 (1.0);
7.5860 (6.3); 7.5638 (4.0); 7.4955 (0.4); 7.4883 (2.7); 7.4837 (3.5); 7.4793 (1.6); 7.4771 (1.8); 7.4706 (3.6); 7.4643 (5.5); 7.4513
(0.4); 7.4467 (0.5); 7.4343 (1.2); 7.4311 (1.7); 7.4231 (3.8); 7.4187 (7.7); 7.4143 (3.0); 7.4114 (2.9); 7.4056 (2.1); 7.4033 (2.5);
7.4010 (3.2); 7.3945 (0.6); 7.3918 (0.6); 7.3880 (0.9); 7.3838 (0.4); 7.3790 (0.6); 7.3691 (0.4); 7.3622 (0.5); 5.0536 (12.5);
3.8939 (2.3); 3.8476 (3.0); 3.5408 (1.8); 3.4942 (1.5); 3.3394 (76.4); 3.3384 (76.5); 3.1986 (0.9); 2.8911 (16.0); 2.7326 (12.8);
2.7315 (13.0); 2.6722 (0.4); 2.6295 (1.4); 2.5258 (1.3); 2.5211 (1.9); 2.5123 (27.0); 2.5078 (56.4); 2.5033 (74.0); 2.4986 (51.1);
2.4940 (23.3); 2.3301 (0.4); 2.3254 (0.3); 1.2394 (1.3); 0.8532 (0.3); −0.0002 (5.1)
Ib-080: ¹H-NMR(400.2 MHz, d₆-DMSO):
δ = 9.9225 (1.7); 8.5731 (3.0); 7.9737 (0.6); 7.7174 (3.0); 7.6491 (2.4); 7.6443 (0.8); 7.6321 (1.1); 7.6270 (4.5); 7.6219 (1.0);
7.6156 (3.2); 7.6108 (0.9); 7.5989 (1.0); 7.5940 (3.4); 7.5875 (0.4); 7.5749 (3.2); 7.5528 (1.8); 6.6614 (0.4); 6.6550 (3.5); 6.6500
(0.9); 6.6382 (0.9); 6.6332 (3.4); 6.6267 (0.3); 5.7272 (2.5); 3.9001 (4.7); 3.8835 (6.0); 3.8375 (1.7); 3.5331 (1.0); 3.4864 (0.8);
3.3281 (56.5); 2.8913 (0.4); 2.7326 (0.4); 2.7313 (0.4); 2.6715 (0.4); 2.6295 (0.5); 2.5251 (1.3); 2.5204 (1.8); 2.5118 (26.5);
2.5072 (55.1); 2.5027 (72.3); 2.4980 (50.2); 2.4934 (23.0); 2.3295 (0.4); 1.9651 (0.4); 1.9484 (0.8); 1.9316 (1.1); 1.9149 (0.9);
1.8983 (0.4); 1.2393 (1.1); 0.9480 (16.0); 0.9312 (15.5); −0.0002 (6.5)
Ib-081: ¹H-NMR(400.2 MHz, d₆-DMSO):
δ = 7.7166 (0.6); 7.6151 (0.6); 7.5935 (0.7); 7.3746 (0.6); 7.3685 (5.8); 7.3639 (1.7); 7.3514 (1.8); 7.3468 (6.3); 7.3405 (0.7);
6.6544 (0.8); 6.6327 (0.7); 6.6059 (0.7); 6.5996 (6.6); 6.5948 (1.8); 6.5826 (1.8); 6.5778 (6.3); 6.5714 (0.6); 5.7665 (5.6); 5.7264
(0.6); 4.2613 (1.2); 4.2129 (2.0); 4.1948 (0.7); 4.1863 (0.9); 4.1755 (1.2); 4.1684 (3.2); 4.1576 (3.4); 4.1506 (3.5); 4.1399 (3.2);
4.1325 (1.2); 4.1222 (0.9); 4.1135 (0.7); 4.0229 (2.8); 3.9751 (2.0); 3.3274 (36.9); 2.8905 (0.4); 2.7320 (0.4); 2.7308 (0.4);
2.5243 (0.9); 2.5196 (1.2); 2.5109 (19.4); 2.5064 (40.2); 2.5018 (52.6); 2.4972 (36.5); 2.4926 (16.9); 1.2339 (7.7); 1.2161 (16.0);
1.1983 (7.2); −0.0002 (5.3)
Ib-082: ¹H-NMR(400.2 MHz, d₆-DMSO):
δ = 10.1178 (2.1); 8.5823 (3.7); 7.9528 (2.0); 7.6615 (2.5); 7.6569 (0.9); 7.6444 (1.1); 7.6395 (4.5); 7.5866 (2.9); 7.5840 (3.3);
7.5757 (3.3); 7.5651 (6.2); 7.5536 (2.1); 7.4980 (2.7); 7.4801 (2.0); 7.4758 (1.7); 7.4578 (1.3); 5.3821 (8.0); 3.8889 (1.4); 3.8426
(1.8); 3.5362 (1.1); 3.4898 (0.9); 3.3290 (37.1); 3.1986 (0.5); 2.8914 (16.0); 2.7326 (13.3); 2.7314 (12.9); 2.6293 (0.9); 2.5251
(0.8); 2.5204 (1.1); 2.5117 (16.2); 2.5072 (34.2); 2.5026 (45.4); 2.4979 (32.1); 2.4933 (15.1); 1.2397 (0.8); −0.0002 (3.4)
Ib-083: ¹H-NMR(400.2 MHz, d₆-DMSO):
δ = 9.9190 (6.0); 8.5811 (3.7); 7.6457 (8.2); 7.6409 (3.0); 7.6286 (4.1); 7.6236 (16.0); 7.5740 (11.6); 7.5519 (6.4); 4.0105 (0.5);
3.9974 (14.4); 3.9795 (14.6); 3.8815 (4.7); 3.8478 (0.4); 3.8353 (6.1); 3.5326 (3.8); 3.4861 (3.1); 3.3291 (94.6); 3.2914 (0.6);
3.1986 (0.7); 2.8909 (1.6); 2.7323 (1.2); 2.7311 (1.3); 2.6805 (0.3); 2.6760 (0.7); 2.6714 (0.9); 2.6668 (0.7); 2.6291 (1.1); 2.5249
(2.8); 2.5203 (4.1); 2.5115 (57.0); 2.5070 (118.7); 2.5025 (156.5); 2.4978 (109.6); 2.4932 (50.7); 2.3385 (0.3); 2.3338 (0.7);
2.3292 (1.0); 2.3247 (0.7); 2.3200 (0.4); 2.2531 (1.1); 2.2344 (2.6); 2.2158 (3.5); 2.1971 (2.7); 2.1784 (1.2); 1.7679 (1.0); 1.7515
(2.0); 1.7394 (2.6); 1.7325 (2.1); 1.7198 (3.4); 1.7103 (1.9); 1.7074 (2.0); 1.7004 (1.5); 1.6965 (1.2); 1.6907 (1.3); 1.6403 (0.5);
1.6210 (1.9); 1.6135 (1.8); 1.6007 (3.6); 1.5890 (2.7); 1.5845 (3.4); 1.5716 (1.8); 1.5654 (3.3); 1.5578 (2.0); 1.5538 (1.7); 1.5447
(4.0); 1.5362 (2.7); 1.5335 (2.7); 1.5260 (3.2); 1.5193 (2.4); 1.5145 (2.0); 1.5053 (1.9); 1.4972 (1.0); 1.4859 (0.5); 1.4776 (0.4);
1.3214 (1.2); 1.3052 (2.7); 1.2980 (1.6); 1.2884 (3.2); 1.2743 (3.0); 1.2575 (2.6); 1.2398 (3.4); 0.8532 (0.7); 0.0080 (0.4); −0.0002
(12.7); −0.0085 (0.4)
Ib-084: ¹H-NMR(400.2 MHz, d₆-DMSO):
δ = 10.0769 (2.7); 8.5840 (0.9); 7.9522 (2.0); 7.6559 (3.7); 7.6512 (1.4); 7.6388 (1.7); 7.6338 (6.8); 7.5762 (5.4); 7.5540 (3.2);
7.4544 (1.4); 7.4497 (2.1); 7.4444 (0.8); 7.4333 (4.7); 7.4297 (4.4); 7.4236 (2.9); 7.4205 (3.3); 7.4154 (0.9); 7.4034 (5.5); 7.3989
(1.8); 7.3887 (1.3); 7.3842 (2.2); 7.3798 (0.5); 7.3722 (1.3); 7.3678 (2.2); 7.3630 (1.0); 7.3579 (0.8); 7.3504 (1.9); 7.3426 (0.5);
7.3364 (1.0); 7.3333 (0.7); 6.5476 (0.6); 5.1708 (10.7); 3.8845 (2.0); 3.8382 (2.6); 3.5338 (1.7); 3.4873 (1.3); 3.3272 (65.5);
3.1982 (0.9); 2.8905 (16.0); 2.7319 (12.9); 2.7306 (13.3); 2.6754 (0.6); 2.6708 (0.8); 2.6662 (0.6); 2.6287 (1.4); 2.5244 (2.4);
2.5197 (3.4); 2.5110 (48.1); 2.5065 (100.1); 2.5019 (131.5); 2.4972 (92.1); 2.4927 (42.7); 2.3333 (0.6); 2.3287 (0.8); 2.3240
(0.6); 1.2586 (0.4); 1.2400 (1.7); 0.8534 (0.4); −0.0002 (9.0)
Ib-085: ¹H-NMR(400.1 MHz, d₆-DMSO):
δ = 9.5288 (1.0); 8.5806 (2.9); 7.9616 (2.4); 7.6678 (1.0); 7.6442 (16.0); 7.6210 (0.8); 4.5137 (0.6); 3.9054 (2.0); 3.8591 (2.6);
3.5537 (2.0); 3.5070 (1.6); 3.3109 (31.5); 2.8996 (8.0); 2.7408 (7.6); 2.5091 (11.1); 1.9775 (1.2); 1.9634 (1.7); 1.9480 (1.8);
1.9331 (1.4); 1.9166 (0.6); 1.6701 (2.0); 1.6537 (1.6); 1.6303 (0.6); 1.6061 (0.7); 1.5818 (1.5); 1.5645 (1.9); 1.5530 (1.6); 1.5185
(1.2); 1.5028 (1.4); 1.4884 (1.6); 1.4734 (1.6); 1.4588 (1.0)
Ib-086: ¹H-NMR(400.1 MHz, d₆-DMSO):
δ = 10.0066 (1.8); 9.9624 (1.8); 8.6084 (3.1); 7.9616 (3.0); 7.6924 (1.8); 7.6695 (12.4); 7.6641 (12.3); 7.6412 (1.8); 7.5060 (4.5);
7.4858 (6.0); 7.3744 (3.2); 7.3553 (5.6); 7.3359 (3.0); 7.1727 (1.7); 7.1547 (2.8); 7.1358 (1.2); 3.9228 (2.6); 3.8763 (3.2); 3.5694
(2.5); 3.5228 (2.0); 3.3124 (51.5); 2.8995 (16.0); 2.7407 (15.1); 2.5092 (15.6)
Ib-087: ¹H-NMR(400.1 MHz, d₆-DMSO):
δ = 9.8081 (2.1); 8.5288 (2.4); 7.9475 (2.2); 7.9261 (2.5); 7.6274 (2.5); 7.6061 (2.2); 6.7036 (2.8); 3.8821 (1.0); 3.8361 (1.4);
3.5488 (1.0); 3.5024 (0.8); 3.3106 (6.4); 2.8988 (0.4); 2.7405 (0.4); 2.5089 (4.2); 2.3512 (16.0)
Ib-088: ¹H-NMR(300.2 MHz, d₆-DMSO):
δ = 8.5701 (0.4); 7.9416 (0.3); 7.9121 (0.4); 7.6570 (0.4); 7.6279 (0.3); 3.3464 (16.0); 2.5347 (1.6); 2.5288 (3.4); 2.5228 (4.8);
2.5167 (3.5); 2.5109 (1.6); 2.4100 (1.3); 0.0211 (5.1)
Ib-089: ¹H-NMR(300.2 MHz, d₆-DMSO):
δ = 9.9959 (0.8); 8.5811 (0.7); 8.5711 (1.8); 8.5550 (1.8); 7.9254 (0.9); 7.8959 (1.1); 7.6710 (1.1); 7.6417 (0.9); 6.9568 (0.5);
6.9408 (0.8); 6.9248 (0.4); 3.9193 (0.4); 3.8576 (0.5); 3.5764 (0.4); 3.3470 (16.0); 2.5340 (1.6); 2.5283 (3.4); 2.5223 (4.7);
2.5163 (3.4); 2.5106 (1.6); 2.0096 (0.9); 1.1949 (0.5); 0.0203 (3.7)
Ib-090: ¹H-NMR(400.1 MHz, d₆-DMSO):
δ = 10.6730 (2.2); 8.5498 (6.4); 7.9615 (3.1); 7.8206 (4.6); 7.8008 (5.2); 7.6499 (0.8); 7.6326 (2.4); 7.6157 (2.4); 7.5947 (6.0);
7.5896 (5.8); 7.5728 (8.3); 7.5506 (1.7); 7.1982 (5.4); 7.1774 (4.9); 3.8443 (2.4); 3.7978 (3.0); 3.4805 (2.3); 3.4341 (1.8); 3.3126
(41.2); 2.8991 (16.0); 2.7404 (15.2); 2.5088 (13.2)
Ib-092: ¹H-NMR(400.2 MHz, d₆-DMSO):
δ = 10.0780 (2.3); 8.6561 (10.7); 7.7492 (9.8); 7.7322 (4.1); 7.7273 (10.9); 7.6475 (0.3); 7.5625 (2.5); 7.5407 (2.8); 7.2280 (11.1);
7.2108 (3.9); 7.2061 (10.2); 7.1153 (0.3); 6.8535 (2.8); 6.8316 (2.7); 5.1778 (0.6); 4.1574 (0.7); 4.1101 (1.0); 4.0276 (1.5);
4.0125 (3.1); 3.9964 (4.0); 3.9797 (3.1); 3.9586 (5.2); 3.9333 (0.4); 3.9118 (6.6); 3.8638 (1.0); 3.7490 (0.3); 3.7332 (0.4); 3.5782
(3.7); 3.5315 (3.0); 3.3344 (100.2); 2.8910 (1.1); 2.7315 (1.0); 2.6716 (0.4); 2.5071 (45.3); 2.5027 (59.0); 2.4983 (45.3); 2.3297
(0.3); 1.2403 (16.0); 1.1265 (3.8); 1.1105 (3.6); 0.8531 (0.4); −0.0002 (1.2)
Ib-093: ¹H-NMR(400.2 MHz, d₆-DMSO):
δ = 8.6468 (7.1); 7.9531 (2.7); 7.7238 (4.6); 7.7024 (5.1); 7.5311 (5.3); 7.5096 (7.8); 7.4131 (5.1); 7.3918 (3.7); 7.1970 (2.3);
7.1782 (2.2); 4.5328 (0.4); 4.5160 (1.0); 4.4990 (1.3); 4.4828 (1.0); 4.4654 (0.4); 3.9460 (2.5); 3.8994 (3.2); 3.5618 (2.3); 3.5151
(1.8); 3.3390 (72.5); 2.8909 (16.0); 2.7318 (15.5); 2.5077 (26.5); 2.5034 (34.9); 2.4991 (26.3); 1.2388 (1.4); 1.1369 (15.8);
1.1200 (15.5); 1.0893 (0.5); −0.0002 (0.6)
Ib-094: ¹H-NMR(400.2 MHz, d₆-DMSO):
δ = 8.6459 (7.3); 7.9533 (1.0); 7.7175 (4.3); 7.6961 (4.6); 7.4812 (1.8); 7.4634 (5.4); 7.4442 (4.6); 7.4054 (2.4); 7.3877 (3.0);
7.3814 (1.1); 7.3616 (4.6); 7.3431 (3.4); 7.1806 (2.0); 7.1620 (1.9); 4.5371 (0.4); 4.5203 (1.0); 4.5038 (1.3); 4.4868 (1.0); 4.4703
(0.4); 3.9436 (2.5); 3.8970 (3.2); 3.5593 (2.2); 3.5125 (1.8); 3.3397 (67.5); 2.8908 (6.4); 2.7323 (6.1); 2.5073 (28.7); 2.5031
(35.3); 2.4990 (25.4); 1.2404 (1.1); 1.1405 (16.0); 1.1237 (15.7); −0.0002 (0.6)
Ib-095: ¹H-NMR(300.2 MHz, CDCl3):
δ = 7.7116 (0.8); 7.7028 (6.5); 7.6962 (2.2); 7.6804 (2.2); 7.6735 (7.5); 7.6649 (1.0); 7.6444 (0.3); 7.6393 (0.4); 7.2986 (33.1);
7.2592 (0.9); 7.2504 (7.5); 7.2437 (2.4); 7.2278 (2.1); 7.2210 (6.6); 7.2125 (0.8); 7.1751 (0.4); 7.1452 (0.3); 3.8189 (2.6); 3.7593
(4.2); 3.7180 (1.0); 3.5662 (2.7); 3.5065 (1.7); 3.5017 (1.2); 3.1525 (16.0); 3.0629 (15.7); 2.6306 (0.4); 2.2966 (0.4); 2.0839
(0.3); 1.6000 (3.5); 1.3252 (0.4); 1.2975 (0.6); 0.9208 (0.4); 0.0494 (1.5); 0.0386 (43.7); 0.0277 (1.6)
Ib-096: ¹H-NMR(400.2 MHz, d₆-DMSO):
δ = 8.6558 (7.3); 7.9527 (2.6); 7.7512 (5.5); 7.7297 (6.2); 7.4903 (3.5); 7.4712 (7.4); 7.4503 (5.6); 7.4319 (6.7); 7.4113 (3.0);
7.3080 (2.0); 7.2900 (3.8); 7.2753 (3.8); 7.2580 (2.9); 3.9573 (3.3); 3.9108 (4.2); 3.5758 (2.9); 3.5292 (2.4); 3.3518 (6.5); 3.3364
(51.4); 2.8901 (16.0); 2.7321 (14.7); 2.5068 (27.2); 2.5024 (35.3); 2.4982 (26.3); 1.2398 (1.4); −0.0002 (0.6)
Ib-097: ¹H-NMR(400.2 MHz, d₆-DMSO):
δ = 8.6497 (14.1); 7.7442 (14.4); 7.7227 (16.0); 7.2421 (12.2); 7.2207 (11.4); 3.9582 (6.7); 3.9117 (8.6); 3.8699 (0.8); 3.8420
(1.3) ; 3.8139 (0.7); 3.5775 (5.7); 3.5307 (4.6); 3.3324 (81.9); 2.9187 (10.2); 2.8903 (2.5); 2.8639 (0.6); 2.8146 (5.8); 2.7312
(1.6); 2.6710 (0.4); 2.5061 (48.9); 2.5019 (62.9); 2.4975 (46.7); 2.3289 (0.4); 1.7964 (4.9); 1.7660 (6.6); 1.6641 (2.0); 1.6273
(3.4) ; 1.5868 (3.1); 1.5490 (2.3); 1.5212 (3.5); 1.4935 (2.7); 1.3148 (3.2); 1.2841 (2.6); 1.2580 (1.3); 1.2392 (2.4); 1.1485 (1.2);
1.1164 (2.7); 1.0922 (1.4); 1.0843 (2.2); 1.0764 (1.3); 1.0613 (0.5); 1.0519 (0.6); 0.8528 (0.4); −0.0002 (1.2)
Ib-098: ¹H-NMR(400.2 MHz, d₆-DMSO):
δ = 8.6531 (16.0); 7.9520 (0.4); 7.7482 (11.0); 7.7266 (12.2); 7.2459 (10.5); 7.2244 (9.7); 3.9582 (5.3); 3.9117 (6.7); 3.5787 (4.6);
3.5320 (3.6); 3.4551 (0.9); 3.4386 (2.5); 3.4211 (2.6); 3.4036 (1.1); 3.3342 (75.7); 3.3102 (3.1); 3.2928 (1.1); 3.0308 (11.0);
2.9067 (10.8); 2.8903 (3.0); 2.7312 (2.4); 2.5062 (32.0); 2.5021 (41.2); 2.4978 (30.7); 1.2389 (1.6); 1.2068 (2.8); 1.1892 (5.6);
1.1717 (3.0); 1.1262 (3.2); 1.1087 (5.8); 1.0910 (2.9); −0.0002 (0.7)
Ib-099: ¹H-NMR(400.2 MHz, d₆-DMSO):
δ = 8.6496 (6.2); 7.9524 (0.4); 7.7338 (2.5); 7.7131 (2.7); 7.3113 (2.4); 7.2910 (4.3); 7.2483 (6.0); 7.2279 (4.0); 3.9515 (2.0);
3.9049 (2.6); 3.7095 (1.2); 3.5682 (1.8); 3.5215 (1.5); 3.3380 (73.1); 2.8901 (2.3); 2.7311 (2.2); 2.5067 (24.0); 2.5024 (30.8);
2.4981 (22.8); 2.3401 (0.6); 2.3192 (16.0); 1.2392 (1.0); 1.1234 (2.3); −0.0002 (0.5)
Ib-100: ¹H-NMR(400.2 MHz, d₆-DMSO):
δ = 8.6520 (8.4); 7.9526 (1.8); 7.7414 (4.0); 7.7201 (4.4); 7.4716 (0.8); 7.4510 (6.4); 7.4436 (6.4); 7.4370 (16.0); 7.3471 (1.0);
7.3405 (1.5); 7.3321 (1.6); 7.3262 (1.7); 7.3116 (1.0); 7.3046 (0.7); 7.2483 (2.0); 7.2333 (1.9); 7.0608 (1.5); 7.0414 (3.3); 7.0222
(2.3); 6.9315 (1.1); 6.9129 (1.7); 6.8948 (0.7); 6.7706 (3.3); 6.7509 (2.9); 3.9540 (2.7); 3.9074 (3.4); 3.7581 (1.8); 3.7423 (1.8);
3.5701 (2.6); 3.5479 (3.3); 3.5302 (3.9); 3.5131 (1.3); 3.3358 (92.4); 2.8901 (10.4); 2.7312 (9.9); 2.5025 (44.0); 2.4983 (33.0);
1.2392 (1.4); 1.1444 (3.7); 1.0195 (3.4); 1.0021 (7.1); 0.9845 (3.3); −0.0002 (0.7)
Ib-101: ¹H-NMR(400.2 MHz, d₆-DMSO):
δ = 10.1068 (0.4); 8.6555 (15.5); 7.9526 (2.2); 7.8284 (0.7); 7.7979 (0.7); 7.7763 (0.8); 7.7499 (14.5); 7.7282 (16.0); 7.2471
(16.0); 7.2254 (14.8); 6.9179 (0.7); 6.8962 (0.7); 3.9598 (6.4); 3.9132 (8.1); 3.5793 (5.7); 3.5325 (4.6); 3.4169 (5.0); 3.4000
(5.3); 3.3838 (2.7); 3.3358 (109.8); 3.3174 (6.5); 3.2996 (5.6); 2.8907 (13.0); 2.7316 (12.1); 2.6713 (0.4); 2.5067 (47.4); 2.5025
(59.7) ; 2.4982 (43.7); 2.3292 (0.4); 1.2387 (2.8); 1.2200 (5.8); 1.2031 (10.9); 1.1861 (6.5); 1.1392 (6.8); 1.1222 (11.0); 1.1052
(5.7) ; 0.8529 (0.5); −0.0002 (1.0)
Ib-102: ¹H-NMR(400.2 MHz, d₆-DMSO):
δ = 8.6604 (2.0); 7.9526 (2.4); 7.7976 (0.4); 7.7784 (0.5); 7.7336 (2.5); 7.7121 (2.6); 7.3432 (1.4); 7.3281 (2.9); 7.3104 (4.3);
7.2935 (1.6); 7.2837 (1.2); 7.2602 (3.0); 7.2485 (2.1); 7.2434 (2.4); 7.2306 (2.3); 7.2260 (2.3); 7.2181 (1.3); 7.2135 (2.0); 7.2079
(1.7); 7.1990 (1.9); 7.1946 (2.1); 7.1863 (3.1); 7.1649 (2.8); 7.1501 (1.6); 7.1320 (0.9); 4.9378 (1.0); 4.9010 (1.3); 4.6897 (1.2);
4.6528 (1.0); 3.9503 (1.2); 3.9037 (1.4); 3.5636 (1.1); 3.5167 (0.9); 3.3311 (10.1); 2.8893 (16.0); 2.7308 (13.9); 2.6287 (0.4);
2.5241 (0.8); 2.5106 (15.5); 2.5063 (30.4); 2.5019 (39.1); 2.4974 (28.2); 2.4932 (13.8); 2.1140 (7.7); 2.0649 (1.3); 1.2392
(0.9); −0.0002 (3.1)
Ib-103: ¹H-NMR(400.2 MHz, d₆-DMSO):
δ = 8.6548 (16.0); 7.9523 (2.0); 7.7474 (13.8); 7.7259 (15.5); 7.5311 (0.4); 7.2246 (15.3); 7.2030 (14.4); 3.9568 (6.3); 3.9103
(8.0); 3.5762 (5.4); 3.5294 (4.4); 3.3331 (127.9); 3.3097 (4.9); 3.2400 (4.2); 3.2222 (6.5); 3.2036 (4.2); 3.1663 (0.3); 2.8904
(11.8); 2.7313 (11.2); 2.7108 (0.3); 2.6710 (0.4); 2.5060 (57.1); 2.5021 (72.2); 2.4978 (53.4); 2.3288 (0.4); 1.6807 (0.6); 1.6626
(2.4); 1.6443 (4.6); 1.6263 (4.9); 1.6082 (3.6); 1.5923 (3.6); 1.5741 (5.0); 1.5560 (5.0); 1.5373 (2.8); 1.5193 (0.9); 1.2684 (0.4);
1.2391 (2.5); 0.9231 (7.1); 0.9049 (14.3); 0.8862 (14.6); 0.8676 (14.5); 0.8492 (7.0); 0.8132 (0.4); 0.7948 (0.6); −0.0003 (1.2)
Ib-104: ¹H-NMR(400.2 MHz, d₆-DMSO):
δ = 8.6521 (5.0); 7.9524 (2.6); 7.7480 (4.0); 7.7435 (1.5); 7.7264 (4.4); 7.2582 (4.3); 7.2365 (4.0); 3.9566 (1.7); 3.9100 (2.2);
3.5786 (1.5); 3.5311 (1.5); 3.5256 (2.0); 3.5087 (3.4); 3.4923 (1.9); 3.3608 (2.0); 3.3440 (4.4); 3.3344 (29.1); 2.8906 (16.0);
2.7316 (14.9); 2.5066 (12.4); 2.5022 (15.9); 2.4978 (11.6); 1.9259 (1.0); 1.9096 (2.0); 1.8926 (2.2); 1.8796 (2.2); 1.8628 (2.0);
1.8462 (1.0); 1.2398 (0.4)
Ib-105: ¹H-NMR(400.2 MHz, d₆-DMSO):
δ = 8.6538 (16.0); 7.9523 (0.5); 7.7453 (13.0); 7.7408 (4.8); 7.7237 (14.5); 7.2448 (14.4); 7.2403 (5.0); 7.2231 (13.5); 3.9574
(5.7); 3.9109 (7.2); 3.5772 (7.0); 3.5590 (4.5); 3.5309 (5.1); 3.4786 (0.5); 3.4099 (4.3); 3.3380 (126.1); 2.8903 (3.2); 2.7315
(2.9); 2.5066 (37.4); 2.5023 (47.8); 2.4978 (34.6); 1.6087 (4.4); 1.5884 (7.8); 1.5577 (6.6); 1.2393 (1.0); −0.0002 (0.6)
Ib-106: ¹H-NMR(400.2 MHz, d₆-DMSO):
δ = 8.6580 (13.4); 7.7593 (12.3); 7.7548 (4.8); 7.7376 (13.8); 7.2759 (13.4); 7.2542 (12.6); 3.9601 (5.2); 3.9136 (6.7); 3.6650
(9.6); 3.6542 (16.0); 3.6414 (14.5); 3.5811 (7.4); 3.5342 (4.2); 3.4282 (4.0); 3.3371 (74.4); 2.8901 (0.6); 2.7313 (0.6); 2.5065
(31.8); 2.5022 (41.0); 2.4977 (30.2); 1.2393 (1.2); −0.0002 (0.7)
Ib-107: ¹H-NMR(400.2 MHz, d₆-DMSO):
δ = 8.6463 (3.4); 7.9536 (2.6); 7.7991 (0.9); 7.7797 (1.0); 7.7268 (3.9); 7.7219 (1.5); 7.7096 (1.6); 7.7049 (4.3); 7.6578 (0.8);
7.6385 (1.1); 7.5534 (0.4); 7.5365 (1.4); 7.5329 (1.7); 7.5148 (6.3); 7.4963 (6.7); 7.4252 (2.1); 7.4074 (2.8); 7.3879 (1.1); 7.2082
(4.3); 7.2034 (1.5); 7.1863 (4.0); 7.1600 (0.6); 7.1552 (0.6); 7.1300 (3.8); 7.1228 (3.7); 3.9409 (1.4); 3.8942 (1.8); 3.5533 (1.2);
3.5066 (1.0); 3.3433 (39.5); 2.8891 (16.0); 2.7315 (15.0); 2.5114 (9.1); 2.5073 (17.6); 2.5029 (22.7); 2.4984 (16.6); 2.4941 (8.3);
1.2385 (0.7); −0.0002 (0.4)
Ib-108: ¹H-NMR(400.2 MHz, d₆-DMSO):
δ = 7.9526 (2.2); 7.7567 (2.8); 7.7521 (1.0); 7.7398 (1.0); 7.7349 (3.2); 7.7285 (0.4); 7.6195 (0.3); 7.5095 (0.4); 7.3137 (3.8);
7.3090 (1.6); 7.2964 (1.6); 7.2918 (3.8); 7.2845 (1.4); 7.2684 (2.7); 3.9558 (1.1); 3.9093 (1.4); 3.5767 (0.9); 3.5299 (0.8); 3.4385
(0.4); 3.3335 (2.9); 2.9171 (0.4); 2.9024 (0.7); 2.8893 (16.0); 2.8702 (1.1); 2.8551 (0.4); 2.7306 (13.1); 2.6287 (0.3); 2.5241
(0.6); 2.5193 (1.0); 2.5107 (12.5); 2.5063 (24.9); 2.5018 (32.0); 2.4972 (22.5); 2.4927 (10.6); 1.2395 (0.6); −0.0002 (2.6)
Ib-109: ¹H-NMR(400.2 MHz, d₆-DMSO):
δ = 8.6560 (4.5); 7.9529 (2.7); 7.7405 (3.2); 7.7191 (3.5); 7.6753 (0.4); 7.6723 (0.4); 7.3926 (0.4); 7.3735 (1.3); 7.3574 (3.3);
7.3396 (1.4); 7.3210 (1.9); 7.3024 (1.9); 7.2636 (3.4); 7.2444 (7.1); 7.2225 (5.0); 7.1292 (1.0); 7.1110 (1.6); 7.0920 (0.7); 4.9952
(1.6); 3.9479 (1.9); 3.9014 (2.4); 3.5622 (1.6); 3.5154 (1.3); 3.3409 (60.1); 2.8904 (16.0); 2.7316 (14.3); 2.5075 (20.1); 2.5032
(25.4); 2.4989 (19.0); 1.2385 (0.8); −0.0002 (0.4)
Ib-110: ¹H-NMR(400.2 MHz, d₆-DMSO):
δ = 8.6655 (4.9); 8.5647 (1.2); 7.9521 (2.6); 7.7653 (5.9); 7.7436 (6.6); 7.6694 (1.6); 7.6475 (1.8); 7.4674 (0.9); 7.4493 (1.6);
7.4180 (0.8); 7.4002 (1.6); 7.3810 (0.8); 7.3557 (0.6); 7.3377 (0.7); 7.3031 (6.5); 7.2813 (6.1); 7.2173 (10.0); 7.2103 (5.1);
7.1539 (1.1); 7.1150 (1.7); 7.0930 (1.6); 5.1769 (3.2); 4.8101 (2.5); 4.6188 (2.7); 4.4050 (0.4); 3.9652 (2.6); 3.9187 (3.3); 3.8983
(0.8); 3.8518 (1.2); 3.8417 (0.9); 3.8275 (1.5); 3.8137 (1.0); 3.6914 (0.9); 3.6774 (1.5); 3.6639 (0.9); 3.5862 (2.3); 3.5396 (2.5);
3.4931 (0.5); 3.3452 (84.8); 2.9525 (0.9); 2.9407 (1.5); 2.9264 (1.0); 2.8902 (16.0); 2.7314 (14.3); 2.5069 (26.3); 2.5027 (33.8);
2.4984 (25.0); 1.2389 (1.1)
lb-111: ¹H-NMR(300.2 MHz, d₆-DMSO):
δ = 8.6482 (1.8); 7.6770 (3.3); 7.6487 (4.1); 7.3685 (4.0); 7.3400 (3.4); 3.9520 (1.5); 3.8899 (2.0); 3.5804 (1.4); 3.5180 (1.0);
3.3499 (16.0); 2.5388 (16.8); 2.5291 (5.2); 2.5229 (6.6); 2.5170 (5.0); 0.0209 (8.0)
Ib-112: ¹H-NMR(300.2 MHz, CDCl3):
δ = 8.7266 (3.0); 7.6778 (0.7); 7.6525 (0.8); 7.4443 (0.6); 7.4193 (1.0); 7.3915 (0.6); 7.2987 (19.8); 7.1643 (0.4); 3.7964 (0.4);
3.7369 (0.7); 3.6606 (0.8); 3.5533 (0.5); 3.4935 (0.3); 1.5824 (16.0); 0.0490 (1.2); 0.0384 (24.9); 0.0276 (1.2)
Ib-113: ¹H-NMR(300.2 MHz, d₆-DMSO):
δ = 8.5497 (1.1); 7.4102 (1.7); 7.3816 (1.9); 6.6158 (2.0); 6.5871 (1.8); 5.7191 (2.0); 3.8558 (0.5); 3.7951 (0.6); 3.4775 (0.6);
3.4159 (0.5); 3.3467 (16.0); 2.5342 (1.7); 2.5284 (3.6); 2.5224 (5.0); 2.5164 (3.6); 2.5106 (1.7); 2.0097 (0.4); 0.0208 (5.1)
Ib-114: ¹H-NMR(300.2 MHz, d₆-DMSO):
δ = 10.1970 (0.7); 8.7384 (0.8); 7.6935 (3.9); 3.9079 (0.4); 3.5713 (0.4); 3.3481 (16.0); 2.5342 (2.6); 2.5283 (5.6); 2.5223 (7.8);
2.5163 (5.7); 2.5106 (2.7); 2.0906 (4.2); 0.0210 (7.6)
Ib-115: ¹H-NMR(300.2 MHz, d₆-DMSO):
δ = 9.3943 (0.7); 8.7485 (0.6); 7.8035 (0.7); 7.7740 (1.3); 7.6949 (1.3); 7.6656 (0.8); 3.9177 (0.4); 3.3452 (16.0); 2.5344 (1.6);
2.5284 (3.4); 2.5224 (4.8); 2.5163 (3.5); 2.5104 (1.6); 1.4355 (3.3); 1.1346 (0.8); 1.1257 (0.9); 1.1137 (0.3); 0.6958 (0.4); 0.6834
(1.0); 0.6742 (1.0); 0.0208 (6.6); −0.0445 (0.4)
Ib-116: ¹H-NMR(300.2 MHz, d₆-DMSO):
δ = 10.5555 (1.1); 8.7852 (1.0); 7.9431 (1.2); 7.9138 (1.8); 7.8615 (0.5); 7.8329 (0.9); 7.8252 (0.5); 7.7998 (0.4); 7.7815 (1.8);
7.7522 (1.2); 7.6419 (0.4); 7.6224 (0.5); 7.6164 (0.4); 7.4984 (0.4); 7.4906 (0.4); 4.0108 (0.4); 3.9489 (0.6); 3.6100 (0.5); 3.5481
(0.4); 3.3435 (16.0); 2.5342 (3.1); 2.5282 (6.7); 2.5222 (9.5); 2.5162 (7.1); 2.5104 (3.6); 2.0097 (0.9); 1.1952 (0.5); 0.0316 (0.4);
0.0208 (12.1); 0.0099 (0.5)
Ib-117: ¹H-NMR(300.2 MHz, CDCl3):
δ = 9.1341 (1.8); 7.9352 (2.2); 7.8661 (1.1); 7.8309 (0.8); 7.7781 (7.6); 7.7502 (5.8); 7.7286 (1.2); 7.6952 (0.9); 7.6649 (0.8);
7.6212 (3.4); 7.5950 (3.6); 7.5463 (0.7); 7.5387 (0.5); 7.5300 (0.4); 7.5207 (0.4); 7.4879 (1.3); 7.4629 (2.5); 7.4423 (2.5); 7.4172
(1.3); 7.3176 (0.4); 7.2989 (23.5); 7.2861 (1.6); 7.2808 (2.0); 7.2564 (2.9); 7.2504 (2.7); 7.2275 (1.3); 4.1858 (0.8); 4.1620 (2.4);
4.1382 (2.5); 4.1144 (0.9); 3.8691 (3.7); 3.8100 (6.1); 3.7387 (0.4); 3.5963 (4.4); 3.5365 (2.9); 2.8084 (0.4); 2.0778 (11.0);
1.6600 (16.0); 1.3156 (3.1); 1.2918 (6.6); 1.2680 (2.8); 0.9196 (0.5); 0.1080 (0.5); 0.0486 (0.9); 0.0377 (23.8); 0.0266 (0.8)
Ib-118: ¹H-NMR(400.1 MHz, d₆-DMSO):
δ = 10.7948 (3.0); 10.7580 (0.6); 8.6131 (7.3); 7.9616 (3.0); 7.7460 (0.5); 7.7157 (5.4); 7.6946 (5.9); 7.3118 (5.7); 7.2906 (5.2);
4.8392 (0.6); 4.8145 (0.6); 4.6664 (1.3); 4.6421 (3.8); 4.6176 (3.9); 4.5932 (1.3); 4.4883 (1.4); 3.9282 (0.4); 3.9104 (2.5); 3.8810
(0.5); 3.8638 (3.1); 3.5568 (2.5); 3.5096 (2.0); 3.3116 (40.9); 2.8998 (16.0); 2.7409 (15.1); 2.5095 (14.8)
Ib-119: ¹H-NMR(300.2 MHz, d₆-DMSO):
δ = 10.6062 (2.0); 7.9871 (2.2); 7.9577 (3.1); 7.8812 (1.0); 7.8538 (1.6); 7.8444 (0.9); 7.8133 (3.7); 7.7997 (0.7); 7.7841 (2.4);
7.7765 (0.9); 7.7704 (1.4); 7.7651 (0.9); 7.7106 (0.5); 7.7058 (0.6); 7.6898 (1.0); 7.6824 (1.5); 7.6761 (1.1); 7.6620 (1.4); 7.6571
(2.2); 7.6506 (1.8); 7.6281 (1.8); 7.6229 (1.5); 7.6034 (0.6); 7.5363 (0.4); 7.5333 (0.5); 7.5275 (0.4); 7.5046 (0.7); 7.4982 (0.6);
4.6456 (0.5); 4.5805 (0.8); 4.3863 (1.0); 4.3220 (0.6); 3.3499 (16.0); 2.5343 (2.2); 2.5283 (4.8); 2.5223 (6.6); 2.5162 (4.8);
2.5103 (2.2); 0.0193 (6.9)
Ic-001: ¹H-NMR(300.2 MHz, d₆-DMSO):
δ = 8.7047 (1.5); 7.7638 (0.6); 7.7262 (0.9); 7.7022 (0.9); 5.7795 (0.8); 5.1256 (1.8); 4.4413 (1.6); 4.1297 (1.7); 4.0618 (0.5);
4.0382 (0.5); 4.0049 (0.4); 3.9427 (0.5); 3.6120 (0.4); 3.3473 (16.0); 2.5287 (3.8); 2.5229 (5.4); 2.5171 (4.1); 2.0102 (2.1);
1.8822 (4.2); 1.2194 (0.6); 1.1957 (1.1); 1.1720 (0.5); 0.0212 (3.8)
Ic-002: ¹H-NMR(300.2 MHz, d₆-DMSO):
δ = 8.7150 (0.3); 7.7664 (0.5); 7.7245 (0.8); 7.7167 (0.7); 5.1401 (1.4); 4.5648 (0.7); 4.1674 (0.7); 4.0053 (0.3); 3.9432 (0.4);
3.3458 (16.0); 2.5282 (3.9); 2.5223 (5.3); 2.5163 (4.0); 0.7937 (0.8); 0.7672 (0.8); 0.0207 (4.5)
Ic-003: ¹H-NMR(300.2 MHz, d₆-DMSO):
δ = 8.7077 (1.1); 7.7686 (2.4); 7.7427 (0.9); 7.7374 (0.8); 7.7095 (0.8); 7.5615 (0.4); 7.5386 (0.5); 7.5195 (0.8); 7.4951 (0.8);
5.1324 (0.9); 4.6286 (0.3); 4.3804 (0.3); 4.0047 (0.4); 3.9425 (0.5); 3.6107 (0.4); 3.3487 (16.0); 2.5287 (3.7); 2.5227 (5.2);
2.5169 (4.0); 2.0102 (1.2); 1.2191 (0.3); 1.1955 (0.6); 0.0210 (4.8)
Ic-004: ¹H-NMR(300.2 MHz, d₆-DMSO):
δ = 7.8403 (2.1); 7.8139 (5.0); 7.7805 (3.3); 7.7545 (1.8); 7.7272 (0.4); 7.7008 (0.5); 7.6540 (4.3); 5.1249 (0.9); 5.0959 (0.4);
5.0699 (1.0); 5.0442 (9.8); 5.0109 (0.5); 4.4406 (0.5); 4.1306 (0.6); 4.0849 (1.2); 4.0613 (3.7); 4.0376 (3.6); 4.0137 (1.4); 4.0089
(1.0); 3.9918 (2.9); 3.9296 (8.3); 3.9000 (5.5); 3.6038 (2.6); 3.5834 (5.8); 3.5538 (5.5); 3.4447 (0.4); 3.3477 (6.4); 2.5344 (9.3);
2.5285 (20.3); 2.5225 (28.4); 2.5165 (20.7); 2.5107 (9.8); 2.0099 (16.0); 1.8817 (1.5); 1.5685 (1.1); 1.3441 (0.7); 1.3207 (0.4);
1.2188 (4.3); 1.1951 (8.4); 1.1714 (4.0); 1.0015 (0.3); 0.9294 (0.6); 0.9051 (1.1); 0.8809 (0.5); 0.0314 (0.8); 0.0205 (31.4);
0.0095 (1.3); −0.0444 (3.7)
Ic-006: ¹H-NMR(499.9 MHz, d₆-DMSO):
δ = 8.7422 (6.2); 8.6169 (10.3); 7.0492 (7.0); 7.0282 (0.8); 7.0182 (2.0); 7.0150 (3.0); 7.0096 (16.0); 7.0027 (3.9); 7.0005 (3.8);
6.9942 (0.8); 6.9877 (2.2); 6.9849 (2.4); 6.9263 (2.9); 6.9239 (3.1); 6.9109 (3.7); 6.9085 (3.6); 6.7842 (2.2); 6.7819 (2.6); 6.7691
(3.5); 6.7669 (3.9); 6.7542 (1.8); 6.7518 (1.9); 6.6754 (3.7); 6.6734 (3.9); 6.6596 (3.6); 6.6576 (3.5); 4.0372 (0.4); 4.0230 (0.4);
3.8593 (3.8); 3.8225 (4.5); 3.4494 (2.8); 3.4124 (2.3); 3.3246 (25.0); 2.5096 (2.2); 2.5061 (4.8); 2.5025 (6.8); 2.4989 (5.0);
2.4954 (2.5); 1.9935 (0.4); 1.9889 (1.9); 1.2330 (0.7); 1.1898 (0.6); 1.1755 (1.1); 1.1613 (0.6); −0.0002 (5.2)
Ic-008: ¹H-NMR(300.2 MHz, d₆-DMSO):
δ = 10.6404 (0.8); 8.8328 (1.0); 8.8281 (1.0); 8.7562 (1.0); 8.4928 (0.8); 8.4586 (2.1); 8.4352 (0.8); 8.4178 (0.9); 8.3069 (0.8);
8.2788 (1.0); 7.8644 (0.6); 7.8587 (0.6); 7.8361 (0.5); 7.8303 (0.5); 7.4760 (0.6); 7.4705 (0.6); 7.4585 (0.6); 7.4530 (0.6); 4.1494
(0.5); 4.0883 (0.7); 3.7852 (0.5); 3.7236 (0.4); 3.3503 (16.0); 2.5343 (1.3); 2.5284 (2.8); 2.5224 (3.8); 2.5164 (2.8); 2.5106 (1.4);
2.1563 (5.0); 0.0189 (4.2)
Ic-009: ¹H-NMR(300.2 MHz, d₆-DMSO):
δ = 8.8857 (6.8); 8.3653 (0.4); 8.0605 (3.2); 8.0545 (3.4); 8.0381 (1.9); 8.0321 (3.9); 8.0302 (3.9); 7.9493 (3.4); 7.9462 (3.2);
7.9399 (2.3); 7.9267 (4.4); 7.9199 (3.9); 7.8927 (12.8); 7.5267 (1.2); 7.5210 (1.8); 7.5029 (4.6); 7.4971 (4.4); 7.4883 (4.7);
7.4803 (9.4); 7.4716 (4.3); 7.4635 (4.4); 7.4582 (4.3); 7.4395 (1.5); 7.4345 (1.0); 4.1077 (4.9); 4.0463 (6.7); 3.7275 (3.8); 3.7247
(3.8); 3.6660 (2.8); 3.6629 (2.9); 3.4362 (0.4); 3.4129 (1.1); 3.3895 (1.2); 3.3556 (16.0); 2.5343 (2.5); 2.5284 (5.4); 2.5223 (7.5);
2.5163 (5.4); 2.5104 (2.5); 1.1333 (1.1); 1.1099 (2.2); 1.0866 (1.1); 0.0186 (8.4)
Ic-010: ¹H-NMR(300.2 MHz, d₆-DMSO):
δ = 8.1948 (0.5); 8.0937 (0.6); 7.8087 (0.4); 7.8039 (0.4); 7.6574 (0.4); 7.6279 (0.3); 4.0464 (0.3); 3.9850 (0.5); 3.3439 (16.0);
2.5345 (1.6); 2.5285 (3.5); 2.5224 (4.9); 2.5164 (3.6); 2.5104 (1.7); 2.0098 (0.5); 0.0208 (6.9)
Ic-011: ¹H-NMR(499.9 MHz, d₆-DMSO):
δ = 13.3942 (3.3); 8.5337 (0.4); 8.2058 (12.6); 8.1327 (16.0); 7.8198 (7.3); 7.8174 (7.1); 7.8023 (9.0); 7.7999 (8.7); 7.6516 (10.4);
7.6340 (8.7); 4.1138 (2.9); 4.0752 (10.8); 4.0482 (14.7); 4.0099 (4.2); 3.5213 (0.4); 3.3787 (66.7); 3.3273 (12.0); 3.2311 (0.4);
2.5111 (12.9); 2.5079 (16.3); 2.5047 (12.8); 2.0798 (0.9)
Ic-012: ¹H-NMR(499.9 MHz, d₆-DMSO):
δ = 10.0398 (16.0); 8.6139 (14.8); 8.0773 (7.3); 7.8431 (5.7); 7.8258 (6.0); 7.8096 (0.4); 7.7867 (2.4); 7.7836 (2.2); 7.7694 (7.4);
7.7662 (7.4); 7.7556 (9.2); 7.7382 (2.6); 7.2148 (0.8); 7.1987 (0.5); 7.1938 (0.5); 7.1731 (5.4); 7.1688 (8.6); 7.1636 (6.6); 7.1588
(2.7); 7.1462 (4.9); 7.1415 (3.5); 4.0224 (0.5); 4.0128 (5.0); 3.9763 (6.1); 3.6587 (3.8); 3.6220 (3.1); 3.3561 (0.3); 3.3369 (0.3);
2.5092 (3.9); 2.5057 (7.3); 2.5021 (9.5); 2.4985 (6.6); 2.4951 (3.0); 1.9887 (0.5); 1.2337 (0.3); 0.0063 (0.4); −0.0002 (6.3)
Ic-013: ¹H-NMR(300.2 MHz, CDCl3):
δ = 7.8806 (3.6); 7.8516 (8.4); 7.8092 (8.2); 7.7793 (11.9); 7.6172 (4.5); 7.3604 (2.9); 7.3366 (2.8); 7.2989 (14.5); 5.3366 (0.8);
4.6230 (1.1); 4.6031 (1.7); 4.5827 (2.3); 4.5624 (2.9); 4.5409 (3.3); 4.5202 (2.4); 4.1675 (0.7); 4.1438 (0.7); 4.1206 (0.7); 4.0987
(0.9); 4.0763 (0.5); 3.9927 (0.9); 3.7959 (4.5); 3.7366 (7.9); 3.5871 (6.3); 3.5278 (3.6); 3.0251 (13.0); 2.9461 (8.6); 2.7204 (0.4);
2.7049 (0.4); 2.0843 (6.2); 1.6782 (3.3); 1.6452 (0.8); 1.6181 (0.5); 1.4245 (0.4); 1.4001 (0.4); 1.3758 (0.4); 1.3260 (9.5); 1.3160
(7.9); 1.3042 (11.3); 1.2929 (8.7); 1.2711 (16.0); 1.2492 (13.9); 0.9933 (0.8); 0.9689 (1.5); 0.9443 (0.7); 0.0488 (0.6); 0.0385
(17.2)
Ic-014: ¹H-NMR(300.2 MHz, CDCl3):
δ = 7.9614 (0.4); 7.9347 (2.4); 7.8920 (0.7); 7.8633 (1.3); 7.8378 (0.4); 7.6488 (0.8); 7.6411 (0.8); 7.4001 (0.6); 7.3926 (0.5);
7.3707 (0.5); 7.3631 (0.6); 7.2987 (18.2); 3.8992 (0.5); 3.8400 (1.0); 3.7023 (0.6); 3.6465 (0.3); 3.5833 (1.3); 3.2033 (3.2);
3.0968 (3.2); 1.5832 (16.0); 1.2917 (0.4); 0.0495 (0.8); 0.0387 (24.2); 0.0278 (0.8)
Ic-015: ¹H-NMR(300.2 MHz, CDCl3):
δ = 7.9119 (2.9); 7.9064 (2.7); 7.8838 (4.7); 7.8779 (6.8); 7.8271 (16.0); 7.7922 (7.6); 7.6259 (3.1); 7.6177 (3.2); 7.3668 (2.4);
7.3381 (2.1); 7.2987 (20.8); 4.4173 (0.5); 4.3893 (0.5); 3.8306 (4.6); 3.7715 (8.2); 3.6279 (5.0); 3.6067 (1.1); 3.5827 (2.8);
3.5718 (3.6); 3.5683 (3.8); 3.5599 (3.2); 3.5350 (1.4); 3.5234 (1.3); 3.4998 (2.7); 3.4761 (2.7); 3.4528 (1.0); 3.2176 (0.4); 3.1937
(1.4); 3.1686 (11.7); 3.1466 (1.2); 3.0738 (11.8); 2.7742 (6.0); 1.6522 (2.6); 1.3553 (3.1); 1.3318 (6.6); 1.3064 (6.7); 1.2932
(6.6); 1.2687 (6.0); 1.2449 (2.9); 1.1606 (1.6); 1.1369 (3.2); 1.1131 (1.6); 0.9412 (1.3); 0.9196 (4.1); 0.8964 (1.6); 0.0486 (0.7);
0.0377 (22.6); 0.0266 (0.9)
Ic-016: ¹H-NMR(499.9 MHz, d₆-DMSO):
δ = 8.7200 (0.6); 8.1566 (4.5); 8.0712 (3.6); 7.8526 (1.6); 7.8504 (1.6); 7.8349 (2.3); 7.8327 (2.2); 7.7531 (2.6); 7.7354 (2.0);
4.0860 (16.0); 4.0132 (1.7); 3.9764 (2.0); 3.6580 (1.6); 3.6211 (1.3); 3.3277 (1.0); 2.5080 (3.6)
Ic-017: ^{l}H-NMR(499.9 MHz, d₆-DMSO):
δ = 8.1772 (4.0); 8.1167 (3.0); 7.8498 (1.4); 7.8470 (1.3); 7.8321 (2.0); 7.8292 (1.9); 7.7613 (2.4); 7.7435 (1.6); 4.1129 (0.5);
4.0825 (16.0); 4.0750 (2.4); 4.0483 (2.9); 4.0099 (0.8); 3.3734 (13.5); 3.3209 (4.8); 2.5041 (1.6); 2.5007 (2.1); 2.4972 (1.5)
Ic-018: ¹H-NMR(499.9 MHz, d₆-DMSO):
δ = 8.6016 (1.0); 8.1760 (3.4); 8.0597 (2.4); 7.8217 (0.9); 7.8188 (0.9); 7.8039 (1.8); 7.8010 (2.0); 7.7713 (2.2); 7.7534 (1.1);
4.6055 (1.6); 4.5950 (3.1); 4.5845 (1.7); 4.0103 (1.4); 3.9736 (1.7); 3.7718 (1.8); 3.7612 (3.3); 3.7508 (1.7); 3.6414 (1.1); 3.6045
(0.9); 3.3198 (2.1); 3.2393 (0.7); 3.1734 (16.0); 2.5084 (0.8); 2.5050 (1.7); 2.5014 (2.3); 2.4979 (1.8)
Ic-019: ^{l}H-NMR(499.9MHz, d₆-DMSO):
δ = 8.6592 (9.4); 8.6190 (2.6); 8.0272 (6.5); 7.6993 (1.4); 7.6964 (1.4); 7.6810 (5.9); 7.6781 (6.0); 7.6690 (7.2); 7.6508 (1.7);
7.3867 (1.0); 7.3844 (1.4); 7.3814 (0.8); 7.3696 (4.9); 7.3679 (4.5); 7.3559 (14.9); 7.3514 (13.0); 7.3384 (3.6); 7.3359 (3.5);
7.3248 (1.6); 7.3186 (2.9); 7.3114 (1.0); 7.3056 (0.9); 7.3017 (0.5); 5.6742 (16.0); 3.9794 (3.3); 3.9428 (4.1); 3.6233 (2.8);
3.5865 (2.4); 3.3255 (15.8); 2.5127 (8.6); 2.5092 (11.6); 2.5057 (8.8)
Ic-020: ¹H-NMR(499.9 MHz, d₆-DMSO):
δ = 8.7187 (5.0); 8.1064 (3.6); 7.7214 (0.9); 7.7185 (0.9); 7.7031 (3.7); 7.7003 (3.6); 7.6938 (3.9); 7.6755 (0.8); 7.3804 (2.4);
7.3668 (8.1); 7.3637 (7.1); 7.3597 (16.0); 7.3516 (5.4); 7.3434 (1.5); 7.3303 (8.2); 7.3165 (4.7); 7.3081 (1.1); 7.3012 (0.8);
5.6872 (8.0); 4.7904 (2.3); 4.7683 (2.7); 4.6031 (2.8); 4.5809 (2.2); 4.2126 (0.8); 4.1741 (2.0); 4.1313 (2.8); 4.0930 (1.2); 3.3257
(17.2); 2.5123 (4.5); 2.5088 (5.6); 2.5053 (4.0); 1.9960 (0.9); 1.2426 (0.7); 1.1823 (0.5)
Ic-021: ¹H-NMR(499.9 MHz, d₆-DMSO):
δ = 8.6436 (10.3); 8.5537 (16.0); 8.0602 (11.8); 7.7524 (2.6); 7.7496 (2.4); 7.7342 (11.1); 7.7313 (11.0); 7.7229 (12.7); 7.7047
(2.8); 4.6695 (7.9); 4.6592 (13.3); 4.6487 (8.0); 4.0345 (6.7); 3.9980 (8.1); 3.9035 (8.7); 3.8930 (14.4); 3.8826 (7.9); 3.6763
(5.3); 3.6396 (4.4); 3.4237 (0.4); 3.3760 (20.0); 3.2849 (69.1); 3.2287 (0.4); 3.1415 (0.4); 2.5599 (8.1); 2.5565 (10.1); 2.5531
(7.1); 1.2894 (1.2); 1.1123 (0.4)
Ic-022: ¹H-NMR(300.2 MHz, d₆-DMSO):
δ = 11.9855 (0.4); 8.6542 (0.9); 8.5781 (1.0); 8.3267 (0.6); 8.3227 (0.6); 8.0454 (0.7); 8.0401 (0.7); 7.8063 (1.0); 7.7926 (0.7);
7.7869 (0.6); 7.7657 (0.9); 7.6248 (0.4); 7.6189 (0.4); 7.5967 (0.4); 7.5907 (0.5); 7.4042 (0.8); 7.3762 (0.6); 4.0379 (0.5); 3.9747
(0.5); 3.9604 (0.4); 3.8989 (0.5); 3.5921 (0.5); 3.3686 (1.7); 2.5346 (4.6); 2.5287 (9.8); 2.5227 (13.5); 2.5166 (9.8); 2.5108 (4.5);
2.0100 (1.0); 1.9297 (16.0); 1.5435 (8.4); 1.2193 (0.3); 1.1955 (0.6); 0.0318 (0.6); 0.0210 (18.4); 0.0100 (0.7); −0.0430 (2.4)
Ic-023: ¹H-NMR(300.2 MHz, CDCl3):
δ = 7.8945 (2.4); 7.8896 (2.6); 7.8659 (4.3); 7.8611 (5.0); 7.8011 (9.9); 7.7710 (6.4); 7.7390 (0.4); 7.6101 (2.0); 7.5506 (0.3);
7.4982 (1.8); 7.4705 (6.6); 7.4473 (16.0); 7.4402 (13.1); 7.4174 (3.1); 7.3588 (2.4); 7.3382 (3.5); 7.3162 (2.2); 7.2987 (10.3);
4.3642 (5.3); 3.8227 (4.0); 3.7636 (6.8); 3.6125 (4.5); 3.5529 (3.1); 3.5011 (6.2); 2.0857 (1.3); 1.7200 (10.2); 1.3480 (0.5);
1.3072 (3.3); 1.2927 (1.9); 1.2683 (0.5); 0.9447 (1.2); 0.9230 (3.6); 0.8997 (1.4); 0.0523 (0.3); 0.0414 (10.6); 0.0304 (0.4)
Ic-024: ¹H-NMR(300.2 MHz, CDCl3):
δ = 7.8872 (3.5); 7.8821 (4.0); 7.8583 (7.4); 7.8532 (8.6); 7.8315 (0.5); 7.8085 (10.8); 7.7795 (16.0); 7.7598 (7.5); 7.6441 (0.4);
7.6167 (3.9); 7.3571 (3.3); 7.3279 (3.0); 7.2987 (17.3); 4.5979 (1.3); 4.1674 (0.3); 4.1439 (0.7); 4.1085 (1.6); 4.0794 (1.6);
3.7986 (5.5); 3.7395 (9.6); 3.5922 (6.0); 3.5326 (3.4); 3.0860 (0.8); 3.0473 (11.6); 2.9694 (8.1); 2.0839 (0.8); 1.8971 (6.3);
1.7503 (2.2); 1.7001 (3.4); 1.6831 (3.8); 1.5995 (1.9); 1.5514 (2.7); 1.5019 (4.3); 1.4593 (4.8); 1.4158 (3.3); 1.3713 (1.9); 1.3443
(2.1); 1.3043 (12.2); 1.2690 (1.1); 1.2259 (0.9); 1.1834 (1.6); 1.1444 (1.4); 0.9418 (4.4); 0.9201 (13.5); 0.8968 (5.2); 0.0490
(0.6); 0.0382 (19.0); 0.0272 (0.8)
Ic-025: ¹H-NMR(499.9 MHz, d₆-DMSO):
δ = 8.6176 (3.5); 8.2292 (12.4); 8.1017 (8.3); 7.8237 (16.0); 7.3326 (1.6); 7.3301 (2.6); 7.3271 (1.2); 7.3164 (7.5); 7.3135 (4.4);
7.3014 (6.8); 7.2786 (3.2); 7.2760 (2.2); 7.2688 (1.0); 7.2643 (3.7); 7.2587 (0.8); 7.2496 (1.0); 7.2332 (6.3); 7.2304 (7.5); 7.2166
(5.6); 5.7624 (3.2); 5.7124 (15.5); 4.0136 (3.9); 3.9768 (4.8); 3.6467 (3.0); 3.6098 (2.5); 3.3279 (7.6); 2.5161 (2.6); 2.5126 (5.5);
2.5090 (7.4); 2.5055 (5.4); 2.5020 (2.5)
Ic-026: ¹H-NMR(499.9 MHz, d₆-DMSO):
δ = 8.2678 (6.2); 8.1718 (4.2); 7.8501 (8.2); 7.3834 (0.4); 7.3678 (4.1); 7.3646 (4.5); 7.3574 (16.0); 7.3477 (1.0); 7.3427 (0.5);
7.3330 (1.2); 7.3271 (2.3); 7.3132 (4.6); 7.2982 (3.7); 7.2758 (1.7); 7.2659 (0.6); 7.2615 (2.0); 7.2560 (0.4); 7.2468 (0.5); 7.2154
(3.9); 7.2014 (3.1); 5.7286 (8.0); 4.7936 (2.4); 4.7715 (2.9); 4.5954 (2.9); 4.5733 (2.3); 4.2408 (0.7); 4.2021 (2.2); 4.1700 (3.1);
4.1316 (1.0); 3.3267 (16.1); 2.5127 (3.4); 2.5092 (4.5); 2.5056 (3.3)
Ic-027: ¹H-NMR(300.2 MHz, CDCl3):
δ = 8.0903 (1.3); 7.8902 (0.4); 7.8163 (2.6); 7.5933 (0.4); 7.2989 (53.4); 3.8695 (0.6); 3.8109 (1.2); 3.6631 (0.7); 3.6030 (0.5);
2.0850 (0.7); 1.7489 (0.4); 1.7372 (0.5); 1.7229 (0.4); 1.7110 (0.4); 1.5948 (16.0); 1.3198 (1.5); 1.3053 (2.6); 1.2926 (3.5);
1.1297 (0.9); 1.1180 (0.8); 1.1036 (0.8); 1.0930 (0.7); 0.9416 (0.6); 0.9198 (1.5); 0.8962 (0.7); 0.1070 (0.7); 0.0484 (2.8); 0.0378
(61.8); 0.0269 (2.6); 0.0155 (0.4)
Ic-028: ¹H-NMR(300.2 MHz, d₆-DMSO):
δ = 10.8739 (3.0); 8.6258 (7.0); 7.3346 (3.6); 7.3280 (4.1); 7.2491 (1.9); 7.2423 (1.6); 7.2213 (2.4); 7.2145 (2.3); 7.0661 (3.6);
7.0383 (2.7); 4.7106 (0.4); 4.7011 (1.0); 4.6730 (16.0); 3.9274 (2.0); 3.8660 (2.6); 3.5187 (1.7); 3.4567 (1.4); 3.3997 (1.1);
2.9097 (1.1); 2.7498 (0.9); 2.5344 (1.0); 2.5285 (2.3); 2.5221 (4.2); 2.5165 (2.4); 2.5106 (1.1); 0.0183 (3.6)
Ic-030: ¹H-NMR(300.2 MHz, d₆-DMSO):
δ = 8.7318 (2.3); 8.5646 (2.6); 8.5603 (2.6); 8.4637 (5.4); 8.2792 (2.3); 8.2512 (2.5); 7.6287 (1.6); 7.6232 (1.6); 7.6008 (1.4);
7.5952 (1.5); 7.5722 (3.4); 7.5662 (3.5); 6.4498 (3.5); 6.4437 (3.6); 5.7779 (0.7); 4.2048 (0.9); 4.1809 (2.9); 4.1569 (3.0); 4.1322
(2.2); 4.0704 (1.9); 3.7768 (1.2); 3.7151 (0.9); 3.3554 (16.0); 2.5346 (1.1); 2.5288 (2.3); 2.5227 (3.2); 2.5167 (2.4); 2.5108 (1.1);
1.3739 (3.4); 1.3501 (7.8); 1.3261 (3.3); 0.0174 (3.0)
Ic-031: ¹H-NMR(300.2 MHz, d₆-DMSO):
δ = 8.7473 (16.0); 8.4311 (15.2); 8.2408 (6.2); 8.2126 (7.1); 7.8519 (4.2); 7.8460 (4.3); 7.8237 (3.7); 7.8177 (3.8); 7.7057 (0.4);
7.6217 (0.7); 7.5945 (3.1); 7.5736 (10.4); 7.5624 (7.4); 7.5357 (3.1); 7.5309 (3.0); 7.5068 (0.4); 7.4965 (0.4); 7.2973 (1.2);
7.2882 (1.9); 7.2800 (1.4); 7.2768 (1.5); 7.2673 (2.7); 7.2586 (2.0); 7.2505 (1.4); 7.2443 (1.6); 7.2374 (1.2); 7.2286 (1.0); 4.1376
(4.0); 4.0766 (5.5); 3.8007 (3.6); 3.7391 (2.5); 3.3694 (15.1); 2.9060 (0.6); 2.7497 (0.5); 2.5343 (1.4); 2.5285 (3.0); 2.5225 (4.2);
2.5165 (3.1); 2.5107 (1.4); 1.2275 (0.4); 1.1009 (0.8); 1.0776 (1.5); 1.0543 (0.7); 0.0109 (2.4)
Ic-032: ¹H-NMR(300.2 MHz, d₆-DMSO):
δ = 8.7546 (7.7); 8.6417 (7.1); 8.6354 (7.4); 8.4388 (14.4); 8.1409 (7.1); 8.1122 (8.0); 7.6919 (4.4); 7.6854 (4.4); 7.6631 (3.8);
7.6566 (3.9); 4.1094 (4.2); 4.0483 (5.8); 3.7566 (3.6); 3.6950 (2.6); 3.3565 (16.0); 2.5339 (2.0); 2.5281 (4.1); 2.5221 (5.6);
2.5162 (4.1); 0.0153 (5.5)
Ic-033: ¹H-NMR(300.2 MHz, CDCl3):
δ = 15.2193 (0.3); 13.1289 (0.4); 8.1127 (1.6); 7.9110 (0.4); 7.8233 (3.6); 7.8196 (3.3); 7.7972 (0.4); 7.7906 (0.6); 7.6445 (1.1);
7.6248 (0.4); 7.5963 (0.4); 7.3635 (0.5); 7.2987 (175.2); 7.2322 (0.6); 7.1941 (0.3); 7.1662 (0.4); 6.9477 (1.1); 5.5809 (0.4);
5.3390 (2.8); 4.5528 (0.5); 3.8675 (0.7); 3.8298 (0.4); 3.8095 (1.5); 3.7352 (0.6); 3.6848 (0.5); 3.6610 (1.0); 3.6027 (0.5); 3.5877
(0.6); 3.5220 (0.4); 3.1599 (0.3); 2.9967 (0.7); 2.9229 (0.6); 2.3922 (8.6); 2.2200 (0.4); 2.0848 (1.2); 2.0673 (0.5); 1.5928 (16.0);
1.4564 (0.4); 1.4245 (0.4); 1.3766 (0.4); 1.3470 (0.5); 1.2934 (2.8); 1.2500 (0.5); 1.2313 (0.4); 1.1937 (0.4); 1.1416 (0.4); 0.9199
(0.4); 0.8936 (0.5); 0.2330 (0.7); 0.1042 (0.6); 0.0910 (0.4); 0.0645 (0.5); 0.0485 (6.4); 0.0377 (189.0); 0.0268 (6.7); −0.0287
(0.6); −0.1611 (0.7); −2.3140 (0.4)

Intermediates of formula 1b

1b-01: ¹H-NMR(499.9 MHz, CDCl3):
δ = 7.7583 (3.1); 7.7416 (3.5); 7.4203 (3.4); 7.4036 (3.2); 7.2605 (14.6); 7.2460 (3.4); 7.2303 (2.6); 7.1956 (1.5); 7.1810 (2.0);
7.1662 (0.7); 7.0459 (2.7); 7.0307 (2.4); 6.6079 (0.3); 6.4822 (6.0); 3.9778 (1.2); 3.9695 (1.2); 3.8684 (0.4); 3.5272 (16.0);
3.4992 (1.4); 3.4027 (0.3); 2.5342 (0.4); 0.0062 (0.5); −0.0002 (13.1)
1b-02: ¹H-NMR(300.2 MHz, CDCl3):
δ = 8.3221 (5.9); 8.2938 (8.2); 8.2700 (0.4); 8.2569 (0.3); 8.2356 (1.1); 8.1671 (0.4); 8.1469 (8.6); 8.1184 (6.1); 7.2989 (24.3);
6.6697 (9.8); 4.0069 (1.2); 2.9469 (2.3); 2.9217 (7.5); 2.8965 (7.8); 2.8712 (2.7); 2.7085 (0.9); 2.3793 (0.5); 1.8118 (0.4); 1.4728
(8.0); 1.4476 (16.0); 1.4223 (7.6); 1.2931 (0.6); 0.0485 (0.8); 0.0378 (25.5); 0.0271 (1.0)
1b-03: ¹H-NMR(300.2 MHz, CDCl3):
δ = 8.3334 (0.4); 8.3272 (0.9); 8.3216 (5.0); 8.3154 (2.0); 8.3043 (0.8); 8.2991 (2.2); 8.2925 (7.1); 8.2873 (1.5); 8.2557 (0.4);
8.2352 (0.8); 8.2326 (0.8); 8.1671 (0.5); 8.1514 (1.4); 8.1462 (7.6); 8.1397 (2.6); 8.1233 (2.0); 8.1170 (5.3); 7.2985 (13.0);
6.6694 (8.5); 4.0057 (1.3); 3.5085 (0.7); 2.9456 (2.2); 2.9203 (7.2); 2.9080 (0.6); 2.8950 (7.4); 2.8828 (0.6); 2.8698 (2.5); 2.7076
(1.6); 1.4718 (7.7); 1.4466 (16.0); 1.4213 (7.4); 0.0473 (0.5); 0.0365 (15.9); 0.0256 (0.6)
1b-04: ¹H-NMR(300.2 MHz, d₆-DMSO):
δ = 8.2520 (2.0); 8.2240 (2.9); 8.1333 (4.8); 8.1049 (3.2); 8.0989 (2.0); 6.9640 (1.8); 3.9226 (16.0); 3.9127 (3.1); 2.6537 (1.2);
2.5289 (2.8); 2.5230 (4.1); 2.5171 (3.3); 2.0089 (0.5); 1.9298 (1.4); 0.0181 (3.8)
1b-05: ¹H-NMR(300.2 MHz, d₆-DMSO):
δ = 8.0994 (13.1); 8.0706 (14.0); 7.4557 (16.0); 7.4268 (14.3); 7.0216 (13.6); 4.8540 (0.4); 3.6210 (0.5); 2.8245 (0.3); 2.5921
(66.2); 2.5561 (0.5); 2.5485 (0.5); 2.5348 (3.7); 2.5288 (8.2); 2.5228 (11.1); 2.5168 (8.0); 2.5110 (3.8); 2.3554 (0.3); 2.0090
(0.6); 1.7789 (0.6); 1.2229 (0.3); 1.1944 (0.3); 1.1302 (0.4); 0.0182 (12.2); 0.0072 (0.5)
1b-06: ¹H-NMR(300.2 MHz, d₆-DMSO):
δ = 8.0812 (11.9); 8.0524 (12.5); 7.9103 (0.4); 7.8818 (0.3); 7.6081 (3.0); 7.6027 (2.7); 7.5942 (5.9); 7.5834 (7.2); 7.5805 (7.8);
7.5747 (11.7); 7.5571 (7.5); 7.5479 (13.5); 7.5342 (11.4); 7.5252 (9.3); 7.5143 (1.1); 7.5060 (0.4); 7.3077 (2.5); 7.3005 (16.0);
7.2944 (5.0); 7.2777 (4.8); 7.2717 (14.4); 6.9618 (12.6); 2.5349 (4.9); 2.5289 (10.5); 2.5229 (14.5); 2.5168 (10.5); 2.5109 (4.9);
1.3762 (0.7); 1.3176 (0.6); 0.0301 (0.5); 0.0192 (17.5); 0.0082 (0.7)
1b-07: ¹H-NMR(300.2 MHz, d₆-DMSO):
δ = 8.0934 (13.1); 8.0657 (14.3); 7.9386 (0.4); 7.9312 (0.4); 7.9094 (1.6); 7.8812 (1.4); 7.8604 (0.4); 7.8268 (1.4); 7.7992 (1.5);
7.5125 (0.4); 7.4797 (16.0); 7.4519 (14.7); 7.4032 (1.1); 7.3757 (1.6); 7.3505 (0.6); 7.3268 (1.4); 7.2982 (1.4); 6.9969 (12.0);
6.3429 (0.5); 6.2563 (1.7); 4.8659 (0.7); 4.6916 (0.4); 4.6178 (0.4); 4.5503 (0.5); 4.5261 (0.5); 4.4924 (0.5); 4.4385 (0.6); 4.4146
(0.6); 4.4061 (0.6); 4.3887 (0.6); 4.3737 (0.7); 4.3474 (0.9); 4.3233 (1.7); 4.3000 (1.8); 4.2759 (1.1); 4.2089 (1.1); 4.0924 (1.7);
4.0134 (2.4); 3.8175 (5.8); 3.7724 (6.0); 3.6814 (5.0); 3.3490 (1.0); 3.2599 (0.9); 3.1912 (0.7); 3.1667 (0.6); 3.1556 (0.6); 3.1134
(0.5); 3.1036 (0.6); 2.9557 (0.4); 2.9420 (0.4); 2.8987 (0.4); 2.8590 (0.4); 2.7487 (0.6); 2.6770 (1.7); 2.6468 (3.0); 2.6101 (1.8);
2.5633 (0.9); 2.5342 (24.0); 2.5283 (51.8); 2.5223 (71.7); 2.5163 (52.8); 2.5106 (25.5); 2.4538 (0.7); 2.2925 (0.4); 1.8354 (12.3);
1.8035 (15.4); 1.7538 (4.5); 1.7147 (3.8); 1.5226 (1.6); 1.4828 (4.7); 1.4484 (10.6); 1.4136 (10.3); 1.3756 (5.8); 1.3494 (3.1);
1.3258 (5.6); 1.3020 (2.8); 1.2870 (2.7); 1.2542 (2.8); 1.2208 (1.2); 1.1891 (1.6); 0.0306 (1.7); 0.0198 (58.5); 0.0089 (2.4)
1b-08: ¹H-NMR(300.2 MHz, d₆-DMSO):
δ = 8.2413 (12.2); 8.2133 (13.8); 8.1085 (1.2); 8.1018 (0.6); 8.0872 (0.7); 8.0802 (1.6); 8.0621 (0.5); 8.0498 (0.9); 8.0335 (0.9);
8.0216 (1.3); 8.0068 (1.0); 7.9293 (16.0); 7.9016 (13.3); 7.8763 (0.8); 7.8517 (0.4); 7.8223 (0.7); 7.7974 (0.6); 7.6447 (0.5);
7.0070 (12.6); 6.3439 (1.0); 6.0577 (0.3); 4.4041 (0.4); 4.3800 (1.2); 4.3563 (1.2); 4.3330 (0.4); 2.5344 (9.4); 2.5285 (20.3);
2.5225 (28.1); 2.5164 (20.3); 2.5106 (9.4); 1.5765 (9.2); 1.3757 (1.3); 1.3520 (2.7); 1.3283 (1.3); 1.2552 (0.9); 1.2499 (0.8);
1.2340 (2.2); 1.2030 (0.4); 0.0296 (1.0); 0.0187 (36.7); 0.0077 (1.6)
1b-09: ¹H-NMR(300.2 MHz, d₆-DMSO):
δ = 8.0596 (10.8); 8.0291 (11.5); 7.7891 (0.3); 7.0816 (10.9); 7.0509 (10.6); 7.0240 (0.4); 7.0068 (0.3); 6.8976 (16.0); 4.7376
(0.4); 4.1309 (0.8); 4.1073 (0.7); 4.0838 (0.4); 4.0608 (0.5); 4.0369 (0.5); 3.7683 (10.2); 3.7527 (16.0); 3.7357 (15.3); 3.4551
(11.8); 3.4379 (15.3); 3.4223 (11.7); 3.3677 (0.9); 3.2844 (0.7); 3.2679 (0.8); 3.2515 (0.5); 2.5339 (5.9); 2.5280 (16.6); 2.5220
(26.0); 2.5160 (21.0); 2.5102 (11.8); 2.3985 (0.4); 2.2921 (0.4); 2.0089 (2.5); 1.9290 (0.4); 1.3315 (0.4); 1.3085 (0.3); 1.2549
(0.4); 1.2438 (0.8); 1.2199 (1.8); 1.1946 (1.8); 1.1706 (0.7); 1.1299 (1.9); 0.0193 (35.0); 0.0085 (3.0); −0.0462 (0.4)
1b-10: ¹H-NMR(300.2 MHz, d₆-DMSO):
δ = 9.1806 (0.4); 9.1339 (0.4); 8.8421 (11.5); 8.1134 (0.3); 7.6898 (0.5); 7.6654 (0.4); 7.6163 (0.4); 7.5634 (5.4); 7.5575 (5.2);
7.5362 (5.8); 7.5302 (5.5); 7.4529 (0.4); 7.4282 (0.3); 7.3742 (0.4); 7.3237 (0.7); 7.3009 (1.1); 7.2652 (11.9); 7.2593 (10.8);
7.2193 (0.5); 7.2116 (0.6); 7.1926 (0.8); 7.1499 (0.7); 7.1229 (1.1); 7.1034 (12.0); 7.0763 (10.6); 7.0540 (3.8); 7.0491 (4.0);
7.0284 (6.8); 7.0242 (6.9); 7.0032 (4.8); 6.9983 (5.1); 6.9822 (0.6); 6.9476 (6.2); 6.9431 (6.0); 6.9219 (8.8); 6.9176 (7.3); 6.8940
(0.6); 6.8729 (0.9); 6.8481 (1.1); 6.8234 (16.0); 6.8143 (6.6); 6.8102 (6.3); 6.7892 (7.7); 6.7854 (7.5); 6.7642 (3.2); 6.7601 (3.0);
6.7151 (0.4); 6.6726 (7.9); 6.6693 (7.4); 6.6462 (6.9); 6.6429 (6.0); 5.7773 (13.8); 2.5341 (7.7); 2.5282 (16.3); 2.5222 (22.4);
2.5161 (16.1); 2.5102 (7.4); 2.0128 (0.6); 2.0083 (0.8); 1.3746 (1.8); 1.3177 (0.5); 1.2470 (0.4); 1.1939 (0.4); 0.0289 (0.8);
0.0179 (26.2); 0.0069 (1.1)
1b-11: ¹H-NMR(300.2 MHz, CDCl3):
δ = 7.2989 (43.0); 5.9753 (1.0); 4.2156 (0.4); 4.1918 (1.2); 4.1680 (1.3); 4.1443 (0.4); 2.8673 (0.4); 2.4681 (0.4); 1.9298 (0.5);
1.8840 (0.6); 1.7126 (0.4); 1.6888 (0.5); 1.6735 (0.6); 1.6297 (0.6); 1.5820 (4.1); 1.5037 (9.9); 1.4938 (16.0); 1.3177 (1.3);
1.2939 (2.8); 1.2703 (1.2); 1.2608 (0.9); 0.0487 (2.1); 0.0379 (57.2); 0.0272 (2.4)
1b-12: ¹H-NMR(300.2 MHz, d₆-DMSO):
δ = 12.1909 (0.4); 8.8835 (0.9); 8.8778 (0.9); 8.1988 (0.5); 8.1925 (0.5); 8.1696 (0.6); 8.1637 (0.5); 7.8360 (1.0); 7.8073 (0.9);
7.1143 (1.3); 2.5343 (3.4); 2.5285 (7.5); 2.5225 (10.6); 2.5165 (7.8); 2.5107 (3.8); 2.0097 (0.3); 1.5516 (16.0); 1.1896 (0.4);
0.0204 (11.2); 0.0094 (0.5)
1b-13: ¹H-NMR(300.2 MHz, d₆-DMSO):
δ = 11.3579 (1.3); 8.0522 (4.8); 8.0450 (5.1); 8.0112 (4.6); 8.0040 (5.1); 7.9314 (4.2); 7.9255 (3.7); 7.9027 (4.3); 7.8970 (3.9);
7.7613 (0.4); 7.7547 (0.6); 7.7301 (0.7); 7.7204 (0.6); 7.7135 (0.7); 7.7047 (0.7); 7.6141 (0.4); 7.1549 (5.5); 7.1261 (10.4);
7.0973 (5.2); 7.0769 (0.5); 7.0483 (0.9); 7.0038 (16.0); 6.3083 (1.6); 6.2214 (0.6); 4.8013 (0.6); 4.1542 (0.4); 4.1307 (1.2);
4.1070 (1.2); 4.0837 (0.7); 4.0605 (1.1); 4.0367 (1.1); 4.0130 (0.4); 3.6429 (0.6); 3.6292 (0.6); 3.6210 (1.6); 3.6131 (0.7); 3.5989
(0.7); 2.5343 (8.6); 2.5283 (20.9); 2.5222 (30.8); 2.5162 (23.6); 2.5103 (12.2); 2.4543 (0.4); 2.0087 (4.6); 1.8013 (0.6); 1.7910
(0.7); 1.7792 (1.9); 1.7673 (0.7); 1.7573 (0.7); 1.2573 (0.8); 1.2436 (1.2); 1.2197 (2.6); 1.1943 (3.0); 1.1705 (1.3); 1.1296 (2.9);
0.0189 (38.7); 0.0080 (2.4)
1b-14: ¹H-NMR(300.2 MHz, d₆-DMSO):
δ = 7.4142 (0.3); 7.3653 (0.4); 7.3372 (0.4); 7.0891 (1.5); 7.0611 (1.3); 3.8440 (16.0); 3.8273 (0.5); 3.8224 (0.6); 3.8095 (0.4);
2.5345 (1.7); 2.5285 (3.8); 2.5225 (5.3); 2.5165 (4.0); 2.5106 (1.9); 2.0093 (0.7); 1.2201 (0.4); 1.1950 (0.4); 1.1304 (0.5); 0.0198
(4.9)
1b-15: ¹H-NMR(300.2 MHz, d₆-DMSO):
δ = 7.8087 (0.7); 7.7832 (1.2); 7.7530 (0.7); 7.2568 (1.3); 7.2512 (1.4); 7.2263 (2.4); 7.2008 (1.1); 7.1956 (1.1); 6.6126 (2.3);
5.0271 (0.3); 5.0076 (0.3); 4.9361 (0.4); 4.8905 (0.4); 4.8486 (0.4); 4.8413 (0.4); 4.8152 (0.4); 4.7874 (0.4); 4.7490 (0.4); 4.3329
(2.3); 4.3097 (7.2); 4.2863 (7.3); 4.2631 (2.4); 4.2236 (0.3); 4.2002 (0.4); 4.1768 (0.4); 4.1313 (0.5); 4.1078 (0.6); 4.0846 (0.6);
4.0614 (1.0); 4.0375 (0.9); 4.0139 (0.3); 2.5344 (6.7); 2.5285 (14.5); 2.5225 (20.2); 2.5165 (14.8); 2.5106 (7.1); 2.0095 (3.1);
1.9293 (0.5); 1.4333 (7.6); 1.4101 (16.0); 1.3868 (7.7); 1.3582 (0.6); 1.3426 (0.6); 1.3309 (0.6); 1.3190 (0.8); 1.3087 (0.4);
1.2951 (0.6); 1.2750 (0.4); 1.2443 (0.5); 1.2193 (1.3); 1.1951 (2.0); 1.1713 (1.0); 1.1302 (2.1); 0.0309 (0.9); 0.0200 (30.0);
0.0091 (1.3)
1b-16: ¹H-NMR(300.2 MHz, d₆-DMSO):
δ = 8.2251 (7.4); 8.2121 (7.7); 7.6936 (0.4); 7.4698 (0.5); 7.4514 (0.4); 7.0963 (5.3); 7.0935 (5.4); 7.0834 (5.4); 7.0806 (5.3);
6.9342 (16.0); 6.7283 (0.7); 4.7456 (0.4); 2.7631 (2.4); 2.5869 (36.4); 2.5341 (1.3); 2.5284 (2.7); 2.5224 (3.7); 2.5165 (2.7);
1.5164 (0.3); 1.3733 (0.6); 0.0126 (3.8)
1b-17: ¹H-NMR(300.2 MHz, d₆-DMSO):
δ = 11.4763 (0.5); 11.4290 (0.5); 8.2436 (5.9); 8.2308 (6.2); 7.8687 (0.3); 7.3937 (16.0); 7.3797 (15.4); 7.3221 (0.4); 7.3083 (0.4);
6.9060 (7.4); 3.2608 (0.7); 2.5342 (1.2); 2.5285 (2.6); 2.5225 (3.5); 2.5165 (2.6); 1.5231 (3.0); 1.4073 (0.3); 1.3698 (1.3); 1.2998
(0.4); 0.0064 (3.7)
1b-18: ¹H-NMR(300.2 MHz, d₆-DMSO):
δ = 9.4065 (0.8); 8.7768 (14.4); 8.3892 (0.3); 8.2250 (0.9); 8.1906 (0.5); 8.1467 (8.8); 8.1196 (9.4); 8.1000 (0.6); 8.0660 (7.5);
8.0399 (7.9); 7.6335 (4.5); 7.6291 (5.0); 7.6097 (9.0); 7.6058 (9.4); 7.5832 (8.2); 7.5784 (7.1); 7.5673 (1.3); 7.5583 (8.6); 7.5542
(8.8); 7.5315 (10.1); 7.5284 (8.6); 7.5183 (0.9); 7.5081 (4.4); 7.5044 (4.0); 7.3311 (0.3); 7.2811 (0.4); 7.2626 (0.4); 7.2420 (0.5);
7.1742 (0.6); 7.1121 (16.0); 7.0461 (0.5); 7.0005 (0.4); 6.9450 (0.4); 6.8889 (0.5); 6.8261 (0.4); 4.3871 (0.9); 4.3634 (0.9);
3.4248 (0.3); 2.5345 (5.8); 2.5286 (12.4); 2.5225 (17.0); 2.5165 (12.2); 2.5107 (5.6); 1.5857 (1.1); 1.4097 (0.5); 1.3827 (1.0);
1.3741 (1.1); 1.3590 (2.0); 1.3353 (1.0); 0.0269 (0.6); 0.0161 (19.2); 0.0052 (0.6)
1b-19: ¹H-NMR(300.2 MHz, d₆-DMSO):
δ = 9.4550 (15.0); 9.1920 (0.4); 8.8064 (9.3); 8.8004 (8.7); 8.6898 (0.5); 8.1794 (11.2); 8.1506 (12.5); 8.1135 (0.5); 8.0851 (0.5);
8.0237 (0.3); 7.9956 (0.4); 7.8925 (0.5); 7.8810 (0.4); 7.8629 (0.4); 7.7924 (0.3); 7.7848 (0.4); 7.7733 (0.4); 7.7196 (7.5); 7.7131
(7.3); 7.6908 (6.4); 7.6842 (6.2); 7.6458 (0.4); 7.6397 (0.4); 7.6252 (0.4); 7.6170 (0.4); 7.5967 (0.6); 7.5708 (0.4); 7.5488 (0.6);
7.5278 (0.5); 7.5145 (0.5); 7.5060 (0.5); 7.4990 (0.9); 7.4861 (0.4); 7.4703 (0.5); 7.4546 (0.4); 7.4197 (0.3); 7.3686 (0.4); 7.0956
(16.0); 6.8889 (0.4); 4.9174 (0.7); 4.7294 (0.4); 4.0447 (0.3); 4.0210 (0.4); 4.0173 (0.4); 3.9937 (0.3); 3.4110 (0.4); 3.3876 (0.5);
2.5345 (6.8); 2.5285 (14.3); 2.5225 (19.6); 2.5165 (14.2); 2.5107 (6.5); 2.2018 (0.6); 1.6114 (4.5); 1.5524 (0.5); 1.4095 (0.9);
1.3736 (4.5); 1.2798 (0.6); 1.2587 (1.0); 1.2562 (1.0); 1.2353 (0.7); 1.1292 (1.0); 1.1083 (1.0); 1.0849 (0.5); 0.0268 (0.7); 0.0159
(24.1); 0.0049 (0.9)
1b-20: ¹H-NMR(300.2 MHz, d₆-DMSO):
δ = 8.3141 (7.6); 8.3005 (8.2); 8.2251 (0.5); 8.2111 (0.5); 7.5557 (3.9); 7.5463 (4.8); 7.5418 (3.2); 7.5353 (5.5); 7.5290 (10.8);
7.5230 (15.3); 7.5153 (5.8); 7.5034 (5.6); 7.4893 (12.2); 7.4866 (14.4); 7.4807 (6.4); 7.4738 (8.0); 7.4669 (14.0); 7.4622 (13.3);
7.4496 (4.3); 7.4449 (3.1); 7.4304 (1.1); 7.4241 (1.3); 7.4185 (1.0); 7.4138 (1.1); 7.3978 (16.0); 7.3842 (15.7); 7.3628 (0.8);
7.3566 (0.7); 7.3431 (0.6); 7.3308 (0.4); 6.9667 (0.9); 6.9428 (13.8); 2.5345 (2.9); 2.5286 (6.2); 2.5226 (8.4); 2.5166 (6.0);
2.5108 (2.7); 1.5153 (3.0); 1.3744 (0.5); 1.2850 (0.7); 1.1876 (0.7); 0.0258 (0.4); 0.0150 (10.5)
1b-21: ¹H-NMR(300.2 MHz, d₆-DMSO):
δ = 8.2986 (6.2); 8.2704 (8.6); 8.1872 (0.7); 8.1574 (1.4); 8.1265 (15.3); 8.0968 (12.3); 8.0780 (3.3); 8.0560 (0.4); 8.0477 (0.7);
6.9915 (8.2); 4.4199 (0.5); 4.3962 (1.6); 4.3726 (1.6); 4.3489 (0.5); 2.7087 (0.4); 2.5347 (3.6); 2.5288 (7.8); 2.5228 (10.8);
2.5168 (7.9); 2.5110 (3.7); 1.5817 (16.0); 1.5206 (0.5); 1.3834 (1.7); 1.3739 (0.5); 1.3598 (3.5); 1.3361 (1.6); 0.0264 (0.4);
0.0155 (11.7); 0.0046 (0.4)
1b-25: ¹H-NMR(300.2 MHz, CDCl3):
δ = 8.0247 (0.5); 7.9984 (0.5); 7.8559 (0.9); 7.6648 (0.9); 7.6381 (0.8); 7.2986 (7.2); 6.6113 (2.3); 5.2168 (2.2); 4.4386 (0.4);
4.4255 (1.0); 4.3914 (1.3); 4.2858 (0.4); 4.2460 (0.4); 4.2314 (1.4); 4.2227 (0.6); 4.1979 (1.1); 4.1785 (0.4); 4.1547 (0.4); 2.0931
(1.6); 1.5323 (16.0); 1.5092 (1.0); 1.3591 (0.5); 1.3373 (3.6); 1.3244 (0.6); 1.3115 (0.4); 1.3006 (0.9); 1.2769 (0.4); 0.0473 (0.3);
0.0365 (9.7)
1b-30: ¹H-NMR(300.2 MHz, CDCl3):
δ = 8.1305 (1.0); 8.1035 (1.7); 8.0791 (1.4); 8.0017 (1.6); 7.9969 (1.8); 7.9743 (1.0); 7.9694 (1.2); 7.9013 (1.4); 7.8967 (1.3);
7.8615 (1.4); 7.8569 (1.3); 7.2992 (3.0); 6.7922 (4.7); 4.1804 (0.4); 4.1567 (0.4); 4.0118 (16.0); 3.9996 (1.4); 3.9925 (1.4);
3.9860 (0.5); 2.0950 (1.9); 1.3597 (0.5); 1.3323 (0.4); 1.3252 (0.6); 1.3013 (1.0); 1.2775 (0.5); 0.9837 (0.6); 0.9596 (1.1); 0.9352
(0.5); 0.0364 (4.0)
1b-31: ¹H-NMR(499.9 MHz, d₆-DMSO):
δ = 13.1861 (2.0); 8.5193 (0.5); 8.3712 (6.2); 8.3302 (0.9); 8.2272 (0.4); 8.1306 (0.8); 8.0872 (7.2); 7.8977 (4.6); 7.8951 (4.6);
7.8801 (5.0); 7.8774 (5.1); 7.8527 (0.5); 7.8350 (0.5); 7.8224 (0.5); 7.8043 (0.4); 7.5514 (0.4); 7.5336 (0.4); 7.5013 (0.6); 7.4796
(5.2); 7.4619 (5.0); 7.4429 (0.5); 7.0715 (2.0); 6.3139 (2.3); 6.2992 (2.9); 3.9814 (1.3); 3.9671 (3.7); 3.9529 (3.7); 3.9387 (1.3);
3.9324 (0.4); 3.9191 (0.6); 3.9058 (0.3); 3.4398 (0.7); 3.3583 (1.7); 3.2545 (0.3); 2.4404 (3.9); 2.4369 (7.7); 2.4333 (10.2);
2.4297 (7.3); 2.4263 (3.4); 1.9236 (2.4); 1.9189 (16.0); 1.1192 (4.2); 1.1049 (8.2); 1.0907 (4.0); 0.8291 (0.4); 0.8143 (0.8);
0.7995 (0.4)
1b-33: ¹H-NMR(300.2 MHz, d₆-DMSO):
δ = 10.9075 (2.9); 10.8946 (2.1); 7.7773 (1.3); 7.7704 (1.4); 7.7489 (1.4); 7.7419 (1.5); 7.6322 (2.0); 7.6251 (2.1); 7.6042 (2.1);
7.5972 (2.4); 7.5669 (3.1); 7.5600 (2.9); 7.5162 (0.5); 7.5095 (0.4); 7.4887 (4.0); 7.4818 (3.6); 7.1373 (1.4); 7.0942 (2.7); 7.0768
(3.5); 7.0660 (2.5); 7.0489 (3.0); 6.6751 (3.0); 4.7333 (15.2); 4.7101 (2.1); 4.7010 (16.0); 3.2887 (0.8); 2.5343 (2.7); 2.5283
(6.4); 2.5214 (29.1); 2.0085 (1.1); 1.3746 (1.0); 1.1938 (0.6); 1.1296 (0.4); 0.0185 (9.1); 0.0076 (0.4)
1b-34: ¹H-NMR(300.2 MHz, d₆-DMSO):
δ = 16.4988 (0.7); 16.3172 (0.7); 15.5230 (0.8); 13.2193 (0.9); 10.8929 (0.7); 9.2665 (2.5); 9.0015 (3.6); 8.9753 (3.2); 8.9573
(2.4); 8.8952 (1.0); 8.8139 (3.7); 8.7948 (1.7); 8.7841 (1.5); 8.7587 (1.7); 7.2689 (7.4); 7.2468 (7.3); 7.1721 (11.2); 6.8495 (1.5);
6.2550 (0.8); 6.1595 (1.4); 4.0856 (1.2); 4.0636 (2.3); 4.0379 (2.8); 4.0134 (2.0); 3.9244 (1.0); 3.8543 (1.3); 3.7983 (1.6); 3.7733
(1.6); 3.7535 (1.7); 3.7445 (1.8); 3.4183 (16.0); 3.2526 (4.2); 3.2110 (4.2); 3.1339 (2.1); 3.0950 (1.8); 3.0730 (1.7); 3.0192 (1.3);
2.9821 (1.3); 2.9231 (1.1); 2.8954 (1.0); 2.8551 (1.1); 2.8481 (0.8); 2.7506 (1.4); 2.5227 (142.5); 2.4426 (6.8); 2.4208 (1.1);
2.3817 (1.4); 2.3596 (1.0); 2.3353 (0.7); 2.3098 (0.8); 2.2885 (1.6); 2.2622 (0.8); 2.0959 (1.0); 2.0102 (8.4); 1.9309 (0.8); 1.7946
(0.8); 1.5566 (1.1); 1.5221 (1.1); 1.3733 (1.4); 1.3340 (8.8); 1.3059 (10.1); 1.2783 (7.9); 1.2233 (2.4); 1.1942 (3.9); 1.1716 (2.4);
1.1317 (0.8); 0.9262 (0.9); 0.9040 (1.2); 0.8821 (1.1); 0.0206 (80.7); −2.9513 (0.8)
1b-36: ¹H-NMR(499.9 MHz, CDCl3):
δ = 8.1719 (3.1); 8.1583 (1.3); 8.1549 (4.0); 8.1371 (0.3); 8.1202 (0.4); 8.1027 (0.8); 8.0157 (4.2); 7.9986 (3.3); 7.2615 (3.8);
6.6075 (5.0); 3.9692 (16.0); 3.9588 (0.4); 3.9549 (1.5); 3.9493 (1.3); 2.6467 (1.4); −0.0002 (3.6)
1b-37: ¹H-NMR(300.2 MHz, CDCl3):
δ = 8.0596 (0.4); 7.8157 (0.7); 7.8015 (3.3); 7.7879 (1.0); 7.7733 (3.8); 7.4637 (3.9); 7.4356 (3.2); 7.3961 (0.4); 7.3075 (1.3);
7.2983 (12.6); 7.2848 (3.2); 7.2791 (1.8); 7.2595 (3.2); 7.2496 (0.8); 7.2429 (1.2); 7.2380 (2.0); 7.2334 (1.2); 7.2230 (0.7);
7.2148 (1.7); 7.2035 (0.4); 7.1898 (0.5); 7.0862 (3.1); 7.0625 (2.3); 6.6465 (0.3); 6.5202 (5.0); 6.3090 (1.2); 4.0153 (0.5); 4.0067
(1.5); 3.9052 (0.5); 3.5931 (0.4); 3.5635 (16.0); 3.5345 (5.5); 3.4432 (0.8); 3.4387 (0.8); 3.3695 (1.7); 2.5711 (2.0); 1.5829 (0.6);
1.3211 (0.4); 1.3099 (0.3); 1.2925 (0.8); 0.0475 (0.4); 0.0367 (12.8); 0.0275 (0.4); 0.0259 (0.5)
1b-38: ¹H-NMR(300.2 MHz, CDCl3):
δ = 7.9612 (1.2); 7.9554 (0.4); 7.9374 (0.8); 7.9318 (1.3); 7.5624 (1.1); 7.5328 (0.9); 7.4992 (0.4); 7.4700 (0.3); 7.2985 (1.1);
6.5524 (1.7); 2.6044 (2.2); 1.5751 (16.0); 1.5703 (8.0); 0.0362 (1.0)

### Biological Data

### Example: In Vivo Preventive Test on Puccinia recondita (Brown Rust on Wheat)

Solvent:
   5% by volume of Dimethyl sulfoxide
   10% by volume of Acetone
Emulsifier: 1 μl of Tween© 80 per mg of active ingredient

The active ingredients were made soluble and homogenized in a mixture of Dimethyl sulfoxide/Acetone/Tween®80 and then diluted in water to the desired concentration.

The young plants of wheat were treated by spraying the active ingredient prepared as described above. Control plants were treated only with an aqueous solution of Acetone/Dimethyl sulfoxide/Tween®80.

After 24 hours, the plants were contaminated by spraying the leaves with an aqueous suspension of Puccinia recondita spores. The contaminated wheat plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test, the following compounds according to the invention showed efficacy between 70% and 79% at a concentration of 500 ppm of active ingredient: Ia-013; Ia-040; Ia-047; Ia-059; Ia-080; Ia-122

In this test, the following compounds according to the invention showed efficacy between 80% and 89% at a concentration of 500 ppm of active ingredient: Ia-016; Ia-031; Ia-054; Ia-055; Ia-056; Ia-057; Ia-065; Ia-066; Ia-071; Ia-072; Ia-074; Ia-075; Ia-078; Ia-084; Ia-086; Ia-100; Ia-110; Ia-115

In this test, the following compounds according to the invention showed efficacy between 90% and 100% at a concentration of 500 ppm of active ingredient: Ia-005; Ia-029; Ia-038; Ia-041; Ia-044; Ia-049; Ia-053; Ia-058; Ia-062; Ia-067; Ia-081; Ia-082; Ia-087; Ia-088; Ia-090; Ia-091; Ia-093; Ia-112; Ia-114; Ib-095; Ic-015

### Example: In Vivo Preventive Test on Uromyces appendiculatus (Bean Rust)

Solvent:
   5% by volume of Dimethyl sulfoxide
   10% by volume of Acetone
Emulsifier: 1 μl of Tween® 80 per mg of active ingredient

The active ingredients were made soluble and homogenized in a mixture of Dimethyl sulfoxide/Acetone/Tween®80 and then diluted in water to the desired concentration.

The young plants of bean were treated by spraying the active ingredient prepared as described above. Control plants were treated only with an aqueous solution of Acetone/Dimethyl sulfoxide/Tween®80.

After 24 hours, the plants were contaminated by spraying the leaves with an aqueous suspension of Uromyces appendiculatus spores. The contaminated bean plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test, the following compounds according to the invention showed efficacy between 70% and 79% at a concentration of 500 ppm of active ingredient: Ia-016; Ia-086; Ia-112

In this test, the following compounds according to the invention showed efficacy between 80% and 89% at a concentration of 500 ppm of active ingredient: Ia-015; Ia-054; Ia-055; Ia-074; Ia-088; Ib-095

In this test, the following compounds according to the invention showed efficacy between 90% and 100% at a concentration of 500 ppm of active ingredient: Ia-004; Ia-005; Ia-006; Ia-031; Ia-041; Ia-044; Ia-049; Ia-057; Ia-062; Ia-066; Ia-067; Ia-071; Ia-075; Ia-076; Ia-078; Ia-080; Ia-081; Ia-082; Ia-084; Ia-093; Ia-100; Ia-110

### Example: In Vivo Preventive Test on Phakospora pachyrhizi (Soybean Rust)

Solvent:
   5% by volume of Dimethyl sulfoxide
   10% by volume of Acetone
Emulsifier: 1 μl of Tween® 80 per mg of active ingredient

The active ingredients were made soluble and homogenized in a mixture of Dimethyl sulfoxide/Acetone/Tween®80 and then diluted in water to the desired concentration.

The young plants of soybean were treated by spraying the active ingredient prepared as described above. Control plants were treated only with an aqueous solution of Acetone/Dimethyl sulfoxide/Tween®80.

After 24 hours, the plants were contaminated by spraying the leaves with an aqueous suspension of Phakospora pachyrhizispores. The contaminated soybean plants were incubated for 24 hours at 24° C. and at 100% relative humidity and then for 11 days at 24° C. and at 70-80% relative humidity.

The test was evaluated 12 days after the inoculation. 0% means an efficacy which corresponds to that of the control plants while an efficacy of 100% means that no disease was observed.

In this test, the following compounds according to the invention showed efficacy between 70% and 79% at a concentration of 500 ppm of active ingredient: Ia-130

In this test, the following compounds according to the invention showed efficacy between 80% and 89% at a concentration of 500 ppm of active ingredient: Ia-007; Ia-022; Ia-031; Ia-063; Ia-091; Ia-096; Ia-106; Ia-111

In this test, the following compounds according to the invention showed efficacy between 90% and 100% at a concentration of 500 ppm of active ingredient: Ia-004; Ia-006; Ia-029; Ia-044; Ia-047; Ia-049; Ia-052; Ia-053; Ia-054; Ia-055; Ia-056; Ia-057; Ia-058; Ia-059; Ia-062; Ia-065; Ia-066; Ia-067; Ia-071; Ia-074; Ia-075; Ia-076; Ia-078; Ia-080; Ia-081; Ia-082; Ia-083; Ia-084; Ia-085; Ia-086; Ia-087; Ia-088; Ia-089; Ia-090; Ia-093; Ia-098; Ia-100; Ia-114

In this test, the following compounds according to the invention showed efficacy between 70% and 79% at a concentration of 250 ppm of active ingredient: Ia-089

In this test, the following compounds according to the invention showed efficacy between 80% and 89% at a concentration of 250 ppm of active ingredient: Ib-005

In this test, the following compounds according to the invention showed efficacy between 90% and 100% at a concentration of 250 ppm of active ingredient: Ia-037; Ia-085

### Example: Phytophthora infestans In Vitro Cell Test

Solvent: DMSO
Culture medium: 14.6 g anhydrous D-glucose (VWR), 7.1 g Mycological Peptone (Oxoid), 1.4 g granulated Yeast Extract (Merck), QSP 1 liter
Inoculum: sporocyste suspension

Fungicides were solubilized in DMSO and the solution used to prepare the required range of concentrations. The final concentration of DMSO used in the assay was ≤□1%.

A sporocyste suspension of P. infestans was prepared and diluted to the desired sporocyste density.

Fungicides were evaluated for their ability to inhibit sporocyste germination and mycelium growth in liquid culture assay. The compounds were added in the desired concentration to the culture medium with sporocystes. After 7 days incubation, fungi-toxicity of compounds was determined by spectrometric measurement of mycelium growth. Inhibition of fungal growth was determined by comparing the absorbance values in wells containing the fungicides with the absorbance in control wells without fungicides.

In this test, the following compounds according to the invention showed efficacy between 70% and 79% at a concentration of 20 ppm of active ingredient: Ic-012; Ic-013

In this test, the following compounds according to the invention showed efficacy between 80% and 89% at a concentration of 20 ppm of active ingredient: Ia-058; Ia-071; Ia-078; Ia-099; Ib-007; Ib-031; Ib-060

In this test, the following compounds according to the invention showed efficacy between 90% and 100% at a concentration of 20 ppm of active ingredient: Ia-004; Ia-030; Ia-031; Ia-052; Ia-053; Ia-054; Ia-056; Ia-062; Ia-065; Ia-066; Ia-074; Ia-075; Ia-084; Ia-104; Ia-144; Ia-145; Ib-002; Ib-059; Ib-063; Ib-068; Ib-072; Ib-079; Ib-082; Ib-083; Ib-110; Ic-025

### Example: Pythium ultimum In Vitro Cell Test

Solvent: DMSO
Culture medium: 14.6 g anhydrous D-glucose (VWR), 7.1 g Mycological Peptone (Oxoid), 1.4 g granulated Yeast Extract (Merck), QSP 1 liter
Inoculum: mycelial suspension

Fungicides were solubilized in DMSO and the solution used to prepare the required range of concentrations. The final concentration of DMSO used in the assay was ≤□1%.

Inoculum was prepared from a pre-culture of P. ultimum grown in liquid medium by homogenization using a blender. The concentration of ground mycelium in the inoculum was estimated and adjusted to the desired optical density (OD).

Fungicides were evaluated for their ability to inhibit mycelium growth in liquid culture assay. The compounds were added in the desired concentrations to culture medium containing the mycelial suspension. After 4 days of incubation, the fungicidal efficacy of compounds was determined by spectrometric measurement of mycelium growth. Inhibition of fungal growth was determined by comparing the absorbance values in wells containing the fungicides with the absorbance in control wells without fungicides.

In this test, the following compounds according to the invention showed efficacy between 70% and 79% at a concentration of 20 ppm of active ingredient: Ia-057; Ia-092; Ib-091; Ib-115

In this test, the following compounds according to the invention showed efficacy between 80% and 89% at a concentration of 20 ppm of active ingredient: Ia-037; Ia-056; Ia-058; Ia-063; Ia-071; Ia-082; Ia-085; Ia-086; Ia-089; Ib-015; Ib-112

In this test, the following compounds according to the invention showed efficacy between 90% and 100% at a concentration of 20 ppm of active ingredient: Ia-005; Ia-013; Ia-052; Ia-053; Ia-054; Ia-062; Ia-065; Ia-066; Ia-074; Ia-075; Ia-078; Ia-079; Ia-084; Ia-104; Ia-106; Ia-110; Ib-001; Ib-002; Ib-005; Ib-007; Ib-023; Ib-031; Ib-036; Ib-038; Ib-040; Ib-092; Ib-093; Ib-094; Ib-096; Ib-097; Ib-099; Ib-100; Ib-101; Ib-103; Ib-107; Ib-110; Ic-012; Ic-013; Ic-019; Ic-025

### Example: Pyricularia oryzae In Vitro Cell Test

Solvent: DMSO
Culture medium: 14.6 g anhydrous D-glucose (VWR), 7.1 g Mycological Peptone (Oxoid), 1.4 g granulated Yeast Extract (Merck), QSP 1 liter
Inoculum: spore suspension

Fungicides were solubilized in DMSO and the solution used to prepare the required range of concentrations. The final concentration of DMSO used in the assay was ≤□1%.

A spore suspension of P. oryzae was prepared and diluted to the desired spore density.

Fungicides were evaluated for their ability to inhibit spore germination and mycelium growth in liquid culture assay. The compounds were added in the desired concentration to the culture medium with spores. After 5 days incubation, fungi-toxicity of compounds was determined by spectrometric measurement of mycelium growth. Inhibition of fungal growth was determined by comparing the absorbance values in wells containing the fungicides with the absorbance in control wells without fungicides.

In this test, the following compounds according to the invention showed efficacy between 70% and 79% at a concentration of 20 ppm of active ingredient: Ia-005; Ia-068; Ia-093; Ib-060; Ib-063

In this test, the following compounds according to the invention showed efficacy between 80% and 89% at a concentration of 20 ppm of active ingredient: Ia-066; Ia-076; Ia-079; Ia-085; Ia-094; Ia-102; Ib-001; Ib-010; Ib-058; Ib-083; Ib-092; Ib-096; Ib-100; Ic-014; Ic-025

In this test, the following compounds according to the invention showed efficacy between 90% and 100% at a concentration of 20 ppm of active ingredient: Ia-004; Ia-013; Ia-022; Ia-030; Ia-031; Ia-032; Ia-052; Ia-053; Ia-054; Ia-057; Ia-058; Ia-062; Ia-065; Ia-067; Ia-071; Ia-074; Ia-075; Ia-078; Ia-082; Ia-084; Ia-099; Ia-104; Ia-144; Ia-145; Ib-002; Ib-005; Ib-059; Ib-079; Ib-082; Ib-093; Ib-094; Ib-097; Ib-099; Ib-102; Ib-103; Ib-107; Ib-108; Ib-109; Ib-110; Ic-013; Ic-015; Ic-023; Ic-024

### Example: Colletotrichum lindemuthianum In Vitro Cell Test

Solvent: DMSO
Culture medium: 14.6 g anhydrous D-glucose (VWR), 7.1 g Mycological Peptone (Oxoid), 1.4 g granulated Yeast Extract (Merck), QSP 1 liter
Inoculum: spores suspension

Fungicides were solubilized in DMSO and the solution used to prepare the required range of concentrations. The final concentration of DMSO used in the assay was ≤□1%.

A spore suspension of C. lindemuthianum was prepared and diluted to the desired spore density.

Fungicides were evaluated for their ability to inhibit spores germination and mycelium growth in liquid culture assay. The compounds were added in the desired concentration to the culture medium with spores. After 6 days incubation, fungi-toxicity of compounds was determined by spectrometric measurement of mycelium growth. Inhibition of fungal growth was determined by comparing the absorbance values in wells containing the fungicides with the absorbance in control wells without fungicides.

In this test, the following compounds according to the invention showed efficacy between 70% and 79% at a concentration of 20 ppm of active ingredient: Ia-068; Ia-073; Ia-076; Ia-081; Ia-083; Ia-095; Ia-116; Ia-141; Ib-005; Ib-036; Ib-074; Ib-092; Ib-100; Ib-101; Ib-104; Ib-105; Ic-010; Ic-012

In this test, the following compounds according to the invention showed efficacy between 80% and 89% at a concentration of 20 ppm of active ingredient: Ia-007; Ia-029; Ia-041; Ia-044; Ia-056; Ia-060; Ia-080; Ia-086; Ia-089; Ia-090; Ia-091; Ia-092; Ia-104; Ia-113; Ia-114; Ia-115; Ia-122; Ib-001; Ib-010; Ib-020; Ib-051; Ib-052; Ib-095; Ib-096; Ib-098; Ic-014; Ic-015

In this test, the following compounds according to the invention showed efficacy between 90% and 100% at a concentration of 20 ppm of active ingredient: Ia-004; Ia-005; Ia-006; Ia-013; Ia-016; Ia-022; Ia-030; Ia-031; Ia-032; Ia-034; Ia-037; Ia-038; Ia-052; Ia-053; Ia-054; Ia-057; Ia-058; Ia-062; Ia-065; Ia-066; Ia-067; Ia-071; Ia-072; Ia-074; Ia-075; Ia-078; Ia-079; Ia-082; Ia-084; Ia-085; Ia-087; Ia-088; Ia-093; Ia-099; Ia-100; Ia-110; Ia-111; Ia-128; Ia-144; Ia-145; b-002; b-058; b-079; Ib-082; Ib-083; Ib-093; Ib-099; Ib-103; Ib-110; Ic-013; Ic-019; Ic-025

### Example: Fungicidal Activity Against Phakopsora pachyrhizi

Cryo-conserved wild-type spores of the biotroph Phakosporra pachirrhizi are humidified in dedicated chamber overnight at 18° C. in the dark. The next day, a solution of spores at 7×10³ sp/ml is prepared in a water based growth medium (DH₂O+0.2 mM MOPS at pH 7+0.01% Tween 20) and spores are distributed in a 96-MTPS (final volume of 250 μL per well) thanks to a dispenser robot. Each molecule is tested at 8 doses (from 20 to 0.000256 ppm final concentration) and accordingly 1.5 μL of each dilution is transferred in dedicated well to end-up with a final concentration of 0.6% DMSO. Wild-type spores and molecules are incubated for 4 hours at 21° C., and 6 images par well are then acquired with Transmitted Light images (Image Xpress Micro microscope, Molecular Devices, Objective 10×, 6 images per well). Detection and quantification of the number of germinated spores per image is performed with a dedicated in-house developed algorithm (MetaXpress software, Molecular Devices). Inhibition of the fungal germination was hence determined by comparing the number of germinated spores in wells containing the fungicides to the ones without active ingredient.

In this test, the following compounds according to the invention showed efficacy between 70% and 79% at a concentration of 20 ppm of active ingredient: Ia-039; Ia-052; Ia-139; Ib-012; Ib-033; Ib-036; Ib-038; Ib-040; Ib-092; Ib-100

In this test, the following compounds according to the invention showed efficacy between 80% and 89% at a concentration of 20 ppm of active ingredient: Ia-028; Ia-029; Ia-037; Ia-080; Ia-081; Ia-083; Ia-085; Ia-089; Ia-104; Ia-106; Ia-140; Ib-010; Ib-031; Ib-099; Ib-103; Ib-106; Ib-110; Ib-114; Ic-016

In this test, the following compounds according to the invention showed efficacy between 90% and 100% at a concentration of 20 ppm of active ingredient: Ia-007; Ia-049; Ia-054; Ia-055; Ia-056; Ia-057; Ia-058; Ia-062; Ia-065; Ia-067; Ia-071; Ia-074; Ia-075; Ia-079; Ia-084; Ia-086; Ia-087; Ia-088; Ia-091; Ia-092; Ib-001; Ib-002; Ib-005; Ib-015; Ib-030; Ib-035; Ib-049; Ib-051; Ib-093; Ib-096; Ib-105

### Example: Fungicidal Activity Against Phakosporra pachirrhizi

Cryo-conserved wild-type spores of the biotroph Phakosporra pachirrhizi are humidified in dedicated chamber overnight at 18° C. in the dark. The next day, a solution of spores at 7×10³ sp/ml is prepared in a water based growth medium (DH₂O+0.2 mM MOPS at pH 7+0.01% Tween 20) and spores are distributed in a 96-MTPS (final volume of 200 μL per well) thanks to a dispenser robot. Each molecule is tested at 10 doses (from 30 to 0.002 ppm final concentration) and accordingly 1.2 μL of each dilution is transferred in dedicated well to end-up with a final concentration of 0.6% DMSO. Wild-type spores and molecules are incubated for 4 hours at 21° C., and 6 images par well are then acquired with Transmitted Light images (Image Xpress Micro microscope, Molecular Devices, Objective 10×, 6 images per well). Detection and quantification of the number of germinated spores per image is performed with a dedicated in-house developed algorithm (MetaXpress software, Molecular Devices). Inhibition of the fungal germination was hence determined by comparing the number of germinated spores in wells containing the fungicides to the ones without active ingredient.

In this test, the following compounds according to the invention showed efficacy between 70% and 79% at a concentration of 30 ppm of active ingredient: Ia-017; Ia-040; Ia-141; Ia-145

In this test, the following compounds according to the invention showed efficacy between 80% and 89% at a concentration of 30 ppm of active ingredient: Ia-022; Ia-072; Ia-101; b-050

In this test, the following compounds according to the invention showed efficacy between 90% and 100% at a concentration of 30 ppm of active ingredient: Ia-001; Ia-005; Ia-013; Ia-018; Ia-020; Ia-041; Ia-093; Ia-110; Ia-114; Ia-128; Ic-005

### Example: In Vivo Preventive Test on Phakopsora Test (Soybeans)

Solvent:
   24.5 parts by weight of acetone
   24.5 parts by weight of dimethylacetamide
Emulsifier: 1 part by weight of alkylaryl polyglycol ether

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous spore suspension of the causal agent of soybean rust (Phakopsora pachyrhizi) and stay for 24 h without light in an incubation cabinet at approximately 24° C. and a relative atmospheric humidity of 95%.

The plants remained in the incubation cabinet at approximately 24° C. and a relative atmospheric humidity of approximately 80% and a day/night interval of 12 h.

The test was evaluated 7 days after the inoculation. 0% means an efficacy which corresponds to that of the untreated control, while an efficacy of 100% means that no disease is observed.

In this test, the following compounds according to the invention showed efficacy between 80% and 89% at a concentration of 250 ppm of active ingredient: Ia-095; Ia-100; Ia-141

In this test, the following compounds according to the invention showed efficacy between 90% and 100% at a concentration of 250 ppm of active ingredient: Ia-005; Ia-041; Ia-093; Ia-110; Ia-112; Ia-114; Ia-115; Ib-020; Ib-050; Ib-095

1. A compound of formula (I′): 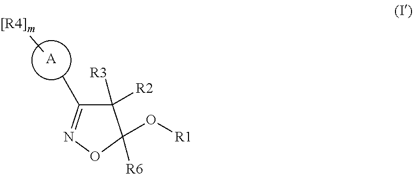
in which
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3 independently of each other, represent a substituent selected from the group consisting of hydrogen, halogen and C₁-C₆-alkyl;
or,
together, form a C₃-C₁₀-carbocyclyl;
A represents aryl or heteroaryl;
R6 represents CX₃ wherein X represents, independently of each other, a substituent selected from the group consisting of hydrogen, fluorine, chlorine, bromine, and iodine atom, wherein at least one X substituent is a fluorine atom;
R4 represents independently a substituent selected from the group consisting of halogen, cyano, hydroxy, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —C(═O)R^{a}, —OC(═O)R^{a}, —N(R^{a})₂, —NR^{a}C(═NR^{a})R^{a}, —N═CR^{a}—N(R^{a})₂, —C(═NR^{a})R^{a}, —C(═NR^{a})N(R^{a})₂, —C(═O)OR^{a}, —C₁-C₆-alkyl-C(═O)OR^{a}, —C₁-C₆-alkyl-O—C(═O)R^{a} and —C₁-C₆-alkyl-N(R^{a})₂,
wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-O—C(═O)R^{a} and —C₁-C₆-alkyl-N(R^{a})₂ substituent is itself optionally substituted, one or more times, in the same way or differently, with R5′,
provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₁-alkyl,
provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₃-C₁₀-carbocyclyl or a 3- to 10-membered-heterocyclyl and R5′ is attached to the carbon atom of R4 that bonds R4 and A;
provided R4 is not attached to A via a nitrogen atom when R4 is a 3- to 10-membered-heterocyclyl,
provided R5′ is not attached to R4 via a nitrogen atom when R5′ is a 3- to 10-membered-heterocyclyl and R4 is C₁-alkyl;
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered heterocyclyl, C₁-C₆-haloalkyl, hydroxy-C₁-C₆-alkyl, aryl, aryloxy, heteroaryl, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R5′ represents a substituent selected from the group consisting of halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃, substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —OC(═O)N(R^{b})₂, and —P(═O)(OR^{b})₂,
wherein said C₁-C₆-alkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b}; or
two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
wherein said C₃-C₁₀-carbocyclyl, 3- to 10-membered-heterocyclyl, aryl, heteroaryl substituent is itself optionally substituted, one or more times, in the same way or differently with CN, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
provided that:
R6 does not represent CF₃ when A represents phenyl, m is 1, R1 is hydrogen and R4 represents hydroxy, C₁-C₆-alkoxy, —N(R^{a})₂ with R^{a} is hydrogen, or —C₁-C₆-alkyl-N(R^{a})₂;
R1 does not represent a hydrogen atom when A represents phenyl, R6 represents CF₃ m is 1 and R4 represents hydroxy, C₁-C₆-alkoxy, —N(R^{a})₂ with R^{a} is hydrogen, or —C₁-C₆-alkyl-N(R^{a})₂;
R5′ does not represent amino or C₁-C₆-alkylamino when A represents phenyl, R6 is CF₃, m is 1 and R4 represents C₁-C₆-alkyl;
m is 1 or 2 when A represents phenyl (as aryl) and R1 represents hydrogen;
R4 does not represent hydroxy, halogen, methyl or methoxy when A represents phenyl and m is 1;
(R4; R4) do not represent (hydroxyl; halogen), (methoxy; methoxy), (methoxy; halogen), (hydroxyl; methyl), (fluor; fluorophenyl) or (methoxy; methoxypropoxy) when A represents phenyl (as aryl) and m is 2;
R2 or R3 does not represent halogen when A represents phenyl (as aryl);
compounds of formula (I′) is not
3-phenyl-5-(trifluoromethyl)-4,5-dihydro-1,2-oxazol-5-yl acetate (873694-74-7);
3-[1-(hydroxymethyl)cyclohexyl]-5-(trifluoromethyl)-4,5-dihydro-1,2-oxazol-5-ol (212615-88-8);
3-[4-(3-tert-butyl-4,4-dimethyl-4,5-dihydrofuran-2-yl)-2-methoxyphenyl]-5-(trifluoromethyl)-4,5-dihydro-1,2-oxazol-5-ol (7898-55-4);
3,3′-(1,4-phenylene)bis[5-(trifluoromethyl)-4,5-dihydro-1,2-oxazol-5-ol] (242461-20-7);
3-(3-thienyl)-5-(trifluoromethyl)-4,5-dihydro-1,2-oxazol-5-ol (1170114-77-8);
ethyl 4-[5-hydroxy-5-(trifluoromethyl)-4,5-dihydro-1,2-oxazol-3-yl]benzoate (1124198-92-0);
4-[5-hydroxy-5-(trifluoromethyl)-4,5-dihydro-1,2-oxazol-3-yl]-2-[(2,2,2-trifluoroethyl)sulfanyl]benzonitrile (1093847-08-5);
5-[bromo(difluoro)methyl]-3-(2-thienyl)-4,5-dihydro-1,2-oxazol-5-ol (1035637-61-6);
3-(5-chloro-3-methyl-1-benzothiophen-2-yl)-5-(trifluoromethyl)-4,5-dihydro-1,2-oxazol-5-ol (883055-08-1);
5-[5-hydroxy-5-(trifluoromethyl)-4,5-dihydro-1,2-oxazol-3-yl]thiophene-2-carbonitrile (656227-15-5);
5-[5-hydroxy-5-(trifluoromethyl)-4,5-dihydro-1,2-oxazol-3-yl]thiophene-2-carboxylic acid (656226-62-9);
3-(2-furyl)-5-(trifluoromethyl)-4,5-dihydro-1,2-oxazol-5-ol (501953-86-2);
3-(2-naphthyl)-5-(trifluoromethyl)-4,5-dihydro-1,2-oxazol-5-ol (328285-44-5);
3-(2-thienyl)-5-(trifluoromethyl)-4,5-dihydro-1,2-oxazol-5-ol (293759-12-3).

2. The compound according to claim 1 wherein A represents phenyl or naphthyl.

3. The compound according to claim 1 wherein A represents a heteroaryl selected from the group consisting of thienyl, thiazolyl, benzofuranyl, indazolyl, benzothiazolyl, benzothiophenyl, benzothiazolyl, pyridyl and pyrimidinyl.

4. The compound according to claim 1 wherein R4 represents a substituent selected from the group consisting of halogen, cyano, hydroxy, C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, —SF₅, —C(═O)R^{a}, —C(═O)OR^{a}, —C(═NR^{a})R^{a}, —N(R^{a})₂, —C(═NR^{a})N(R^{a})₂, —C₁-C₆-alkyl-N(R^{a})₂ and —C₁-C₆-alkyl-C(═O)OR^{a} with R^{a}, wherein said C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl substituent is itself optionally substituted, one or more times, in the same way or differently, with R5′.

5. The compound according to claim 1 wherein R1 is a substituent selected from the group consisting of hydrogen, C₁-C₆-alkyl and —C(═O)R^{a}, wherein R^{a} represent a substituent selected from the group consisting of C₁-C₆-alkyl, C₁-C₆-haloalkyl, C₃-C₁₀-carbocyclyl, C₁-C₆-alkyl substituted by C₁-C₆-alkoxy and aryl, said C₁-C₆-alkyl substituent is itself optionally substituted, one or more times, in the same way or differently, with R5.

6. The compound according to claim 1 wherein R2 and R3 are independently selected from the group consisting of hydrogen, halogen and methyl.

7. The compound according to claim 1 wherein R6 represents CF₃, CF₂Cl, CF₂Br or CHF₂.

8. The compound according to claim 1 wherein m is 1 or 2.

9. A composition comprising at least one compound of formula (I) according to claim 1 and at least one agriculturally acceptable auxiliary.

10. A product comprising a compound of formula (I) for controlling phytopathogenic fungi: 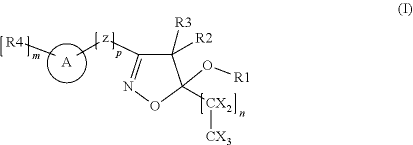
in which:
R1 represents a substituent selected from the group consisting of hydrogen, sulfinyl, sulfonyl, C₁-C₆-alkyl, C₁-C₆-haloalkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3 to 10-membered-heterocyclyl, hydroxy-C₁-C₆-alkyl, aryl, heteroaryl, nitro, —C(═O)R^{a}, —C(═O)OR^{a}, —C(═O)N(R^{a})₂, —C(═S)N(R^{a})₂, —S(═O)₂R^{a}, —S(═O)₂N(R^{a})₂ and —P(═O)(OR^{a})₂, wherein said C₁-C₆-alkyl, C₁-C₆-haloalkyl, C₁-C₆-alkylthio, C₁-C₆-alkylsulfinyl, C₁-C₆-alkylsulfonyl, C₂-C₆-alkenyl, C₂-C₆-alkynyl, C₃-C₁₀-carbocyclyl, 3 to 10-membered-heterocyclyl, hydroxy-C₁-C₆-alkyl, aryl, heteroaryl substituent is itself optionally substituted, one or more times, in the same way or differently, with R5;
R2 and R3 independently of each other, represent a substituent selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{a}, —C(═O)OR^{a}, —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —OC(═O)N(R^{a})₂, —NR^{a}S(═O)₂R^{a}, —S(═O)₂R^{a}, —S(═O)₂N(R^{a})₂, —P(═O)(OR^{a})₂, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂ and —C₁-C₆-alkyl-P(═O)(OR^{a})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —C₁-C₆-alkyl-N(R^{a})₂, —C₁-C₆-alkyl-C(═O)R^{a}, —C₁-C₆-alkyl-C(═O)OR^{a}, —C₁-C₆-alkyl-C(═O)N(R^{a})₂, —C₁-C₆-alkyl-C(═S)N(R^{a})₂, —C₁-C₆-alkyl-NR^{a}C(═O)OR^{a}, —C₁-C₆-alkyl-NR^{a}C(═O)N(R^{a})₂, —C₁-C₆-alkyl-NR^{a}C(═S)N(R^{a})₂, —C₁-C₆-alkyl-NR^{a}C(═O)R^{a}, —C₁-C₆-alkyl-NR^{a}C(═S)R^{a}, —C₁-C₆-alkyl-OC(═O)N(R^{a})₂, —C₁-C₆-alkyl-NR^{a}S(═O)₂R^{a}, —C₁-C₆-alkyl-S(═O)₂R^{a}, —C₁-C₆-alkyl-S(═O)₂N(R^{a})₂, —C₁-C₆-alkyl-P(═O)(OR^{a})₂ substituent is itself optionally substituted, one or more times, in the same way or differently, with R5;
or,
together, form a C₃₋₁₀-carbocyclyl or 3- to 10-membered-heterocyclyl;
A represents aryl or heteroaryl;
X represents, independently of each other, a substituent selected from the group consisting of hydrogen, fluorine, chlorine, bromine, and iodine atom, wherein at least one X substituent is a fluorine atom;
Z represents a C₂-C₆-alkenyl group, itself being optionally substituted, one or more times, in the same way or differently, with R5;
R4 represents independently a substituent selected from the group consisting of halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —SF₅, —C(═O)R^{a}, —C(═O)OR^{a}, —C(═NR^{a})R^{a}, —N(R^{a})₂, —NR^{a}C(═NR^{a})R^{a}, —N═CR^{a}—N(R^{a})₂, —OC(═O)R^{a}, —S(═O)₂R^{a}, —C(═NR^{a})N(R^{a})₂, —P(═O)(OR^{a})₂, —C₁-C₆-alkyl-N(R^{a})₂, —C₁-C₆-alkyl-C(═O)R^{a}, —C₁-C₆-alkyl-O—C(═O)R^{a}, —C₁-C₆-alkyl-C(═O)OR^{a}, —C₁-C₆-alkyl-S(═O)₂R^{a} and —C₁-C₆-alkyl-P(═O)(OR^{a})₂,
wherein said C₁-C₆-alkyl, C₁-C₆-haloalkyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, C₁-C₆-alkyl-N(R^{a})₂, —C₁-C₆-alkyl-C(═O)R^{a}, —C₁-C₆-alkyl-O—C(═O)R^{a}, —C₁-C₆-alkyl-C(═O)OR^{a}, —C₁-C₆-alkyl-S(═O)₂R^{a} and —C₁-C₆-alkyl-P(═O)(OR^{a})₂ substituent is itself optionally substituted, one or more times, in the same way or differently, with R5′,
provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₁-alkyl (i.e. methyl),
provided R5′ is not —C(═O)N(R^{a})₂, —C(═S)N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —OC(═O)N(R^{a})₂, —NR^{a}S(═O)₂R^{a}, or —S(═O)₂N(R^{a})₂ when R4 is C₃-C₁₀-carbocyclyl or a 3- to 10-membered-heterocyclyl and R5′ is attached to the carbon atom of R4 that bonds R4 and A;
provided R4 is not attached to A via a nitrogen atom when R4 is a 3- to 10-membered-heterocyclyl,
provided R5′ is not attached to R4 via a nitrogen atom when R5′ is a 3- to 10-membered-heterocyclyl and R4 is C₁-alkyl (i.e. methyl);
R5 represents a substituent selected from the group consisting of halogen, cyano, azide, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —C(═NR^{a})N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —Si(C₁-C₆-alkyl)₃, substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R5′ represents a substituent selected from the group consisting of halogen, cyano, azide, hydroxy, mercapto, sulfinyl, sulfonyl, amino, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
wherein said C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, —Si(C₁-C₆-alkyl)₃, substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-haloalkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, OC(═O)N(R^{b})₂, and —P(═O)(OR^{b})₂;
wherein said C₁-C₆-alkyl, C₁-C₆-haloalkyl, C₁-C₆-alkoxy, C₁-C₆-alkylthio, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy substituent is itself optionally substituted, one or more times, in the same way or differently with R^{b}; or
two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂; wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
n represents an integer of 0, 1, 2, 3, 4, 5, or 6;
m represents an integer of 0, 1, 2, 3, 4, or 5; and
p represents an integer of 0, 1, 2, 3, 4, or 5.

11. A product comprising a compound of the formula (I) according to claim 10 wherein R4 is a substituent selected from the group consisting of halogen, cyano, hydroxy, amino, C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, nitro, —SF₅, —C(═O)R^{a}, —C(═O)OR^{a}, —C(═NR^{a})R^{a}, —N(R^{a})₂, —C(═NR^{a})N(R^{a})₂, —C₁-C₆-alkyl-N(R^{a})₂ and —C₁-C₆-alkyl-C(═O)OR^{a} with R^{a} as recited in claim 10, wherein said C₁-C₆-alkyl, C₁-C₆-alkoxy, C₁-C₆-alkylthio, arylsulfenyl, C₁-C₆-alkylsulfonyl, arylsulfonyl, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, substituent is itself optionally substituted, one or more times, in the same way or differently, with R5′.

12. A method for controlling phytopathogenic fungi which comprises applying at least one compound of formula (I′) according to claim 1 or one compound of formula (I) to plants, plant parts, seeds, fruits and/or to soil in which plants grow.

13. A compound of formula (I′): 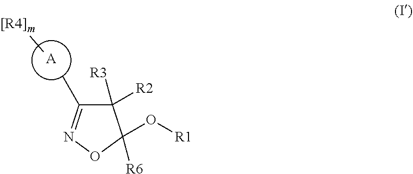
in which:
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl;
or,
together, form a C₃-C₁₀-carbocyclyl;
A represents aryl, heteroaryl, C₃-C₁₀-carbocyclyl or 3- to 15-membered-heterocyclyl group;
R6 represents CX₃ wherein X represents, independently of each other, a substituent selected from the group consisting of hydrogen, fluorine, chlorine, bromine, and iodine atom, wherein at least one X substituent is a fluorine atom and provided R6 is not CF₃ or CHF₂;
R4 represents independently a substituent selected from the group consisting of halogen, hydroxy, cyano, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —SO₂N(R^{a})₂, —C(═O)R^{a}, —C(═NR^{a})N(R^{a})₂, —OC(═O)R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —NR^{a}═C—N(R^{a})₂, —NR^{a}S(═O)₂R^{a}, —OC(═O)N(R^{a})₂, —C(═NR^{a})R^{a}, —C(═O)—O—R^{a}, —C(═O)N(R^{a})₂, —C(═O)NR^{a}N(R^{a})₂, —C(═O)N(OR^{a})R^{a}, —C(═S)N(R^{a})₂, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁—C-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a},
wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5;
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
provided that compounds of formula (I′) is not:
5-[bromo(difluoro)methyl]-3-(2-thienyl)-4,5-dihydro-1,2-oxazol-5-ol (1035637-61-6);
5-[bromo(difluoro)methyl]-3-(4-methylphenyl)-4,5-dihydro-1,2-oxazol-5-ol (1224442-78-7);
5-[bromo(difluoro)methyl]-3-(4-methoxyphenyl)-4,5-dihydro-1,2-oxazol-5-ol (1035637-62-7);
5-[bromo(difluoro)methyl]-3-phenyl-4,5-dihydro-1,2-oxazol-5-ol (1035637-60-5).

14. A compound of formula (I′) 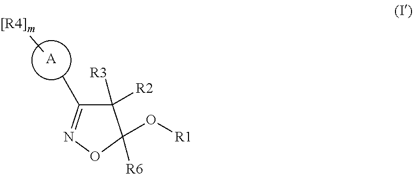
in which:
R1 represents a substituent selected from the group consisting of hydrogen, C₁-C₆-alkyl and —C(═O)R^{a}, wherein said C₁-C₆-alkyl substituent is itself optionally substituted, one or more times, in the same way or differently, with R5;
R2 and R3, independently of each other, represent a substituent selected from the group consisting of hydrogen, halogen and C₁-C₆-alkyl,
or,
together, form a C₃-C₁₀-carbocyclyl;
A represents aryl, heteroaryl, C₃-C₁₀-carbocyclyl or 3- to 15-membered-heterocyclyl group, provided A is not phenyl or not a 5- or 6-membered aromatic heteroaryl;
R6 represents CX₃ wherein X represents, independently of each other, a substituent selected from the group consisting of hydrogen, fluorine, chlorine, bromine, and iodine atom, wherein at least one X substituent is a fluorine atom;
R4 represents independently a substituent selected from the group consisting of halogen, hydroxy, cyano, C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —SF₅, —SO₂N(R^{a})₂, —C(═O)R^{a}, —C(═NR^{a})N(R^{a})₂, —OC(═O)R^{a}, —N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —N═CR^{a}—N(R^{a})₂, —NR^{a}S(═O)₂R^{a}, —OC(═O)N(R^{a})₂, —C(═NR^{a})R^{a}, —C(═O)—O—R^{a}, —C(═O)N(R^{a})₂, —C(═O)NR^{a}N(R^{a})₂, —C(═O)N(OR^{a})R^{a}, —C(═S)N(R^{a})₂, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁—C-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a},
wherein said C₁-C₆-alkyl, C₁-C₆-haloalkyl, C₂-C₆-alkenyl, C₂-C₆-alkynyl, C₁-C₆-alkoxy, C₁-C₆-alkylthio, arylsulfenyl, C₁-C₆-alkylsulfinyl, arylsulfinyl, C₁-C₆-alkylsulfonyl, arylsulfonyl, C₃-C₁₀-carbocyclyl, 3- to 10-membered-heterocyclyl, aryl, aryloxy, heteroaryl, heteroaryloxy, —C₁-C₆-alkyl-C(═O)OR^{a}, —C₁-C₆-alkyl-N(R^{a})₂, —C₁-C₆-alkyl-3- to 10-membered-heterocyclyl, —C₁-C₆-alkyl-C(═O)N(R^{a})₂, —C₁-C₆-alkyl-NR^{a}C(═O)OR^{a}, —C₁-C₆-alkyl-O—C(═O)R^{a}, —C₁-C₆-alkyl-NR^{a}C(═O)N(R^{a})₂, —C₁-C₆-alkyl-N(OR^{a})C(═O)R^{a}, —C₁-C₆-alkyl-C(═O)N(OR^{a})R^{a}, —C₁-C₆-alkyl-NR^{a}C(═S)N(R^{a})₂, —C₁-C₆-alkyl-NR^{a}C(═O)R^{a} and —C₁-C₆-alkyl-NR^{a}S(═O)₂R^{a} substituent is itself optionally substituted, one or more times, in the same way or differently, with R5;
R5 represents a substituent selected from the group consisting of azide, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —Si(C₁-C₆-alkyl)₃, —C(═O)R^{a}, —C(═O)OR^{a}, —C(═O)N(R^{a})₂, —C(═S)N(R^{a})₂, —C(═NR^{a})R^{a}, —C(═NR^{a})N(R^{a})₂, —NR^{a}C(═O)OR^{a}, —NR^{a}C(═O)N(R^{a})₂, —NR^{a}C(═O)R^{a}, —NR^{a}C(═S)R^{a}, —NR^{a}C(═S)N(R^{a})₂, —NR^{a}C(═NR^{a})R^{a}, —OC(═O)R^{a}, —OC(═O)N(R^{a})₂, —NR^{a}S(═O)₂R^{a}, —S(═O)₂R^{a}, —S(═O)₂N(R^{a})₂ and —P(═O)(OR^{a})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, —Si(C₁-C₆-alkyl)₃ substituent is itself optionally substituted, one or more times, in the same way or differently with R^{b}; when two R5 substituents are bound to a common carbon, they may form together C═O, C₃-C₁₀-carbocyclyl or 3- to 10-membered-heterocyclyl;
R^{a} represents, independently from each other, a substituent, which is identical or different, selected from the group consisting of hydrogen, halogen, cyano, hydroxy, mercapto, sulfinyl, sulfonyl, amino, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{b}, —C(═O)OR^{b}, —C(═O)N(R^{b})₂, —C(═S)N(R^{b})₂, —NR^{b}C(═O)OR^{b}, —NR^{b}C(═O)N(R^{b})₂, —NR^{b}C(═O)R^{b}, —NR^{b}C(═S)R^{b}, —OC(═O)N(R^{b})₂, —NR^{b}S(═O)₂R^{b}, —S(═O)₂R^{b}, —S(═O)₂N(R^{b})₂ and —P(═O)(OR^{b})₂,
wherein said C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy substituent is itself optionally substituted, one or more times, in the same way or differently with R^{b},
two R^{a} when attached to a nitrogen atom may form together with the nitrogen atom to which they are attached a 3- to 15-membered heterocyclyl;
R^{b} represents, independently from each other, a substituent, which is identical or different, selected from the group consisting of hydrogen, halogen, cyano, —OR^{c}, —N(R^{c})₂, —SR^{c}, —S(═O)R^{c}, —S(═O)OR^{c}, —S(═O)₂R^{c}, —S(═O)₂OR^{c}, C₁-C₆-alkyl, C₁-C₆-alkoxy, C₁-C₆-alkylthio, C₁-C₆-alkylsulfinyl, C₁-C₆-alkylsulfonyl, C₁-C₆-alkylamino, C₂-C₆-alkenyl, C₂-C₆-alkynyl, C₃-C₁₀-carbocyclyl, 3- to 10-membered-heterocyclyl, C₁-C₆-haloalkyl, hydroxy-C₁-C₆-alkyl, aryl, aryloxy, heteroaryl, heteroaryloxy, nitro, —C(═O)R^{c}, —C(═O)OR^{c}, —C(═O)N(R^{c})₂, —C(═S)N(R^{c})₂, —NR^{c}C(═O)OR^{c}, —NR^{c}C(═O)N(R^{c})₂, —NR^{c}C(═O)R^{c}, —NR^{c}C(═S)R^{c}, —OC(═O)N(R^{c})₂, —NR^{c}S(═O)₂R^{c}, —S(═O)₂N(R^{c})₂ and —P(═O)(OR^{c})₂,
wherein said C₃-C₁₀-carbocyclyl, 3- to 10-membered-heterocyclyl, aryl, heteroaryl substituent is itself optionally substituted, one or more times, in the same way or differently with cyano, halogen, C₁-C₆-alkyl, C₁-C₆-haloalkyl or C₁-C₆-alkoxy;
R^{c} represents, independently from each other, a substituent, which is identical or different, selected from the group consisting of hydrogen, aryl and C₁-C₆-alkyl;
m represents 0, 1 or 2;
provided that compounds of formula (I′) is not
3-[1-(hydroxymethyl)cyclohexyl]-5-(trifluoromethyl)-4,5-dihydro-1,2-oxazol-5-ol (212615-88-8);
3-(5-chloro-3-methyl-1-benzothiophen-2-yl)-5-(trifluoromethyl)-4,5-dihydro-1,2-oxazol-5-ol (883055-08-1);
3-(2-naphthyl)-5-(trifluoromethyl)-4,5-dihydro-1,2-oxazol-5-ol (328285-44-5);
3-[1-(hydroxymethyl)cyclohexyl]-5-(trifluoromethyl)-4,5-dihydro-1,2-oxazol-5-ol (212615-88-8).

15. A product comprising a compound of formula (I′) as recited in claim 13 for controlling phytopathogenic fungi.
